SEQUENCE LISTING (9 sequences)

SEQ ID NO: 1 - DNA, 138127 bp, Pseudorabies virus
ggcccagctctcccccgagcgcggatctgtgaaaaaaaattttccctcccccgcgttttccattggggtgaatggggagggggtgggggatgggcactcactgagaattggcaaaatgccaatttatattctggcactttgccaaaccacatacttggcaccctgccaaccccaatccccctcctaccttggcaccctgccaaccccaatccccctcctaccttggcaccctgccaaccccaatccccctcctaccttggcaccctgccaaccccaatccccctcctaccttggcaccctgccaaccccaatccccctcctaccttggcaccctgccaaccccaatccccctcctaccttggcaccctgccaaccccaatccccgccccggtcctcccatcgcttttggcacggcgccaaccccgatcccccgccatcgccttttcccagtccttggctcgctgccaaccctagagggcgagacgcggtgctgcggtcgggcccacgggcggcgggtggttggagagggcgatgggcagggacgggcgcgcttgtggtggggacggtgtcggtgcccgcgacggtggcgcccgagagggttcgccttggatgcgatgggggccctccccattcaccccaatggaaaacgcgggggagggaaaattttttttcacagatccgcgctcgggggagagctgggcccagtttttttggggggagggggtgggggaaaagagctgggcccggctctgggtgggggacaacccccgagggagagtgtggccggtggccagactagaggagagccccgccgggaccggtgaggagagagggcctttccccccagccccctccgctcccccctttccccccagccccctccgctcccccctttccccccagccccctccgctcccccctttccccccagccccctccgctcccccctttccccccagccccctccgctcccccctttccccccagccccctccgctcccccctttcccccctttctaagtggacgccgcgggcgcgcgggcccccacacggcgttcgctctcgtcccggccggtcgggccgccgggacggggcgctcggcgccgcgatgaccgacgcctcgtcgcggcccggcggggaagagccgatggaggtggacgagcacgtcgggccggagcccgagcccatggaggtggacgagccccccgcaccgccgtcggcaccggcaccgacggcggcgggcaccatcctcgcggtgggcttcacccagcccgcgcaggtcctgcgggtgtacccgatcggttgaggtgcgaacgatgggcgggggatgcccggggtcggacgcgctcacgtggtgccccacccaccccacgccgcgctctttttcccgggccgcccgaccggatcctcccgcagaggcggaggtgaggccggagagagacagggagagatgatggacgcggagaggggagcggccgccgcggggcgcgtggaggtggagctcgatgcccggccctccggcgccggagggcgcggcgcgagcccctcgcgccgggcgcgggacccccgaaccagcccgtgctcccggggccgagccgaccggcgttcccgggacagaaccggcgacgtcaccgggccgcgggcagcgcggcccgccaccctgggtattaacgtcccgggcaccgccgtccgccgccagccgccgtgggaggcagacatgcctccacaacgagcccgcggggctccgccgcgccgtcgcggcagcgacccgcccgatccaggcagcctcgccgggcggccctcgcccggggggagaggaggcggagggcagcgccgccccctctcgcgcagcagctcgctcacgtccgtcgcctcggcgcccgtggagacgcccgtcgtcgcggaggctccggggctcggcgcccccggctccaggccgccctcctacggggacgtcgtccgcgtcgggccgcgcccgcaccgatcgccggacacgccgctgtttgcccggggcccgcccccgtcctactcggagacgctcctgttcgacccgcccgcgtacgcggtgaccatcccggacccgccggcgtacgagcccaccgtcatcgggccgcacccgccgcgcccccgcgactggatctcctcgccctcggtggtgcagccgtcgctgctgggccccttcagccagtgcctcccgcgggtgacctgccccgactgccgctaccccgaagaccgcccgatggtgctcgtgggcttcctctgggggggactgctcctgctggtgggcctcgtgtttctgatcctgctcccggtgctccgggagtccgtcgtgtttccctgatcaataaactcggctcgcccgccccatcgatattcgcgtctcgtgtggtgctgcgacgaggggggaagaggtgggacgcgaagagatgaagggatcggggggaaagacggcgagaggaggagcgggagtgtgcggaagggacggtgaagcggcggtggtggagggagatggggagatggagggagatggggagatggagggagatggggagatggagggagatggggagatggagggagatggagggagatggagggagatggagggagatggagggagatggagggagatggagggagatggggagatggagggagatggggagatggagggagatgggggagatggagggagatggagggagatggagggagatggagggagatggagggagatggggagatggagggagatggggagatggagggagatggagggagatggagggagatggagggagatggagggagatggggagcgacgacgacagactcgcgcacgccagagaggtacggttcaacggttttattcaaaacaggtggttgcagtaaaagtacttcccgtgcatgtacacggggacgagggtgtagctgcacgtcaggcggcaggtgggggcgtcgcagccggtcttgtgggcgtccagcgcgtccatgatcccgccgaggcaggcgccgggcacgtactcctccagcggggcgggcgagtcccccaggtcctcgggcagcacgcagctcgagccgcgctcgacgacgcgggcgatgcgggccagcatcaccagggtgtaggtgacgatgcagcgcacgtcccgcaggcggcgccggcgcacgagctcgtcgacgggcgtgtcgcggaggtagcagcgcaggaacgggccgagcttcaggcgcaggttctggagcaccgcccccgcggtggccacgatggggtccagcgtgcgcagcgtgagccccttggtgaggatgagcttgacccacgtgagcgtctcgtccgccgaggcgagggactccgtcaggccctcgttcaggagcaccatctcgcggagcacgcgcgccgcctcggtggcgtgcggccgcacctcaaacaggcggtgcaggtcggccccgtggaacatgagcgtctcccacgtgacgcgccgcccgtccggggtgaactgctcggcgccgaactcgaggacggcggcccacggcgacgagcccgcggcgcggaagccgtcgttgaacacggggcgggagaggtcgcgccgcgccgaggcgaagaggtcgttgacgcgcgtcgcgccgacccgcgcgtgcgtcgcggagacggtggcgcgcgcgctctccgacaggcggtcggggggcgcgtcgggcccgctgtcgggacgatccgcctccggccgccgccgctggtgctgctgctggcgctgctgctgctgttgctgtcgctgccgctgtcggagggcctgtcgggcggcgcgcccgccgcgacccccgcgccggcgccggacggccccgagccgcgcccggacgggcggccgctcggaggggcgcgcgcccaggcgctcgcggaccgggcgccgctcctcgctccccgaccgggacgcctcgctgccgctgctgttgccgctgtcctccatgaccgtggtgcgagcggggccgcggtgtgccgccggcgggcggcggggcgcctttaaccccgcgcccccgcgcgaccttcatccaaatatggtaatgtggcgctcgtagtgcagaaacagcaacacgccccctaccagcaggaggaggtacagcgccttggcaaagatcccggcgagcacggtcgagcagcaggcgccgcagacgccgagggcggcgcgcccgccgtcggcgcgcgcgggcggggccgtcgcgccggagacgcgcgcgatgtgcggcagcgtcgccgccaggagctccagcgccccgaccgccagcacaaagagccacacgagggcgcgggcgtacagcgggaagacgagggcgcacggggcgcgcgtgacgccgagggtcgtgaacagcgccatgcgcgcccccaggcggagcccgatctcgaggaggatgagcgcgatcagcgtggggtggcggtgcgccagcgtgatggggtcctcgcggaggtcgcggctgagcgcggcgcggcgcgtcgcgagctcggccatgtaccaggcgaacttggtgtagctgcagccgatgaccgtggcggcgagcacgctcgccgcgtagttgagcgtgtacttctccggggtcaggaactcggcggggtcgcggaaggggccgaacatgcgccgctcctggcgcgcgtacacgaaggccgcgtagaggagccacgcgaggaccgccaggcgaaagtgcacgtcccacaggtaggcgcggcagtcgcggcgcgcgtacacgacccgcgcgcggtcgcgcagctccgggctcagcgcgcccgtctcgttggggcccacgtagatggccgtggcgttgaaggcctcccagtccggcgcgccgccgccggcgtgctcgccgagcggcagcgccgcgtacacgacggggtggggcaggcgggcgacggtcgcgtagtacgcgcccaggccggcgtacgcgcccatcaccccgaggacgaggaggtgcagcgggcgtccgccgaggagcatgattgccccacctccgcggtgaaaatggtgcgcgtcgcgttgctgccgcactttgtagcgaagcactgctgactcagcgagatgcacagccggtcgtgggcgtcgacgctcagggccacgaaggtgcgggccggcgggctgcgcgcgtgccgggcccgcaggcagctgaagcgcgcgggcccgcagagctggaagagcacccagtcgggcttggccaccacccgctgcgcggtcgcgcccgcgtactcgtgcgcgcgcccgttgaagtgcgcgtcgaggtgccgcagcacgggcgccatggccacgtccgccacgaaggcctcgagcgcgcgccggtcctccgcgtccacggccaggccgcgcagcatgtccgccagcggcgtgcgccgggcgaagaaggcctcgcggttccgcgccgccttgcgctcgaagaagctctcgtactccccgccgaggctgtgcagcacgcgcgtgacggcgtgcgccgggcgggcgtggaagtgaaagttgcgcgggtcgtcgaagcccgggggcgcgtccccgaaggggagcaggcgcccgtggcagccgccgccgtcgaccttggcgaagaagggcagccgcaggctgcgcccgtgcgcgtacacgcccgtgtccacgaaggtgaagtcctggaggtacgggcccatgggctcgataaagtcccgctccagcagcacggcctgctggagcacacgcgccaggccgcggaccgtgtcggcgccggccaccgcgtacggcgacggcacgggcgtgcacacgcggaagcccatcttggcgcggcagccgcacgccgggcgctcgtccgccgcgggggcgcgcgcggggccgggttcggggtcgggggcggcggcggaggaggagccggggatcccggccccgacctcgtcgtcgtcgcacgtgtacacctcggcgggggcgcccgcgtcccagtccgcccagtcctcgccgtccggcgcggcgccaaagtcggcgggggcggcgacgtcgtcgtcccagggcgccccctcgaggccgtcgtcggaggaggccgcggcggcggaccccgcggcgccggcgcccccgggcggacaggcgctcttgtacacgtagcacgggtgcgcggcccagtcgccgtcggcctcggggaagagcagcgccagcgcgtcgagcaccgcgcggcggaacccgcgcatggccgcctcgagcgtgcccgcgggcaccgggcgcgcgaggcgaaagtcgacgtcgaggacgatgttggtcacggccagccgggcgttgaacacctcgtggcggctcacgtacatctgcgccgccggctcgtgcgccgccatctccggcgccgcgtcgcgccgcgccgtcgcgcgcccccaggcgtccccggccagcaccgcgaaggcctggcgccggtgcgccagctccacgcggtacaccggcgccggggggcgcgcgtccggcaggccgagcagggcgcccagcgccgtgcgcgcgcgcccgccgcccggcgcggcggccagctccagcaggcggcgcaggacggtgccgtcggcgcccggctgctgctgctgcgagtgcgcctgctcctcgtcgggggcgcgcacccagccgtggggcgcgaggcgctcctcgacggcggtcacgtggcgcgcgaggccggcggccgcgtcgcagagcccgcgcggcgcgtcggccgcgagcgcggcgtacgtgcggtcctccacgtagccggcgcccacggccggggcgaggcgcgcgacgctcggcgtcacgttctgggcgacgtagtcgcggatgttgagctgcgcccgcacgtggcggaagaaggtggcggccgcgcgctccccgaggggcgagcggcccacgggcgccgcggggtccgtgacgttgacggcgtccaggtgctcgcggagccgcgcgcggttgaagctctcaaagtgggccaggtagacgtacgtcacgaactcgcggtcggggagcttgaggctgccgcggtcgtgggcgatgtagtcccggaggacgcccatctcggcgcggtcggcgcgcacgcgctcgtcgacgtagcgcgcggcgcgcgccgccgcgggcccccgcgcgtagccgctcaggcagcagaagcgcgagagcgccgcgaacgagagcagccccgcggggtccaggccgctggggttgggccccacgcgcgggttgtacgtgagcagcaggtccttgagggcccgcaggtcgtaggccggccgcgcgtggaagaggtggaggcgcgtggccagcaccagcgtcttctcctcgggcccgaagcgcgccacgtaccagaagggcgtgttgtggttcccgtagaggcgccggaaggcggccagcgtctgggtctcgtggtgcacaaagagcgaggtcagcccgcggcgcccgaccgcgatggagcgcagcgggcgctccggcgcgtactcggcgttgccggtgcgcgggcgcatctgctcgaggaccatggtcagggccgcgatgaggccgtcgtggagcgcgaaggtggccgcctcgtccatggcctcgaggacgtcccgcgccgcgaggggctccccgcggcagagcgcgcggacgaccgccggggtcgcggcggcgacgaacacgggccgcacgcggacgctggccacgtgcccggtgaggcagaacgtgacgccggggcggcgcgctaaaagctcggcctcggcgcgctcgcccggcagtgagaaggcctcgtccagcccggcgccgtcccacgcgtacgagacgacgtagaccgcccccgagggctcctggcccgtgagcagcatcagcgagtacgtgatcgcgcagccgtccgtggcgtacaggacgcggatcatgctgggcggcatcttctccgggtggtgcgggcgccgcggccgtgggagggcttatgagccgccggacgaactccgcctccgggagtcgctggccgtgctcaacgtgatcctccccgccccgctcgccgccgaggacgtgatggcctcggccgaggccgcgcgccgcctggcccgcgcggacacgctggcgcgcacctaccaggcctgccagcgcaacctcgagtgcctggcgcgccacgaggcctccggcgacgacggcctggacgccgtggtcgcggcgcacgcggccaacgcgcggcggctcgcggacacgtgcctggcggcgctcatgcacctgtacctctcggtcggcgccgtcgacgcggacacggacgacctcgtcgagcaggccctgcgcatgaccgccgagagcaacgtggtgatgtccgacgtggccgtcctggagcggacgctgggactggcccggcggcggcagccggcgcccgtccgggccgcgcccgccggggtgggcctgcccgcccccgtcccgcccgccccgcgcgtcgccgccccgccggccccgcagaccccgcccgaggaggaggcggatgcggaggcggaggaagacgaggctggcggggaccgccccgaggaggacgcggtcccgctgctgcccccgcgcgcccccgtcaagacgcgcccgcccgagaagaagaaggtccggaggctggccgaggcctgcaagatgtagacggagagcggggggagagagagggcacgcgcgaagagaagagacgaggggcgaccgagaagaaggggacggagaccaccgggtgtttttttttcttttcaataaacatctttattgaaaaaaagagaagcgtcctgcgtgtgttgtgtgcgcgcgcccgggtgccatcgccatcgtcccggcgcgggccaccgcggcctcacaggcccgtggagccgaagccgcgggtcccgcgcgcgctgctcggggcgacaaagtcccgcgtgaagagccacgcggggcggtgctgcatcggggtgcgcggggtggcggggaacggggaccgccccgtgatccagctcagcggctcgcgcgtcagcaccagctgcgccacgcgctggcccgcctccagggtcaccgggtgcgcgccgcggttctggatccggaagcggcacgggccgctctcccagggcgtggggaagacgacgatgccgcggaggttgagggacgagcgcccgaagacgtaggcccagtggcggccgtccgtgcggtgcaccggcagcgccacggtctccgcgccgccggggggcagcaccagctcgcgcgggcaggggatgtcgtatccggcgtcctcgtcgcgcttcggcgcaaagacctcgaagaagggcacgtccgtggcgatgcccggcgcgaggcgcagcggcgtgcaataaaactgcgcccgtgggcggcccggggctacaatcgcctgcacctcgccgcgaaagcccgcgtccacgatcccgttggtggtgcgaccgcccccgcgctgcgcgacgagcatcgcgtatccggtgggcagcgccaccttgaggcccaggggaaccttataaaatccctccggccccggactccggcacaccagcagccgccctccgtccactgtcacgggctcgctcgcagacaccaggatggtctcttcctccgcccggctctcgctgacgctcgtcgccgcgctctgcgcgctggtgacgcccgcgctctcttccatagtcttttccgaggggtcgctgccgctgctgcgcgaggagtcgcggaccagcttctggagcgccacctgcgccgccaggggagtcccggtggatcagccgacggccgccgccgtgacattttatatctgcctcctggcggtgctcgtcgtcgccctgggttacgccacccggacgtgcgcccggatgctgcacacctcccccgcggggaggcgcgtataaagatcgatccgcacgcgcccgatcccactcgctcgccatgtccagctcgagaaagacccgggtcgccgccgacgagaccgcctcgggggcgcgccgccgcgccggcagcgcctctcgcgcccgggccgccgccccggccgtcccgaccgccgcgacccccagacgcccctcggcctacgacgacggcttctcctaccggtctgccccgtcctacgacgacgatgactactacggctacgatggccacggctcctcccacgccccccgcgccgccaaggtgacgcccgcggccgcctcgcgggcctcgaccggggccaagagcgcctcggccgccaagacccccgcggccaagaccgcccgctcggcctcggccgccgccccggccgccgccgccaccaccaccgccgccgccgcggaaccggccgcccggcgcgcctcgacgcgggccgcccccggggagaacctcgacgtgggccgccgccggctcgccttcagcgacaggccgtgcgaggccaacgtgccctggcgcggcgcgacgcacgccttcaacaagcgcatcttctgcgcggccgtggggcgcgtggccgaggcccacgcccgcgccgccgccgagtccctctgggacatgaaccccccgacgacggacgcggcgctcgaccgcttcctgcaggccgcggtggtgcgcatcaccgtgtgcgaggggctggacctgatcgaggcggccaacgccgtcctggacgagagcaccccggggcggaagggaaaagtgtataaataaaggcgttgcaaatggcaccccgctcagtcgtccgctgtcttgcttgtgggggggtcggtgaggatgcgcgacgaggagtgcgtggtcgcgttcgacgaggccctgctggggggcgcggccccgggccggccgcccgtggtggccgtcccgcgggcggccagcccggacgcgctgtaccagcgcctcatcagcgacctggccttcgacgagggcccggcgctgctgagcgccatggagcgctggaacgaggacctgttctcgtgcctgcccggcaacgaggacctgtacgcgcgcatggtcatgctctcggcctccgcggacgaggtggcggcccaggtccaggcgccgacggcggacgcgagcgtcgacctcggggtgcccggggcggagcccatgccgcgggtcccggccgtggaggaggagctgcccgcgtacgtggcggccgtggagcgcttcttcgtgtccacgctgcgggcgcgcgaggagcggtacgcgcgcctgctggacgcctactgccgcgcggtgctgcgctacctgcacgcgcaggcgcgccgcgacgagggccggctccggcagctggtgcgcgggcgctactaccgcgacgtcgcgcgcctggcgcgcctgctctttctgcacctgtacgtggccacggcgcgcgagctctcgtggcgcctgcacgcggaccaggtggtggcccaggacgtgttcatgtccctgcgctacgagtgggaccagacgcggcagctgacgtgcctcttccacccggtgctgttcaaccacggcgtggtgctgctggcgggcgcgccggtgccggccgcgcagctgcgcgccgtcaaccgccgccggcgcgagctgggcctgccgctcgtgcgcgccggcctcatcgaggaggacggcgccgacctggtcgaggagccgccgttctccgccgcgctgccgcgcgccgccggctacctctcgcaccaggtgcggatcaagatggaggcctactcgcgcgagtaccgcgaccacacgtactgccgcccgccctcgcccgtggccagctacggcagcaccgcggaggccctgctgccgcccccgtcgccctcggccgtgctgccctgcgacccgacgccgccgccgcgcgtctcggccgccccgctcatcaccacggtgacgctggccgaggccgaggaggacggcgcgctgacgacggcggcgcccgcggccgcggtcgcgaccgcgacggtcgcctcgcccgggcccgccacccccgcctaccacctcatcccccgcgacgcgctcaaccgcatgtttgagatgtgacgccgcgccccgcgcgcgcggtcggatctgcggcgcccgaccacctgtgggcggggcgtataaagccgcgggccggcccggagggggggcatttgccgcagcgaccaccatgagcgacgcggggcccgtgcggcgcccgaggcgctcgcaagagcagcgcttccagccgttcccccgcgcggggctcctggtgcacctgcaggacgtgctcgtgggcgaggtgcggcgcccggacttccgccctccgcccgacgaggagagcagcgaggaggaggagcccgtctggaccgacgacgaggaggaggacgagggggaggaggggggagcccggcgcggggacggggagggggacgacgacgtggacgtggacgacgaggaggaggagagcgaggggggcgcgtggtccgacggggagctgtacgtggcggccgacggcgacgcgtgggacgcggaagaggacgaggacggggacgaggatgaggatgatgactatgacgatgacgacgacggctatgacggccgcggcgccgcggcctcccgcgccgcggtcgggccccccgccgacgtggactacctgtcgcgcgccctccgcggcatggagacggcctccggccccgacgagcgggaggccttcacccgctacgccggcacgctctaccgccgccagttccagcccgggcggccggggtaccggcccccgcggccgcgcgacccgccgcctcccccgcccccgcgggcgcggccgccgctgacgacggcgacgacggccgcgcccgccgtcggcaccgccgccccctgcggcggcgacgactccgcgccgcgcctcgagcgggacccggacgccgcgtacgccagctggacgcgcgacatgcccgacgggacctttctggccatgccgtcgtgggtggcgctgcgccgcgagccccgcgggcccccgggccgccccgtcgggcccccggacatcctgcgccgcgccccgcgggccctgaccttcacgccgcaggcggcctcggcggcgctcgtgagccgcgaccaggacgcctgggaggcgctggtgccgctgcaccacctgcagatgcactcgtggggcggcagcgcgccgccctcgcgcgggggcgtgtacgcgacgcccgcgcccgagacgcgcggcgtgtggcgccgcgcgctgcggcaggccatcgccctgcaccacgccatgttcctggcgccgctgcagtcctcgacggtcggcgcgcccgcgctcgcgggcgaggcgctggccttcgcgctggacgccgcggcgcgcgcggccgtcaaccacgacgccgcggcgcgcgagctccccgagcgcgcggcgcgcgcgctgcgcgccgtcggccggcgcgcgcccgcggcctcggggccgcggcccggcctcttccgcgccttctgcgggtccctggcctactggccggagctgcgcgtcctcagcgggcaccccgaggacgtccgcttcgcggccttccacctggcggtggccgaggtgtacctgctggcgcgcgcccacggccagaacccgggcttcacggccgaggagcgcgagctgctcgcgcccatgttcacgctgaccgtgctcgcggtgcaccacgcgctgcgctggctcacgacggccgtggcgcgcgccgtctcggacatccccgacgacgaggccttccgcgccgtgcgctcgcgcgtgccggcgtcgctggtgccgctgggcagcatcgcgctctcggacgccgagtacgccgtgctgaacgcgaccgaggtggcggcggcgcgcgacgccacgggcctgggccaggccgtgtcgctgggctacgccgccgcgcgctccgcgctcacgggcctcatgcgggcccacgccggcgggagcgacgccctcgtcgccttcgcgctggtgctgcagcgcctggcgggccacgcgaacctgctgctcaactgcctgctcggcgccgccgtccacggcggccggccggtgcgcgtgtacgaggcggcgctggacgactacgcggagctcatggacgcgctcgacccgctggtgcgcgcgtgcccgctcgcggagttctgggagcagcgcgacgccgtcatgcgccagctgcggctgacgccggccccgggcccccccacgggcggcaagcggctcgtcatcgcgcccgcgctgccctcggaggacctggacgggctcgtcccgcccacggccgtgcgccccgcccacttggggccggacgtcgacctcgccgaatacgccgcgcgccacccggagctggtgggcgtgccggaaccccgagcccatttatcggcgcgccggccgccccggggccgctaacccgtccctgcctccgccatgatccgccgcgcccgaggaacgcgccgcgcttcgtggaaggatgcctcgcgccgcgtcaccgaggggcgcacccgcgccagctgcctgctggcggcccccggggaggtgctgacggcggccgtggcggccctgcgcgaggcggccgagcgccagtgcgcgcccgcgctcttcggcgcccagcgcgcctcggccctggcgctcgtgcgcagcaactacgcccccgagtccgtgatcacggcgtgctcggcggagccgcaccgcgaggcgtacgcgcgcgcggccgaggccgcgctgaagggcatctcggcggacgtggcctggcgcgcggtgctggccacctactggcgctacctgaaggccagctcgggcgtggaggtgcgctcggacgacgagcgcgacgcccccaccgtcatgctgctgtggtcgacgttcggcaagacgctgtcgaagcacccgttcaagcacaaggcccagagcgccgcgtacggcgccacgcgcgccgcgctcgccgaggcgaccgaggccgtggagcggtacgcctacttcatgcgcccgctcgaccccatggtctccagcccccagacgagcgtgcgcctgcacgagatgctggcgtacgcggccacgctgtaccgctggctgctgtggatgatggacacggtcgacgcgcgcgtgctccgccacctggggcgcacgccgcgggtggtgcgcggcccgcgcgagacgatgtcgcccgaggcgctctttgggcggcaccgcgccggcgggcccgccgtggcctcgggctcgggcgaggtgctcgtgctgacgagccacatggcgacggtgtttgacgccctggagatcctcggcgccacgtgggccgagacgccctggaagagcggcgtgtgcggcctcacggccgccgtcgtggccgccgtggacctcgtgaccttcgtgcaccaccacgcgcagaccctcatcaacctgacgctcgccggctacgtgtgctggctcgacgacgggatggaggacccgtacctgcgcgcggcgctgcgcgcgcagtgccgcttccaccacctgatcggcgacctggcccccacgagctcgtcgcacgcctggggcgccatggagcgcggcacgctcgcctggttcaactacgccatcgcgcgcagcctggcctcgtacggcgggcccacggagcgcttcgcgcgcgtgctggcgacgacgggccagcgggacatgcgcttcctggccgtcgcccccgcgcagccgccgctgccgcccctgccgcgggcgtcgcggacgccctcgccgccgccccgcgaccgcgacgcggagccgccccacgactacgtggaccccctcgccgagtacctgaactggcccgtgcccgcctcgccgcccccgctccccgaggggcccccgagctcggacgacgagctggaggtggacggcggcgggcggcgcccgctccggcgcagccgcgacgccgccacgtacgtgaaccgcaaggacattgcccgcgcgatggcgggggcggaggacgaggacgaggatgaggaggaggacggccggtacccgcgccgcgacggggacgccacggcctcgacgagctcgtcgcaggagagcgccgccgacgaggagttcccgcccgggatcggcgtgcgcgacggcgaggaggacgacgaggacgagggggtggacgaggaggaggaggaggaggacgtgtacgtcggccccgacggtaccggcgaacggggcgccgccggcggcggcaacgccgcggacgacgacgacggcgccccctgggccccgctgacgcgccacgggagcatgcgcaccagcttccggcgcggggtccgcgcggcccagcgcttcgtgcgccgccggctctcgcgcacgagcgccgaggcggccccgcgggcctccggcgactcagcctccgcggcggcccccgccgccgcctccgcccgcggcgagaccgaccacgtgtaccagcacccccgcccgcggacccgcgcggacgacggcctgtaccagcaaccccgacccgtcatcgacctcaccggccaccgcgcgtcgcgccgcaagagctggcgcgtgtgacggctcccacggactcaccgttgtattgttttcccccgttgcccccctgtgtggaaataaagtttttttctaattctgtacacaaggcgtgtgtgtcgtggcggtcgtggtcgcgggacgggagggcggcggggagaggggagacgagaggggagaggggagacgagaggggagaggggagacgagaggggagaggggagacgagaggggagagggacgaggcggacgcgagacgtgcgatcaacggcatctttatttttttccatctgcggggagggggctagggggcgtcggggtcctcgttctcgaggcgctggtagtgccggcggcgcgtggccatcgccccgacgcggctggccagcagcgcgggcccgctgttcttcttgcgcgccttgtgctcctgctgctcgagggccgacacgatggacatgtaccggatcatgtcccgggcctggtccagcttggcctcgtccacgtcgtcctcttcgacgccgtcctccttgagcgccttcgtcgtgacggggtacagggccttcatggggttgcggcgcaggcgcgagatgtgccggtaggccaggaaggccgcgaccaggccggccagcaccagcagcccgatggcgagcgccccgaaggggttggacaggaaggacaccatgccgccgacggccgagatcacggcccccgtggcgcccaggaccaccttgccgacggcggcgcccacgtcgccgaggccctggaagaagttggcgatgccgcgcagcagcaccacgttgtggtccaccttgaccacgcggtcaatgtcgtagaacttgagcgcgtgcagctggttgcggcgctggatctcgctgtagtccaggaggcccgtgtcggcgagctcctcgcgcgtgtacacctcgaggggcaggaactcgcggtcctcgagcagcgtcaggttcagggtcacccgcgtgctgatcgtctcgggcacctccaccatgcgcacgtagctgtagtcctcgtagtacacgtacccgccgcccagcttaaagtagcgccggtggttgccggtgcagggctcgatgaggtcgcgcgagatgaggagctcgttgtcgtcgccgagctggccctcgatcacgcccgtgccgttgtgctcgaaggtcaccagcgggcggctgtagcacgtgccgcgctcgccgggcacgcgcatggagttctgcacgtacacgccgccgcgcacctccacgcaccgcgagatggccatcacgtcgccgagcatgcgcgccgagacgcgctggcccagcgcggccgtggccacggcgctggggttcaggcgcgacatctcgccccacagggtgcggtccttgttctgcagctcgcaccaggcggccgcgatgcggctcagcatgtcgttcacgtgcgcctggatgtggtcgtaggtgaactgcaggcgcgcaaactcggccgagcccgtggtgatgcgcaggtgccccgtgccgttgacggccggcggctcgggcgtccccgccgggccgggggagcgccgggcccgacgggcggccgcgggggacgcggggcccacgacgccggcgaggccgaggcgctcgagctcgcgcgcgtacagctgcgccagctcgttcgagatcagcgggcggaaggccaccacgaagcccccgcgggcgaggtacacctcgggcttgtcgccggccagcacgtgcgtgttgttgtagcgccgccggtagatggcgtcgatggcctccgaggcctcgcggaggacgcagtcgcccaggtgcacgcgctgcaggtcgagctgcgtgacgtcgctgacgaaggaggcgcccagggcccgcgacgtgaagcggaaggacccgtcgcgcgtctcgtcgcggatcatctcctcggcctcgcgccacttggccaggctgcacacgcgccgcgtcttgggggcccagtcccaggccaccgtgaagtgcggcgtgcgcagaaagttgcgcgtcacgctctcggaggcgcggaggcgcgagtccaggtcgatggggtagtagtgctccacctgctggaagcgcccgggcgcgtagccgatgtgctccccgtgggccccctcgcgcaggccgtagaagggggacatgtacacgatgtcccccgtggacagggcgaaggagtcgtaggggtacacggagcgcgcctccacctcctcgacgatgcagttgacggaggtgcccgtgtggtagaagcccgcggcgccgatcttggtgtaggtgtcgttggtggtgtgccagccgcgggtgccgagcgcgttcaggcgcgaggggcgcaggtccacctcgacggggttctcgtcgcggtcgaaggcggtcaccttgtggttgttgcgcacgtactcggccttggagacgcacttgccgcggcggtcgatcacgtccgtgatctcctgcacggggacgggcacgcggtccgtgaagcggttcgtgatggccgcgtacgtgctcccggaccacacggtcgtgacgatgacgttcttgtagtagatgtgggccttgaacttgtgcggggcgatgttctccttgaagagcacggcgatcccctccgtgaagttgcgcccctgcgagtactcggggcaggcctgctcgggctccaggcgcaccaccgtggagccggacggcggcgggcagacgtagaagcggtcccgctcggtcgcggccgcgcgcacggccgtgcgcgcgtccaggtcgccgtactcgccgtcgggggcgtccgaggggccgggggagacggccccgtcgatctcctcgagggactcctccgcggagaagccgtctggggtggcgcccgtcccgggcgcgggcgaggccgaggcggcccgcgtcacggccgccgcgccgcacgtcggggtcgcggcgagcgccagcagcagcagcgctagcgcgacggcgccccgcgcagctgcagcgtggtgtggagcaggccaaagacgtccgaggccagcaccgccgtggtgcccgggccgatgcccgcggggcccgcgccaaagaccgccaccagcgggcatgacgcctcgtaggtcaggtacagcccgtcgcgtccggcgaagcgatccgcgcggaggacctcgacgtccccgcagtggaagacggacctgaagactgcaaccgccagcaccagctcgcggatatatcgccaggcctgccgctgggtggcggttacgccggacgtctggaagcggtagaagccgcggaactcgctgacgcaccaatcgcgggccaccatgaagcgcgccagctcctccttcaggtgcggcaggaggcccacgttctccaccgcaaagtacagcgccgtgttggggggctgggcgaaggcgtgcgtgtcgtgcgcgaagagcggcccgttgacgagctcgaagaacttgtgcgtgagcgccgggaggagcgcggcgtccacggggtggcgctgcaccgtggccttcatgaagctgtgcgcgtcgaagcagcccgtctcgcgctcgtccacgacggtgcccgcgcgcgccacggcctcgcagaacgcgcgccgcgcgcggaacccggcggccaccgccacgtacgtgtgcagcagcgcgtcgccgtacacgttcacgcgcagggtcttctccagctcgcgccgctgctcgcgcacgcactgcgccaggctcgccgccgagcgccgcgagaggcggtccgcgtacaggcgccgccggcgctcggcgtcgcgcagggccgcggccggcaggtccgcccacgccacggcgtccgccggcgggggcgcgccgggcccgtcctcgccgccaccgccccccgcgccgccgccggcgtccgcgccccccgcggcgccgccctcggggtccgcgtcgtcgccgccctcgaggaggcgctcgagggcgtcgcggttggcccggtccgggtccagcatgcgcagcatgggcgtggacatgtggtggtcgtagcacgcgcggatgagcgcctcgaggcgctcgtcgggcgaggccgcacagccgcccaccagcagcccgtcgatgatgtcgagctcgcgggccgcgcgggcgcggtcaaagtgctcggggcgccgcccgaagagcgcgagctccacggccgccgcgcgcaccgcggcgcggcgctcgcgccgctccaggtcgtccaggttgtgcgtgaacaggtcgagggcccgcaccgcctggttagtggacgccagccagaactgcagctcgctcacggcgtacaggcgccgcgaggcgggccggaacacgtcgtgcgcgtccaggagcgcgtccgcgcgccggcgcgccgtctcgcccgcgtccccggcctcggcgcccgccgccgccgcctccgcctcccgggcctcggccgcgtccacggcggcgagcgccgccagcgcgcgcccgcggcgcgcctcgtcgagcccgcgcacgtgcggcaggttcttggccacgtcctcggggtccacgcgcaccgccagctgccgcgtgaggtggtcgcacacgcagccgagcaggcgccggtgcgtggcctcgccctggttggccgtcacgcacagctcctcgaagcacaccgcgcacggctgcgccgggtcgtacagctccggcggcaccacgagcccgccgccgatcgtgcgcgcgaggaactcgtccacggccgacaggtccaccgcgtggggcgtgatcaggtggcagtagttgagctgcttgagcaggttctcggcgtcgtggaggaactgcagctccgtctccacgcgcccgccgtacgtgtcgagcgcgacgcgcgcgtggaagcggcacgcgccgcccatggtgcgctcaaagtaggcctcggcgtcgtcgccggcggcggccagcgcccccagcacgcggtccccctcggcccgcgcgtacgcgagcgccaggcgcagcgcgcacgtgagcggcgtcatctgcgcctccagcggcagccgccgcgcgaggtagcggcacatcagcgcgttcagcttgacgcgcggggccagctcgcgcaccaccgcggggtcgcagcgcttcagcatctccagctgaaacacgtacgtctgcacctgccccaggaccgccacgagccgccgctccgccatggccgagtccgcccgctcgctccctccctccgcccgcccgtccgccgcgagagaggccacgggacgcgcgtgtgtgtctgtcaaacgcggtcatctttattgatacagccgggcacccgcgcccgtcccctcctcctcctccccccatcacagcatgtcgaaggtcagccgcttctcgggcggggcgcccatgtcaaacaggtcgtcctccgggggcgggcgcttgccgcccagctccgcgggcgccagcgaggccggcgcgagccccgcgcccgcgtccccgcccgcgccgcaggccccaaagtcgaaggcggtctcctcgccgccgccgtccgcgcccgtctggccctcgaggtcgcgcaccagctcggcggccgcctccacgctccagcgcccgtcgcggtcggccacgcgcccgttgatctccgccagcgcggccgccaggaactcgtcgtccacgatcgcccgccagtcgtcgggctccaggtgctgcgtgcgcgccccgagcgcgtgcagcacggccgcgtacacggccgtctgcaccagcggcccgccgtcggccaggatggtcttgacgtgctccccgagcgagtggtcgtgcagcccggcgccgccgcccgtctgcgagcacgtgaagcccacgcgcgggcaggccagcacaaagtgccgcgtgcggtcaaacaccatcagggggcacacgtgcttgccgccgttgagcccgctccagttccccgcctggaagacgcggttgttgccggccatgccgtggtacttgctgacgcccaggcccagcaccaccagcgggcgcgaggccatgacggagcggagcaggtggctggacgtgtacgtggccacccacgtgtcgtcgcgcggcgcgtccagcacctcgcgcgcggcgcgctccgcgtcggccgcgtccgccagcgggcgccgcacccaggccatgacggcggccgggtcccgcgggcgcttcgactgcatcgtgatccccgtgaggctgttgatgaagaactgcttgtggtcgcagtagcgcaggatcaggttggccaggtagaactgggccagctccgccacggtgcccggggtcaggttcacgtagttgatggcgccgtagtccaccgagaagcgcttcacggccgcgatggtctcgatgtcgtccttggtcagcagccgcgccggcagctggttgcgctgcagcagcgtccagaaccactgcgggttggggctctgcccgccgggcggcttgccgcgcgggaacagcgtcgcgtggtgctgcttcagcaggaagcccagcgggccgttgagcacgtccacggccgtgtccgggcgctggtaggcgcccgcgagcccggccacgcgggcgcgcgcggcgtccgagaggctgcccgagcccgtcgagaacatgacccggctcttgacgcgcagctcccgcagcacctcgaagctcacgcgcgccaggtccccgtcgtggtcgcgcgccgggggctccgcgctgcccttggtggggtccggggccacgatgccgtcggcgagcgtcaccgtcaccgtcttggccgtgacgaagcccccgttgagcatgtccaccacgcggcggcgcagcaccggctggaactggttgcggaagttgcgcccctccacgggctgcccgtggagcacgccgtggcactggctcagggccaggtcctgcagcaccgccagcagcgtgcgccgcgccaggaagctcgtcgccgggcacagcgcgcccgagtacgggtccagcgagagggacatggtgtggttggcgtcgtagagcgcgtcgcggagcttgaagtcgcgcgtctccacgagcgtgcgcacgaagcggtccgccgcctgctcgaccgtgtcgcgcagggcgcgcagcgcgtgccggaagccggcgtggtccgtcaccgtctcgccgatgcgcgcgccggccacgtccgccagcagccggtccacggcggcgcggtacgtgtcctgcatgatcgccttcgggggctcgctgtcgttggggcgcttgagggccccgtacgaggcgtagttgccgagcacgtcgcagtcgctgtaggcgctgttcatggtcccgaagacgcccatggggctgcgcgtcggcgcgccgaagcgcgggaggcggtggcgcaggcggtgcagcgtcgtgtgcgcgcaggcggggcgcgaggcgcggtcgcagagcgagcaggggacctcggcgtcgaggctgccggccacgtagcgcacggcgtccacgtcgccgcggcccacgaacgtgcccgcgtcgcagcgctccaggtagaacagcacgcgcgccagcagctgcgggcagaagccgcacgccatcaccaggtggtccatggcgaagtcgtggttgcccggggccgtcgcgggctgcgaggccgtgtgcggcagcacgcgcccgtccttgtccgtctgcgggttcccggccaggtagggcgccgccacctggtagaagcggttaaacgaggggccggcgccctccttgccgccgccgtcggcggcgcccgcgtcgtccacctccgtcaggtacagcaccgagttggagctgaacaccagcgcccccacgaggccggccgcgcggctcacgtacgcgccgagcgccgcggcctgcgagacgggcgcgcccgcggaggcggccgcgccgccgccgccgtccttggcgcccgcctcggagagcacgggccagtcgtccagcgcggggggctcctcgtcgtacacgccggcggcgacgagcgcctccagcgacagcgccgcgtcggcgctcatgacggacgccatgcggcgctcgaggccgccggcggccgcgtcgccgccgcgccgccgctgctcaaagagcgtgaaggtcacgtcggcgggcagcacggcgctctcgtggttctcgtcgaaggccaggtgcgccgcgccgcgcgccaccgcgtccaggttccgcacgcgcgtggccacggccgccgggcccagcacgtagccgtgcagcaggcggcagagcgggccgttgaagaaggggcgcgggtacacaaactcctcgctgatggagcggttcttggcgttgaaggggtcggccacgagccggttgacgtccggcatgaagaggtgcacggggtagagcggcacgcgccacgcctcgtgcccgttgatggacaccgtgcccgcgcccccgtagtgcaggaaggcgttgcacaggtacaccgcctccttgaagccgtcggccaccacgaggtaggccgcgtgcgcgtccgcggccaggccgagccgcgcgcacacctccgcgcccgtcgtctcctcggcgttgtccaccggcgtccggtaggccgagaacccgaagcgggcgcgcgccgcctcgcaggcgcgcgagagggcgggcgcggcgctgctcgggggcacgcagtccccgttgtggaagaagaacacgttggggtggaagtggttgggcgtcagcttcagcgtgagcccgccgccgagcccggtggtccgggcgcccgccaccacggccacgtgcccggcgaagccggcctcgacggtcaggccgcgcaccagcggcgccaccgccgcggcgtcgtcggcgctgcgcgccacgagcagcgccagcaggtcgcggcgcagcgcctcgatcggcgtcacgtacacgtagcccagcggcgcggcgcgcacggtcacggtcttggccgcggcctccatcgtcgcgggtgagacgagacgagagcgggagcgggagcgggagcgggagcgggagagagagaaggcgcggcggacggggtcgagtctcggtcccctctccgggcggacgggtccggcggcggggtgaagagagagacggaggtgtcggcgccgaggggcgaacgcgacgtcggcggcggtggtagagcccggcccgccccggccgggtcttaaagcgtggggccccgccccgccggggcgtggcgccccgccccggagcctatacctcggccggcggcgttcgcacctccgcgtgggacgagcgatggcggcgcggcagggcagctacgtgacgcgcctgagcgagttcaagttcatcgcgccgcggtgcctggacgccccggaccagcgcggcgtgcacgtgggcacgctcgcgcgcgagcccacggtctactgcggcggcgccacgcggcccatcctccgcggggagcccttctggccgcggcgcgtcgccgcgtgggacggggcccccgagcggcccgtgagcccgcgcttcgagcgcttccacgtgtacgacatcgtcgagagcaccgagtacgccagcgcggacccgcgcttccccaacggcacggtggtcacgctgctggggctcagcgcctgcggcaagcgcgtggccgtgcacgtgtacggcgtgcgccactacttcttcctgggcaaggccgaggccgacgccgccctcggcgtgacgagcgccgagcagctcgcgcgcgcgctctcggcggcggcggcgcgcggcccgcgcctcgggcccgccgacgtggacgcgcgcgtcgtcgacgccgcgcccgtctactactacgacgccccccggcggcccttctaccgcgtctcgagcaacagcgggcgcctggtcgcgcacctccgcgagacggtctgcgccggcctggtgacgcacgaggccggcgtggacgcgaccacgcgcctgctgctcgaccacgacctgcccagcttcggctggtaccgcctgcggcccggccccgcgggggagcgcgtggtgctgcggcaccagcgcctcacgtccagcgacgtggaggtcaactgcacgcccctcaacctggcccgcgacgaggacggcccgtggcccgactacaagctcctgtgcttcgacatcgagtgcaaggccggcgacgacgcggccttccccgccgccgagaaccccgaggacctggtgatccagatctcgtgcctgctgtactcgctcgccacccagcggctggagcacacgctgctcttctcgctgggctcgtgcgactcggacgaccccgcggtgacggtcctcgagttcgacagcgagttcgagctgctgctcgccttcgtgaccttcctgaagcagtacgcgcccgagttcgccacggggtacaacatcatcaactttgactgggccttcgtgcacaccaagctcacggccgtctacgggctggccctcgacggctacgggcgcttcaaccgcggcggccagttccgcgtctacgacgcgggccagaacagcttccagaagcgcagcaaggtcaagatcaacggcctcgtctccctggacatgtacgccgtcgccgccgacaagctgaagctgccgagctacaagctcaacgccgtggccgaggaggcgctcggcgagcgcaagctggacctggactacaaggacatcccgcggtactacgccgcgggcccgcgcgagcgcggcgtcatcgggcgctactgcgtgcaggactcggcgctcgtgggcaagctcttcttcaagttcctgccgcacctcgagctctcggccgtggcgcggctcgccaacatcacgctcgcgcgcgccatctacgacggccagcagatccgcgtgttcacgtgcctgctgaagctcgcgggctcgcgcggcttcgtgctgcccgacaagcgccgcgacatcgcggacgaggacgacggcggcggctaccagggcgccaaggtgctcgagcccgactcgggcttccacgtggacccggtgctcgtgctggactttgccagcctgtacccgagcatcatccaggcgcacaacctgtgcttcacgacgctcgcgctcgcgcgcccggcgggcctgcgcgaggacgagttcagcgccttcgaggtcaacggggagcggctctactttgtgcacgcgggggtgcgcgagagcctgctctcgatcctgctgcgcgactggttggccatgcgcaaggccatccgcgcgcgcatcccgacgagcgcgcccgaggaggccgtgctgctggacaagcagcaggcggccatcaaggtggtgtgcaactcggtgtacgggttcacgggcgtggccaacgggctgctcccctgcctccccgtggcggccaccgtgacgaccatcggccgcgacatgctggtggccacgcgcgactacgtgcagacgcgctgggccacgcgcgagctgctcgagcgcgacctgcccgcgcgcccgcccgcgggcgagtacgccgtgcgcgtggtctacggggacacggactcggtcttcatccgcttctcgggcatcgcgtacgacgacgtgtgcgagctcggggagctcatggccgcgcgcatcacgcgcgacctcttccgcccccccatcaagctcgagtgcgagaagaccttccggcgcctgctgctcatcacgaagaagaagtacatcggcgtcatcaacggcggcaagatgctcatgaagggcgtcgacctggtgcgcaagaacaactgcgccttcatcaacgcgtacgcgcgccgcctggtggacctgctctttggcgacgaggccgtctcggcggccgcggccgcgatcgcgcgcgcgcccgcggcgcgctggctggagcgccccctgccgcgcggcttcgccgccttcgggcgcgtgctggccgaggcgcacgcgcgcgtcgccggcggcgccggcctggacgtggccgacttcgtcatgacggccgagctcagccgccccccggacgcgtacagcaacacgcgcctgccgcacctcaccgtctaccacaagctggccatgcgccacggggaggtgccgagcgtcaaggagcgcgtgtcctacgtcatcatcgcgcccacccccgaggccgagcgcgacgcgcgggccgtgcgcccgggcctcgccccggggaagctgctcgtcagcgacctggccgaggaccccgcctacgtggtcgcgcacggcgtgcccctcaacaccgagtactacttctcccacctgctgagcacgctcagcgtcaccttcaaggccctctttggaaatgacaccaagatcacggagaggttgctcaagcgctttattccggaaaccgccccgggggacgcgcccttcgagcacgaggccttcgcgccgctcacgggcgaggggggcgaaagtcttcaaacgctgcgtacaatcttttgtactccaacagcagctccccgtcgaagctgatgtcccgcatcttgcaataaatgtcgctcgcgctgacggcgggcacgtcgtccgcgggtctcccgtctcccttctttcgcaccaccaacacgaacatggtctgccacacgtgcgcgacgatgcggtggccggcgcaggccccgagcaggcggtccagcacgcggtggtgcacgtgcaccgacttctcggggaacaccacgtacatcatgagcgcgccctgcgcgtccgggtcctcgaagaagagcacgcgcacgtcgcgcaggcccccgtcgcgcagcacgtagtaggcgaaaaagagctccgggtgcgcgagcacctcctcgagcgcgcgctccgaggggtcccgcgcggccaccgaggccaggaacacgcggtactcgtagatgctgttgagctgctgcacgtaggccagcacgagcgccgcgcggtcgctgcgcccgagccgcggctcggcggcggcgcacgtcgggcagcagccgccgaggcccaggtagtagcccatgcccgagagcgacaggcagttgtcggccaccgcctggccgaggtcgaagggcagcgtcacgggggccgtcttgatgagcgggttcgagaggccgcgcacggtggccacctcgtccgagggctgcgcggccgcgtaggcaaagtagggcgagtagcgatcgcggaccgcgcgcgccagcgtcttgcgccgcgcggccgccgacttgcgccgcaggagtcgccgccgctcaaacatagctgccgagcgtgtgctcgagggagacgagcgcgaggccgtggcgccgcagcagggcgtgtgcgtcgcgcaggaaggcggcgcgggcagtgggcttcggcgggaacagctggtacgctttaaatacgtacgccagggtccacagctccccgcacacgcgcccctcgaagcggttctcggcggcgatctgcgccttgcgcaacgcgtcgccgcgctcaaagagcacgcgcgggtccgcctggcgctcggccacggcgcacagggggtcgcagaagaggtgcttgtagacggcgcgcgggcccgcggcgcgcatcagcgccaggaagcgcgcgtccgccgcgaagccctcgagcacgggctcgatgcagtcaaagaggcccgcgttgttctccgagtagctgagcacgcggcgccggaagcggcgcagcgccagccagtgcgcgcgcgtgagcagcatgttgcagagcaggcactcgccgtgcgtgcggttgcggccgcgcacgtgcttcagcgccagcgccagcacgggccccagcgtggcgtccaccgcgcggtagcgcgcgtacgccgcggcggtgttgcgggccaggaagccctcgtgctccgcccagtactcgtccacggcgcggtgccggcgctcctcggcggcgtccgtctgcgcgttgggctcccccgtggccacgttgcccagcagcagcatggcgaggtcggcgtagccgggcccgccggcgtccgcgtcctcggccccgcccccgcccccggcggcggcgacggcggcgcgctcgtacgcgtcgtcctcgcgctccgccgccgcgcagcacccggcgcacgcctcgtcgcccgtgtggtccttccagccccccaggctcggcggcgcgcgcgcgtccgccacggtgaaaaacatcgcgcgcgtcgcggcctggaccagaaaggactggttcgagaagcggatcttgtcgccgacggtgcggaagcagccgtgcaggaaaaagtgccgctgcacgtccacgagcgtgagcacgcgcgtggcgaaggcgcgctgcagcgacgcgcgcccgcgaaagtgcttgtccatcacgtacacgaactcgaaggcggccacgaagacggccgtggcgcacggggggcaggccagggccttggcgcacagcgccgcgtagtccgccagccagggcggggccagcgagcgctcgcggcggtacacgtcgatcgtgcgacacacgcggcagggctcgtccagcgccagcggcgccgcggcggcctccacgaagcccgcgggcgagtcctcctcgtccagcatcacgtgggcggcgaagatcagctcgcggaagagggcgtcgttcttggtcagcagcgcggggtcgaaggccgtgtacggcgcctcgaaggcgtcctcgctccagcccatggcgtgcgacggcggcggcagcggcggcggcgcggtcgatctgcgcgcggcgatcccggaggccgcgctgcgcgacttcgacgtggactttctcgaggccaactacctgcccgcgcgggtgcgcgtctggttcgaggacgtgatgccgcgcgagctggaagtgatcctccccacgacggacgccaagctaaattacctggcccacacgcagcgcctggcggcggcgacgagcgagcgcgactgtgcccacggggaggcgctgcgggcgcggcgcgatcgcttcgccgcggccgtcaacaagtttctggacctgcaccaggtcctgcgcgacggggtggagctgggcgccctataaagtccgccgcccggccccgggagctccaccagccccaccaccaccgccactgccaccgcagcccgcccgcccgccgggaagaccgccgccgtcgccgcgtcgacgtcgacgtcttagccgcgccgctctccctcgccctcccggcccacggcgacgacaacgacgacgaccatgagcggcaccctgatccagcgcctgaagctcatcctctccggggggaacctgcgctgcagcgacggcgagacggcctgcgaccccgagcggcccccgacgcggtgcgtcttccaggtccacgggcaggacggctccaacgacaccttcccgctggagtacgtgctgcgcctcatgcgcagctgggcccacgtcccctgcgacccctacgtgcgcgtgcagaacacgggcgtgtcggtgctcttccagggcttcttcttccggcccgccgacgcccccctggccgcgatcacggccgagcacaacaacgtgatcttggcctcgacgcacagcaccggcatgagcctctcggcgctcgacgacatcaagcgcgccgggggcgtggacatgcggccgctgcgcgccatgatgtcggtgagctgcttcgtgcgcatgccgcgcgtgcagctctcgttccgcttcatgggccccgacgacgcctcgcagatgcagcgcctcctggaccgcgccgagctgcggcagcgctcggtctcccgccccggcggtggcgacgacggctgctcgggcgagggcccctcgccccgcgcgcccatccgcccacccgtcctctcgcccgtgcccggacacgcggccgccgccctcgtgggccaggccgcgtaccctccgcccacgcggttcccggcctcgctgctgcacgccctcctggggctgcggcgcctcgccggctacgccgtggcgtgcgtcaccggcgctctcgcgatagtcatcatcctgaacatgcgttaaaggcggccgcgcgcgcgggggcgcgcacagacgcgcgctccccgccgagccatcatgtccttcgacccgaacaatccccggacgatcaccgcgcagacgctcgagggcgccctgcccgtggacattctgctccggctcaaccgcgcgacggggctgcatatggatgcggccgaggcgcacgcgatcgtggaggacgcccgccggaccctgtttatcggcacctccctggccctcgtgaacctgcggcacgcgcacgacaagcacctcgtggagcgccagcccatgttcgccaccagcgactacagcagctgggccaggcccacggtcggcctgaagcggaccttctgccctcgcccctcgccctagccccgcgcgatcaataaagcgagccgcgttgcaccgtcctctcgtctctcgtgtgttgtctgggtggggtttcccccgagatcccccgatctccccgccgggtctttgggtcccccaccgagttccccaccgagttccccaccgagttccccaccgagttccccaaatgcccaccaccaccaagtctttgggtcccacaacccccaagttttggggtctcgggtccgagtcccccgagtcccccgagtcccccgagtcccccgagtcccccgagtcccccgagtcccccgagtcccccgagtcccccgagtcccccgagtcccccgagtcccccgagtcccccgaacacaccaccgcagagacaaacaggttgggtaataaacaatttattaaccaagaatcaggcggacctgaaataggtccacgttgaaccggcgggcgctgcccttgaggttcgcccgcgtctcccgcaggcggcgcgcgatcaggcggcacgcggcgatgagcagggccagggccccaagtccggtggcgcgcatgtaccgcctgttgatgagagtgtccgactccaccacgcgcggggggagcagctcctgggccagagagacgtcctcgtcctcgtcgtcctcatcgtcctcgaactccgcgccccaggggcccggctcgggcgcctcctcggcgggctcgcgggtcaggtccgcccgggtgggccagacgcggccgaggccgcccaggggagagtccggcggtgggggtctctgctgctgccgcggcggcggaggagggtgggcgggagggacgagcgcggccaggtcgtcgaaccacggcgggggctggtggcggtccgccagcggggagcgcggggccggcggcgcaacctccggcgggagctcggggacggtggtgggcgcctcagacagcggcggccagggcagtcgctccagcggcgagcgcggcggggtcgccggggcctcggcctcttctccctcgctcgcccatagcggctgagggggcggcggcagcggtgatggcggcagcggcggcgcctctcctccggggaacgggaacgggaacggctggtccgagggcggcggggtgaagagcgcgggtgggccggcggggtccctcggctcgggccacgaccacggcgcctccggggggcgctgctgagacggctgcggcggcagcggcggaggtgtgaacaggagcctcgggggctccgcgggcttctcgggctccgccggcgccggtctcgtctgcgccggggcccgggcgggctcggccctcggcggcgccggggccggccgcgaagatggtggtggtggtggcggcggcggctggggtggtggtggagcggcggcggaggaggccgaggaggagatggtggtggcggtggaggaggaggtggtggtggcggtggcggtggtggtagaggccggtgctgttgttgctgttgttgttggagctgagacggcggcggggggccgcgcgctctcgggggcggcggccagcgacgtcggggccgccagcggagaccgcggcggctgggtctgcgccggggccggagtctccggcgaggccggcagcggcaccgcgacgccctccagcggcacagactgcggcggcgggatgtggagacggaggtgcccggccggcgggcgcgcgacggcggcggctcgcctcactggtcctccgggggctgggaccacatctccgagcccgactccgagggcggggacggctgctgctgttgctgctgccgccgccggtgggcccgctggagcagtcgccggcgcttgggcttgggcttgaggcgggcgcggttcagctcccccgggaggacggccctcgggttgagccgcgggcggaagactcccggtctggcgcgaggctgatacgggccttcgcggaacaagagccatggattttcggcgactgtggtctctaatacccaccgacgttcgcgccttggtgcgtagaacagcggagggaaggggctgcgccactggggccgcggggagatgtcccgcaggttgatctccacggctcgcatccgaagccgggccaccgtctcctcggcgctggccaccactcccgggggctccaggaggtgctcgggccgctgctccggctgctgggggggaacctccgcgggagccatcgctcggtgttccggccggggaagaagaggaggacgaggcggcttctcgggacggagtcgaggcggtggaggaggacgcggacgagagctccgagtccgatgtcccgggaggagagcgggtccgacgctggtctggtccgaccgtgtgttcgcacttgtctgcgcaccccgctttatagcgttcgtcatcgagggggcggcccggggaggcggtccccgccaccctctcaccaaatttggtgtctcggacgtccggcggggcatcggccctcggcgctacgatgcggtcaccgtcgggtttctgggccgatggggcgggcccgggcgcggggggccggaaggtcgccgtcggggcagatgggggtggcggggcgggcggggcggcgggcccgggggtcgtcgtcggggcagatgggggcggcggctgtgggggagcgggcggggcggggggtgccgggggcgcgggcggggcgccccggggcttggcggccggctgggacgatgggggctgagcggcggcggcggcgacggtggtggtcgcggcgggctgtggcgggggaccctgtgacggaggttggggccggggctggggctgggtctgggggatgggggccttggccgcggcgggctgcgccggtggggcctgggtcgggggcgccggggccgacgggggagcctgagtctggggctggggttggggggtcggcttcgccgtcgcggcgggctgcggcgggggcgcctgggccgggggagccggggcctggggggtctgctccgccggcgcggcgggctgggtctggggggccttctcgggcttcgggggcgccgcggggtaaggcgggttgatgatcggcggcgggggctcgacgcgcgccttcagcgagggctggatctttcgcggcgacgagtgcggcttgggggtgggctcctggggccgctgcttgggggccgcggcggccttggcggccttcttggaggcggtggcggcggggggctcctgccgctgctgctgctgctgttgttgctgctgcggcggcggcggttgcgagggcgccttgggggcggcggggggctcctgctgccgctgcggcggcggctgctgcggctgcggcggcggcggcgccctggtgagcggggtcgcctccacgggcgcggcgggcggcggcgccggcggagccagcttgacgagcggggtcgtctccgcgggcgcggcgggcggcggcgccggcttggcggcgggggccgcctccgcggggggagccggtggcggcggcgccgggggggccagcggcgggagcgcgggcgggtcgaacggcggcgacgggggcagcggcagcgcgctggccaccaggtcgtcgccgagcaggtccagccacgggttggcgtcaaagtcgtcgtccgggacgaacccgtcgtcctcctcggccagctcgggcggcagcccggggatcggggtcgcgggccgggtgggcgtcgccgacacggcgtacgggaacgggtcgcccaggttgcggaagctcggccggccgtacagctcctccgcggtggccgggtccacgcgggcgtcgcccatgatgatggaggtgaacagcgggtcccgggggtccatccccggcagctggtcctccgtcgtctccaggaaggggatctcccgcagcagcgggtcgtcctcgagcgtgaagcccacgcgccgcggcggctcgggcggcgcccagagcacgcgcgacaccgccagctgcgagtccaccagcacgaggcaggccggcgtgccggccaggggccgcgccgcgatcagcgccgagagccgctccagctgcggcgtcaggcacgcgttctcgatggggttgtcgtcgagcgccagcaggcgctcgccccaggagctgaggtccgtggacacgccgcggtgcagcgcggggtcgcgcaggtcgcacagcgtcacggccagggtcaggcccgagccgggcgagtacacgtccaggttctccatggcgatcaccagcggggccccgacgagcaccgccgccgcggccaggtccatggcgctcacgcgcgtcgcgctgctcggcggcgagcccgtgacggtgaacgtcatgcccgtgcccacgggcgcgtacaggtcgccgggcgccggcgcgccgagcgccgcggcctcgcgcacgtgcgccgcggcgtccaggcgctccgtgaccgcggcgtcgtacgcggcgttcgcctccggctgcagggcggaccacagcgccgagagcagccgcggggggatgcacatgcgcgccagcacggtgaaggtggccagcgcgcccggcacggcgcgcgccagccgctggcccgtctccgtgtgcgcccagggcgcccacaggtccgtctccgagaagcggccctcggtccagtcggcgacgcgcagcgtgaacagcgcctgcccgtcgcgcagcgggcgccgcggcgggcgcgcgcccgcgtccagcgtgtccaccgcgtccagcgcgcggtccagcaccgcgtccagcgtcttggccatgtactcgtgctgcccggccaggtccacgcgcgtaaagttgaacaggtgcgccggctcgccggccaccgcggccaccatcaggtcggggagggcgagccgcaccgccggctccagcggcggctgcacgacgcggtccagcgccccgaaggtgcgcgagagcgtcgtggccagcacggccgcgtacgtctccgcggcggccttggccgccgagtcccagtcggcgcggcggtggcgcacgaagcgcgcgaaggtggcgaagcccgtgtcgcgggccttctcgtagttgagccgcaggtggatgatctcgttcatgaggcgcatgcccgccacggtcgccgtctccatgagcccctcggtgagcggcgggcgcagctcggcgccctcgtcggtgcacagcatggccgccagcttgtcgccgaccgcgcggtagcacacctggtactccacgggcacgttgttgtcgtccaggaagaccgtggggaacaccgcgtccgcggcgctgcgcgcctcgcgcaccgtgcagatgacgtcgcggtcgcccaggcgcgccgccacgcgctgcttcaggcgcgcgcacggcgccgtcgaggcgcgcggccgcgccggcgtcggccccgggccgccgtaggccgcgtagaactgcagccgcagcgtgagcagctcgcggtgcgcggcgtacttgggcgggacccagcgcggcagccgctccgcgcgcgcgtcgaaggcggccgccagcgcgcgcagggcgtcgtgctgcagctcgggcccgatgcgcgccgccatgcggtcgagctcgcgcatgtcctcctcgacggcgccggcgtcgcggaagaagtcgcccagggccccggcgcgcgccgagtacttgctctcgatgcgccccacgaacttggcgggcaggcgcgggtagacggcgtcgctgcgcagcgccagcacctccatggtcgccgtctgcagcgcgcgcgcgcgggcctcggccaggcgcgcctcgtcctcggaggcgccggcgcggccgcgcgcgcgcccaaagtcctcccaggcctcgtcccaggcgacctcggcggccgtcagccgctgccgcagcgtgtccgcctcctggcgcagcgtctgcagcgcgtcgatgcgctcggcgaactcgtccatgggcccgcggccgtcgacgcgcgtcgtcagcgggtgcgtgtcgatgatgagccgcgcctggtgcagccactcgacggcctgcacgtccagctcgggcgcctcctcgacggcgccgagcagcagcgccgcctgcgtcaccagctcctccgcggaggcggcccggtcgatgttgagcgaggccggcggcggcgtcacccgcagcaggttcttgaggttggccagctcgagggcgccgtcgcggtcgcgcgccgccgccgcgtccaccacctcgcgcagcaggcgcgtggcgcgctccgtggccgcggcctggtccgcctccgccgcggcgaggccgtcgcgcgccgccgccaggtcgcggcgcacgagcgcgcgcatggcctccgcgtacgccgtgcgctcgaagcgcgcaaagtcgaagcgctccaggtaggtcacggcgccgcgcagcagctcggtgctcggcacgtgcaggtgcacgccctcgtcgagcgcgcgcgccgcgtcggcggtcgcgcgcacgtacgtcacctcctcgagcgcgcggcagatctcggcgccgcggctgccgccggcctgcagcacgtccgcgcggcgcgcgcccagggcggccagctcggcctcgtactcggggaagccgcggcggtaaaagtccacgtacttggccaggcgcggcacgtgcgccatgtccgccccgacgaagcccacggcgtcgtagtagctgaagcgccccatgccggcggcggcgcgggccagcacgccgccggcgatgcgcgccagccgcgagagcggctccgtgtcggcgccgaagagcgagccgatgacgggcgccgcgacggcgtagtcgtcgagccaggtcaggcgctgcagcgcctgcagcggcgggcccgccgccgcgccggcgttctcgggcaggtgcgggttgaaggcgaacacggcgtccagcgccgtggtcacggtctcggcgttcgcggcgagcgcgcgctcggcggcggggcgcagctccgcggcgtcgtagccgcggtccgccgccacgtcgcgcaggcgcacgaactcggccgcgtcgaaggcgtgctgctcggcgcggcgcagcgccgcgtcggcggcgcgcgcccagagctggcgctgcgcgcgctccacctccgccgcgcggcgcgcctccacggccgccacggccgcctccagctcgccgacgcgctggcgcgccgcgagcgccatcttctgcagctgcggctcctccacggcggccgcgcgcgccacctccagcgccaggtgccgcgcctcgaccagcgtcccctcgtcgggctgcgcgcgcagccgcccgagcatctccacggccgccgccacggccgccgacagcgcggcctgcagcgccgccacgcgctcagactcgcgcagcgccgcggcgcgctcctcgcactgccgcgccagcggctccaggaagcccagctccgggtgctccacggcggcgcggagcacccccgcggcgcgcgccgccttctccgccgtgtccgcgtccgccagcgccgccgccaccgcgtcggcgtgcgcgaggaagaagcgcgtgatcggcgggttgtccgcgagcgcgttgaccagcgcgccgagcggctgcccggcgcccaccagcgcgccggggtcggaggtcagcagctcgcggtagcgcgccaccagccgccacagcgccgcgaccaccgtgccgggcacgggcgtggccgtgatgtgcgcgagcaggtccaccgccgggcccagcgtggcaaagtcgccgagcagcgcgcgcagccgcatgtagaggtcgttctgctcggcggcgcgctgctgcgccgcctgcaccagcgtgcgcagctcggcgtcgcgccgcgcgagccgcgcgcgcgtctcctggctcagcacgtggccgctcatgccgcggatgtgccgcagcaggtcctccatcgcgcgcacgcgcgtctcctcgtccggggcggccgccagggcggcgtccatggcgtcgaagcgcgccaggtcggcgtcggccgaggcgcgcttggccgcgcgctcgttgatctggttcacgtcgcgcgtgagcgtgtccagctcgcgccgctccagatgcccgtgcgcctgcgcctcggtcagcagcgcgaaccaggccccgaggcgctcgtccgtgctcacgtcctcgcccgcctccagcagctgcgccagcagcgcgccgtcgtgggcggcgggcgccgcgtccagcgcgcgcagccgctcctcggtggccgccagcgagtcgacgaggcggcgcaggcccgccaccgcggcgttggccacgcggaagcgctgcgcgccgttgcggtccatcagcatggccttggcgctgtactgcgcgccgcgcagaaagtactcgcggatggcgtcgccgaccatcagcgccagcttgcgcggcagcttggccgcgcgctccttgacgtcgcgcgtgttcggctcggccgcgccgggcggcgcccccgcgtccacggcgtcaaagttggcgtgcgcggcctcgagcgccgcctcgagcgcgctcacgtcgcgcgcgcgctcgagcgcgcgctgcgactcgcgggactcgcgcgcccgcgcgcgcgcaaacaggccccgcgcgcgcgacaccaccgtctggtcgtagacgccgccggggtcccgcgccgcggcctccatgaagcatgccgagagctgcgcgtaggagcccccggactcgagcgcgtccagcgcccggtgcatggcctcgttcgcgggccgcgcgacgcgcgccgccaccagcgccagcttggccacgggcgcggacgcctcgccgccggccgcgtccagcagcgcgcgcgcctccgtcagcaccatgtgcgtggcggccacaaagtccccggccgggccgcgctccggcagcgcggccaggacgggctcgaagagcgccgcgacggcgcacggcgcctccgcgtgggtgcgggcgccgttctccaccagaaagtcctgcaggtgcccgaagaggagctcggcgaagcgctccagcagcccggcgcgcccggggaacaggctccggcccgcgcacgcgaccacgtgctcctcgagctcgtcggcgtgggcctgcagcgggcccaggtcgagcgggaccagctccagctcccgcacggggaagcccatgggctcgcgggtgcgcgggcggtacttgcgcgacggccgcccgccgaccgtgtgcgtgctgctcaggtcctcctggctcgccggccacgggggccggcgccgcttgggcatgctcggccgcttctggacccgcggcggcgcggcgagggacgagggctggggctgggactggggcaccgccgtcgagaggggggcggcggggacgggggcgacggcgaggacggggatggtcccgacggcggcgacggcgggggcgcccccggggccgcgcggcggcggcgcggcggccgccgggtccgggggcagggccatcgggtgcacggagggcaccgcccgctccacgtacggctcgtccacgaggtacgtctcgcccacgccgtagagcgcgggcacggcggcgaactggtcgtggggcaggaagaacacgaaggccccggcccagcggagctcgctggcggcgtagcccgcgacgtgcgcgtacacgtccgccgggcgcaggcgcgccacgaaggcctgcgtgatctgcccgtggccgtgcgggtcgaagacgtagacgacgtcgccgcggcggtagaggcccaggcccacggcgccgaccacgaccagcgtgaaggactcggcgcgctgcgcccagaggccgtcgaagaaggcgcgcgccgtgatctgcgtgctctggaagccctcgccgggcggcgtgaagaagttgcactcgccgtgcacccgcgagaagacgcagtgcagcgcggcgccgccggcgccctcgtagacgatcttgttcgggagctcgctgatggcgcacatgcccccgtcgggccccgtccacgcctggccctcccgcaggcacgcgtccacggcgtccgccgtcagggcggcctccacgccgttcgtgaagaccaggcgcaggaaggagagggaggagcgcaggcacgagacgcccgacccgggccccaggtcggggtcatactgattacgatagccgacgaccaccgcgtcggccgtcatggctgaaataacacacgcgcgtgggggaaaaatctttttatttggacacggcgtcgacctcggccaggagccgatccacgggcacccacgtttgcgcgagcaggggcacgcccgcgcccgcgtcgtcgggcgcgcgcgcgccgcggcgcagcacctccaggttcacggcggcccagtcgcgcacgttgacgctcgccgagagcggcacggcgtcggggcccgcgtcgccgtcggtcgccacctcgtggtagtcgcccaggacgcggtccgcggcctcgcgcagctccccgggcgtgaaggcggccgcgggcggcgcctgctcctcgcgcgcctcggtccaggtctcgcggagcgtcgcggcgagcgccagcacgccgcgctcgtcgacggcgcggcgcagcgcggcgctgaccaggtcccagcgcgccaggaagcgctccacgggccgcagcacggtggcggcctgcaggcggccggccgtgagggcggcgctcgtctgcagcttgctcaggcgcgcgtggtcggcgcacagcgtccggaacacgggcgcctgcaggcccagcgcctcgcactcgcgcagcgcctccgtcgtctgcgccatcacgccgcgcagcgcccccgcgtcggcgcgcatgtccgagcggtgcgtgttaacctcggcgtggaaggcgcccacggcgtccacgacgtccgccaggcgccacgtgcccaggcagggcccgccctcgccgggcgcgaagacggaggccgtgcgcccgcggaagacgaagcggcaggtgaagaggcgctcgagcgccaccacgcgctcggccacggcgcccgcggccgtggccatcgagtccagcaggtcgcccgtgtaggtggccaggtcctccaggcggcgccggagggcgcgcgccatcaggtccccgaggaaggcgctggtggccagcgcgggcggcgcgggcaccggcgcctcgagcacctcggcggccgcgcgcacgaccccgtgcagctcgtgcgcgagcgcctcgacgttgcgcgcgtgcgccaccgtcaggtcctcgcgcgccagcgccgccacgagcgcggcgagcttggtgcggtagccgctgacgttggggatgaggccggcgccgtagtcgcgcgcccacgtcagcacctgcgccagctccgcgcggcggtcgtaggaggccgtcaggaggcagttgtaggcgtcggccacggccgtcacgacgaagaagggcgcgaaggacgggtcggccatggcctcgaggtgcaccatggacgggtagttcacgaggtccgcggcggcggcgaggtcgacgagcgccgcgctcgtcggcgcctccatgggcgtctcgccggcgccgtcggcgcgcaggccccagcggctctgcgcgcggaagccgagccacgtgccgagggcctcgcgcgccgcgctgtgcgccagcccggcgcggcgctcggccaccgccacctcgcgcatcagcgcggcgcgcgcggcgcccacgtgctccgtgggcgggcgcggcgccatgcagtggcgcaccacgtgctccacgcccttctcggggagccccagcgccgccgtcacgtccccgtagaggggctccagcgtctccgcgacgtccgtgtaggcgcgcacgtagtccagcccgggcccgtagtcgtcgaggaggcggtgcacgacgccgccgacgaccgccgtgcagccgacggcgcgctgcgcgtccgtcaggcggggcgcgcggtccagcacggcgcgctcgaggaggtcgcagtggcggcgcgtgaagcccgcggtcacgagcgcctccacgacctgcagcggcgaggcgctggcctgcaccgcctcccacacgcccgtggcgtccacctgcgcgtggtcgaggtccgcggcggcggccacgtgcacggcctgcgccccggggtccagcagcgccagcacgccctggtccgtgcgcaggcgccgcgccacggtgcccgggcgcagctcgcgcacgtacgcgtccccgtgcgcgagggccgcggacacgcgcacctcgacggccgtgcgcggcgtggcgttgacctcgtccacgggcaccagcgccgcgtcgtccacggcccgcgccgcctcggcgaactgctcgcgcagcgtcgggttctggcggtcgtggaagaactcgtcggcgatcgtgagggcaaagagcgcgcgcgtcagcggcttcaggccctcgttctgcatctggcgcaccgccagcgccagcggcagcgcccagttggtgcgccgcgccacgtggcgcaggccgtcctgcacaaagtccatgtcgtagagcgtggcgaagcggggctgcacgagcgcgcgcgccgtctgctccgggggcacgacgcggggcgcggccaggacgtcgtcggcggagcgcgcgcccgccagggcctcgtcgaaggcggcgcaccactcgctcacgaggcggaacatggcgtcggggccgaactcggccgaggtgcgcagcccggcctcggtgagcgcggccgtggtggcgtcggcgttgacgacccccaggagctccagcagcggggccgtgccctcgtcccagtcgtggcgcaggcgccacagcgccaggccggccaggttctcggcgagcacggccgcctcgcaggtgccggtcgcggcgtaggcgcggcaggccaggcgcagcgcgcgcgcccagagcccgcgcacccccgcgggcgtctcgcggcccggggccatgaagaactccaggatgtgcgactgcaccaccgtggcgacggcatggtcggcctcctcgagcgcgcgcacgagcgcctccattataagagggtccgcgcgcgggcgccgccgcgcactcgaccccgcgcggcgcgacgatgagcgtgcagatcggcaacgggctgctgatggtggtcgcgccgggcacactaaccgtgggctcggcgcgcgcgcgccttatacgccaggtgacgctggcggacttttgcgagccccaggccgagcgccccgggctggtggtgctggcgctgcggcaccccgcggacctggccggcgccgcctacgcggccacgccgcccggcaagaaccaccgcgacctggaggaggcgtggctggccctcgacgagggcgggcgcggcctcggcggcgacggcatccgcgcctccgtcgtctcgctcaactttttggtggcggccgccgagaacggggacgacgcgctgcgcgcccacgtgacgaccaactaccgcgaccggcgcacggccgcgcgcctcgagcgcttcgcggccgtgctgcgcgccatgatccggagccacgtgttcccgcaccgcgcgctgcacgtcctcggagggctcctgggccacgtgacgcaggaccggctggccagcgtcacgtgcgtggcgcgcggcgaccaggaggcggcgcgcaccaacgacatggccgcgcgccgctcgcaggtgcacgtgcccgcgtgcgcgctgatggacgtggaccgcgagctgcgcctcggcggcgacgacgggctccgcttcgtgtacctggtcttcgtctacacgcagcgccaccgccgcgaggcgctgcgcgtccacgtggcggtgagccgcctgcccgagctcggcgacgccctcagcttcctcctggccggcacgcgcgtcgacaacgcgatccacggcacggacgaggccgacgcccccgccgcgcccgccgccgccgcggccttccccgcgtacctgttcaacgacccgcgcagcgcgcgctgcccgacgggccggctcaacacgcccgccgccgaggcgctgcccgtgtgggcccccgacatgcgcggccgcgccacccggaactcgtgcatgtacgccgcctacgtgcgcctcggcaccgtcgagcgcgtcgtgcgccgggccgagcgctgcggctcggtggacctgccgctggcccacatggagcgcttcacctgggacgtgggggcgtgggaggagtgtttcttctgaaaaaaacggggggaggtgtgcgtgagacgggtctgatgttgctgacgagactaataaaaagcgggggatacgcgcgcgcgtgtgtccccccgttgacccttcatgccgcgtccgcgtcgtgtgtgactcacccccatcgtctctcccgcccgcgatcccggcccgtcccgactcgtcccgcccgcccagacgcgtcccgtcacgcccccgcgcacgccccgcccgccgggcgcggttttcccccctttccccccattggcggcctccgccgctaaaagcccgcgcgccccgagctcgggactctccgcgcactccccgtttcgagcacacaccgtcatggcctcccccgtcgtgcccgccgccgccgccgccgctcccggcgcggactccttcctcgacgccgtctgccccgaggacgtcgcccgggccctggccgccgagctcgaggcgctccgcgccctcgggcacgacgtcggcgcgcccgcgcccggggcctcgcgtcgcgaggtggcgctgtttgtcacccgcgccgtggacgggctcaaggccttctcccgggtggacgagcgcgtgtacgtggcctgcggcaagctggtgcacctgcgcgtgcgcagccgcgaggcggacctggacgcctggctcgcctcgccggagctcgcgctgatccccgcggtggcgggcgccgtgcgccggcaccgcgcgcgcgtggaggcggccctccgctggttctggcgggaggcctacccggcgctctacgcgcgcggcctgcagtcggcgctcaagtacgaggagatgtacctggcccgcctcgagcacgggcgctgcgaggccatggaccagttctttgtgcgcctcgccgccgccgccgccacggcgacgcggcgcgccatggcgctcgtgctctgcggctcggacgcgtggccggaggtctttgacgcgtactttcgcgcgctggccacgcaggcgatcgtgcccgccaccccgctcatgctcttcgccggccgcgcgcgcggctcgctcgccagctgctacctgctgaaccccctgccgcgcaccaccgaggaggcggtgcgcgccatcacggacgaggtggcccccatcctgctgcgccgcggcggcgtcgggctctcgctgcagagcttcaaccgcacgccctcgggcgactgcacgcgcggcatcatggccgtgctcaaggcgctggactcgatgacggcggcgatcaacagcgacagcgagcgccccacgggcgtgtgcgtgtacgtcgagccgtggcacgcggacgtgcgcgccgtgctgaacatgcgcggcatgctcgccgccgacgagagcctccgctgcgacaacatcttcagctgcctctggacgccggacctgttcttccagcgctaccagcggcacctggacggcgagcgcgccgtcaagtggacgctctttgacgaccgcgcctcgcacctgacctcgctgcacgggcccgactttgcgcgcgagtacgagcgcctggagcgcgccggcctcggcgtcgagtccctgcccatccaggacatggccttcctcatcgtccgcagcgccgtcatgacgggcagccccttcctcatgatgaaggacgcgtgcaaccggcacttccacacggacacgcgcggcgccgccctcgccacgtccaacctctgcacggagatcgtgcagcgggccacgcccggcgagaacggcgtctgcaacctggccagcgtgaacctgcccgcgtgcctggcgggcggcgccttcgactttgccgcgctgcgccgggccgcgcgcgtcgccgccgtcttcgtcaacgccatgatgcgcatcgggaactacccgacggaggcctcggtcgagggcgtgcggcgcagccgctcgctgggcatcggcctgcagggcctgcacacgaccgtgctggcgctggacatggacatggccgacccggcggcgcggcgcctcaacgccgccatcgccgaggagctgctctacggcgtcatggacgccagcgtggagctctgcgagcgcggcctgcgccccttcgacggcttcgagcacagccgctacgcgcgcggggtcatgcccttcgacgcctacgagcgcgtctcgctccgcgagccgatgcgctgggacgcgctgcgcgtccgcatcgcggagcacggcgtgtacaacgcgcagtttgtggcgctgatgcccaccgtgtcctcgtcgcaggtcaccgagagcagcgagggcttctcgcccaccttcaccaacatgttcagcaaggtcaccatctcgggggagctcctgcggcccaacctgccgctcatggagacgctgcggcgcctgtttccgcgcgagtgcgcgcgccgggacgccgtggcgcggctggagcgcgcgcagtggtccgtggccgcggccttcggggagctgcccgccgggcacccgctggccaagttcaagacggccttcgagtacgaccaggagctgctcatcgacatgtgcgcggaccgcgccccctttgtggaccacagccagtccatgtccctcttcctgaccgagcccgccgacgggaagttacacgcctcccgcgtcatgggcctcctcatgcgggcatataacctgggcctcaagacgggcatgtactactgcaagatcaggaaggccaccaacaacggggtgttcaccggcggggacctggtctgcacgagctgccacctgtgagcgcgcgccatggagtacttttacacgtcccagtgccccgacatggaccacctccgctcgctgagcgtggccaaccgctggctcgagacggacctgccgctgggcgacgacgccaaggacgtggccgcgctcagcgaggccgagctcgagttctaccgcttcctcttcgccttcctctccgccgcggacgacctcgtcaacgtcaacctgggcagcctctcggagctcttcacgcagaaggacatcctgcactactacatcgagcaggagtgcatcgaggtggtgcactcgcgcgtgtacagcgccatccagctgatgctgttccgcggcgacgccgcggcgcgcgagcgctacgtgcgcgcggcgctgcgggacgaggccatccgccgcaaggtggagtggctcgactcgcgcgtcgccgagtgcgcctccgtcgcggaaaagtacctgctcatgatcctcatcgagggcatcttcttcgcctcctcgttcgcctccatctcgtacctccgcacgcacaacctgtttgtggtgacgtgccagtccaacgacttcatcagccgcgacgaggccatccacacctcggcctcgtgctgcatctacaacaactacctgggggacgccccgcgccccgacgaggcccgcatccaccagctcttcgccgaggcggtggagatcgagtgcgagtttctgcgggcgcgcgccccgcgcgacagcctcctgctggacctgccggccatcatctcgtacgtgcgctacagcgcggaccggctgctgcaggccatcggcgcgagcccgctctttggcgcgcccgcccccgcggcggactttcccatggcgctgatggtcgccgagaagcacaccaactttttcgagcggcgcagcaccaactacacggggaccgtcgtgaacgacctgtagcgcccagcggcggcgacccccgctctcccccctcctcctctctctgcgacaaaacactaataaagcgttgagacactagcgcgcgcctccggccgtcatccttggggcgagggtgggatggggaggtggaggcgggatgggagagtgtggggagagtgtggggagagtggggtggggagagtgcggggatgatgacgggggaagggagatggggctgacgggggtggactgggactggggaaggatgacggcggccggcgcagagtgggaggcggtgaggtggacgggggacggaggggaggaccgatgtgggacggcagaggggcggcagaggggcggcagaggggcggcagaggggcggcagaggggcggcagaggggcggcagaggggcggcagaggggcggcagaggggcggcagaggggcggcagaggggcggcggcgaggtgagacggagcccgcggacggagtggccggcggggggaggcgacgacggagagagaaggtgggggagagggcgagcatcacacgggccgcacaaccgaggatttttttcaagttgttttttattttctcctatgggcgttacagtcgtcccagtacgtcatgaggacggcctggtagttcggaggcggaggtatgtgcttccaaaacaggcccgccagctccttcgcccgcgtctcgttcttcacgtgccgcagcagcgagccaaagaccttgttcacgtccgagggctcctgcacgatggggacgcgccgcagcacgctcaggtgcccgtgccgcgccggcgtgagcatggccaccacgtgctggatgaactcgcgctcggcgcggcgcgagcgcgcgtccccggccgccggcagcagcgcgagcgccttgccggcgtcgcgcatgatgtcggggcagcgggacgcgtacttgagcttgacgtcctcgggcgccacctcgcggccctccagcgactcggccacctgctgcaccgagtccacgtccggcgcccggtgcaggtccgtgtggcagcgcacgaacgcggccaggaactccgagtagtccacccccaggctcgcgaggacgtcgcggcagcgcagcaccagcgggaacatgggcgcgatgtcgaggatcatgtcgcacccggtgaggatcatgtccgtgtcggtcgtgtgcacctgcgcgaccgtgttggtgtggtagaggttggcgcagacgtcgtccgcctccatgtccgacacgtccacgtaggcgtagcccatgtggcggatgaggttgacgcagaggcggtggacgatgcgcggcgcgtgcagcatcgtgctccagcgcggccgcggcggcgcgtcggcggccccgtcgccgtcggcgcgcgccgccgcggccatgatggccttggcgccgtgcgcgacgcggccgttcccgaagatgccgcggtccgagacgaagatggggaagtaggtgcgcttgtgcagcatgcgcagcaggcgcagcaggcagcgggcggtcgtggtcgcgttgtcctcggtcgtctcctggtagtgcttctccatgagggtgtacatgacgttccacaggtcgatggcgatgggcgtgaggaccccgggcggcgtggagatggcctccgagcggaccagacgatggcgatgtgcgtactttaaaaggccaaagagccccatgtctccgctcgcacaccgaggcgccggtctaaaatacgcaggtggcggggcggggccacccgcgcatataagccgggcggtgattggtctggcgcacaccgcccgcccgagcccgctcgcccgccgcgatgtcgctgttcgacgacggcctcgaggacctggaccgccaccccacccacgcgcaccacccggcgcaggtgatccacgacgggcccttcgtgctggaggacggcgagcccctgcagcgcaccggcatgctggtgctcagcgacgagcacctggagcacgcgcgcgccgccatcgccccgctcgccgcgcacctcgcgcacgccttcctcgtcttcagcgaggccgggctgctggtgcacgccagcgtgcgcggcgagcaggtctacgtgaccctggccccggaccagttcagcacgttcgtgtggagcgggccccaggccgtgttcctgggcaacgtcgacggcagcggcggcgtgctcgacgcgctcaaggtcgaccggcggcggaccgtcttcaacgtcaccttcgaggtgtacggcgccttcccggcgcggctgctgacgcggcgcgcgtactttgcggacgcgggcctcctcgcggcggggcccggctccccgagcgtcgcctgcgtctacaagcacgagttcaacgactactgcatcatgctcccctcgcgggcgcccgacgtgagcctgacgctctcgcgcccccaggtggccaagctcgccgccgtggcgaagggcgccgcggccgggacgaccttcgcgctcgcccgcggcctcgacttctccgtctcctccagcgccggggtcgtgaccttcccggcgcgcgaccacgacgggaccgccgtgctggagcgcgccagccggcggcgccagggcgtcgacgcggtcggcgcgacggagcccttcgccatgacgctcgaggcggcgcacgggctgctgacgctgctgcagcggctgcgggccgggaacgccgagctcacgttcaactttttcacgacgccgcggcaggcgcccctgttcagcgtgaccacctgcggcccggtgcgggcgaccaccttcttcttctgcgcgcccgccgaccccgccaccgtgcccgccgcccccgagggcgccgccgccaccgtcgccgccgcctgcggggcgggcgcgtccgccgcctcccccgccgcgggggacaagcggcccgccgccccgcgcatgtacacgcccatcgccaagcgcccgcggaccgcctcgggggaagggggccacgcctacggagatttattctaataaagtgagcaggtgtataaaagagaggtcgcacctccgcgtctttactcgccgtcgcgatgatctcccgggggaacggagggggttgccgccccggcgagccatgctggcgctgcgcgctcgagtccacccgctgcatcacgctgatgggcgtgctcgtcgcgctcctcgccgcctgcatgctgtccgtcccgcccgcggcgtcgacgatgctgctcggcgtcgccagcctcatggccatgctgcgcctgcccatgcccctcgtggaccggttcatccccgcgtgcatgggcctccagctcgtcggcgccgccgtcttcgccgcgggctgggcgctggccagccgcgacgcgatctcggccggcgtcctgctgtgggccgtctgcgcgctcatctcgcacatgtacaacgtcgtctgcgtcgccagcgggcccgacgcgcactaccgccccgcctgcctcgtcatgggcgtcgccgcggcctgcggcgcggcgggggccctcgtgaacgtgcgcaccgaggcgcgcctcggcatcgccctggggctggccgtcacgtgcgccacgaacaacgtcgcccgcagcctgcgcggcacgtgcacctacgtcgccagccgcgcccggttcctggccgcgcccgcggacctggggcgcggctacagcgtggagaacgcggacgccgaccccaccgccgagcccgagcggcgcgtctacgaggcgaccgtcccgcccacccacgtctacgccggcagcatcgcgctcttcgccctcgtcttctcggccgcctcctccctccagtggatggtctcccagatggtgggccgcggcaaccagctcgtctcgcccaccaccgcggccgcggccggcgccgccgggttcctggacgcggcggcgctctcgctctttgtgcgcccgagcacgcgccacctctcggtggccgtcaagggcgcgcacgcgctcctcatcctcgcggccatcgtcctcacggccgccggcgagcccatgggcgtgcccatcagcctcgcggcctccaccggcctcgcgctgctcgaggcggaccacgtccgcctgcgccacacccgcgcgtaccggctcgccgccgcgcacgtgacgcgggccctgctcgtgcaggcgtacgtgaccgtcgccatgtgtgccactagcattaaatccgtttcctgattcacgcccacgcccgtgtcgtttttaaaaccgcgatggggggacggggggccattcgcacgcgccatggcatcgctcgcgcgtgcgatgctcgcgctgctggcgctctacacggcggccatcgccgcggcgccgtcgtccacgacggcgctcggcacgacgcccaacgggggcgggggcggcaacagcagcgcgggcgagctctcgccctcgccgccctcgacgcccgagcccgtctcggggacgacgggggccgcggcctccacgcccgccgccgtctcgacgccccgggtcccgccgccctcggtctcgcgccggaagccccagcggaacggcaacaggacgcgcgtccacggcgacaaggccacctcgcacgggcgcaagcgcatcgtgtgccgcgagcggctgttctcggcgagggtgggggacgcggtcagcttcgggtgcgccgtcgtcccgcgcgccggggagaccttcgaggtccgcttctgccgccgcgggcgcttccgctcgcccgacgccgaccccgagtactttgacgagcccccgcgcccggagctcccgcgggagcggctcctcttcagctccgccaacgcctccctcgcccacgcggacgcgctcgcctccgccgtcgtcgtcgagggcgagcgcgcgaccgtcgccaacgtctcgggcgaggtgtccgtgcgcgtggccgcggcggacgccgagaccgagggcgtctacacgtggcgcgtgctgtccgccaacggcaccgaggtccgcagcgccaacgtctcgctcgtcctgtaccaccagcccgagttcggcctgagcgcgccgcccgtcctcttcggcgagcccttccgggcggtgtgcgtcgtccgcgactactacccgcggcgcagcgtgcgcctgcgctggttcgcggacgagcacccggtggacgccgccttcgtgaccaacagcaccgtggccgacgagctcgggcgccgcacgcgcgtctccgtggtgaacgtgacgcgcgcggacgtcccgggcctcgcggccgcggacgacgcggacgcgctcgcgccgagcctgcgctgcgaggccgtgtggtaccgcgacagcgtggcctcgcagcgcttctccgaggccctgcgcccccacgtctaccacccggcggcggtctcggtgcgcttcgtcgagggcttcgccgtctgcgacggcctctgcgtgcccccggaggcgcgcctcgcctggtccgaccacgccgccgacaccgtctaccacctcggcgcctgcgccgagcaccccggcctgctcaacgtgcggagcgcccgcccgctgtcggacctcgacgggcccgtcgactacacctgccgcctcgagggcatgccctcgcagctgcccatcttcgaggacacgcagcgctacgacgcctcccccacgtccgtgagctggcccgtcgtgaccagcatgatcaccgtcatcgccggcatcgccatcctagccatcgtgctggtcatcatggcgacgtgcgtctactaccgccggtccgcgctgtgacgcccccgcccgccccgaatcaataaacgaccgcgcgttgaaccgagccatcgcgtccggttgtttgtttgtgcgtgtgtgtgtgagctctcgcgcgtccccgggccccgctctctcccgtcccactccactctcctctcccgcaccctccccacacacacgcccggcccgcaataaaccacacgagacgacggcgtgttgtaccggcgcgaccgggccatctctttattggtcgtcggggaacagggacctggccggggtggggggtgtgggcggcgggcgagcgacgccgtcctcatcggtgggacatcatctgggagacgaagatgtcggcgccgcgctggcaggccacggcggccgagctggcgtcgatggtgccggcggagacgtctgccggggccgagaccgaggcctggacgggcggctggggggccgaggcgggcgcggccggggcggcggcctgaacgggggcggccgcggcggccgccgctgctggagccggagcgggggcggccgcggcggcggcggcggcgggcacggccaccggctgggcctgcaccaccaccggctgctgcgggaccacctgggcggggacgtgcgcgacgggggccgccagaccctggaggcccggcaggcccgggagcgccggcacggcgtgcgccacgggcgaggggggcggcgcgtgcagccccggctggtgggcgtacatcggcgggtacagcgccccggcggcgtggtgcgggtgctggtgcgcgtgtcccacgacggccccggggggcaggccgggcggcagcagctgcggcggcgtcgcgggcgcgtagtacggcgtcgggtagcggagctgctgggcgcgcaggtggctgatctcgcgctggagcgacgagaccacggcggcgaggtcgctcgcggggggcggcgcctccccggggtagtacgcgttgtcctgcgcgtagtcgtcgtagcggcgcttccgggcggggggccgcggctcgggcgagggcgagcggcggtggcggcggtccatggcggccgagacgatggcgccgagctgggactccaggctgggcgccgggcgctggttcaccaccagctggttgtactgcgccgcgggcacgaagatgtagtcgccgggcgctgcgctcggcggggctgccatggccgcactgggcccgggcgccggggaggccccgcttttaggcagaagccccgcccacatcgtcgcctgcaggtaggtgtgtccgcgcacgccggcctcgcggcggcgcgccgcgaccagctcccagcggtcgcgcagcagcatgttgttcacggccgtcgacagcagcgcgcgcgtgagcgcctcctcgctcatgtgccacacgcgctcgcggtccggcgactcggcggcgcgcgccagcagctcggagcgcgcccgcgccgagagctgccggaacggcgccacggcggcctcgggcgaggcgtcgtacaccacgatggtgcccacgcgccgcccgatcacgcagagcgccacgtgcgcaaagagcgtctcgtcgggggcctcgccgggcgccaggcgccgcgaggacagggacgccgagggcaggtagttgctcagcaggtacagcagccgctcctcgtccgagagccgcatgtccccgaagaagtccgggcccacggcgcgggcgagcacggcccccagctgcgggcagttgacgacgccgaggaagaaggggccgcggtcgtcgtcgaccacggccagcacggcgccgatgtcgcagcgcgggcggtggtcgatgttgatgggcagcgggcccgccgggggcagcgcggcggccacgacctcgcgcgtcagcgcgagctccccgccgtccctatcgtacagggccaggtagccggacacgtacacgggccccatggcggcggcggtggcgacggcggcggcgcgcgcgcggaggagactgcgggctcaggcggcggcgaactgcgggatatagcccaggcagagaaagtacaggaggtcgtagtcgctggagacgccaaactgcccgaggcgcttgtccgccgcgttgcgcaggtgcggctgctgcagcagcaggcccagcccctcctcgtactgcacggccacggcgtcgtgcgccgcgatgacctcgttgacgggggccgtcacgcgctggcggttctgcagctccagcgccaccaggcgcaccaggctgagctggtggcgctgcacgcgcgcgcggatcgagtgcgcgtccagcgccagcgcggccagcccggggaagagctgcgtgagctccgtgcgctcgtccgcgcggtaggccacgacgacgtagtgggccagcaggaagcgcaggttgccgtcgccgctgcgcgaggcgggcgcgtccccgctggcgccgcgcggcggcggcgcgttgggcaccagcgcgccgagctggaagttgttgcgcaggcggtccccgtacacgttgccgttgtgcagcaggcgccgcagcagcagcaggcccgtgatggtgttcaccgtggcgcgcagcagcggcgcgtcctcgccgaggaagaggttctggccgcggcccaggaaggcggccgcggcgcggttgacctcgtccacgaagcccgcgccgtcgtcgacctcgttggcgccgacgttcgcgccggggaccacgcgcaggcgcgccagcgcggcgagcacgctgtggccctccagcgcgtagcgcccgtggagctgctcgcgcggcacgcgcgcgtagttgtagcggtacgagatgcgcccgccctcggccagcgcctggctcagcgcgtccaggtactcggggatccgctccagcaggtccacgatgcccagcggcgcgggcgccgccgccgcggcggcggggccctgccgcgcgcggtgcaccaggtgcaggcacagcgcggcgctctcgagcgccgagtagtgccggttgcccacgtacaggcgcccgcaggcctgcagcgccgtggtcgtcacgcccatgaaggtgcgcgacatgcggccgtcgcgcgagtcgatggcgcgcgagacctgcgggcgctccgcgacgagcgtggcctggagcgtggcgtaccacgtgccgaagagcacgccggaggcggcgccgcgcgcggcgtacaccatggccaggaagtccagcgagaggttggcgtcgtaggcgagcgccacgtcgttcttggcgatctgcacctcgcggccctcgtccgcggccgcgaccgcctcgggcgcctcctcggcggcgcgcgccgcgtccgcctcctcggcggcgcgcgccgtctcctcgagcaccacgagcgcctcggccacgcgctccacctgccgctccagcggccgcagctgctcgtccacctcggcctcgaggcgcgcgcccgcggccatggcgttgtccagcgccgcggcggccgcgcggcggcgcgcgttcgcgtgcgccagcgcgaggcgcgcgtcgaggccctcgccgaagcccgggcgggcccagaagcccacggggaacgggggcgcgatgaagtggcgcgcgctgcccgggatcgcagcggcctcgaaggcgaaccacgcgcggtccatggcgcggggggacatgggccgcgcggcggcgcgcgccgccttatcatccccgctccccgccgccgcccggcccggccccgcgcgcgccgcgatcgcgatcaccgccgcggcccggcgacgtactcggcgaggccgcgcacggtcgcggccatcgcgctcgcgttgccgcgcgtctgggtgcagggcaggcgcgtcacgtcgagcacgcgcatgctccgctgggccacaaacaccagcaggggcacgagcgtgatctcctcgccgcccgggggcacggcggcggcgaggaggcgcgccgagtcgcgcagctggcacagcccctcgtgccgctgcccgcgcttgctgggcgtgttgaggttccgggggaagcggcacgtcttgagctcgatgaggaagcacaggtgcgggcccgcccccagccgcaccacgcacacgcagtcggggcggcgcaccccgaggttgacttcaaaggccagggtcaaggacgccttcttaagcgtctctcggggaagcccgaagagactctcgccgtacgcggacgggtcgcgtcgcaggcgttcgtagaagcggttgtggcagcggatccccgcccggaagcgcgccgggatgcgcatcctccggatctacctcgacggcgcctacggcaccggcaagagcaccacggcccgggtgatggcgctcggcggggcgctgtacgtgcccgagccgatggcgtactggcgcactctgttcgacacggacacggtggccggtatttacgatgcgcagacccggaagcagaacggcagcctgagcgaggaggacgcggccctcgtcacggcgcagcaccaggccgccttcgcgacgccgtacctgctgctgcacacgcgcctggtcccgctcttcgggcccgcggtcgagggcccgcccgagatgacggtcgtctttgaccgccacccggtggccgcgacggtgtgcttcccgctggcgcgcttcatcgtcggggacatcagcgcggcggccttcgtgggcctggcggccacgctgcccggggagccccccggcggcaacctggtggtggcctcgctggacccggacgagcacctgcggcgcctgcgcgcccgcgcgcgcgccggggagcacgtggacgcgcgcctgctcacggccctgcgcaacgtctacgccatgctggtcaacacgtcgcgctacctgagctcggggcgccgctggcgcgacgactgggggcgcgcgccgcgcttcgaccagaccgtgcgcgactgcctcgcgctcaacgagctctgccgcccgcgcgacgaccccgagctccaggacaccctcttcggcgcgtacaaggcgcccgagctctgcgaccggcgcgggcgcccgctcgaggtgcacgcgtgggcgatggacgcgctcgtggccaagctgctgccgctgcgcgtctccaccgtcgacctggggccctcgccgcgcgtctgcgccgcggccgtggcggcgcaggcgcgcggcatggaggtgacggagtccgcgtacggcgaccacatccggcagtgcgtgtgcgccttcacgtcggagatgggggtgtgaccctcgcccctcccacccgcgccgcggccggatggagaccgcgacggaggcaacgacgacggcgtgggagggggctcggggcgcgtataaagccatgtgtatgtcatcccaataaagtttgccgtgcccgtcaccatgcccgcgtcgtccgtgcgcctcccgctgcgcctcctgaccctcgcgggcctcctgaccctcgcgggcctcctggccctcgcgggggccgccgccctcgcccgcggcgcgccgcagggtgggccgccctcgccgcaggggggtcccgcgcccaccgcggcgcccgcgcgcgggcccaccctgttcgtcctggacggcgacggctccgcgtggttcgtcttccagctcggcgggctgggggcgctcaacgacacgcgcatccgcgggcacctgctcggccggtacctcgtctcgtaccaggtggtgcccccgcccgtctccgcgtggtactttgtgcagcgcccgcgcgagcgcccgcgcctctcggggccgccctcgggcgcggagctcgtggccttcgacgcgcccggcgtccggcgcacgtacaccacggcggcggtgtggcccgcggaggtggccgtcctcgcggacgcggaggcgcgctgccccgcggccgtcttcaacgtgacgctgggcgaggccttcctcggcctgcgcgtcgcgctgcgctccttcctgccgctggaggtcatcatctccgccgagcggatgcgcatgatcgcgcccccggcgctcggctcggacctggagccgccgggcccgcccgcgggccgcttccacgtgtacacgctcggcttcctctccgacggggccatgcaccagacgatgcgcgacgtggccgcctacgtgcacgagagcgacgactacctcgcccagctgtcggcggcgcacgcggccgccctggccgccgtggtgcagcccgggccgtactacttttaccgcgcggcggtgcgcctcggcgtggccgccttcgtcttctccgaggcggcgcgccgcgaccggcgcgcctcggcgccggcgctcctgcgcgtcgagagcgacgcgcgcctgctctcgcgcctgctcatgcgcgcggccggctgccccgcgggcttcgccgggctcttcgacgggcgcgccgagcgcgtcccggtggcgcccgcggaccagctccgcgccgcctggaccttcggcgaggacccggcgccccggctggacctcgcgcgggcgaccgtcgccgaggcgtaccgccgctccgtgcgggggaagcccttcgaccagcaggcgctcttcttcgccgtcgccctgctgctgcgcgccggcggccccggcgacgcgcgcgagaccctgctccgcaccacggccatgtgcaccgcggagcgcgccgccgcggccgccgagctcacgcgggccgcgctctcgccgacggccgcgtggaacgagcccttcagcctgctcgacgtcctctcgccgtgcgccgtctcgctgcgccgcgacctcggcggggacgccaccctggccaacctgggcgccgcggcgcggctcgcgctggcgcccgccggggccccgggcgcggcggcggcgacggacgagggggcggaggaggaggaggaccccgtcgcgcgcgccgcgcccgagatccccgccgaggcgctgctcgccctgcccctgcgcgggggcgccagcttcgtgttcacgcgccggcgcccggactgcggcccggcgtacacgctcggcggcgtggacatcgccaacccgctcgtgctcgccctcgtcagcaacgacagcgccgcgtgcgactacacggaccgcatgcccgagtcccagcacctgccggcgacggacaacccgtccgtgtgcgtgtactgcgactgcgtgttcgtgcgctactcctccgcgggcacgatcctggagaccgtcctcatcgagtccaaggacatggaggagcagctcatggccggcgccaactccaccatccccagcttcaacccgacgctgcacggcggcgacgtcaaggccctgatgctcttccccaacggcaccgtggtcgacctgctgtcgttcacgtcgacgcggctcgcgcccgtgtccccggcctacgtggtggcctccgtcgtgggggcggccatcaccgtggggatcctgtacgccctgttcaagatgctctgcagcttctcctccgagggctattctcggttaataaacgccaggtcgtgaggcccgcgccgggccgcgaacccagactctctgcgtgcgcgtgtttttccttgtcgggcgcaggggagacgggaggggggagagagacggcacgggcgccgtccgtcggggagacggcgggatgacattacgagagtcgggtggaggggatgcggggagcgccgtccacgggggaaggctgctggatgacataattagagccaggggtgaggatcccctttcccccgttccccgagtatacaccactctctcccccgcacacatttcccaccgggcgccgaacgtggattgcaccccggtccgcgctacccggttgcgtgtgagacgggcggagagccgccctcgccccggtcccccaaaaactcctgcggatgcgtggtccgacgaagcctccgcggatatgtccgccgtaccctcatccccctctctcccacagccctctctcccacagccctctctcccacagccctctctcccacagccctctctcccacagccagactcccgcctcccccgtccccgatcgccgaacgcgttcaagaattttttacccgctccgaggaaggggtctctaaggggtctctaaggggtctctaaggggtctctaaggggtctctaaggggtctctaaggggtctctaaggggtctctaaggggtctctaaggggtctctaaggggtctctaagggggaaccagagagtaaagaccggagagtaaagaccagagacacattttacaatttcgttgtacccgggtttattgaaaactttgttcaagagttaaagtgtctgccgcttgccggtgcgccgcgccgaggccgcgatccgtccgccgtggaccgggggatccgtggggcccgcgggcctggattcgacgggacggcgggtccgagcgacggcggtccggcggcgtttcggctcgcggtcctcgcggcggcgacgatcctggtccgggggatacagcggcgacggcggcgctccgggttgtggcgccgggctccggctccccggtcttttatacccacacctcgtgaatattcatgaatggttggtatgtgcgaatttttcggcgttcgcacttgttgtatataaaatattatatcctatatatattagcaattggtgcgaacgtgacgtggcccaatcattttcctcggatttgcccatgtgaccgagtttccaatagctccgcccggatcgatcaccgggccgatattttgccgataatccatcaatattttcttcggccgacgcgggttccacccgggccgtcccgccggggagcccggcccgggcgcgggactctgctctccgggcgccgcgcggcgtcgagaccgcgccgggacgatggcgtctgcgcatgcgcgggtggaggcgagtggccgggaatcccctctcgtgccgagccccgcccatcgtcgcgtgtcctctccctcacccccttctcccccgaccgggcgacgatgggccgcgtcctcgccacgatggaggagaggcgctcacgcacgcgcgggtacgcgggagttgcggggatcggggaccgggacgatcggggtgtgcggaccgtggcgcgtcggggggctgggtgcgagcgggggttgggcgcgcgcgtgtgtgggggggggggacgggaaactggggacgggggaacccgccgaggcgcgcgtgcgcgcgcacgcgtgtgtgtgccgcggacagagacggacgggagaacggacgtgtgatttgtttaacggtttttattgaggacgatggagatgttggcgcgggccaggcgcgcccgaaacagctgaaacaggtactgggccccacggagggccgcgggcgagtcgccgaaggcggtcaggagcgccgcctcggcgcagacggcggacgtgagccgcagcgcgcggcccacgggcgcggtcgcggcgacgaggccccggccggcgtagagcccgtccagcacccgcgccccggccgcctccaccagctcgcacacgccgggcgccggcgcgttcgcctccgcgtcgctcatcatgaccgccagctccaggcccgtcgccgcgtcctcccgggcgtgcttcgtcgtcggcggtgaggcccccgccggggccgcggagacgatgggcgcgagcgcgggggcgtgcaccacggcggcgttcacgagcatccgcatcacctcgccgaagacggtcgcctcgcgcagcacgcggttgacggcgtgggccacggcgctcgcggcccgagaggcgccgccgtcgccgttgccgttgcctccgccgcctcccccctcgccgccgtcctcgtcgtcgcccgcgcccgccccggggcgctcgtccagcgtgtcgagcatgccctccagcgcggcgtagcagctcggcaggtgccagcgggccaggatgaactggtacagcgcgaaccgctgcgcgcccgtgagcgacgtggagcccgtcccgaagaaggggtccgccgcgacggcggacacggcctcgaccacggcggccgaggcgtcgtccgaggcgcacgcggcccgccgcaggatggcgtccgccccggccacgaggtcgcagagcatctgcagggacaggctcccgccgcgcgtcgcccagatgcgccgcgtcgccgggatgtagcgcacctgcacaaagtcgctgaccaccgtcgcgcgcagcgcgcgctcggcgtaccggcgcgggcgcacgctcagcgtgggggccgcgcgcccgcccttcaccaccacggccgtgggccgcgtctcgccgctgagcggggggcgcacgccggggatgccgtcaaagagccccgccgtcagcacctgcagcgagctgggcagctcggagaggtacgcctgcaccagggcgaagcaggcctcgttcacggcgtacacgaagcccgaggaggggtgcacgatggcgggctcggccaggagctccaggacacggtcctgctggcggtcgtgcacgagcgtcagcgagagcgccgacgccggcagcgagaccatgcactcgacgaggtaggggtcgtacacgtccagctccgcgcggccgcagaggtccagctccgtcagcggcagcagcagccccagggcgtccacgaagacgtcgtcgctcccctgcggggcggcccacgcgcggccgaggcgcgccagctccgtgcgcacgtagttggcgacgacgcggttccccggcgcgttcccgcgcagcgtgagcccgaacttgccgatctcccgcgagcgccggtgcacggacacgatgcagcccccgtgcacaaagtacgcgcggtccccgccgtcggcgatgtagaacagcaccccctggtgcacgatcgtgctctggtactcaaactccatggcggcggacacacgcggcgcggcaaatgggtgagcgaggcggcgcgaccactcctttaaacccgccggggggcgcctcccgccgcacacacgcggcggcgcggggatggaggacgcggcggcggtggacgtggacgcggcgtcggacgcgacgacgacggcggcggtgggggagaaggacgcgctgctgagctcggcctttgtgggcgcgcgcccgccgcgcccgcgcttcagctcgcacgtggtgagcctcctggtcctggcgctcgcgctccggcccgcgtgctgcctcgtcctggcgctccacggatcgcgggccaccctcgcggcgctcctcaccgcgctggccttctacgcgcgcgcggccgtctgcgccgccctcgtggcgcggaacgtggcccgggaccggatgccgctctcgccggcgcagcaggtggcgctggggctgctggccgcggcgcggctggccttcttgtacgtcgccctggacgcgggccgccattacgcgcccgccctcgccggggcgctgtacggcgccgactgcgtctgtgacgcgctcgccttcctgctgccgcgcgcgtacgcgcgctccatcatgcattaaaaggcggccgcgacgcgcccgcgccattcttcccccgccgcgccccagcgaccgcgcacacgcgcccgtccgcggtcgtcgctacacgatggagcgcccggccatcctgccgtccgggcagatcctgtccaacatcgaggtgcactcgcaccgcgcgctcttcgacctcttcaagcgcttccgctcggacgacaacaacctctacggcgccgagttcgacgcgctgctcggcacctactgcagcacgctctcgctcgtgcgcttcctggagctcgggctctcggtggcgtgcgtgtgcaccaagttcccggagctcagctacgtggccgagggcaccatccagtttgaggtgcagcagccgatgatcgcgcgcgacgggccgcacccggccgaccagcccgtgcacaactacatgatcaagcgcctcgaccgccgctccctcaacgccgccttctcgatcgccgtcgaggccctcgggctcatctcgggcgagaacctcgacggcacgcacatctcgtcggccatgcgcctgcgcgccatccagcagctcgcgcgcaacgtgcaggcggtgctggactcgttcgagcgcggcacggccgaccagatgctgcgcgtcctcatggagaaggcgccgcccctgtcgctgctggcgcccttcacgctctacgagggccggctcgcggaccgcgtggcctgcgccgcgctcgtgtcggagctgaagcgccgcgtgcgcgacgacaccttcttcctcacgaagcacgagcgcaacaaggacgcggtcctggaccggctctcggacctggtgaactgcaccgcgccctcggtggccgtcgcgcgcatgacccacgcggacacgcaggggcgccccgtcgacggggtgctggtgaccacggccggcgtgcgccagcggctcctgcaccacgtgctgaccctggcggacacgcacgcggacgtgcccgtgacctacggcgagatggtgatcgccaacaccaacctcgtgacggcgctggtgatgggcaaggccgtcagcaacatggacgacgtggcgcgctacctgctcggcggcgagccggcccccgacgacggcaagcccgtgggctcggcgcgcgtgcgcgcggacctcgtcgtcgtcggcgaccgcctggtgttcctcgaggccctcgagaagcgcgtgtaccaggccacgcaggtgccgtacccgctggtgggcaacctggacgtgaccttcgtcatgcccctgggggtcttcaagccggccgccgaccgctacgcgcgccacgccggcagcttcgcgcccacgccgggcctcccggacccgcgcacgcacccgccccgcgccgtgcacttcttcaacaaggacggcgtgccctgccacgtgaccttcgagcacgccatgggcaccctctgccacccctccttcctggacgtggacgccacgctggcggcgctgcgccaggagcccgcggaggtgcagtgcgccttcggcgcctacgtggccgacgcgcgccccgacgccctcgtcgggctcatgcagcgcttcctcgaggagtggcccggcatgatgcccgtgcgcccgcgctgggccgcgccgccggccgccgaccagctgctggcgcccggcaacgccgacctgcgcctggagctgcacccggcctttgacttcttcgtcgcgcccgaggtcgacgtccccgggcccttcgccgtgccccaggtgatggggcaggtgcgcgcgatgccgcgcatcatcaacggcaacatcccgctggccctgtgccccgtggactttagggacgcgcgcggcttcgagctcagcgtggaccggcaccggctggccccggccacggtggccgcggtgcgcggcgccttccgcgacgccaactaccccatggtgttctacatcatcgaggccgtgatccacgggagcgagcgcaccttctgcgcgctcgcgcggctcgtggcgcagtgcatccagagctactggcgcaacacacacaacgccgccttcgtgaacaacttctacatggtcatgtacatcaacacctacctgggcaacggggagctgcccgaggactgcgccgccgtgtacaaggacctgctggagcacgtgcacgccctgcggcgcctcatcggcgagttcacgctgcccggggacccgctgggcaaccagccccaggaggagctcaaccacgcgctggcggacgccgcgctgctgccgccgctcatctgggactgcgaccccatcctgtaccgcgacgggctcgccgagcgcctgccggagctgcgcgtcaacggcgcgcactttcagcacatcctgtgggtcgagatggcccaggtgaactttcgcaacgtcggcggcggcctggtccacaaccggcccgtgcggaacgagaaccagcccctgcacccgcaccacgacgccgagtggtcggtgctgtccaagatctactactacgccgtggtgcccgccttctcgcgcggcaactgctgcaccatgggcgtgcgctacgaccgcgtgtaccagctggtgcagacgatggtggtgcccgaggcggacgaggaggtgggcgcggacgacccccggcacccgctgcacccgcgcaacctcgtgcccaactcgctgaacgtgctcttccacaacgcctgcgtcgccgtggacgcggacgccatgctgatcctgcaggagacggtgacgaacatggccgagcgcacgacgccgctcctggcctcggcggcgcccgacgcgggcatggccacggtggccacgcgggacatgcgcacgcacgacggctcgctgcaccacggcctgctcatgatggcctaccagcccaacgacgccacgctgctcgagggggccttcttctacccggcgcccgtgaacgcgctctttgcctgcgccgaccacctcggcgccatgcgcgacgtgggcgccgaggtgcgcgccgccgcccagcacgtgccctgcgtgccccactttctcggggccaactactacgccacggtgcgccagcccgtggcccagcacgcggcgcagagccgcgccgacgagaacacgctctcgtacgcgctcatggcgggctacttcaagatgagccccgtggccttcacccaccagctgcggcgccagctgcaccccggcttcgccctgacggtggtgcgccaggaccgcttcgccacggagaacgtgctctttgcggagaaggccagcgagtcgtactttatgggccagatgcaggtggcgcgcaccgagagcggcggcgggctgcacatgcagctcacgcagccccgcgccaacgtggacctgggcgtgggcttcacggccgcgtacgcggcggccgcgctgcgtgcccccgtgacggacatgggcaacctgccgcagaacctcttcgccacgcgcggcgccccgcccatgctggacgcggacgcggacgactacctgcgccgcacggtcaacgccggcaaccgcctggcgcccgtgcccgtcttcggccagatgctgccccaggtgcccgtgggcctggcccgcgggcagcagtcggtgtgcgagttcatcgccacgcccgtctcggtcgacctggcctactttcggcgcgcgtgcaacccgcgcgggcgcgccgccggcgaggtgcacggcgaggaggggctgatgttcgaccacagccacgcggaccccgcgcacccccaccgcgccaccgccaacccctgggcctcgcagcggcactcgtacgcggaccggctctacaacggccagtacaacatgagcggcccggcctacagcccctgcttcaagttcttcacgcccgccgaggcggtcgccaagagccgggggctggcgcggctcatcgccgacacgggcgccgccgcctcgccgacgagcaacggcgagtaccagttcaagcggcccgtgggcgccggcgagctcgtcgaggacccgtgcgcgctcttccaggaggcgtacccgccgctgtgcgccagcgactcggcgctgctgcggacgcccctcggcgccgaggagcactttgcgcagtacctcatccgcgacgagtccccgctgaagggctgcttccagcacgcgagcgcctgagccgccggaccgataccggaccgccgccgaaccgtcgtctccgccctgccccctccccccccgactgctctgtcacccgcccccaccctcccgcgccggccgcgcgccgcgcgtgtatataaactgtgtacacggcacccgcccgcgtcagtgtcttctttcgctcgcgtcgccatggaggtggacattgcgctgccgacgctgtccccgggggatctgtcggccctgcagaggtgcgagggccgcgtggtgtttctggagacgctgcggcgtcacgcgacgctgcgcgaggtcgcgctgccctgcggcggcgatgtgctcgcggccatggccgcgtaccggcggcgcttcgcggccgtcatcacgcgcgtgacgccgcaccgcatgctggccacgccgctcggcgtcggcggccgcgggcagtcgctggtgctgcagaacacggggcccttcgacctgaccaacggggaccacgtgtgcctcgtcccgccgctgctcggcgacgagtgcctgcggctcacgtccgccaacctggagctgcgcttccccatgacgctgccgctcgcccaggcccgcgagctgacggcgcgggtggtggcgcgcgccgtcgagacgctgcgcggcggcgcgcccgcgcggggcgccgacgtggtcttcaacaacggccggcgctaccagctgcccccgccgcaccgcgacaacgccgaggcggccacgcgctcgctggtgctgaacatgatcttcctgctcaacgagggcgccgtgatcctgctgtcgctcatcccgaacctgctgacgctgggggcgcaggacggctacgccaacgccgtgatccagctgggcagcgccacgcgcgagctcggccagctggtgcggcagcccccgccgccgctgccgcaggaccacgcgcggcgcttctgcgtgttcgaggcgctggaggcctggatcgcctcggcctcgcggctgggcgacacgctcggcacgcggcccgtggcgcgcgtgtgcatcttcgacggccccccgaccgtcccccccggcgagaaggccgcggtggtggaggtgtgacggcgcggcgccgagggggcccgtgtttcccccgccccaccccacccccctgcaataaaaaatccgcgaaggaaaacaaagaaacgagtcgtgcgcgtctggcgtttattttccgggtcggggaaaatgggggcgtcggggggtggagggcggacggtggacggtggatgggggaagccccctcaggcaaacagcccgtcgggcagcgtgctcaggtgcacggccatgacgagggccacggcgaggtcgtcggcgcagccgccgcgcttgccggtgaagacgcggtgctcgccggcccccgaggccgtctccacgaggttcccgagctgggcgttcaggtactcgacggggtcggcgcgcgcgcccacggtcacggacacgagctcctgcgaggccatgacggcgcccgagttgaactgcttcacgaaaaagtcgaaggcgggggtcttttgcttctgcagcaggaagaaggggtagcgcacggcgcccgcgcccgcgcccggggcgccgtggtggtagaagaccacggccgggtcggcgacgacgcgcgcggcgcgcagctccgcgagctcgcggcgcatggcgcccacgatggccacggccgagtcctggctgctgttgccctcgacggccacgcgcaccgccgagaaggcgccgtcgtgcagcgccagcacgcgcgccaggcagcgcacgacgcagcgcgcgatctcgtcggcggaggcgccggtgagcgcgcgcaggaaaaagtgctccaggccgagcacgatccagctgcgccggtagctgcccacgacggccacgccggtgccggaggcgcgcgcgttggccgtgaaggccgggtccacgtacacgtgcagcgtgccggccagcagccggcggttggccaccgaggaggggcggtagagcaggaagcgctcgccggcggcgcgcgtgaggacgggcgcctcgcgcgcggccacggcgccgccgaggatctcctgcatgaaggagtccgggagcagccgctcggccgtggtgcgcgtggcggcgtccatggtgatgaagacgggcttgttgagcacgtagcacgagcaggcggtggcgtcggtgtgcgccaccacgcggcgcatgtgctcgtcgcagacgtacgtgaccacgttgagcatgtcggtgccgcggaggttggtgaggaagctggtgctggccttgccggtgttggtggaggagacgaagatgatcttgcagctggcctggttcatgaagccgaggatggtctgcacggcatcggggcgtataaagttggcctcgtcgacaaagagcaggttaaagtcctgtccgcggatcccctgtaacgacacgcggggagagagagagggggggacggtgagcggcgccgcgatggacgcccacatcgccaacgagaccaagcagcagatgacgcgcttcgcgcccgcgctcgtgcacgtcatcgtgcccgaccccctcctgcggcgcgcgggcgtggacccgctcgcgcccttcgccgcccacgcgcagacgcgctaccacggctcgggcgtctgcgagccgtgggtctccgtgttcgcgggccacgtgcagacgggcgccgtcgagagcgtgctgacgctgccgccgctgcagcgcgcgcgcggccccggggggctgttcgtctcgctgcccctggcgctcggcgcccactttgacggcttcacggcggtggcgctgcgcgtgggcgcgcgcgagctgatcttcacctacgacgagctcctgccggcccgcacgcgctacaacgtggacggcgagcgcctggagcgcctgtgccgccagttcgccaactacgcgcgcgcgcggcgcgtggccccggaggtcgcggccgcggggggccacatcgacgcgctgctgcccccggcggcggccaccatcgacggcgaggcgcagctgacgcgcgggggcttcgacgacccggcggcgccgcacgcgcgcgacgccgaccgcgagatcctgtcgctggtgcgccgcgcgaggccgcgggcggcggcgacggcggcggcgccgcctggcgcgacgagctcctgctcacgccccaggacccccggccgctgacggcgctcgactggctggacgcgggctacgccgcgctcgcgggcggcgacgcgccggcgcacgtgtggcgccggcggcccgtgtccctggtggcgcgccgccactaccagaccggcgagacgttcgtcgtcgtcgcctacgagcactccaccgcctggggcggccggcgcgggcccgcgagcgagcccctggcgcgcgtgctcgccgaggagtgcgagcgccacggcgtcgagcacccgcgcgccctgcccgcggaggcgcggctggagctggtgcggcggcacgccgagctggccgtgccgctgggggacgaggagccgccgctgccggtctttgacgccaccgcggagctggtgctgctggagcgcttccgcaacgcctgcgtgcgcgcgctgctggccggcgtccgcgagtccgtgcgccgcgagccgcgcatgcgccagatcatcgagttcgcgatccgcccgcgcgaccgcgaggccgtcctcgacgtggccgggcgcgccccggcgctgctggacgcgttcgcgcggcgcctcgagcagacgccggcgcgggagatggtcgactcgggcctgatgacggccgccgcggcgcacctcgccgcgcgcgccaccgccggctacgtgaccttcgagagcggcccgctgctgggcggcgttttcctcttcgactactacagcgccggcggggaggttataagggtgacgcgggccccgccggccgtggcagtggagccggcgacgcgcgggcagttcgcgtgccgcttccgcggcgcgtcgcatcgctgtctcccgggcgagagctacgcgtacctctgcgtcggcgtgtcgcgggacctgcgcgcgctggtggtgctgcccggcggcttcggcttcttcgcggccctgcggctggagtggcccgccgcgctggtggaccccgtgctcgagcgcctgtgccgccgcgtgtagcgagggtgggcgcgacgagcgccgccatggaggtggccgcggccctgacggaggactttgccgcctggcggctgctgcgctccgactcgcgcgtcaaggtctacgccgccctcggcgtggtgggcgcgcgcctggccgccgtggcgccgggcgccgcgaccgtcgacgcgcgcgtctacctgacgcgcccgcgggccctgcgcctggcccagggccgcttccacgtcgtggtcctgctcaacgacgccgcctacgcgctggtggcggccgtgaccacgaccacgctccgcgggagcggcggcgagctcgtgcgcctgacgctgggcgacgcgagcctcgaggccctgcccgccgacctgcccgtcgccgagcccgtgcccgccgcgccggtgagccgcctggacctggacgccgccgagcccgtgtccgcggcgcccgcgcgccgccgcgagtgcgtggtcctggcccccggcgcgtggtggtcgcgcgaccgcatctactttctgcagatggacccggcgctgctggcgctgtgccccgccggctggcgcgcgcgccacctcggcgccgtgctcgccgggctgctgagcccccgcgacgacgacggcggcagctgctgccgcgagtgccgcgtggaccacgtggacgcgctcaacgcgacgccgcaccccgacggggcgggcgcggcctgcctgtgcgccgcgccgtgcctgtggcgccaggcggacaagcgggagctgcgcgcgagcgactcgggcctcttccgcgtgctgttcctggacgccgtgcgcttcgtgcgcatgctgccccggcgcaagatcgtcgacgtcgcctccgagctcatcggcgggctggacgcccgcgggcgccacgtggtggtgaacgacgccggctggcgcctggtggccctggacccggacgcgagccacgcgctcgtgtgcggctgcccgctgctgcgcgccctctgcgaccccccctcccgcgccatccccgagctaataaacgactattgaaataaaaaaatgggtacttgcgtttgtgttgtggctggaggcgaacacgatggtgctgcgcgcgccgtcggggaaggtgacggtgatgttctcgcccttgacgtggtccacgcgcgcgtcgcggcaccagcggcgcaggcgcgcgtggatctcctcgaagacgggctccgtggccttgcggatgtgggccgtgtagccgacgcggatgccgcggaaggtggccagcgcgagggcgatgagcgggacgaggaaccaggtcttgccgtggcgccgcggcacgaggaagaccgtgacgcgctggcgaaagtggcgcaccgcgtcctcggagaagagcggcgtgtcgaaggccgccagcaggtaggcggtggcgcgctcggcgtggtccccgagcagcaccgtggccagaaagtacaccgcgtgcatgaggatcatcttctggaagagctccagcgtgccgcgccctccgccgccgccgccgcctctgccctttgccccgcttccagttccacctccaccgccgccgccgtctccgctctcgccgcggccctcctcggcggcgtcctccggctcggggcggccgggcgcgacccccgcgcccctcttctccgcggccccgtccccatcgtcctccgcctcctcgtcgccgtcgtcgttggccccggtcccggccccggccgtcccgagggcggccgtcgcggcgtcgcggaaggagcgccgcgacaggagcgcgaagcggttgacgaagcgccggagctggcggaaggccccggacgaccgcatggcgtccaccgccgccatggcgctcgtgtacgcgttgcggtggaaggccaggcgctcgccgtcgtaggcggcgaactcgagcgcctcggccgcgcgcgcgagcgccgggtccacggcggcgcggccgcgcgcgcccccgaagagcgcggcgcgcgcgagcccggcgaagatccgcgcgctctcgcagcagttgtgcagcgtgccgaccgccgggaccaccgtctggtggcgctgtggggcggcgatggcaaagttgaaaaagcgggcctcctcggcgcagtcgcgctcactggcctcgcgcttgcgcttgaggtccgcgtagtagcgccgcgtcgcgtcgcccaggccgaacatgttcgcctcggaccgccgggagcgccgcgtgcgcctcgcggaggcctttcagcgcgagagcgtcttcaaggcgcgcaccgtcgagcttctgcgcgggcgcgccgacaagaaaaaccccgagttcgtccgggcctttatggcggccaaacaggcccggcgggatgtagagcgccacctgcggctggccgcccgggtggagtctgtggaacaaaaagcgcgcgcgctgcaggcccgcgtggaagcccaggcggccgtccgcggggtcctggacaggcaccggcggttcacgcgggcggactttgccgaggcgctcgacgccgcggaggacgcgctcgtggccggcgaggaccggctcgacgacgcggccgcgctcgacgaggactgggccggcggtggggaccctgacgaggacgagggggaggaggcggacgaggccctgctgacccaatggctgctggaggaggcggcggaggcgtgagccggaccgcgctggcgcggccgcccatccaccgcggcacgtccgcgccctgcggcgacggcgacggcgacgaggcgtcccggctcctgggccgcgcgcagccgcgcgaggccccctacctgatcccgcgcccggacggggacctcgccgtgccggacgacctgcagtacgcgaccttcgacctcacgggcgaccccgtggccgtcggggccggctcgtacggcagcgtgctcgtgtacggctcggtggccgtgaagacgctccgcgccggcttcggccacgaggccgtcatgacgctgctggccgcggaggcgacgcgctccgccggcgtccgcggcgtggtgcgcctggtggggctctcggcgccgctgcgccagctcgtgttcccggcctacgagatggacatggacgcgtaccgccgctcgctcacggcgcgccccgggcacgtggtgcacgccctggggcgcgtcttcgccgagctcgggcgcgcgctcgtgttcctcaacagccgcgggctcagccacctggacgtcaagggcggcaacatctttgtgcgcacgtgcggcaacatggtcgtgacggccgtcatcggggactttagcctcatggccctcaactcgcgcagcacgctcgcggacccccgcttccgcctcgcgcgccgcaaggcgctgaagctcacgtcgctggcgcggagcccgccgacgggcgtcctcctggggcatgcgcgcgaccggcccacgggggtgctgatggactttattaacgggcgcccgccgccgccgggccccctgccgtacgaggtgggcctggcgatcgacctgtgcgcgctgggccacgtgctgctggacgtggcgctcggcctgcgcccgcagcgcggccagacgctgacgcgcgagtacgccgtggaggtgctcgcgcgccgctgcgtcctcttcgcggccctgctcccgccgggcagcggccccgccgccgaggccctcgccgaggacatcctcgaggaggagctggccgccagcttccgcgagtgcgtggccagctcgcgccccggcaaccagcccccgcgcacggtggccccgctgctcgagctcgtggcccggttctgcggcgaggatggcggcgctcgttttgccgaactcgctgcctgaggagctggccgcgcgcaccttcctgcggttcctgcgcgcggcgccgcgccccgcggccggcggcgcgccgctggcgtaccgcctggcgtacgtgcacgacctgctcgtcgagctggcgcgccacggcctcgccgcccccgacgcggccgcggcggcctttggcggcgcgcgcccgccgcccgcgcccgcggacgtccccgccgcggcggcgcgcgccgccatcctgacggtggaggcggccacgcgcgcgcagagcgagagcgacctgtggacgctgctgcgccgcgggctggccacggcctcgaccgtgcgctggggggcggacggcccgcgcttcccgccgacgtggtgcgaggccagcacggcgcgctgcggcacgcccgacaacgcggcgctcatctttggccgcgtcaacgagcgcgtggcccgcgccgccgtcgcggccctgtacgccgaggcgcccacgccggacctgcccgcggcggcgacgggcggcgacgagggcgacggggacggggcgaaggaggagatgttcaccttcgacgagacgggcgcgcccccgccgggtcacgacctcttctcgtgcgggctgctgctggacccgcgcaccggcatggtcggggcctcgctggacctgctggtctgcgaccgcgacgcgcgcgggcgcctggcgccgcaccgcacgcagacggagatgctcttcttcgagatcaagtgccgcgccaagtacctgttctcggcggacgacgcgagccccacggcgcgcgcgtacgcgcgcctgctcgggcgccccgacgcggacacgctgcgcggcttcctgaactccatcgcccggccgggcgtggagttcttcgagggcgccccgggccccggcgaggcgctggcgacggccgaccccgcgtggcggcgcgcgggcgccgaggacgcccccgcgacgcgtcggaggtgtggcgccttcgacgcgcgccacgtcgccgcgaacgcgcacgcccagtcggaggtctggctatttagcgacccggtggacggccgtcaggacattgtgccctgggcctcgggcgagcgcgcgctgagggtgcccgtgttcgcgaacccgcgccacgccaactttcggcagatcctggtgcagagctacgtggtcgccggcgtcttccccgaccgccccgcgcgcccccacctggccaccttcttcgggcgccgccgccgcccctgcgagcagaaccggacgctcgacctcgcctcgctgtgcgacgtgccgcccgcctgcgccgtgcccgtgctgctgatcgtcacgcccgtctccgtgtgcgaggaggcgttcgaggacctgcggacgcgcgccgaggaggcctttcgcgccaccgcgtcgcggacatgggacagtgctgctgccgattctccagcaaccgcgtcgtgacgagctccggcgacgtgctgaccttcgacgcggacgcgttcgaggactttgagctcgagcccatggtcggggagcccggccccgtgcgccccaaggccccgtaccgcgtgtcgcgcgggaacctccgtgaagcctcgcgggcgtactgactgcaataaacccgtttgtcatactatcgcatcgttcccgtctggtgtctcggggcgggagggacgatgggacgggggagacggaggacgggggagacggaggacgggggagacggaggacgggggagacggaggacgggggagacggaggacgggggagacggaggacgggggatgtggcgggttgggtgtgagacgatggccgaggacgggggaagagggccgtggggcggggcgagatggaatggcgcaccggccgcaaagtgggacgatggggacgggcctcaacgcgggacgagggcggcggggcgggaccggggacaaggacaagggcggcgagagacggacacgagaggaccatgtgcgatcgaaacgaacgtttattcaaagccgaggttctcgtacacgacctcgtcgccgtcgtcaaactcgtcttcggtcatcccgggggccagcaggtactcttcgttgccccggccgtgcaggcgcttctgcaggtaccgccgggccgtcgtccgcgccttgtcgacgttggcgtagaaccggctgccccgggcgcggtgggcgatgcaggcgcggaccaggcgcacgacgatcatcgccagggcgatcagcgacaggaccgccaggacggcgcgcgtcccgatgacgacgccgtgcgtctccccgagggcctcgctgaacttggcgaagtagcggtgggcggacacgccgagggccgcgctggccacgagcacgcagaaggccggcgtggggagcacctgcacgtagccggacacgacgacctcgacgaacgccatcagcgccagcgccagcaccgtgaacacggccagggcgacctccgccgtcttccagaggctgatgtggaagctgttggccaggagcacgccgagcatcagcccgaccacggccgcgcccaggccgaccgcgccgaggacgacgttcgtcacgacggcgcgcgcgtgcaccgccacgcggcgcagcggggggctggccgccagggcggcgcgcacgtcacccgcgcccttgcaggcgtggcacgccccaaagagggcgagaaacacaaaatgggtgatgtaggcggcggcggccagacccgcctgcttgtgggccatcagcagcaccacggcctggagggtccaggcgcagagcgcgccgagcatcagggtccccggggcggccaccgtcgacgccgcgtagagcaccacgctgggctggaacgccagccggccggcctcgcgccgcagcaccacggcccccgcgaccgcgtacagggcgtgcgccagcagcacggtggccgtgaagccgaagaagctggtcacggtgggcgtttccagaaacagggcgggggccacgagcggctggcgcgtccacacgccgccggaggtgtcgttgagcgtgtcgtagtcggccacggtcgcgtaaaagcacgggaaccccatttccgggagcgaggcgaagataagggtgaggaccagcgtcagggcggccagggcgaacccgcagacctctatcagccaggagcgccagctcacggcctcggcgttgcgcggcccggacatggcggcggacgcgggcgagcgccgcggcgcgaccgaggaggacggcggggcgttcgcctcgagcgtgtcgctcgcgcggatgctatacggttgcgacctcccggccgtggtgcgcagccgctggccgggcgtctccctggacctgcagcgcgacgcgcccgtcgagcttccgtccccgcacgataccgcctgccgccgcgtcctcgtggcgcgcgctccgatgggctcgggcaagacgacggcgctgctgaaatggctctccgcggcgctggcggccactgatatgagcgcgctggtcctgtcctgccgacgcagcttcacccgcaccctggcgagacgaatggacgacgcgggcctgggatttgtgacgtacttcgactcggacgcatacgtcatgacgggccggccctaccgccggctgctggtgcagatcgagagcctgcaccgcgtggacgagcacctgatcaacaactacgacgtgctcgtcgtcgacgaggtcatgtccacgctggggcagctctactcgcccaccatggcgcggctggcccgcgtggacgccctgctcgcgcggctgctgcgcggctgcccgcgggtcctcgtcatggacgccacgatcaacgcgcagctcgtggagctgctcgtggagctgcgcggcgagcccagcgtgcacgtggtggtgagcgactacgccacggccgccttcgcgagccgccgctgcctcgtgctccggcacctcggggccgaggtcgcggcgggcgcggcgggcgcgcgggaggacggcggcggcgacgggagcgaggacgcggcgcgggccgggagccccgccccggtgacggcggcggcgacgacgacggcggtggaggcggcgggggcggcgggggacagcttcttcggcctcctgggcgcgcgcctcgccgcgggggacaacgtctgcgtcttctcgtcgacgctggccttctccgagctcgtggcgcggttctgcgcgcgcttcacgccctccgtgctggtgctcaactcgcagcggccccccgaggacgtgggccgctgggccgtgcgcgccctcgtctacaccaccgtggtcaccgtcgggctcagcttcgacgcgccgcactttcacagcatgttcgcctacgtgaagccgatggcgcacggcccggacatggcctccgtctaccagtccacggggcgcgtgcggcggctgctgcgcgacgagctcttcgtgtacgtggacggctccggcgcgcgcggcgagcccatcttcacgcccgtgctgctgaaccacgtcgtgggcgcgggctggccggcgcgcctgtcgcaggtgacgaacctcgtgtgcgcgcagttccagcgccgctgccgcccggcgttcgcggcggcgcgcgggatgcgcctcttctcccgcttcaagttcaagcacctgtttgagcgctgcacgctgacgagcgtcaacgacagcctcaacatcctgcacgcgctgctggagaacaaccgcctgcgggtggcgctcgagggctgcgagccgccgctgacggcgcgcgccttctgcgacttcctgcgcgacgcgcgcctggacgccttcgccagccagcaggtgctgcggcagctgcgccccccggaccggccggtggcggccgacatcgcggacagcggcgaggtggccaccttcgtggagaagtacctcgtggccgacgtgcccgaggacgagctgcaggagctgctgcgcgcgctcgccaaccccgtgacgcgggagcagtttgtgggcctggccgtgctcggcgcgtgcgcgcgcgtccccgaggcgctgcgcagcgagcgcgtcttcggggccgtgtacgggcactacgcctcgggggccgtgcccgtggtggccgacgggcgcctggagctggcggcgctggcccccgacttcaacgtgcccgcgcgctgggccctcacgcggcgctgcgcgcgcgtcgcggaggccgccgggctcttcgagggcgcctcgccggaggtggactcggccgcggtcgcggcggcggcggccgacgccgagctggcgccgctgctgctcgaggtgctgcggtgccacgtgctggacgcgacgacggcggcgcggcgccccgtgcgcgcggccctgagcgcgctgggggcgggcgggggcgcgggcccgctgagccgcgggcggcacgccgcgctcgtgttcaaggtgatgtgggaggaggccttcggcgtgcgcgtcggccgcagccggcagaccttcccggggcccacgcgcgtgaagaacctgcgcaaggccgagatcgccgcgctgctgcgcgacgccggcctggacccgcccgcgggcgccacccaccggcagctgtacgccctgctgatggaacgccgggaggactttgcgggggagaggtataaactgcgcctgcccgcctggagccggctcatgtacctgacccagggcggcttcgacgcgccgctggacgccgcgctctcgctcgtgcccgcggaggcgtggccgcgcatcgagggggccgtggactttgcggcgctatgacggccgcggcgggggagcgcgtgtgcgcggccgggatctacacggcctggacggagcccggcgcgccgggcgagctccacgcgctggcgcacctgctgtgccgcgacggggccggcgcgtacgcggcgcgcttcgtccacgtgacggcggagccgtggacgggctcggcggccgcggtggccgcggcgctgcgcgacgcgagcggcacgggggcgctggcggacccggcgctgtggcgcggcgcgcacggcgcggtcatggcggccctgcgccgcgccctcccggcgctggccttctacgagccgctgcgcttcgagacggacccggccacgaacctggtcacggccgcgacgccggtggccgggggtggcggcgaggagaggggcgagggggagaagagcgcggaggaggacggggacgacgaggacgaggccggcccgcgcgcgggcatcatccacgtggacgccgacgtcgtcgtggaccgcgacgccgcgcgcgcgcacgccaccgaggcgtggctgccgcacgcccagctgcgcgccatggccggctgcgccgcgctctccgtcgagatcacgacgcggcggaccagcttcgcgcgcgcctacgccgaggccggcgcgccgccgctgaagcgccggggcgagatgggcgacctctttgacgtgcgcgagacggtgctgcggccccggggccggcgcgtggcggcgcgcgtgctcgtcccgcgcggcttcgactgcctggtggcccacggcgtgagcaacgcggcgctggtggcgctctaccgccggtggcacgcggccgcgtacggcgcgggcgacgcgaccccgcccgtcttcgccttcctggggcccgagctcgcccgcgagggcgcggacgaggactactactgcgcgctgggcttccccctgtttgcgacgatcaaggtggccgtggccgcgccggaggcggtccgcggcgcgctcgccgcccaccggctcacggacgggctgtggcccgcgctcggcttccgcgccttccacgccctcgggcccgtgacctgcggcctgcgcctgcgctgcgacgtctggccggcggggcgcgtgaccagcgccgtggagcacccgtgcgccctccgcggggcctggctggccaagtttgacttcgcggccttcttccccaacctgtttctggccctgtgcccgggccacgcgcggctgcggcgcgccgtgcgggcgcgcgcggtcaagccggcgctcgtggccttcttcggcgggctcaagcacacgtgcccggaggcgtaccacagcgtcatcgcgctggccaacggcgtctcgcgcgccgtcgaggcggccgcggccgaggccggcctggtcacgtgcgcctacgtcaaggacggcttctggggcgtgctcggcggcgtgcccgcggacgcgccggaggcggccgccgccgccgcggccgagcgcgtgcgcgccgcctgcgaggccgccgcggcgcggcacctggccgacgcggggaccgccgcgacggacgtgacgctgcgctgcgagggcgtcttcgacgccgccctggcctggtcctgccacgcctactggctgcggcggcgcgagggccccgcggactttgtcggcttcccggcgcgcagcggcttcggccgcgccgccaaggccgcgctggacgccctgctgcgcgaggccgtcgagggccgcgcggacgccggggccgcgcgcgccgcctgcgacgggctcgtcgagcgggccttcgcctcgcgccacgaccccgacttctgggccgccccgggggacgccctcgcccccgccgcgtgcgccggggcgaccgccgggcgcggcggcgcgcggtgctcgcggcgctgcgtgcgcctgcgcggagccggcccggtgccctacgagcagctgcgccccccgctgatcctgccctggatagactgcctggcccacatggagcccgtcttcgcggccctcgtcggcatgtacaaccgcgcgctcgaggcgctgagcgacggcgacgccccaccgccctttgtgtttacatacacaccggacgcgtttctgtttggttaaacaataaagtctgtttattgtccgaacgagccacccggcgtcctgttgtcgtccgcggtcggggatggtgggggagagggaaggggagagggagggggggagggcgaggggggagaggaggggggcagatggagaggggaaaggggaaaggggggtcggggcggggaggtggaggaggcggaggaggggggaggcgcgcttcactcgtagcgcacaaacatggtcttctcggtgagcgcccgcggcatcgtcgtccaccagcggtacacggcctcggccatctcgggcgtgcgcaggggcgcggtgagcgcgccgccggggcgcgtccgcaccagcccgtcgggcggcggcgcgtcgcccggcaggccgagcaggggcccgaacccggcggcgaagagggcgtccacgacgggcggggtccggggggccgacagcaggcgctgggcgatggggtagtggggcagcacgcgcgtctcgtcgagcgggctgaggtgggccgcgaacatgagcgtgttgagcgcgcgcgtggcgccgacgtgcagcaggcggcgcagcttctgcgccggcggggcgcgcgcgcgctccagccaccggcccaggcgcaggcaccgcgcggcgtccgcctcggccgcggggcagttctccaggaacatctgcagcacgcagagcgcgaactccgtcgggtcctcggggcgcaccagggccaggcggtgcggcagcgccaccggcggcacggccagcgagagcacgcggtcctcggcggccgtgaccaccgcgaaggcgaacccgcggaaggcctcgccggcgaggcagcggcggcggtactcgtcgcaggacaccgtctcgccgctctcgaggcgcagcgtcagcgcggcgccctcgacgcgcgcgtcagtcaccgaggcgctcgtgaagaccgggatcagcccgggcacctcgcgcacctcgcacaccagccgcgggagggcgtgccaggccagctcgtccatcacggcctccatcgcggacccgctcggtcgcccggcgcacgtccgccgcgaacagcgccgcgatctcggtcaggtaggcctcgaggcgcgtcttcttaaacaccacgccgcacaggtcctcctccgagcggtacacctcctggctggtgatgaggaagccgaggtggcgcacgcgcaggatggagaagaagtacggcgcgaggatgagcgagatggagctgttggagtacgagacggacacctcctggccctggttgttggccacgcgcgtcagcttgaagcagcggatcagctcctgctcccagagccgcgagaggcgctccacgtccgactcgtacggcggcacgtagcgcgactggaagctgttggccacgtaggcgtcgtcgcccatggcgcgcgtgatgtcgatgacctcgtggccgaggtcccgcggcggctggccgtccgcggcgccggcgcccgggccgggcgcgcgccccccggggccggcgccgcggtccgcgcccgcctgcgcggcgcgcagggcctgctcgcgcaggcgccgcacctcctgctcgcgctcgcgcacctgctcgcggaggcccccgttgtccgccttcaggctctcgatggtcttgaagaggttgttgacgtagccctcgagcatgccgttgatgctgttgaccacggaggtgcggaaggcgtcctgcaccggctgcgcgccggcgccgtggtgcccgaagcccggctgcgacgtgtccacgctgtccaggatgcgcgccccggtctcgtcgaggtacgagcgcaccgtctccgtgatgtcgccgatgtggcgcatgcccttcatgttcacgatgaggcgcacgaggcgcgccgcggccgagcccgccgggtcctcgccgagcatcttctccacggcgccggccgggtgcccctcggcgggcttgcgcccgacgagcaccttcaccggcgccgtgttgagcagctggcagaggcgcgcgtgctcgcgcagcgcgtggcacgccagcacctcgccgtgcacgcgctgcgccggcgagtcaaagagcagctcgccgtcgcgccacagcggctgccagagcaccaggcactccccgcgctcgcccgtgagcgcgtcgcgcgccagcacgcgccggtgccggggctcgtagtagagctgcacgcggtcgtagtccatgatggtgacgccgcgcatggcctcggcgagctcggccacctcgtccaggccctggcccagcacgctgccggcgacgcgcaggtgcccggccaggtcgtgctcgtccacgcagcggtcgcgccgccgcttccccccggggcgcgcccggcgcaccgggaccacgccgaggcacacgatccagtcgatgtacttggcgaagctggccgtgccgttggactcgctggccgagaagcaggccgcgatgccgcgcacaaagtccaggagggccatctgcagcgtgcggtaccaggtgtcgaagaggcgctcggccacggcgggcgcctcgtcgccgaagcgccgcgccagcgcctcggcgccgaggccgcgcgcgcgcaggtgcgccatccagtcccgggccacgtcctcgtagcgcgccgcgttgagcgtgcgcgtgagcagcgtggactggatctgccgcacggcggcctcggtggagcgcaccgagttgtagatgccctggccctcggtgtagcccaggttgcccatgaggatctccttgaagaccatggtgcgcggcgtggggtggatgaggatccgcgagggggcgcccaacaggttattagcctcctccgcggcgccgccggggcagagcccgtccgcagccgccgccgccgtcgcagccgacatggccgccgccgcagagggtccgcgcgccaccaacgccacctatctgaactttacgtccatgcacggcgtcgagccgatcgtggagcgcgtccgggagctcgccgcgacgcccgcggaggcgccgccgccgctggcgtggttcaagtccctcgccgcggcggacaacccggtggatatagaggcgctggagctgcccttcgccgcgtacctgatcagcggcaatgccggctccggcaagagcacgtgcatccagaccctcaacgagaccatggactgcgtcatcacgggctcgacgcgcgtcgccgcccagaacgtctacgccaagctctcggcggcgtactcgagccgctacgtgaacaccatcttccaggagttcggcttccgcgggaaccacgtgcaggcgcagctcgggcgctacagctacgcgtgccccacgagcccgccgacggtgcgcgagctgcagaagcgcgacctggtgtactactgggaggtgctgcaggacatctcgcggcgcgtgctcgcggggagccacgaggagtttgcgcgcctgcgcgccctggagcggctcacggggcgcgcggcggagcacctggccttcgcgtgccacggctcgctgccggcgttcgcgcgcagcaacgtcatcgtgatcgacgaggccgggctgctcgggcggcacctgctcacggccgtcgtctactgctggtggctgatcaacgccgcgtacgacacgacgcagtacgcggcgcgcgcgcgccccgtgctcgtctgcgtgggctcgcccacgcagacggactcgctcgagtcgcgcttcgagcacgcgcggcagctgtgccgcgtgcgcgccagcgagaacctgctcacgtacctgatcaccaaccgcgcgctgcgcgagtacacggacctctcgcgcaactgggccatcttcatcaacaacaagcgctgccaggagtacgagttcggcgagctcatgaaggcgctcgagtacggcctcccgctgacggacgagcacctgcgcctcgtcgacagcttcgtggtgcccgaggcctacatcaacaacccggcgaacctgcagggctggacgcgcctgtactcatcgcaccgcgaggtcagcgcgtacatgagccgcctgcacgcgcacctcaaggtggcgggcgacgcgcagtttgtggtcttcacgctcccggcctacaccatcgtgcgcacggcggccttcgaccggtaccgcgaggccacgcagcagccgcacctgacgctggaccgctggctcgcggccaacgccggccgcatcaccaactactcgcagagccgcgaccaggacgccgcggcgctgcgctgcgaggcgcgcgcgcagcagggcgtcgtgctcgcccgctgcgaggtcacgtacgtgctcaacagccaggtggccgtcaccacgcgcctcaagaagctggtcatcggcttcagcggcaccttcgaggccttcgcggccgtcctgcgcgacgacgccttcgtccacgcgcagggcggcagcgccgagtacacgtaccgcttcctgtcgagcctgctgtttagcggcatgatcgccttctacaacttcctgcagcgccccgggctggcgcccgaggccgtcacggcggcctaccggcgcctggccgccgtgacggcggcggcgctgcgcgtgcccgaggagcactttgacttctcgggcgcggcggcgcccgccgcccccgcgggccccgcgggccccgggggccccgggggccccgggggcgcgcccgcgtccgcgccggcggacgacgacgacgacctctttgcggcgctcagcgagaacatgctggacatgctctactgccactacgactttgcgcgccccgagaccacgagcgaggtgtacgcgcagttcctgatgctcaagacgctcttcgccgaccgctacgcggcgctctccgagctcttcgggcccgcgttcgcgcgcgcgcccttcgagacgcacgtggacagcgtctcggtgcgcgggtgcgaggtgtttgtgggggggctgcgcggcgcgctgctctcgacggcgctgcagacggacagctacacgctcgtgggctacacgcacgcgacggcgccggccttcgccgaggagctggcgcggcgcaagctgcacgccggcaccgcggagctgctcgcctcgctggacacgccgcgcgtggtgctgcgcgaccagagcggcttcctgtccatcctcaacgtcaacctcagcgactttgtcgagtcgctggacgacctggagctggacatggccacctacgtcgactacggcatcagctccaagctggccatgaccatcgcgcggtcgcagggcctgagcctggagcgggtggccgtgtgcttcacgcgcgccaacctgcgcatgaacagcgtctacgtggccatgtcccgcgtgatctcgtcgcgcttcctgcgcatgaacatgaacccgctgcgcgaggagcccgagcgggacaacggcatcagcgagcatatattggccgcgctgcgcgacggcgcggtgcacatcgtgtactgacccgccgtccacccgcccgagggggccgagggaccgagggaccgagcgagaccgaccatgacggcgctggtggcctactcgttttacgaaataaagctacccggcggctgggcgcagccgggctgtgggcagaccgtgtgcgagtacgagcgcggcgtccgcgtcatggccacggacgggtgcacgcgctgcgacgcgctcgccccgggccgcgtcacgatccagcacgggccggtgctcacggtcctggccgtggacggggagccggagcggtgctcgtacgtgttcgcgcggacctggccggcggcccccgagggcgcgctcgtgatgcccttctcgacctggagctgcgccgagcgctcgcgccgcctgcgcgggcccgcgggcggcctgctggccaccctcgtcgcggagcgggccctgcacgtcaccatcaccgcgtaccggcccgacgtgctccgggacgccctgcgcgaggcccgcatcctggagtgagaggcggcgagggccgggcgagcggaggaggccgccgccatcaccatcgccgccaccgtcaccatccccatcccactcccatcccaccccgacaataaacgacgacggccgcgcgtgcgcggaaagagagaccgaaccggttgtgtcttttgtctggtccgtgggcatccgccctttattgatcgcggtagcaacaggaggcggggggcgcgcgccggccccgggacggggtccgggggcgcgggagggcggccgccgaggggacggcggcgtgggccgaggcgtggacgctgcgctggcggggtctcggcagctggggcgtcggggcggccgtcgccgaggcgttgatgctccgttgccggggccgcggcagctggaccgccggggcggccgtcgccgaggcgtggacgctgccgcggcggcgcgggggtgcggccgcgcacgactcgggtccgtccgcggccgtcgccgaggcgtggacgctgccgcggcggcgcggggcccgctgctgttgttgctgctgctgcggatgctgagagcggtggtggtgctgctgctggtggtggtggtgggtaggctggacgacggtggcgtagacggggtccggctcggtcgcgccgcgctggcgcgagcgcccgcgggcggtgcccaggccgccggcgcgcaggggcgcctcctcgtcggggggggcgcgcgaggaccgcggggcccgcgtctcgcccccgccggcggagacgcggcgggaggccgcggccaggcgccgggcggccgtctcgttggcctgcaggaaccggtggtagtcatcgaccgtggccacggacgcgcgtccgaaggtgctcatggcgccgcaaacacgttgtcccggtgcagccgcaccggctcgggggtgatgaactcgtggatgaacaccggcgcggccggccgcgttcgtccgctcgtcgggcgcgcgcggggctcgcgtgggcgtcgggcgcgcaccgcggaccggagctgatcgcgtacgcgcgccgcgtgggctctgcggcacaggataaacatctgcaggcttttatggccgcgcccgggggcgccgcgcgggccgcgcgcgccgcgcgcaaacgcggacgtcttggtgcaggcgacggggaggccggccagggccgcggccaggtcccggcggatcgtgtccgtcagccgccggcgcccgagctcgtccacggacgcgaccataaacagcgtgtcaaactccacgtgctgcgcctggcgcgggcccgccgccgccgtcgtcgtcgtcgtcgtcccttcgtcccccccatcttcctcgtcttccgtctcctccttctccgtcgccttcccctcctcgccgtcttccacgtccccctctccgtcttcgctcccgcgcaccccctcccggggagggggtgcgctctccgggggcgcgtccgcgtcctccgggcacggcccgggggactcgacctcctccacggcccatccccactcgcgcagcaccgccaggaccggcagcgcgctggccggggcgccggcgagcgcgggctccatcatccgctctcctccgtccatcccaccccccctccccgcctcctcgtcctccgccggcggtgtgcgcgcacgcccgactcagtccacgctccagtcgacgggggcccggcccgtctggacgaggtacgcgttcgcctctccaaagtgcgggcaggtcctgaagggcgtgcgggccagcggcgacgggtggctgaaggtgagcaccttgtggcggcgcgggtccggcgcgcaggccttttgagcgtgggcgccccagagcatgaacaccagcttggggcgggtctcgcagagccgctggacgacggcgcgcacgagccgcgcccagccgagcggggcgtgggagccggggaccccgcgccgcacggtcagcgtcgtgttcagcagcagcaccccgcggcgcgcccaggcctccaggcagccgtgggcgggcgcgggcagcgtcgggtacgtcgcccggaccgccgcgaagatgttggccaggctcggggggatcggcaccccgcgccggacgctgaaggccagcccgtgggcctggcccggcccgtggtacgggtcctggccgatgatgaccaccttgacgtcctcgggcgccgtgaggcgcgtccaggcgaacacgtcctcgcgcgcgggcagcacctcctcggtcaggcagcggccgcggtactcggccagcagcaggcgcgcgtacggcttggcgagctcgggctccagcagcgcgcgccacgagggggccacgtcgaactcggcggcgaaggcgtcccacgtgggcgccccggggtcgggggccggggcggaggcgtcggcccccgtggctgttgtcgccgccgacgccgcgggcgtgcccttcgcgggccgggcggcggcggagggtcgggccggcttcgaggccccggcccctgcccctgcccctgcccctgcccctgccccggcgggggctgctgctgcagcggcggtggtggcgttagaggcagaagcggcggcggcaggcacgacgaggcgcaccccgggcggcaggccgcagggtctcttactcggcggggggccctccattatagcgcagcggcggcagggagtccttgtgcacggccacctctccgagctccttgagggccgcctcatttaagggctctccgacggccagggcctggatggcgacaaaggggttgaccaggtaggcggtgccagacgcccacaggatgacctccggcggcgagcagtagggcttcacgaccacgctcgtgacggcgttcatgtccggcggcagcgagtacgcgatgtagggatggcagcgcgatcccacgggcggctcgaggtccagcagcgggtgatcctcatcgtcccactccagctcctcgcgccgaggatggcgagtggggggcgcgggcgtgaccggggcctcgaagaaggcatcgagcccacgggggtttcccgcgacgccggggccaggaatccccagcgccgccgaaaaaaacaccagcaccgccacgagcggcgacatcgcggctggtgtatcctttttcggcgcggggtcccggcttttatctccggaaagaggaaattgagaaataaactagacatccgcataatcccctttgatttacggaggggaaatacgtgccacgtggcggggggcggggcaactggagcgtggcgaagcggaacggggtgcgatggggcgcatcgaggcgcggacgcgtctgggggagtctggggggagtctggggggagtctggggggagtctggggggagtctggggggagtctggggggagtctgggggaattccagctcggcgcgatcgccgcatcgcgcaaagtccggctgcagtaaatttactgcggatgcagttcccggggcggccgccccggccagatggcgctgcagtatagctactgcggctgcagtttactacagttgcagtaccgcgcgccaccgctaaatactacagtagatttaccgcggccgcggtaaactgcagtttcctgcggccgcggtaaactgcagtatcgcgcggtaaattgtagtctggcagccgcgcgttactgcaattagcggtggctcccgacactctggccaattggtgctaatgggccgtgatggtccacgtgggggtgatgtaaccgccgggccccggttgggcactcagacagtggccgggcgccaggccaaagtgctgtctgagtgccactttatgactttgtttttctcaaacaacatcaattatggatgcacatcgtgtatataatccccggtccgcgctccgcccacccatcacagcagccgcggacgctgcgcgccggagcggtccatctcgccagccagccaaccagccgagccgcccggccgacccgagagccccgagagccagactccctcagccatagaagacaccgggcgggagagacggactgaaaaaatatatctttttttattttgtccgggcctggagacccgcagcaggagcggaggtgggtgtggggccgggagccggagcaggaccgggaacaggaacaggaacaggaacaggaacaggagtggggccgggagcaggagcaggagcaggagcgggagccgaagtgggggcaggagcggcggcggccgcagcagcaacagggtcgccccagtccgcggcgaggaagagggagctcagtcgtcgtcctgggtgaggtcgatgaagatggtagcggagcggggggatcccgacgagctagacgccggaggcccgccccggggggcggcggtctcgggggcagaggcagagggcgacgggcgccgcatcgaggaggagggtgaagacgagggggaggagcgagccgaagcggcggtgttcgccgaccccgggccggccccggcccccgaggcaccatgctgcgcagaggacccctcgccggacgatggcgcctccggagtctcgccctgggcctgtccgcccgtccggccgcgccgcaggcaccgagtccgtctctgcgcgcgcctcagcacggccgcccgtcgggccctgcgcggggagcgcctgggcgccggcctctggtcgtccgcggactcggaggcctccgtcagatcctccgtgtgcaccccgctgctcgaggcgcccgagtcttcctcgtcgggggaagacacctcagagtcagagtgcgcctcggactcggacgtgtcgatatagttcacaccctggtggctcatcggggctcgctgcatccgccgcatccactgcgccgatatgtcaaacagcgtatcgacgagggcgtgggtgtttgccccaaacatggggagcatggcctcggtcacgcgctggcggttcatcccgtgctcctggataatctcgacgatgttgtccacgacggcctcgcggatggggtcgctctcgatgaccgtcgagacctgcccataaagccagttgaagatggggactctggggcgggcgcgagacccagacccggagccctgcccttcggcctcctcgtggcgcacctcctcggtatagtcttcaccccagatgaccgcgaagcccccccctaccggctcatcctcttccccgtcgacatccgtcgccccctccacgggcgtctccacaaacgaagcgtcgctgtccacgtggtggaggatggaggtgacgcgggtcttgcacagcgggcaggcggtgctcgtcagggtccagcgctggatgcagtccagacagaacttgtgcatgcacggcagcgtctgcgcctcggtggccgcgacgtccaggcagatggggcagtccatgacggtgagtggcggtagcaggcgctgcggcggtggcgaggggcttctggggcggtagaagccaaagatcccccaggcgctggaagcctcggcggtggtcgtccgtctccgagagaccgtgcagcccatggtgtcgagggccccgttttttaaccgggtgcccggcccctttgtttgtcttcgcccggggcgtccacgcccccttccgcccgccccgtcggggctccccacgccccctcgggccgtcccgggacgatggccccggcgcgcgccgtcgtgggaagcgggccctcccgcggtgggtcgacggctcctgggtctgaaagcggcgctacggatcccccgctctcacccctgggtccgtcgccgccccgcggtgcgtgcgacgcccgcgaacccccggcccacaacacaccggcaccaccgccaccatcatcatcccccctctccacagacacgctcgcactcgggccacacgctcccgtcctccgccgccaccctcccagcctcccagcctcgcgggtcctcgggacgaccgccgctcaccccgacgggggggggacggggacagggagcgaggggggaggaggacgggggggggaagaggggaacgcgagcgcggcgcaccccgtcgccgtccgtggtggcgtgcccgccaccccgtccgtccatccatccgtccatccaacccccttctcctttttccgcctcccgtcccacggcacggccccatcgacgccacaccgcgcggtgcgcgctctccagccgtcccatccccccacaccccccacgcttcccccattcgcttctcatcccgtcagacctgcgccatggagtttcgaggacgggcgcggggcaacggtggtgagaggcgagccgggggggtcgagtgggcctcgcactttacgcgcacacgcccctctcgcgcaccacacaccaccgcacatcgccgaggtcgggcgagagagggcgccctccccgcgaccccgctcgccccggtcccgacgccctcgcacccccacagcaccccctttcaccccccccccccccgcttctccccgctctctccgcgcgatcgcacacacaacacccgcgcgcatgctcggcttcttcacacggaggacaccatgggcagcagatggtacggtcagccgccggccgccgtgcgcatgtgtcgggctgtcgcggccaagagtatcagcgtggcttttttgttggtcagggtgtgaaaaaggcacgctgatgcgtccgccgctgcagcggagctcctcggcagcgagatcatcgaccaccaccaccacatcgctcttctcgcccgcgctccccccccgccccccccgccatcgcccgcgccccttccccactcccccacccccgccccctctcaccccaccgccaccccacaccatcccccacccccccgtccaccctcaccccccatcgcgtcgcgagcgctctcacacatgcttacatgcacacgcgcccctccctcctcggcgcccccgcggcgggaccgtcgcaaccctctcccccgccgtcccacccgtccgcggccctcctctccttttccccctccccttccccttcccatccccgttcccccctgttccccatttccccgctccctccaccatcgactctcatcgacgggcttctccgccatcatcaccaccaaccaccaatcaccatctcccgccgccgcccggacggcgacccgcggtgctcccgggggcgcgttcccactcacaacccggtggtttcgatggccgcccccggggggttgatggggatgggcgctcgggggtgatcgtctgctaccaaaccgcaccaccgtccccctgtccctcatccacttctcgacggccggctccccgcggggcgggggctgcgggggggtgggtgggtgatagagcaggcgggcgggtctggacgggcggaggggggggtcccggcccctctccctcccctctcttcccccctcccctaccctctctccgtgccgactcgacgagcgggggcgaggcagggtgcgtaccgacccgcctaccaggcaggctagaagcctcacgctgctgcttggcagcgggtgggtacccaacccctcccctcaggacgcgaccgaccggtgtggagagagaaagatgcccgtcccccctgcccccaccatccccgatttttcggttcctctccccgtcgcgccgtcgggcccctcgtcgccaggggtttttttggtggaaggcccaccggggtgaggggggggggggtggtggtgtgtgcgcgacggaacgggccgcaggaggatcggggtgtatgcacggtcgtccccgtctcaaacttcctcgtgtcccctgttccccatccttccacccatacattcatccgtgcggtggtggggatgagtggatggatggaggcgagggacgcggtggaccatgccaggcaagacctgcgtcactcgaagaaagggtgcctggattggccttcgcttctttgccggccacgagcgacggccaccaccgcccccccgacgggctctcttttcccccctcccgcttctcctctttcctctccggcgcgtaccccaccgttttcccgacgaggttcgagaccccccccaaagcaaagcgagagtaccccacacccgcacgcactccccggtgtcgctccttttcccccccggcagcgccggaaccgggtgcaggcggtgggggcgaagattgggttgggtgagagactagaaccggtgttctcaacccttctggagccctaccctctgtgcctggactttccagcgcgcgacacgggggggagcgggcacgcggcggtggcgatggtggcgacggcggtggctgggcgcgatcgaggagatgtggaggggtgccaagcgcccgccgccctcccccgcatctcttctctctcgcacacgcacacgacccgcggatggcgaggatgtggggggagaggtctcccggcgttagtgggtgctatttgattctcctctaaccccctctccctcgcggccccctcgaccatcgtcgtgcagcggcgacggcggctcccaccccgtctctctcccccacacgacctcgacgcgtcccgccaccccgcccagtcacgtcccaccccccggggagaggggagagggatgcacgggggtgggggtccccgccccctctctctgatcccaagacggtgtttgtccccgtctccggctcgcacgcacacatccccgccgggcgggccccggcgggatgcccggccagacaaaccgagggggaggagagggggaggaggaggaagtataggcctctcggggacgggcggcgcgtggcgccggagaggggggtggcggggaagagggaaacctagaaggcggcccatcccccgagatgacgagagcggagagaggcgctaaacggcgcctggggagtttgggggaggaggagacggcgcgcgaggagctgggggaggaggaggaggaggaggaggagacggcgcgcgaggaggaaaatggccggtccaacccttgggacgagaggtctcgaatcttgacaccatttcccctcccctccaccccactcccgtcgcggcttcgcgcggggaggaaaataatccgatccgacccccggggcgagaggctcgcgatcccacccccccgctccccgggggccgcgaaaaaagggggcggggcttaaagggggggggctaatttgcataaatttgcatgcatttgcatagccccgccccctcatttgcataaattagcatggcccctcccccccatttgcataaattagcatggcccctcccccaatttgcatggccccaccccctcatttacataacccctcccactcatttgcatgcatttacataacccctccccctaatctgcataacccctccccctaatctgcatatgacaacttcctccggaagtgacgctctggcccctcccccctcatttacatatgaccgcttcccccggacgtgacgccgggctcctcccacccccccccccccatttgcatatgaccgcttcccggacgtgacgtttttgccgatgacgacttccgcctcatttgcgtatgaccgcttcccgcgcgcaacttccgccggacgtgacgccggtccccacggatctcatctgcatatcgcgccggccccttccgcttcccccggacgtgacgccggcttccggggcgcggacggggcgggctccgcggatcgcatcggcgcgccgagcctgccccttccgtcgcaccgggggtccgcgggcgggggcttccgctccgcggcacccgccccattggctcccctcgcgccacgcggtcgccgtcgtcatcgtcccgccggccaatcgggtggcagcgggggaggctgggagtggggacggaagacggaagccagatgaacctgttccgcccgctctcccaccgcctttcccgcccctccacgacgaccacccgggaccaccaccaccaacaacaccaccacccccccccacgcacacactttactactatcaccaccagggggcgatggttgcaatggcagttccctgtactgaccaccaccgtgtttttttctctctctctctttcctccccccccccccccctcgaccaccgcaggaccaccatcgtctaactcccacccgggaccaccgggaccctcgggaccatctacctcccaccaggacccgccgggaccaccaacaccgtccacctcccaccaccaccaccatcaaggacccccaacatccccaagaccctctacttcttcccacccagacccaccaggaggaggacccccatctgctgagacccaccaggacccaccgggaccctcatctgctgagacccaccaccaccaccaagaccctccaggaggaggacccccatctgctgagacccaccaggacccaccaggacccccatctgctgagacccaccaccaccaagacccaccaggaggaggacccccatccacttcttcccatcaccaccaccaagaccctccaggaggaggacccccatcacccccaccaagaccctccacctcttcttcttcctcccaccagggacccccatccacaagaccacctccgccccagagaccaccgccaagatggccgcctccatctccccaaaaaatctcagagactcgagctggttcagaaaatacagcacaaactttattttctcactctgaaaataaactcttttctcacccgatgggagaaggaggagaaggggaccgggggaccgcgggaggaggaggaggaggagaaggggaccgggacgatcctccgccgccgagccctccgccgcggccgccgccgccgcttccaccaccgccgccaccaccgccgccaccaccgccgccaccaccgccgccacctccgccgccgccgcagccgcctccggccgggggatccgcgcggaggagaaggagaggaggaggaggaggagggccgccgggccggggaggcaggcgccggggaggcaagcgccgccgggccgaggggaccgaggccgccgccgcggacgcggaggaggaggaggaggaggaggacggggacgaggacgaggaccgggccgggggcgaggggagagaagacggaggagaagggcctcgaggagccggtggaggggccggagagtcagagtcagagtccagccgggccgagggggcgccccgctcagcggagcagcaggtaggggttgccggcgtcctcggcctcctcgtcgtccgagatggcctccaccttgatgggcccgagcgggccgcggggccggccgtcgccgccgcggaagtcgacgatctccaccgcggcagagtcctccccgtcctcgccgggcccccgggcgcccgagggccggtgggtctccacggcgccgccggcggcgcggacgctggtctcgaagggcgcaaagtcccagcgcacggccggcggggcgcccgcggccgcgagggcgcccggggccagcaccagcggggcggcctcggcgtcgggctccagcagcgccgcggcgcagaaggcgcgcagctcggccggcaggccctcggggccgcggagctcggcgaggccccggcgaccgcaggagacgaagacgggccgcagcggggcgccgagcccccagcggttggccgcgcggtgcccgaaggcggcgcccgcgtcaaagtccgggtccccgagcccgagcgcggagcgctggcgggccatgtccttgcagccgtccacggtggggagcacgcgctggcggtaggcgcgcggcggcagcgggaccggggtccggggcccggcgcgggtgctcaccgtgtagcgcacgttgtcctggcggcagaggcgcagcggctcggccccggggtgcaggcgggcgaaggaggcctccacgcgggcgaagcaggccgggcccacgatggagctcgagtccaggacggccgcgcgcagctcgcggcactcgggccagcgcacggcgcactgggcggccgggtccaggcgggcgcggacgtagacgtggtagtcccccacggccgggccgtccgcgggccagtcctcgatggtgtccagcacgatgagccggcgccgcgccgcgccgagccgcgagcagaggtactcgacggcgccggcgaagccgaggtcccgcgccgagagcagcagcaccccctgggcgttgaggcggccgatgtcggggcgcccggtccagttcccggcccaggcgtgcgagtccggcgtgcagaggcggtgggcgaaggcggcgagcagcgccgagaggccgccgcggcgcgggtcccaggccgggcgcggggcgccctcggcgggctcggcgcagagctcctcgtggggcagcgggtcgtagagcaccaccacgcgcacgtcctcggggtcggctatctgccgcatccaggcggcgcggcggcggagcggggcgcccgcggccccgcggcgcgcggcgatgtgcgccagggcggccgggtcgaaggtgagcgccgggcgccagagttcggggaagacctcctggtccacgaggacgcgggccacctcgggcgggcagtaggcggcgagggccgcctcggagggccgcggcgtgtgggtctcgccggccgggacgcggcggaagccgccgtcgggcgcggggtgctcgggcatgggcccgagcgggcgccggagccggtcgtcctcggaggaggaggaggaggaggacagcagcgcgggagcggggtccggagcgggcccgagtccgagggagcggcgcttgcgccggggcccccggtcctcttcgtcgtcgcggtggccgtggccgtccccgcggagggccgagccggagagcccctcgtcctcctcgccgtccccggggcggcgggccccgggcgcgcggcgcttcttcttgcgccgctcgggcgctgggtccgggccggcggcgggggagctggcgtagccggaggagccggagaggccggacttggtgccggggctgccggacttggtgccggagctggacttggtgctggagctggacttggtgctggcggggctggagggcccggagccggggaggccggagggggcgcccgccgccgccggcgccggcgctgggacgacgaggccgggctgctcgggccagagcgggggcaggccgggcgcgggctccgcgggcccgggccgcgcggcggcctcggcgagccgggccgcggccacgttggccggggcgaagagggccgcggcgtaggtccaggcggcctcgcgggcgcgggccccgtccacgctgtagcgcaccagcggcgccacggtgcgggcgacgagggcgacagagtccgcggcctgctgccgctcggccgggccggccccggggatcgcgtcgcggagcgcgagcagcgcggcggtcacctcctcgaggcaggcgggcccgagggcggccggggcgcgggcgggcgcgggcagccggagcgggacgggcagcaggcgctcgaggacgccgcggcaggccaggacgcaggcgtccgccagctcgcggggcacgcggccgggctgcgcggcggcgaaggcggcgcggacgcgggcgcagagggcctcgacggtcgcctccccggcgcgggggtccgcggcgcggcccgggtaggccatgtcggcgtaggcccggcggaggctctgcaggatgaaggtcttctgggtgcgatcgtagcggcggctcatggccacggcgctcaccgcgtgcggcagggcccagagcgggtcctgggcggccatggcgtccccgatgtgcggcagcggcggggtcacgctgccggtgatgaaggagccgtggccgtggggcgcgtggacccggcgctggcagaactggttgaagcgctggtcgggggcctgcatccgcgggttctgcagccaggacatggcctcgccggcggccccgctgtagatgaggcgcacgagggcctcgtgctgcttcctcgagtcccccatctccgggatgaagacgggcacgggcccggccgcggcgcggtagcgggccgcggcctggcggacgtcgtcctcgtcccagagcccctcgcgggagtccccggcgccgccgtagcggacgcggccgtcggccggagggtcggagccgggccagggctccccgagcggggtgagcagcggcccgtcggtcggcgggggcccgtcggccatgagcgagaggtggttgttggtggagcggcgcttcctgcgcgggggccgggcgggctccggggccggggccggggaggccgcggcggaggaggaggaggaggaggcggcggcggaggaggccgagggccgcggggccgcggcgggcgccggcggagacggtggcggcccggcgcgggcgagtggggcgccggcccggactccttcgtcttcttctccctcggaggaggacgaggacgaggaggacgaggaggacgaggacgaggaggaggccgagcgccgcgcggcggcggcggcggaggcccggggggcggagggcgagcgggccggggagaggtccgagtcgctgccgctgctgctggagctgctgaagccgcggccgcggcggagggcgccctctccggcgcggcgccggcggggctgtctctgcgggggcgccccgccgtccccggcgaggccgagtccgtcctcgtccttctcggggtcgcgggcgacgggctcgacggcgacggtggtggaggagctggagttggagttggagttggaggagacggggctccgggcgccaagcggccgaggatcgagccgcctcgcggcggcgggctcgtcgagcaggggctcgcggtgctggtgatggtgacgaccgcggtcccctccggcggagggggcgccgccgccgccgagcgccgagaccggcccggcggcgggggaggctggggaagcgggcccccgccgtgccggcgctgcggccaccgctgctggctgtgctggtggcgccggggtccgaggccgcgccgccggcccgggctcaccgaccgggtcccccctcgcgggggaccatctccgcggggctgccgaggggccgggggagccggaggaggaggccggggaggctgcggagggggacgggcgcccggggccgccgggggccccggcctctgccgctgcgagtgctgccggggtcggcggccggggcccggagccggcccgggaccggggcccgaggacgaggtgaccgtgctcggagccctgatggagagcccgaccgggggacccggcggccggggacccgggctcgtcctcctcctcgtcttcgtcgtctagcaccacgatctcgcccgagccccggcggggctgctgctgctgctgctgctgggccgaaggaggacggggcggcctcgtggctccggccgcggccgcgaggacggcggcctcggcctcggcctcgccggcatcgtcggagaagaggccgccggggccaaagaggagatcctcgccggaggggccgcggcgccgggagccctggctgccgccgtcggagccggccgcggtcccctcttcctcggccgccgcggccgcggccgccgccgccaggagctggctgaagttgccctcggtctcgatgaagtcaaagagatcgtcggccatggtctcgatcggttctcggggtgtctctctctctctcgcgcgccgggagccgagctcggagagcgggtgctggagggtgagagggagccggaggagcgggcgcggcagaagaagaagaaggagagggcgaagaaggagaagaagaagagggctctcctctctcttccccccggtgggtcgggccgtccgtcgagggctgatagccgccggagagccggagtcttcagagtccgcgccggagccgagacgatcggatcccctcgggttggcagagaacgatgctgtccgtacctgcaccgcagtgaagtgctacgatggagaccgtgcctataaactccccgagaagagcccgcccccaggtaagcggaccaatggccgattttcgccgcggacttccccgacggccggccaatgggatttttctcgcccgcttcctctcgcgtctgctttgcatgcccggcccaagatggcggccgccggccaatgggatttcgcgaggaacttcctcgcgaggaccatttgcatgcccggcccccgcggcggccatcttgcccactcgacggccaatgggatttctctcgcccgcttcctctcgcgtctactttgcatgtccgtcccccgcggcggccatcttggtcgacggcggccaatgggatttctctcgcccgcttcctctcgcgtctactttgcatgcccggccccgagggcgccatcttggcccctcgacggccaatgggatttctctccctacttcctctcgcgtctactttgcatgcccggcccccgcggcggccatctcggctcgcccgggccaatgggcgcgcggaggcgtctcccgcgcgcctctgatttgcatgcccggcccgctctgcggccatcttggccgcgggcggccaatgagattgtccgaaaatccctcgcgcgggcgcgaggcgcatgctcggcacgcgacccacccccgtggtgctagcgagccaatcagatgattttcggggaagcttccgtgtgcacgtcatttgcatgctcgccccacgtggccgccctcggccaatggggcctcacggtgcaagcttccgtgtgtctgcacgtggtccgcatgtgttgtggtggtctctcagtgttttgtggtggtctctgtgttgtgtggtggtctctcagtgttttgtggtggtctctgtgttgtgtggtggtctcaccgcctccccctgccactcgcgagacccccgagacccccgtttccccctccccgagacccttgaggccccccgcgacccccgcggtcgccccacccgcgcctgcgcgctcggcgcgcgctccgagggcgcccccagccggtcggagagacgagcggaaccgccgtcggaccggggaccggcgaccggacccgaaccgggaagcgacgccggggcgggagaaccggacccgaacctcgagcccggacccgcccggacccggaagggaggagccggacagccacgccttggatacttttgtcgcccacccacccccccccctctacctctctctcccggtcccccctcccaccccacgagacacgccccagagtgaaaaaaataaaagttgttctcgttgcaccgtcttccggctcgtgtcgtccttccgcggtacctcgggcgggcgggagggggcgcgagaccggctcggcctcgtcgggagagagggagttgggggggaggggagccaagatggcggcggggcgtggcggggcgtggcagaggggaggggggggggtcggagcgcggaccccgcccggtgggggggcgggtgcaaaggggcggggcccaaaatggacctcgggccgggaccccgggggcgctccgggagacgaagagggccgggcccccttcccggcggggagggccgggccggcgcgccgggacgcccctccgggggaaagagtgtccccgcgcgggcgccgcgtcccgccccgagcccccgggggcgcgcgggcctcggtcgcgcccgccggacgcggaggcgcgaggcccccgccccccggggggtccgggacgggggggcccaaaatcgggctgggggacggcgggggggtttgctccggaggtctgcgggtgtgagtgtgtgtgtgtgtgcggggagagaaggctcgctctctctctcggagggccgggcgggtggagggttgggcggacggatggtcggtcggacggggcgggcggggagggtgtcgtccgtgtgtctgtgcgcggggagagtgtgatgtgggggtgtgcgagagagagagcgtgcgtgtgggtgggtgtgagggccggtgcgggtatgggggcccgtgcgggtgggagggtccgtgtgggtgtcggtggtgcgggtgttgctggtgataccggtaccgacggtggtggccgggcgggcggtctctccgtctccgtctccgccgtcgcctcggtccgaggaggggggcgcccgggcgtgtctcgcctttcccccagttcgcctcctccttctcctccttctcctccttctcctccttctcctccttctcctccttctcctccttctcctccttctcctcctctccccgcccccgccccctgtcccggtcccggtcccacgcgcgcgccccggggaagggtcgggcgatggccgccgccaccgccccacccttcccccggaccccctcccgcccgccggggcgccccgcgtgctccgggggcgccggccggccatccccccacacctcccctcctccccgtccccaacccgccccgcactcgcccgacactcggcccgcggcacgcgcgcccagaccttcctctcccccctccaccgcccgccctcgccccctccgtctcatgccacccgcggcagcatcccccctccccgtcgtcctcagccaacagccaacagccaacagccaacaacccacagccaacagcctcactctcgcgctctccccgtcgcgggaaaaaaacacggcaccacaggcgggagcggggtcgctcgggggaaggtttcattcaaacgacaggcgacaggaggggggcccccgcgcgggtcgggccttcttccccatccccgccggcccccctctttctctctctctcttcttccgcgtcttcttccgcgtccgcttcggccccccgcggccgcggcgtgggagagcggggcgggtggggaaggaagaggggacgggggggagaagaagaaacggggaagcgggggggaaggaacagacggggaaggggggggaaagggaaacggggaaacggggaaggggccggcggaggggaagcgggagggagaggcgcggcgccccgcgcttccccttcccgcttccccctccgcccggccgcgggcgcccgggagacgggagcgaaaggaggagaggggaggaaaaaaagtctagagcggggaggagaagtcaccgtagtagtcatagtagtagtcatagtaatatagtaggagtaggagtagtaggagtagtagtagtaggagagtctgtgcttaccgagagaggagaagaagaagcagaagccggggcgcttcctccccggcggcccgggaagaaagccccggcgcctcgggccccggagatggggacgcggagaaggaaacacccaagggcggaggaggaggaagaaggagaggaagggaaaaaaaaacctcgagagcggcggcggcggtcgccgcggccaggaggaaaaacggggggagcgggagcggctcccgagtccgggaaggaaaggcgggcggaggaaggcgggtgtggaggaggaagcgctcgcgcccctcgctctctctccccccgtccccctctccgcgtctcttccccgcgtcctctctctgcgttcgccggtcgcggcgcgggcggcggctgcagaggcggctgcggacgcggaggggggcgaggggacacagaactctttttgtcctcctcgccggctcccaggcggcgggcgcgtcccgcgtccccggcgtcatcacgcgggaccccccggtccccgtgacgcaggacgcggggaggggaggggagggaggggagggggtcggggggaagggaggggggggaggagggggagagcgggtgacgccgcgggtgggccgaggccggcggggagggaggaggaggacgcggcggcggcggtgaaggaggagagccgcccggcgcgcgcccgccgccgcaggcgcgtccccggcgcgggcgcctctccacgccccgctccgcccggcgcccaatggcgcggccggctcggcggcccggcccttcctccgccctcctccttccccgccgccaccgccgcctcctcccttctccctcctccctcccctcccgccgcccgcgcgcccgcgtgcgctcgtgccggcgcggcatccccgcccaggcggcgggggagcgcggagcgcgccgcctccccgtgatcacgttcgcgcaccgcggcgccagcaggaaaaaaaaacaacaacacagagacacacctccctctccgtctccctcgccttctccgttttggggggagaccgatgccggtgccacatacacatacacacgcgcgcgcaccccgtcccccccccttccccttcccctcgccccggccccccggctcggtgccggagaaaagggggtgccggcgcggaccggggtcgggcccgcggcgccggggggtgggcgcgcgtgtccgtgtgagagtttacggtgtgggtgggtgtcacggtggagcgagctggggggccccggctcgctcgctcgctcgctcgccaggccggcaggccgggggcccggcctggacgcgggccacactgtgcggcggaccgtgccaagtttagggcgctgcgacccagagagagcgccgagctaaaagctattttatcgccgctgcggtttcccctggcgcggcgcctagacaccgtttctgcagacagagccctctctaatggcgcgtccgggaaggcttctcccgggccgcgagggccgcggtggcgacccccttcctcccccctcctccccgtccccgtccccgtcccccgcccgctctccccgggccgccgcgataccgcgcgggcgataccgcgcgggccacccgcgggcgctaccgcgccgctccgctcgccgcctcttccccctcctccaccgccgccagtcctcctcgctcctctccgcccacgcaactcctcccgcccgcgtgcccccggggccatcggctggaacaccctgagccttcgctgagtgctgcccccgccccgcccgcggcactccgggcccgcgtgagagggtacagagcgaacccggccccgaccccctccgcccgccgggggaggcttcccggaaccagcgcggtcatcaaccccctcccccacccacccgcacagcgcgcacgccgaccgcccgcgtttgctacgatccctgcgcccgacggggccgcgggacggcgcgcgcggcacggaacgagaggggggcgggggtgcgggcgagtgagcgtgcgcgaaggggggagagggcgcgtgggcgagagggacggggcggggcggggtggggggagcggcggctcgccctccgtcctcccctccttcctctccccgtcgactcgcgtcgcggcgattcgggccgcatcgaggcgcgtcggggttttcgcgccctcgcgccgccgccgcctcctcatctttcgtggttaacacacaccacacatacacacaccctcccccgcgccgctgtctgtggcgcccgtcgtctctcccgccggcgcaaccccgggccctgagagctttctccgctttctatttcggccccggcctccccgacctctctcacccgccctctcgcgctctctttcccacacacacacacataacaagaggctcgccctcgttccatcatctcggcgtactcgccgcctcgtacactacatccgtctaccgccgccagcaaccctttttttcccccgctcactcctcttcccccctcctccttcccccctcccgcctctccattcatcaatctcgtccaggccgcgggcccattcaccatcgccgcggtccgagctcggccaggagagatggatggatggattgggggaaggaggggggcggggagcgcgagcgggagaaggttggcgaaggagagagcgagagcgggaactggagagggggctgggtggggtgaggcgagtgaccgccaccgccctctaccaccaccacccctcccctccctctcctctcctctctctccccggtcccgtgtgcacacacagagagtgtttttttttccttcttcttcttcccggttcctcttcgtcctccggaccctcggtgccgcgatcccccaccccgccccccttcctcctccccccatttcccccgcccccaattcccccactcctcccaccagcccccgccgccgcggccgcagccgccgcgatccgtcctccccattcatcctttttccagaggccattcatcgccccgctcatccgtcttcatcgccgcggagggctggctcgaggccagcgcgccctttcctctcccggcgcgcccgctccctccgcgtcccctctccccgccgccccctcggccgcaccgagccttctccgcgcggtcccgtctctcccctccccccatcccgccccactccccgcccccctctccccctccctctcccgccggtgttttcccacgcccacactcactttctccgctcccaccccaccctcctgactcacccgtcccgggggattccctgtcccccggggcgcggccgacccggcggtgacccgtccccgccccgccgcgcccctttttcctcgagagcgggcgtccgggaagggagaggtcgcggcgggtcacgcgcgctcccgcgacttcccggcccgcgatgcggtcccggaaatttccgggacggtcgcggcgaccgtctcggggcccgcggcgccgccaactcgagtcttggcacgcgcccagcgttcgcacttgttgtatataagatataatactagtatatattagcaattggtgcgaacgtgacgtggcccaatcaaatctctccacgggccccacgtgcccctattggcaatttatcgaccgctttccgaagggatcgaaccccgcggcacccgcctccggggacgcgccggccaatgggggagccggacccgcgtcccctgagcccacgtgtgaggccccggccaatgggcccgtcggcccggccccctcgacggggcgataaaggggtgcgcggcggcccggccgacggcactcgccgccccaacgacgccgacggggatcgaccgggagaggcgcggagcgcgccccgagccgacgaggagccgcgccccgctcgaggacgcccggacgacgacggtgacgtcggcatcggcctcgacgacgacgatcgcgggacccgacggtaccggatgcgggggatcccccggtcggggtgggccgggagtgggcgtggcacctcctcccccgtcccccctccctacccaccccgcgcgatcgcgctcatcgtatccgttctcgtttcaggtgccgcatcgttccggaagaaccgcctcgggggtcgcgcgtccgtcgcctaggtgagtcggggggcttcccgggtgcggctcccccctccccctccccctccccgggacgggtcgggtgggtaggtgggcgggggtcgggagggatcgggggaaggggagggggtcgtggctcacgctgacgtcatgcgttttcttccccccccccacagggacgcgagcgggaccggctggtcgaccacccatccaatccatccaatccatccagccacccacccgtcggggcggccgggctcgggtccggttccgggtcggcgtcggggagcctcgcccacccacccggccccccgtcccagcccagccccccgcacacccccatcgcaccccctcccaccccaccccgttcccaccaccccgcccgacccaccgcgtccgccgcgcccatggaccgggtctgggccgactggtacgagcccgtgccctccccgccgttctcgcccgtcgacccgcccgggccccggcccgcgaccccggtcccggggagcagccccccgtcccccgcctcgacccccacgccccccaagcgcgggcgctacgtcgtcgagcaccccgagtacgggcccccgcccgaccccgaggaggtgcgcgtccacggcgcgcggggccccggcgccttctgcgcggccccctggcaccccgacacgcggcgcctcggggccgacgtgaaccgcctctttcgcggcatcgccgtctcggccgccgacgtgacgggcgacacgcgcgccctgcgccgcgccctctttgacttttacgccatgggctacacgcgccagcgcccctcggccccctgctggcaggccctgctccagctctcgcccgagcagagcgccccgctgcgcagcgcgctgcgcgagctcaacgagcgcgacgtctacgacccgcgcgtcctctccccgccggtcatcgagggcccgctctttggggaggagtgcgacgtcgacgaggacgacgccggctcggacaccaccgtcgcgtccgagtttagcttccgcggctcggtgtgcgaggacgacggggaggacgaagaggaagacggggaagaggaagacgaggacgaggagggggaagaggaagaagacgaggaggaagaggaaggggacgaggacggggagacggacgtgtacgaggaggacgaggacgaggacgacgaggacggggacgggctctgtgaggacgaggacgaggaggacggggacgactttgacggggccagcgtgggcgacgacgacgtgtttgagccccccgaggacggctcggacggagagggctcgggctcggacgacggcggggacggggaagaggaagaggaagaggaagacggggacgaggacgaggaagaggaggacgaggaagaggaggacgaggaagaggaggacgaggaagaggaggacgaggaagaggaggacgaggaagaggaggacgaggaagaggaggacgaggaagaggaggacgaggaagaggaggacgggggggaagacggtgaagacggcgaagaagacgaagacggagagggcgaggagggcgggaaggacgccgcccgccgggggacgcgcgccccgacgcggcccgccgccgccccgtgaggcgggccgcgccccgctctgttcttcctccacccccaccgtccgccccgtcgtttgcccctttccccttccccccccccctctgttgtgccctcaataaacacggcggcccgccgctcgaacctcaactctctcgtctctcgggcgttttttcccctcccggccctcgtggggaagggagatggggggatgagacggggtggagggagggagatgggagtgagacggggggatgagacggggggatgagacggggagcgagccgcggaaccctcccgcggggggcgagagtcggacgaagcgggggcaggggaggggggggacggggtgggggggcggggtgggggggggacggggtgggggggggacggggtggggggggggaccgccggtccggccgacgccgggcgccggacgccggacgacgcggtcgcgcacccagcgcaccacacgcgccgcgcaccatcgtcggcggcgcgcgggggggggatggggggatgggggatggggggaagtggcgtcagtggtgacgtcggtgaagcgggagggggaaaggggtgagagtgtgtgggtggtggcggcgcgagtctgcgggatggtgccgaggggagccggacggtcacggggagggaggatgtggcgcgcgtggggcgtgggccgcctctgccgcggtcgcgagccgccaacgccgccgccaccgcctcccgcccaaagtgatatcgcggcgtgccgggacggccggcgctcattcacaccgcacccgttcacccgcgtccccgcgggcaagcacgcacacacccggtcgcgcatcatgctggcgatgtggagatgggtcaccaagaggtcgcggctccgccgaggccacgcccatcttgggggaaataaaggagtccggggaatgtgttccttataccttgccgggctcagcagggggttgtcgcgcgtccacgcccagcgctcgcacgcagcaacaatggccgacgccggaatccccgacgagatcctgtactcggacatcagcgacgacgagatcatcatcgacggcgacggcgacagcagcggggacgaggacgacgatgacggggggctgacgcggcaggccgcgtcgcgcatcgccacggacctgggcttcgaggtgctgcagcccctgcagtcgggctcggagggccgcgtcttcgtggcccgccggcccggcgaggcggacacggtggtgctgaaggtgggccagaagccctcgacgctgatggagggcatgctgctgaagcgcctggcccacgataacgtcatgagcctgaagcagatgctcgcccggggcccggtgacgtgcctggtcctgccgcactttcggtgcgatctgtacagctacctgaccatgcgggacgggccgctggacatgcgcgacgccgggcgcgtgatccggtccgtgctccgcgggctcgcctacctgcacgggatgcgcatcatgcaccgcgacgtcaaggcggagaacatcttcctcgaggacgtggacacggtgtgcctgggggacctcggggccgcgcgctgcaacgtggcggcgcccaacttttacgggctcgccgggaccatcgagaccaacgcccccgaggtgctcgcgcgcgaccgctacgacaccaaggtcgacgtctggggtgcgggggtggtgctcttcgagacgctggcctaccccaagacgatcaccggcggggacgagcccgcgatcaacggggagatgcacctgatcgacctcatccgcgccctcggggtgcaccccgaggagttcccgcccgacacgcgcctccggagcgagttcgtccggtacgccgggacccatcgccagccgtacacgcagtacgcgcgcgtggctcgcctcgggctgcccgagacgggggctttcctgatttacaagatgttgacgtttgatcccgtccgccgcccttccgctgatgagatactcaactttggaatgtggaccgtataaaacggcccggctccgagcggtaggacacacacacctttgcgcatctccacagctcaacaatgaagtgggcaacgtggatcctcgccctcgggctcctcgtggtccgcaccgtcgtggccagagaggcccctcgggagctctgctacggccaccccgtccacgacgaccggcggcccgtcgggcccgcgaccgacgcccagcccgtgaacccgctcgcccccgccaacgccaccgggacggactactctcgcggctgcgagatgcgcctcctggacccgcctctcgatgtctcgtcccgctccccggaccccgtcaacgtgaccgtcgcctggttctttgacggcggccactgcaaggtgcccctcgtccaccgcgagtactacggctgccccggggacgccatgccctccgtcgagacgtgcaccggcgggtactcgtacacccgcacgcgcatcgacaccctgatggagtacgccctcgtgaacgccagcctcgtgctgcagcccgggctgtacgacgccggcctgtacatcgtcgtgctcgtctttggcgacgacgcctacctcggcaccgtctccctgtcggtggaggccaacctggactacccctgcggcatgaagcacgggctcacgatcacccgccccggggccaccctcccgcccatcgcccccacggccggcgaccaccagcgctggcgcgggtgcttcccctcgaccgacgagggcgcctgggagaacgtgaccgccgccgagaagggcctgtccgacgactacgccgactactacgacgtgcacatcttccgcctggagtctgacgacgaggtcgtccacggcgatgcccccgaggcccccgagggcgaggaggtgaccgaggaggaggccgagctgacctccagcgacctcgacaacatcgagatcgaggtcgtgggctcgcccgccgctcccgtcgagggcgccggcgacggcgaggaggggcacggggacgaggaggacgaggagctgacctccagcgacctcgacaacatcgagatcgaggtcgtgggctcgcccgcggccgcccgcttcttcgccgcctccaccaccccccgcgcccccacccgcgcggccgagatcacgaccatgaccacggtcaccaccgtgcggacgaccgaggaccccagcggcatcaccgactgccgccggagcgacttcgtctcgccctctgacatcttcgtgacccccaccggcagccccgccctgctcctgggcttcctgggcagcgcgctcgcctcgcgccccctgcacctgacggccggggagacggcccagcacgtgcgcgaggcccagcagaagagccgccacgtccgctccctcggcggcctccagctctcggtcgagaccgagaccaccaacaccaccaccacccagacgggcctgtcgggcgacatccgcacctcgatctacatctgcgtcgccctcgccggcctggtcgtcgtgggcatcgtcatcatgtgcctccacatggcgatcatcagggcccgggcccggaacgacggctaccgccacgtggcctccgcctgacccggccccgcccgactcccccgcgattcccccctctctctcaccgggtgtccatcttcaataaagtatgtctcaaacacctaatttgcgtacggccttgcttacggggggtgcgatccacgcccagcggtccataaaattgggtcggcgccccaggttcccatacactcacccgccagcgccatgctgctcgcagcgctattggcggcgctggtcgcccggacgacgctcggcgcggacgtggacgccgtgcccgcgccgaccttccccccgcccgcgtacccgtacaccgagtcgtggcagctgacgctgacgacggtcccctcgcccttcgtcggccccgcggacgtctaccacacgcgcccgctggaggacccgtgcggggtggtggcgctgatctccgacccgcaggtggaccggctgctgaacgaggcggtggcccaccggcggcccacgtaccgcgcccacgtggcctggtaccgcatcgcggacgggtgcgcgcacctgctgtactttatcgagtacgccgactgcgaccccaggcagatctttgggcgctgccggcgccgcaccacgccgatgtggtggaccccgtccgcggactacatgttccccacggaggacgagctggggctgctcatggtggccccggggcggttcaacgagggccagtaccggcgcctggtgtccgtcgacggcgtgaacatcctcaccgacttcatggtggcgctccccgaggggcaagagtgcccgttcgcccgcgtggaccagcaccgcacgtacaagttcggcgcgtgctggagcgacgacagcttcaagcggggcgtggacgtgatgcgattcctgacgccgttctaccagcagcccccgcaccgggaggtggtgaactactggtaccgcaagaacggccggacgctcccgcgggcctacgccgccgccacgccgtacgccatcgaccccgcgcggccctcggcgggctcgccgaggcccaggccccggccccggcccaggccccggccgaagcccgagcccgccccggcgacgcccgcgccccccggccgcctgcccgagccggcgacgcgggaccacgccgccggggggcgccccacgccgcgacccccgaggcccgagacgccgcaccgccccttcgccccgccggccgtcgtgcccagcgggtggccgcagcccgcggagccgttcccgccccggaccaccgccgcgccgggcgtctcgcgccaccgctcggtgatcgtcggcacgggcaccgcgatgggcgcgctcctggtgggcgtgtgcgtctacatcttcttccgcctgaggggggcgaaggggtatcgcctcctgggcggtcccgcggacgccgacgagctaaaagcgcagcccggtccgtagcctccgcagtaccggcgtcgatgatgatggtggcgcgcgacgtgacccggctccccgcggggctcctcctcgccgccctgaccctggccgccctgaccccgcgcgtcgggggcgtcctcttcaggggcgccggcgtcagcgtgcacgtcgccggcagcgccgtcctcgtgcccggcgacgcgcccaacctgacgatagacgggacgctgctgtttctggaggggccctcgccgagcaactacagcgggcgcgtggagctgctgcgcctcgaccccaagcgcgcctgctacacgcgcgagtacgccgccgagtacgacctctgcccccgcgtgcaccacgaagccttccgcggctgcctgcgcaagcgcgagccgctcgcccggcgcgcgtccgccgcggtggaggcgcgccggctgctgttcgtctcgcgcccggcctcgggggacgcggggtcgtacgtgctgcgggtccgcgtgaacgggaccacggacctctttgtgctgacggccctggtgccgccgagggggcgccccgtccccacgtcgccgcccgcggacgagtgccggcccgtcgtcggatcgtggcacgacagcctgcgcgtcgtggaccccgccgaggacgccgtgttcaccacccagcccccgcccgagcccgagccgccgacgacccccgcgcccccccgggggaccggcgccacccccgagccccgatcggacgaggaggaggagggtgacgcggagacgacgacgccgacgctgaccccggcgcccgggaccctggacgcgaacggcacgatggtgctgaacgccagcgtcgtgtcgcgcgtcctgctcgccgccgccaacgccacggcgggcgcccggagccccgggaagatagccatggtgctggggcccacgatcgtcgtcctcctgatcttcctgggcgggatcgcctgcgtggcccggcgctgcgcgcggaatcgcatctaccggccgcgacccgggcgcggatcggcggtccatgcggcgcccccgcggcgcccgccccccaaccccgtcgccggggcgcccgtcccccagcccaagatgacgttggccgagctgcgccagaagctcgccaccatcgcagaagaacaataaaaaggtggtgtttgcataattttgtgggtggcgttttatctccgtccgcgccgttttaaacctgggcacccccgcgagtctcgcacacaccggggttgagaccatgcggccctttctgctgcgcgccgcgcagctcctggcgctgctggccctggcgctctccaccgaggccccgagcctctccgccgagacgaccccgggccccgtcaccgaggtcccgagtccctcggccgaggtctgggacgacctctccaccgaggccgacgacgatgacctcaacggcgacctcgacggcgacgaccgccgcgcgggcttcggctcggccctcgcatccctgagggaggcgcccccggcccatctggtgaacgtgtccgagggcgccaacttcaccctcgacgcgcgcggcgacggcgccgtgctggccgggatctggacgttcctgcccgtccgcggctgcgacgccgtgtcggtgaccacggtgtgcttcgagaccgcgtgccacccggacctggtgctgggccgcgcctgcgtccccgaggccccggagatgggcatcggcgactacctgccgcccgaggtgccgcggctccggcgcgagccgcccatcgtcaccccggagcggtggtcgccgcacctgagcgtcctgcgggccacgcccaacgacacgggcctctacacgctgcacgacgcctcggggccgcgggccgtgttctttgtggcggtgggcgaccggccgcccgcgccggcggacccggtgggccccgcgcgccacgagccccgcttccacgcgctcggcttccactcgcagctcttctcgcccggggacacgttcgacctgatgccgcgcgtggtctcggacatgggcgactcgcgcgagaactttaccgccacgctggactggtactacgcgcgcgcgcccccgcggtgcctgctgtactacgtgtacgagccctgcatctaccacccgcgcgcgcccgagtgcctgcgcccggtggacccggcgtgcagcttcacctcgccggcgcgcgcgcggctggtggcgcgccgcgcgtacgcctcgtgcagcccgctgctcggggaccggtggctgaccgcctgccccttcgacgccttcggcgaggaggtgcacacgaacgccaccgcggacgagtcggggctgtacgtgctcgtgatgacccacaacggccacgtcgccacctgggactacacgctcgtcgccaccgcggccgagtacgtcacggtcatcaaggagctgacggccccggcccgggccccgggcaccccgtggggccccggcggcggcgacgacgcgatctacgtggacggcgtcacgacgccggcgccgcccgcgcgcccgtggaacccgtacggccggacgacgcccgggcggctgtttgtgctggcgctgggctccttcgtgatgacgtgcgtcgtcgggggggccatctggctctgcgtgctgtgctcccggcgccgggcggcctcgcggccgttccgggtgccgacgcgggcgcggacgcacatgctctctccggtgtacaccagcctgcccacgcacgaggactactacgacggcgacgacgacgacgacgaggaggcgggcgtcatccgccggcggcccgcctcccccagcggagacagcggctacgaggggccgtacgcgagcctggaccccgaggacgagttcagcagcgacgaggacgacgggctgtacgtgcgccccgaggaggcgccccgctccggcttcgacgtctggttccgcgatccggagaaaccggaagtgacgaatggacccaactatggcgtgaccgccaaccgcctgttgatgtcccgccccgcttaaataccgggagaaccggtccgcccgcattccgacatgcccggcgccgcctccgtcgacatggacacgtttgaccccagcgcccccgtcccgacgagcgtctcgaacccggccgccgacgtcctgctggcccccaagggaccccgctccccgctgcgcccccaggacgactcggactgctactacagcgagagcgacaacgagacgcccagcgagttcctgcgccgcgtgggacgccggcaggcggcgcgtcggagacgccgccgctgcctgatgggcgtcgcgatcagcgccgccgcgctggtcatctgctcgctgtccgcgctactcgggggcatcgtcgccaggcacgtgtagcgagcgagcgagcgaacgggagcgggggcccgcccccatccgccgcgcccaggagaggggggagagagcggggggttgggcgcgccacgtggtgtgggcacggactcggacttgtcacaataaatgggccccggcgtgtccgggcgcacacagcagccttcctctcctccgcgtctctgttccgcccgtctctcgccggactcttcttctccaccgcctccaccgtcgcagttgtcgcgagcgcgttcgcaccatgggggtgacggccatcaccgtggtcacgctgatggacggggccgggcgcatccccgccttcgtgggcgaggcgcacccggacctgtggaaggtgctcaccgagtggtgctacgcgtcgatggtgcagcagcggcgcgccgccgacgagaactcgccgcggcagcacgtggtgctgcgctcctcggagatctcccccggctcgctggccctgctgccgcgcgccgtgcgccccgtcgtgcggacgcggtccgaccccacggcgccgttctacatcaccaccgagacgcacgagctgacgcggcgccccccggcggacggctcgaagcccggggagcccctcaggatcagcccacccccgcggctggacacggagtggtcgtccgtcctgaacgggatccagtacctgaactcgggggcccggggcacggcccccgtccacctgtggatcctgggcgccgccgacctctgcgaccaggtgctcctggccgcctcccgcagcaccgccgccggagcctcccacgcccagacgggcgcgcgcctgacccggcgccggcccgggctgacggacgccgacgccctggacgtgatcgtcgccgggatccaggcgacccgcgccatgttcgcgcgggtccacaaccgctcctggcgccacgccggcgagtggacggaggccctgcactcccagatcgtgacccggggcgacgtgcgccggcgccgaggcgggcgcggcaacggacgcgagcgcgccccgcgatgtaccatctcctagacggcaggatctctccgcgtcccccacccccccaaaaaacaaacaataaacgctctcgctctggcacccgatgacacgcctccgtcctctctctccctcccactgacgccacccctcccctgcccccgcttcgtccgactctcgccccccgcgggagggttccgcggctcgctccccgtctcatccccccgtctcatccccccgtctcactcccatctccctccctccaccccgtctcatccccccatctcccttccccacgagggccgggaggggaaaaaacgcccgagagacgagagagttgaggttcgagcggcgggccgccgtgtttattgagggcacaacagagggggggggggaaggggaaaggggcaaacgacggggcggacggtgggggtggaggaagaacagagcggggcgcggcccgcctcacggggcggcggcgggccgcgtcggggcgcgcgtcccccggcgggcggcgtccttcccgccctcctcgccctctccgtcttcgtcttcttcgccgtcttcaccgtcttcccccccgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcgtccccgtcttcctcttcctcttcctcttccccgtccccgccgtcgtccgagcccgagccctctccgtccgagccgtcctcggggggctcaaacacgtcgtcgtcgcccacgctggccccgtcaaagtcgtccccgtcctcctcgtcctcgtcctcacagagcccgtccccgtcctcgtcgtcctcgtcctcgtcctcctcgtacacgtccgtctccccgtcctcgtccccttcctcttcctcctcgtcttcttcctcttccccctcctcgtcctcgtcttcctcttccccgtcttcctcttcgtcctccccgtcgtcctcgcacaccgagccgcggaagctaaactcggacgcgacggtggtgtccgagccggcgtcgtcctcgtcgacgtcgcactcctccccaaagagcgggccctcgatgaccggcggggagaggacgcgcgggtcgtagacgtcgcgctcgttgagctcgcgcagcgcgctgcgcagcggggcgctctgctcgggcgagagctggagcagggcctgccagcagggggccgaggggcgctggcgcgtgtagcccatggcgtaaaagtcaaagagggcgcggcgcagggcgcgcgtgtcgcccgtcacgtcggcggccgagacggcgatgccgcgaaagaggcggttcacgtcggccccgaggcgccgcgtgtcggggtgccagggggccgcgcagaaggcgccggggccccgcgcgccgtggacgcgcacctcctcggggtcgggcgggggcccgtactcggggtgctcgacgacgtagcgcccgcgcttggggggcgtgggggtcgaggcgggggacggggggctgctccccgggaccggggtcgcgggccggggcccgggcgggtcgacgggcgagaacggcggggagggcacgggctcgtaccagtcggcccagacccggtccatgggcgcggcggacgcggtgggtcgggcggggtggtgggaacggggtggggtgggagggggtgcgatgggggtgtgcggggggctgggctgggacggggggccgggtgggtgggcgaggctccccgacgccgacccggaaccggacccgagcccggccgccccgacgggtgggtggctggatggattggatggattggatgggtggtcgaccagccggtcccgctcgcgtccctgtggggggggggaagaaaacgcatgacgtcagcgtgagccacgaccccctccccttcccccgatccctcccgacccccgcccacctacccacccgacccgtcccggggagggggagggggaggggggagccgcacccgggaagccccccgactcacctaggcgacggacgcgcgacccccgaggcggttcttccggaacgatgcggcacctgaaacgagaacggatacgatgagcgcgatcgcgcggggtgggtagggaggggggacgggggaggaggtgccacgcccactcccggcccaccccgaccgggggatcccccgcatccggtaccgtcgggtcccgcgatcgtcgtcgtcgaggccgatgccgacgtcaccgtcgtcgtccgggcgtcctcgagcggggcgcggctcctcgtcggctcggggcgcgctccgcgcctctcccggtcgatccccgtcggcgtcgttggggcggcgagtgccgtcggccgggccgccgcgcacccctttatcgccccgtcgagggggccgggccgacgggcccattggccggggcctcacacgtgggctcaggggacgcgggtccggctcccccattggccggcgcgtccccggaggcgggtgccgcggggttcgatcccttcggaaagcggtcgataaattgccaataggggcacgtggggcccgtggagagatttgattgggccacgtcacgttcgcaccaattgctaatatatactagtattatatcttatatacaacaagtgcgaacgctgggcgcgtgccaagactcgagttggcggcgccgcgggccccgagacggtcgccgcgaccgtcccggaaatttccgggaccgcatcgcgggccgggaagtcgcgggagcgcgcgtgacccgccgcgacctctcccttcccggacgcccgctctcgaggaaaaaggggcgcggcggggcggggacgggtcaccgccgggtcggccgcgccccgggggacagggaatcccccgggacgggtgagtcaggagggtggggtgggagcggagaaagtgagtgtgggcgtgggaaaacaccggcgggagagggagggggagaggggggcggggagtggggcgggatggggggaggggagagacgggaccgcgcggagaaggctcggtgcggccgagggggcggcggggagaggggacgcggagggagcgggcgcgccgggagaggaaagggcgcgctggcctcgagccagccctccgcggcgatgaagacggatgagcggggcgatgaatggcctctggaaaaaggatgaatggggaggacggatcgcggcggctgcggccgcggcggcgggggctggtgggaggagtgggggaattgggggcgggggaaatggggggaggaggaaggggggcggggtgggggatcgcggcaccgagggtccggaggacgaagaggaaccgggaagaagaagaaggaaaaaaaaacactctctgtgtgtgcacacgggaccggggagagagaggagaggagagggaggggaggggtggtggtggtagagggcggtggcggtcactcgcctcaccccacccagccccctctccagttcccgctctcgctctctccttcgccaaccttctcccgctcgcgctccccgcccccctccttcccccaatccatccatccatctctcctggccgagctcggaccgcggcgatggtgaatgggcccgcggcctggacgagattgatgaatggagaggcgggaggggggaaggaggaggggggaagaggagtgagcgggggaaaaaaagggttgctggcggcggtagacggatgtagtgtacgaggcggcgagtacgccgagatgatggaacgagggcgagcctcttgttatgtgtgtgtgtgtgggaaagagagcgcgagagggcgggtgagagaggtcggggaggccggggccgaaatagaaagcggagaaagctctcagggcccggggttgcgccggcgggagagacgacgggcgccacagacagcggcgcgggggagggtgtgtgtatgtgtggtgtgtgttaaccacgaaagatgaggaggcggcggcggcgcgagggcgcgaaaaccccgacgcgcctcgatgcggcccgaatcgccgcgacgcgagtcgacggggagaggaaggaggggaggacggagggcgagccgccgctccccccaccccgccccgccccgtccctctcgcccacgcgccctctccccccttcgcgcacgctcactcgcccgcacccccgcccccctctcgttccgtgccgcgcgcgccgtcccgcggccccgtcgggcgcagggatcgtagcaaacgcgggcggtcggcgtgcgcgctgtgcgggtgggtgggggagggggttgatgaccgcgctggttccgggaagcctcccccggcgggcggagggggtcggggccgggttcgctctgtaccctctcacgcgggcccggagtgccgcgggcggggcgggggcagcactcagcgaaggctcagggtgttccagccgatggccccgggggcacgcgggcgggaggagttgcgtgggcggagaggagcgaggaggactggcggcggtggaggagggggaagaggcggcgagcggagcggcgcggtagcgcccgcgggtggcccgcgcggtatcgcccgcgcggtatcgcggcggcccggggagagcgggcgggggacggggacggggacggggaggaggggggaggaagggggtcgccaccgcggccctcgcggcccgggagaagccttcccggacgcgccattagagagggctctgtctgcagaaacggtgtctaggcgccgcgccaggggaaaccgcagcggcgataaaatagcttttagctcggcgctctctctgggtcgcagcgccctaaacttggcacggtccgccgcacagtgtggcccgcgtccaggccgggcccccggcctgccggcctggcgagcgagcgagcgagcgagccggggccccccagctcgctccaccgtgacacccacccacaccgtaaactctcacacggacacgcgcgcccaccccccggcgccgcgggcccgaccccggtccgcgccggcacccccttttctccggcaccgagccggggggccggggcgaggggaaggggaagggggggggacggggtgcgcgcgcgtgtgtatgtgtatgtggcaccggcatcggtctccccccaaaacggagaaggcgagggagacggagagggaggtgtgtctctgtgttgttgtttttttttcctgctggcgccgcggtgcgcgaacgtgatcacggggaggcggcgcgctccgcgctcccccgccgcctgggcggggatgccgcgccggcacgagcgcacgcgggcgcgcgggcggcgggaggggagggaggagggagaagggaggaggcggcggtggcggcggggaaggaggagggcggaggaagggccgggccgccgagccggccgcgccattgggcgccgggcggagcggggcgtggagaggcgcccgcgccggggacgcgcctgcggcggcgggcgcgcgccgggcggctctcctccttcaccgccgccgccgcgtcctcctcctccctccccgccggcctcggcccacccgcggcgtcacccgcgctcccccgccgcccccccctcccttccccccgaccccctcccctccctcccctcccctccccgcgtcctgcgtcacggggaccggggggtcccgcgtgatgacgccggggacgcgggacgcgcccgccgcctgggagccggcgaggaggacaaaaagagttctgtgtcccctcgcccccctccgcgtccgcagccgcctctgcagccgccgcccgcgccgcgaccggcgaacgcagagagaggacgcggggaagagacgcggagagggggacggggggagagagagcgaggggcgcgagcgcttcctcctccacacccgccttcctccgcccgcctttccttcccggactcgggagccgctcccgctccccccgtttttcctcctggccgcggcgaccgccgccgccgctctcgaggtttttttttcccttcctctccttcttcctcctcctccgcccttgggtgtttccttctccgcgtccccatctccggggcccgaggcgccggggctttcttcccgggccgccggggaggaagcgccccggcttctgcttcttcttctcctctctcggtaagcacagactctcctactactactactcctactactcctactcctactatattactatgactactactatgactactacggtgacttctcctccccgctctagactttttttcctcccctctcctcctttcgctcccgtctcccgggcgcccgcggccgggcggagggggaagcgggaaggggaagcgcggggcgccgcgcctctccctcccgcttcccctccgccggccccttccccgtttccccgtttccctttccccccccttccccgtctgttccttcccccccgcttccccgtttcttcttctcccccccgtcccctcttccttccccacccgccccgctctcccacgccgcggccgcggggggccgaagcggacgcggaagaagacgcggaagaagagagagagagaaagaggggggccggcggggatggggaagaaggcccgacccgcgcgggggcccccctcctgtcgcctgtcgtttgaatgaaaccttcccccgagcgaccccgctcccgcctgtggtgccgtgtttttttcccgcgacggggagagcgcgagagtgaggctgttggctgtgggttgttggctgttggctgttggctgttggctgaggacgacggggaggggggatgctgccgcgggtggcatgagacggagggggcgagggcgggcggtggaggggggagaggaaggtctgggcgcgcgtgccgcgggccgagtgtcgggcgagtgcggggcgggttggggacggggaggaggggaggtgtggggggatggccggccggcgcccccggagcacgcggggcgccccggcgggcgggagggggtccgggggaagggtggggcggtggcggcggccatcgcccgacccttccccggggcgcgcgcgtgggaccgggaccgggacagggggcgggggcggggagaggaggagaaggaggagaaggaggagaaggaggagaaggaggagaaggaggagaaggaggagaaggaggagaaggaggagaaggaggaggcgaactgggggaaaggcgagacacgcccgggcgcccccctcctcggaccgaggcgacggcggagacggagacggagagaccgcccgcccggccaccaccgtcggtaccggtatcaccagcaacacccgcaccaccgacacccacacggaccctcccacccgcacgggcccccatacccgcaccggccctcacacccacccacacgcacgctctctctctcgcacacccccacatcacactctccccgcgcacagacacacggacgacaccctccccgcccgccccgtccgaccgaccatccgtccgcccaaccctccacccgcccggccctccgagagagagagcgagccttctctccccgcacacacacacacactcacacccgcagacctccggagcaaacccccccgccgtcccccagcccgattttgggcccccccgtcccggaccccccggggggcgggggcctcgcgcctccgcgtccggcgggcgcgaccgaggcccgcgcgcccccgggggctcggggcgggacgcggcgcccgcgcggggacactctttcccccggaggggcgtcccggcgcgccggcccggccctccccgccgggaagggggcccggccctcttcgtctcccggagcgcccccggggtcccggcccgaggtccattttgggccccgcccctttgcacccgcccccccaccgggcggggtccgcgctccgacccccccccctcccctctgccacgccccgccacgccccgccgccatcttggctcccctcccccccaactccctctctcccgacgaggccgagccggtctcgcgccccctcccgcccgcccgaggtaccgcggaaggacgacacgagccggaagacggtgcaacgagaacaacttttatttttttcactctggggcgtgtctcgtggggtgggaggggggaccgggagagagaggtagaggggggggggtgggtgggcgacaaaagtatccaaggcgtggctgtccggctcctcccttccgggtccgggcgggtccgggctcgaggttcgggtccggttctcccgccccggcgtcgcttcccggttcgggtccggtcgccggtccccggtccgacggcggttccgctcgtctctccgaccggctgggggcgccctcggagcgcgcgccgagcgcgcaggcgcgggtggggcgaccgcgggggtcgcggggggcctcaagggtctcggggagggggaaacgggggtctcgggggtctcgcgagtggcagggggaggcggtgagaccaccacacaacacagagaccaccacaaaacactgagagaccaccacacaacacagagaccaccacaaaacactgagagaccaccacaacacatgcggaccacgtgcagacacacggaagcttgcaccgtgaggccccattggccgagggcggccacgtggggcgagcatgcaaatgacgtgcacacggaagcttccccgaaaatcatctgattggctcgctagcaccacgggggtgggtcgcgtgccgagcatgcgcctcgcgcccgcgcgagggattttcggacaatctcattggccgcccgcggccaagatggccgcagagcgggccgggcatgcaaatcagaggcgcgcgggagacgcctccgcgcgcccattggcccgggcgagccgagatggccgccgcgggggccgggcatgcaaagtagacgcgagaggaagtagggagagaaatcccattggccgtcgaggggccaagatggcgccctcggggccgggcatgcaaagtagacgcgagaggaagcgggcgagagaaatcccattggccgccgtcgaccaagatggccgccgcgggggacggacatgcaaagtagacgcgagaggaagcgggcgagagaaatcccattggccgtcgagtgggcaagatggccgccgcgggggccgggcatgcaaatggtcctcgcgaggaagttcctcgcgaaatcccattggccggcggccgccatcttgggccgggcatgcaaagcagacgcgagaggaagcgggcgagaaaaatcccattggccggccgtcggggaagtccgcggcgaaaatcggccattggtccgcttacctgggggcgggctcttctcggggagtttataggcacggtctccatcgtagcacttcactgcggtgcaggtacggacagcatcgttctctgccaacccgaggggatccgatcgtctcggctccggcgcggactctgaagactccggctctccggcggctatcagccctcgacggacggcccgacccaccggggggaagagagaggagagccctcttcttcttctccttcttcgccctctccttcttcttcttctgccgcgcccgctcctccggctccctctcaccctccagcacccgctctccgagctcggctcccggcgcgcgagagagagagagacaccccgagaaccgatcgagaccatggccgacgatctctttgacttcatcgagaccgagggcaacttcagccagctcctggcggcggcggccgcggccgcggcggccgaggaagaggggaccgcggccggctccgacggcggcagccagggctcccggcgccgcggcccctccggcgaggatctcctctttggccccggcggcctcttctccgacgatgccggcgaggccgaggccgaggccgccgtcctcgcggccgcggccggagccacgaggccgccccgtcctccttcggcccagcagcagcagcagcagcagccccgccggggctcgggcgagatcgtggtgctagacgacgaagacgaggaggaggacgagcccgggtccccggccgccgggtcccccggtcgggctctccatcagggctccgagcacggtcacctcgtcctcgggccccggtcccgggccggctccgggccccggccgccgaccccggcagcactcgcagcggcagaggccggggcccccggcggccccgggcgcccgtccccctccgcagcctccccggcctcctcctccggctcccccggcccctcggcagccccgcggagatggtcccccgcgaggggggacccggtcggtgagcccgggccggcggcgcggcctcggaccccggcgccaccagcacagccagcagcggtggccgcagcgccggcacggcgggggcccgcttccccagcctcccccgccgccgggccggtctcggcgctcggcggcggcggcgccccctccgccggaggggaccgcggtcgtcaccatcaccagcaccgcgagcccctgctcgacgagcccgccgccgcgaggcggctcgatcctcggccgcttggcgcccggagccccgtctcctccaactccaactccaactccagctcctccaccaccgtcgccgtcgagcccgtcgcccgcgaccccgagaaggacgaggacggactcggcctcgccggggacggcggggcgcccccgcagagacagccccgccggcgccgcgccggagagggcgccctccgccgcggccgcggcttcagcagctccagcagcagcggcagcgactcggacctctccccggcccgctcgccctccgccccccgggcctccgccgccgccgccgcgcggcgctcggcctcctcctcgtcctcgtcctcctcgtcctcctcgtcctcgtcctcctccgagggagaagaagacgaaggagtccgggccggcgccccactcgcccgcgccgggccgccaccgtctccgccggcgcccgccgcggccccgcggccctcggcctcctccgccgccgcctcctcctcctcctcctccgccgcggcctccccggccccggccccggagcccgcccggcccccgcgcaggaagcgccgctccaccaacaaccacctctcgctcatggccgacgggcccccgccgaccgacgggccgctgctcaccccgctcggggagccctggcccggctccgaccctccggccgacggccgcgtccgctacggcggcgccggggactcccgcgaggggctctgggacgaggacgacgtccgccaggccgcggcccgctaccgcgccgcggccgggcccgtgcccgtcttcatcccggagatgggggactcgaggaagcagcacgaggccctcgtgcgcctcatctacagcggggccgccggcgaggccatgtcctggctgcagaacccgcggatgcaggcccccgaccagcgcttcaaccagttctgccagcgccgggtccacgcgccccacggccacggctccttcatcaccggcagcgtgaccccgccgctgccgcacatcggggacgccatggccgcccaggacccgctctgggccctgccgcacgcggtgagcgccgtggccatgagccgccgctacgatcgcacccagaagaccttcatcctgcagagcctccgccgggcctacgccgacatggcctacccgggccgcgccgcggacccccgcgccggggaggcgaccgtcgaggccctctgcgcccgcgtccgcgccgccttcgccgccgcgcagcccggccgcgtgccccgcgagctggcggacgcctgcgtcctggcctgccgcggcgtcctcgagcgcctgctgcccgtcccgctccggctgcccgcgcccgcccgcgccccggccgccctcgggcccgcctgcctcgaggaggtgaccgccgcgctgctcgcgctccgcgacgcgatccccggggccggcccggccgagcggcagcaggccgcggactctgtcgccctcgtcgcccgcaccgtggcgccgctggtgcgctacagcgtggacggggcccgcgcccgcgaggccgcctggacctacgccgcggccctcttcgccccggccaacgtggccgcggcccggctcgccgagg

SEQ ID NO: 2 - DNA, 145312 bp, Pseudorabies virus
ggcccagctctcccccgagcgcggatctgtgaaaaaaaattttccctcccccgcgttttccattggggtgaatggggagggggtgggggatgggcactcactgagaattggcaaaatgccaatttatattctggcactttgccaaaccacatacttggcaccctgccaaccccaatccccctcctaccttggcaccctaccaaccccaatccccctcctaccttggcaccctgccaaccccaatccccctcctaccttggcaccctgccaaccccaatccccgccccggtcctcccatcgcttttggcacggcgccaaccccgatcccccgccatcgccttttcccagtccttggctcgctgccaaccctagagggcgagacgcggtgctgcggtcgggcccacgggcggcgggtggttggagagggcgatgggcagggacgggcgcgcttgtggtggggacggtgtcggtgcccgcgacggtggcgcccgagagggttcgccttggatgcgatgggggccctccccattcaccccaatggaaaacgcgggggagggaaaatttttttttcacagatccgcgctcgggggagagctgggcccagtctttttggggggagggggtgggggaaaagagctgggcccggctctgggtgggggacaacccccgagggagagtgtggccggtggccagactagaggagagccccgccgggaccggtgaggagagagggcctttccccccagccccctccgctcccccctttccccccagccccctccgctcccccctttccccccagccccctccgctcccccctttcccccctttctaagtggacgccgcgggcgcgcgggcccccacacggcgttcgctctcgtcccggccggtcgggccgccgggacggggcgctcggcgccgcgatgaccgacgcctcgtcgcggcccggcggggaagagccgatggaggtggacgagcacgtcgggccggagcccaagcccatggaggtggacgagccccccgcaccgccgtcggcaccggcaccgacggcggcgggcaccatcctcgcggtgggcttcacccagcccgcgcaggtcctgcgggcgtacccgatcggttgaggtgcgaacgatgggcgggggatgcccggggtcggacgcgctcacgtggtgccccacccaccccacgccgcgctctttttcccgggccgcccgaccggatcctcccgcagaggcggaggtgaggccggagagagacagggagagatgatggacgcggagaggggagcggccgccgcggggcgcgtggaggtggagctcgatgcccggccctccggcgccggagggcgcggcgcgagcccctcgcgccgggcgcgggacccccgaaccagcccgtgctcccggggccgagccgaccggcgttcccgggacagaaccggcgacgtcaccgggccgcgggcagcgcggcccgccaccctgggtattaacgtcccgggcaccgccgtccgccgccagccgccgtgggaggcagacatgcctccacaacgagcccgcggggctccgccgcgccgtcgcggcagcgacccgcccgatccaggcagcctcgccgggcggccctcgcccggggggagaggaggcggagggcagcgccgccccctctcgcgcagcagctcgctcacgtccgtcgcctcggcgcccgtggagacgcccgtcgtcgcggaggctccggggctcggcgcccccggctccaggccgccctcctacggggacgtcgtccgcgtcgggccgcgcccgcaccgatcgccggacacgccgctgtttgcccggggcccgcccccgtcctactcggagacgctcctgttcgacccgcccgcgtacgcggtgaccatcccggacccgccggcgtacgagcccaccgtcatcgggccgcacccgccgcgcccccgcgactggatctcctcgccctcggtggtgcagccgtcgctgctgggccccttcagccagtgcctcccgcgggtgacctgccccgactgccgctaccccgaagaccgcccgatggtgctcgtgggcttcctctggggggggctgctcctgctggtgggcctcgtgtttctgatcctgctcccggtgctccgggagtccgtcgtgtttccctgatcaataaactcggctcgcccgccccatcgatattcgcgtctcgtgtggtgctgcgacgaggggggaagaggtgggacgcgaagagatgaagggatcggggggaaagacggcgagaggaggagcgggggtgtgcggaagggacggtgaagcggcggtggtggagggagatggggagatggagggagatggggagatggagggagatggagggagatggagggagatggggagatggagggagatggagggagatggagggagatggggagatggagggagatggggagatggagggagatggggagatggagggagatggggagatggagggagatggggagatggagggagatggagggagatggagggagatggggagatggggagatggggagatggggagatggagggagatggggagatggagggagatggagggagatggagggagatggggagatggagggagatggggagatggagggagatggggagatggagggagatggagggagatggggagatggagggagatggggagatggagggagatggagggagatggggagatggagggagatggagggagatggagggagatggggagatggagggagatggagggagatggagggagatggggagatggagggagatggagggagatggggagatggagggagatggagggagatggagggagatggggagatggggagatggggagatggagggagatggagggagatggagggagatggggagatggggagatggggagatggggagatggagggagatggggagcgacgacgacagactcgcgcacaccagagaggtacggttcaacggttttattcaaaacaggtggttgcagtaaaagtacttcccgtgcatgtacacggggacgagggtgtagctgcacgtcaggcggcaggtgggggcgtcgcagccggtcttgtgggcgtccagcgcgtccatgatcccgccgaggcaggcgccgggcacgtactcctccagcggggcgggcgagtcccccaggtcctcgggcagcacgcagctcgagccgcgctcgacgacgcgggcgatgcgggccagcatcaccagggtgtaggtgacgatgcagcgcacgtcccgcaggcggcgccggcgcacgagctcgtcgacgggcgtgtcgcggaggtagcagcgcaggaacgggccgagcttcaggcgcaggttctggagtaccgcccccgcggtggccacgatggggtccagcgtgcgcagcgtgagccccttggtgaggatgagcttgacccacgtgagcgtctcgtccgccgaggcgagggactccgtcaggccctcgttcaggagcaccatctcgcggagcacgcgcgccgcctcagtggcgtgcggctgcacctcaaacaggcggtgcaggtcggccccgtggaacatgagcgtctcccacgtgacgcgccgcccgtccggggtggactgctcggcgccgaactcgaggacggcggcccacggcgacgagcccgcggcgcggaagccgtcgttgaacacggggcgggagaggtcgcgccgcgccgaggcgaagaggtcgttgacgcgcgtcgcgccgacccgcgcgtgcgtcgcggagacggtggcgcgcgcgctctccgacaggcggtcggggggcgcgtcgggcccgctgtcgggacgatccgcctccggccgccgccgctggtgctgctgctggcgctgctgctgctgttgctgtcgctgccgctgtcggagggcctgtcgggcggcgcgcccgccgcgacccccgcaccggcgccggacggccccgagccgcgcccggacgggcggccgctcggaggggcgcgcgcccaggcgctcgcggaccgggcgccactcctcgctccccgaccgggacgcctcgctgccgctgctgttgccgctgtcctccatgaccgtggtgcgagcggggccgcggtgtgccgccggcgggcggcggggcgcctttaaccccgcgcccccgcgcgaccttcatccaaatatggtaatgtggcgctcgtagtgcagaaacagcaacacgccccctaccagcaggaggaggtacagcgccttggcaaagatcccggcgagcacggtcgagcagcaggcgccgcagacgccgagggcggcgcgcccgccgtcggcgcgcgcgggcggggccgtcgcgccggagacgcgcgcgatgtgcggcagcgtcgccgccaggagctccagcgccccgaccgccagcacaaagagccacacgagggcgcgggcgtacagcgggaagacgagggcgcacggggcgcgcgtgacgccgagggtcgtgaacagcgccatgcgcgcccccaggcggagcccgatctcgaggaggatgagcgcgatcagcgtggggtggcggtgcgccagcgtgatggggtcctcgcggaggtcgcggctgggcgcggcgcggcgcgtcgcgggctcggccatgtaccaggcgaacttggtgtagctgcagccgatgaccgtggcggcgagcacgctcgccgcgtagttgagcgtgtacttctccggggtcaggaactcggcggggtcgcggaaggggccaaacatgcgccgctcctggcgcgcgtacacgaaggccgcgtagaggagccacgcgagggccgccaggcgaaagtgcacgtcccacaggtaggcgcggcagtcgcggcgcgcgtacacgacccgcgcgcggtcgcgcagctccgggctcagcgcgcccgtctcgttggggcccacgtagatggccgtggcgttgaaggcctcccagtccggcgcgccgccgccggcgtgctcgccgagcggcagcgccgcgtacacgacggggtggggcaggcgggcgacggtcgcgtagtacgcgcccaggccggcgtacgcgcccatcaccccgaggacgaggaggtgcagcgggcgtccgccgaggagcatgattgccccacctccgcggtgaaaatggtgcgcgtcgcgttgctgccgcactttgtagcgaagcactgctgactcagcgagatgcacagccggtcgtgggcgtcgacgctcagggccacgaaggtgcgggccggcgggctgcgcgcgtgccgggcccgcaggcagctgaagcgcgcgggcccgcagagctggaagagcacccagtcgggcttggccaccacccgctgcgcggtcgcgcccgcgtactcgtgcgcgcgcccgttgaagtgcgcgtcgaggtgccgcagcacgggcgccatggccacgtccgccacgaaggcctcgagcgcgcgccggtcctccgcgtccacggccaggccgcgcagcatgtccgccagcggcgtgcgccgggcgaagaaggcctcgcggttccgcgccgccttgcgctcgaagaagctctcgtactccccgccgaggctgtgcagcacgcgcgtgacggcgtgcgccgggcgggcgtggaagtgaaagttgcgcgggtcgtcgaagcccgggggcgcgtccccgaaggggagcaggcgcccgtggcagccgccgccgtcgaccttggcgaagaagggcagccgcaggctgcgcccgtgcgcgtacacgcccgtgtccacgaaggtgaagtcctggaggtacgggcccatgggctcgataaagtcccgctccagcagcacggcctgctggagcacacgcgccaggccgcggaccgtgtcggcgccggccaccgcgtacggcgacggcacgggcgtgcacacgcggaagcccatcttggcgcggcagccgcacgccgggcgctcgtccgccgcgggggcgcgcgcggggccgggttcggggtcgggggcggcggcggaggaggagccggggatcccggccccgacctcgtcgtcgtcgcacgtgtacacctcggcgggggcgcccgcgtcccagtccgcccagtcctcgccgtccggcgcggcgccaaagtcggcgggggcggcgacgtcgtcgtcccagggcgccccctcgaggccgtcgtcggaggaggccgcggcggcggaccccgcggcgccggcgcccccgggcggacaggcgctcttgtacacgtagcacgggtgcgcggcccagtcgccgtcggcctcggggaagagcagcgccagcgcgtcgagcaccgcgcggcggaacccgcgcatggccgcctcgagcgtgcccgcgggcaccgggcgcgcgaggcgaaagtcgacgtcgaggacgatgttggtcacggccagccgggcgttgaacacctcgtggcggctcacgtacatctgcgccgccggctcgtgcgccgccatctccggcgccgcgtcgcgccgcgccgtcgcgcgcccccaggcgtccccggccagcaccgcgaaggcctggcgccggtgcgccagctccacgcggtacaccggcgccggggggcgcgcgtccggcaggccgagcagggcgcccagcaccgtgcgcgcgcgcccgccgcccggcgcggcggccagctccagcaggcggcgcaggacggtgccgtcggcgcccggctgctgctgctgcgagtgcgcctgctcctcgtcgggggcgcgcacccagccgtggggcgcgaggcgctcttcgacggcggtcacgtggcgcgcgaggccggcggccgcgtcgcagagcccgcgcggcgcgtcggccgcgagcgcggcgtacgtgcggtcctccacgtagccggcgcccacggccggggcgaggcgcgcgacgctcggcgtcacgttctgggcgacgtagtcgcggatgttgagctgcgcccgcacgtggcggaagaaggtggcggccgcgcgctccccgaggggcgagcggcccacgggcgccgcggggtccgtgacgttgacggcgtccaggtgctcgcggagccgcgcgcggttgaagctctcaaagtgggccaggtagacgtacgtcacgaactcgcggtcggggagcttgaggctgccgcggtcgtgggcgatgtagtcccggaggacgcccatctcggcgcggtcggcgcgcacgcgctcgtcgacgtagcgcgcggcgcgcgccgccgcgggcccccgcgcgtagccgctcaggcagcagaagcgcgagagcgccgcgaacgagagcagccccgcggggtccaggccgctggggttgggccccacgcgcgggttgtacgtgagcagcaggtccttgagggcccgcaggtcgtaggccggccgcgcgtggaagaggtggaggcgcgtggccagcaccagcgtcttctcctcgggcccgaagcgcgccacgtaccagaagggcgtgttgtggttcccgtagaggcgccggaaggcggccagcgtctgggtctcgtggtgcacaaagagcgaggtcagcccgcggcgcccgaccgcgatggagcgcagcgggcgctccggcgcgtactcggcgttgccggtgcgcgggcgcatctgctcgaggaccatggtcagggccgcgatgaggccgtcgtggagcgcgaaggtggccgcctcgtccatggcctcgaggacgtcccgcgccgcgaggggctccccgcggcagagcgcgcggacgaccgccggggtcgcggcggcgacgaacacgggccgcacgcggacgctggccacgtgcccggtgaggcagaacgtgacgccggggcggcgcgctaaaagctcggcctcggcgcgctcgcccggcagtgagaaggcctcgtccagcccggcgccgtcccacgcgtacgagacgacgtagaccgcccccgagggctcctggcccgtgagcagcatcagcgagtacgtgatcgcgcagccgtccgtggcgtacaggacgcggatcatgctgggcggcatcttctccgggtggtgcgggcgccgcggccgtgggagggcttatgagccgccggacgaactccgcctccgggagtcgctggccgtgctcaacgtgatcctccccgccccgctcgccgccgaggacgtgatggcctcggccgaggccgcgcgccgcctggcccgcgcggacacgctggcgcgcacctaccaggcctgccagcgcaacctcgagtgcctggcgcgccacgaggcctccggcgacgacggcctggacgccgtggtcgcggcgcacgcggccaacgcgcggcggctcgcggacacgtgcctggcggcgctcatgcacctgtacctctcggtcggcgccgtcgacgcggacacggacgacctcgtcgagcaggccctgcgcatgaccgccgagagcaacgtggtgatgtccgacgtggccgtcctggagcggacgctgggactggcccggcggcggcagccggcgcccgtccgggccgcgcccgccggggtgggcctgcccgcccccgtcccgcccgccccgcgcgtcgccgccccgccggccccgcagaccccgcccgaggaggaggcggatgcggaggcggaggaagacgaggctggcggggaccgccccgaggaggacgcggtcccgctgctgcccccgcgcgcccccgtcaagacgcgcccgcccgagaagaagaaggtccggaggctggccgaggcctgcaagatgtagacggagagcggggggagagagaaggaggggacggagaccaccgggtgtttttttttcttttcaataaacatctttattgaaaaaaagagaagcgtcctgcgtgtgttgtgtgcgcgcgcccgggtgccatcgccatcgtcccggcgcgggccaccgcggcctcacaggcccgtggagccgaagccgcgggtcccgcgcgcgctgctcggggcgacaaagtcccgcgtgaagagccacgcggggcggtgctgcatcggggtgcgcggggtggcggggaacggggaccgccccgtgatccagctcagcggctcgcgcgtcagcaccagctgcgccacgcgctggcccgactccagggtcaccgggtgcgcgccgcggttctggatccggaagcggcacgggccgctctcccagggcgtggggaagacgacgatgccgcggaggttgagggacgagcgcccgaagacgtaggcccagtggcggccgtccgtgcggtgcaccggcagcgccacggtttccgcgccgccggggggcagcaccagctcgcgcgggcaggggatgtcgtatccggcgtcctcgtcgcgcttcggcgcaaagacctcgaagaagggcacgtccgtggcgatgcccggcgcgaggcgcagcggcgtgcaataaaactgcgcccgtgggcggcccggggctacaatcgcctgcacctcgccgcgaaagcccgcgtccacgatcccgttggtggtgcgaccgcccccgcgctgcgcgacgagcatcgcgtatccggtgggcagcgccaccttgaggcccaggggaaccttataaaatccctccggccccggactccggcacaccagcagccgccctccgtccactgtcacgggctcgctcgcagacaccaggatggtctcttcctccgcccggctctcgctgacgctcgtcgccgcgctctgcgcgctggtgacgcccgcgctctcttccatagtcttttccgaggggtcgctgccgctgctgcgcgaggagtcgcggaccagcttctggagcgccacctgcgccgccaggggagtcccggtggatcagccgacggccgccgccgtgacattttatatctgcctcctggcggtgctcgtcgtcgccctgggttacgccacccggacgtgcacccggatgctgcacacctcccccgcggggaggcgcgtataaagatcgatccgcacgcgcccgatcccactcgctcgccatgtccagctcgagaaagacccgggtcgccgccgacgagaccgcctcgggggcgcgccgccgcgccggcagcgcctcccgcgcccgggccgccgccccggccgtcccgaccgccgcgacccccagacgcccctcggcctacgacgacggcttctcctaccggtctgccccgtcctacgacgacgatgactactacggctacgatggccacggctcctcccacgccccccgcgccgccaaggtgacgcccgcggccgcctcgcgggcctcgaccggggccaagagcgcctcggccgccaagacccccgcggccaagaccgcccgctcggcctcggccgccgccccggccgccgccgccaccaccaccgccgccgccgcggaaccggccgcccggcgcgcctcgacgcgggccgcccccggggagaacctcgacgtgggccgccgccggctcgccttcagcgacaggccgtgcgaggccaacgtgccctggcgcggcgcgacgcgcgccttcaacaagcgcatcttctgcgcggccgtggggcgcgtggccgaggcccacgcccgcgccgccgccgagtccctctgggacatgaaccccccgacgacggacgcggcgctcgaccgcttcctgcaggccgcggtggtgcgcatcaccgtgtgcgaggggctggacctgatcgaggcggccaacgccgtcctggacgagagcaccccggggcggaagggaaaagtgtataaataaaggcgttgcaaaaggcaccccgctcagtcgtccgctgtcttgcttgtgggggggtcggtgaggatgcgcgacgaggagtgcgtggtcgcgttcgacgaggccctgttggggggcgcggccccgggccggccgcccgtggtggccgtcccgcgggcggccagcccggacgcgctgtaccggcgcctcatcagcgacctggccttcgacgagggcccggcgctgctgagcgccatggagcgctggaacgaggacctgttctcgtgcctgcccggcaacgaggacctgtacgcgcgcatggtcatgctctcggcctccgcggacgaggtgccggcccaggtccaggcgccgacggcggacgcgagcgtcgacctcggggtgcccggggcggagcccatgccgcgggtcccggccgtggaggaggagctgcccgcgtacgtggcggccgtggagcgcttcttcgtgtccacgctgcgggcgcgcgaggagcggtacgcgcgcctgctggacgcctactgccgcgcggtgctgcgctacctgcacgcgcaggcgcgccgcgacgagggccggctccggcagctggtgcgcgggcgctactaccgcgacgtcgcgcgcctggcgcgcctgctctttctgcacctgtacgtggccacggcgcgcgagctctcgtggcgcctgcacgcggaccaggtggtggcccaggacgtgttcatgtccctgcgctacgagtgggaccagacgcggcagctgacgtgcctcttccacccggtgctgttcaaccacggcgtggtgctgctggcgggcgcgccggtgccggccgcgcagctgcgcgccgtcaaccgccgccggcgcgagctgggcctgccgctcgtgcgcgccggcctcatcgaggaggacggcgccgacctggtcgaggagccgccgttctccgccgcgctgccgcgcgccgccggctacctctcgcaccaggtgcggatcaagatggaggcctactcgcgcgagtaccgcgaccacacgtactgccgcccgccctcgcccgtggccagctacggcagcaccgcggaggccctgctgccgcccccgtcgccctcggccgtgctgccctgcgacccgacgccgccgccgcgcgtctcggccgccccgctcatcaccacggtgacgctggccgaggccgaggaggacggcgcgctgacgacggcggcgcccgcggccgcggtcgcgaccgcgacggtcgcctcgcccgggcccgccacccccgcctaccacctcatcccccgcgacgcgctcaaccgcatgtttgagatgtgacgccgcgccccgcgcgcgcggtcggatctgcggcgcccgaccacctgtgggcggggcgtataaagccgcgggccggcccggagggggggcatttgccgcagcgaccaccatgagcgacgcggggcccgtgcggcgcccgaggcgctcgcaagagcagcgcttccagccgttcccccgcgcggggctcctggtgcacctgcaggacgtgctcgtgggcgaggtgcggcgcccggacttccgccctccgcccgacgaggagagcagcgaggaggaggagcccgtctggaccgacgacgaggaggaggacgagggggaggaggggggagcccggcgcggggacggggagggggacgacgacgtggacgtggacgacgaggaggaggagagcgaggggggcgcgtggtccgacggggagctgtacgtggcggccgacggcgacgcgtgggacgcggaagaggacgaggacggggacgaggatgaggatgatgactatgacgatgacgacgacggctatgacggccgcggcgccgcggcctcccgcgccgcggtcgggccccccgccgacgtggactacctgtcgcgcgccctccgcggcatggagacggcctccggccccgacgagcgggaggccttcacccgctacgccggcacgctctaccgccgccagttccagcccgggcggccggggtaccggcccccgcggccgcgcgacccgccgcctcccccgcccccgcgggcgcggccgccgctgacgacggcgacgacggccgcgcccgccgtcggcaccgccgccccctgcggcggcgacgactccgcgccgcgcctcgagcgggacccggacgccgcgtacgccagctggacgcgcgacatgcccgacgggacctttctggccatgccgtcgtgggtggcgctgcgccgcgagccccgcgggcccccgggccgccccgtcgggcccccggacatcctgcgccgcgccccgcgggccctgaccttcacgccgcaggcggcctcggcggcgctcgtgagccgcgaccaggacgcctgggaggcgctggtgccgctgcaccacctgcagatgcactcgtggggcggcagcgcgccgccctcgcgcgggggcgtgtacgcgacgcccgcgcccgagacgcgcggcgtgtggcgccgcgcgctgcggcaggccatcgccctgcaccacgccatgttcctggcgccgctgcagtcctcgacggtcggcgcgcccgcgctcgcgggcgaggcgctggccttcgcgctggacgccgcggcgcgcgcggccgtcaaccacgacgccgcggcgcgcgagctccccgagcgcgcggcgcgcgcgctgcgcgccgtcggccggcgcgcgcccgcggcctcggggccgcggcccggcctcttccgcgccttctgcgggtccctggcctactggccggagctgcgcgtcctcagcgggcaccccgaggacgtccgcttcgcggccttccacctggcggtggccgaggtgtacctgctggcgcgcgcccacggccagaacccgggcttcacggccgaggagcgcgagctgctcgcgcccatgttcacgctgaccgtgctcgcggtgcaccacgcgctgcgctggctcacgacggccgtggcgcgcgccgtctcggacatccccgacgacgaggccttccgcgccgtgcgctcgcgcgtgccggcgtcgctggtgccgctgggcagcatcgcgctctcggacgccgagtacgccgtgctgaacgcgaccgaggtggcggcggcgcgcgacgccacgggcctgggccaggccgtgtcgctgggctacgccgccgcgcgctccgcgctcacgggcctcatgcgggcccacgccggcgggagcgacgccctcgtcgccttcgcgctggtgctgcagcgcctggcgggccacgcgaacctgctgctcaactgcctgctcggcgccgccgtccacggcggccggccggtgcgcgtgtacgaggcggcgctggacgactacgcggagctcatggacgcgctcgacccgctggtgcgcgcgtgcccgctcgcggagttctgggagcagcgcgacgccgtcatgcgccagctgcggctgacgccggccccgggcccccccacgggcggcaagcggctcgtcatcgcgcccgcgctgccctcggaggacctggacgggctcgtcccgcccacggccgtgcgccccgcccacttggggccggacgtcgacctcgccgaatacgccgcgcgccacccggagctggtgggcgtgccggaaccccgagcccatttatcggcgcgccggccgccccggggccgctaacccgtccctgcctccgccatgatccgccgcgcccgaggaacgcgccgcgcttcgtggaaggatgcctcgcgccgcgtcaccgaggggcgcacccgcgccagctgcctgctggcggcccccggggaggtgctgacggcggccgtggcggccctgcgcgaggcggccgagcgccagtgcgcgcccgcgctcttcggcgcccagcgcgcctcggccctggcgctcgtgcgcagcaactacgcccccgagtccgtgatcacggcgtgctcggcggagccgcaccgcgaggcgtacgcgcgcgcggccgaggccgcgctgaagggcatctcggcggacgtggcctggcgcgcggtgctggccacctactggcgctacctgaaggccagctcgggcgtggaggtgcgctcggacgacgagcgcgacgcccccaccgtcatgctgctgtggtcgacgttcggcaagacgctgtcgaagcacccgttcaagcacaaggcccagagcgccgcgtacggcgccacgcgcgccgcgctcgccgaggcgaccgaggccgtggagcggtacgcctacttcatgcgcccgctcgaccccatggtctccagcccccagacgagcgtgcgcctgcacgagatgctggcgtacgcggccacgctgtaccgctggctgctgtggatgatggacacggtcgacgcgcgcgtgctccgccacctggggcgcacgccgcgggtggtgcgcggcccgcgcgagacgatgtcgcccgaggcgctctttgggcggcaccgcgccggcgggcccgccgtggcctcgggctcgggcgaggtgctcgtgctgacgagccacacggcgacggtgtttgacgccctggagatcctcggcgccacgtgggccgagacgccctggaagagcggcgtgtgcggcctcacggccgccgtcgtggccgccgtggacctcgtgaccttcgtgcaccaccacgcgcagaccctcatcaacctgacgctcgccggctacgtgtgctggctcgacgacgggatggaggacccgtacctgcgcgcggcgctgcgcgcgcagtgccgcttccaccacctgatcggcgacctggcccccacgagctcgtcgcacgcctggggcgccatggagcgcggcacgctcgcctggttcaactacgccatcgcgcgcagcctggcctcgtacggcgggcccacggagcgcttcgcgcgcgtgctggcgacgacgggccagcgggacatgcgcttcctggccgtcgcccccgcgcagccgccgctgccgcccctgccgcgggcgtcgcggacgccctcgccgccgccccgcgaccgcgacgcggagccgccccacgactacgtggaccccctcgccgagtacctgaactggcccgtgcccgcctcgccgcccccgctccccgaggggcccccgagctcggacgacgagctggaggtggacggcggcgggcggcgcccgctccggcgcagccgcgacgccgccacgtacgtgaaccgcaaggacattgcccgcgcgatggcgggggcggaggacgaggacgaggatgaggaggaggacggccggtacccgcgccgcgacggggacgccacggcctcgacgagctcgtcgcaggagagcgccgccgacgaggagttcccgcccgggatcggcgtgcgcgacggcgaggaggacgacgaagacgagggggtggacgaggaggaggaggaggaggacgtgtacgtcggccccgacggtaccggcgaacggggcgccgccggcggcggcaacgccgcggacgacgacgacggcgccccctgggccccgctgacgcgccacgggagcatgcgcaccagcttccggcgcggggtccgcgcggcccagcgcttcgtgcgccgccggctctcgcgcacgagcgccgaggcggccccgcgggcctccggcgactcagcctccgcggcggcccccgccgccgcctccgcccgcggcgagaccgaccacgtgtaccagcacccccgcccgcggacccgcgcggacgacggcctgtaccagcaaccccgacccgtcatcgacctcaccggccaccgcgcgtcgcgccgcaagagctggcgcgtgtgacggctcccacggactcaccgttgtattgttttcccccgttgcccccctgtgtggaaataaagtttttttctaattctgtacacaaggcgtgtgtgtcgtggcggtcgtggtcgcgggacgggagggcggcggggagaggggagacgagaggggagaggggagacgagaggggagaggggagacgagaggggagaggggagacgagaggggagaggggagacgagaggggagaggggagacgagaggggagaggggagacgagaggggagaggggagacgagaggggagaggggagacgagaggggagagggacgaggcggacgcgagacgtgcgatcaacggcatctttatttttttccatctgcggggagggggctagggggcgtcggggtcctcgttctcgaggcgctggtagtgccggcggcgcgtggccatcgccccgacgcggctggccagcagcgcgggcccgctgttcttcttgcgcgccttgtgctcctgctgctcgagggccgacacgatggacatgtaccggatcatgtcccgggcctggtccagcttggcctcgtccacgtcgtcctcttcgacgccgtcctccttgagcgccttcgtcgtgacggggtacagggccttcatggggttgcggcgcaggcgcgagatgtgccggtaggccaggaaggccgcgaccaggccggccagcaccagcagcccgatggcgagcgccccgaaggggttggacaggaaggacaccatgccgccgacggccgagatcacggcccccgtggcgcccaggaccaccttgccgacggcggcgcccacgtcgccgaggccctggaagaagttggcgatgccgcgcagcagcaccacgttgtggtccaccttgaccacgcggtcaatgtcgtagaacttgagcgcgtgcagctggttgcggcgctggatctcgctgtagtccaggaggcccgtgtcggcgagctcctcgcgcgtgtacacctcgaggggcaggaactcgcggtcctcgagcagcgtcaggttcagggtcacccgcgtgctgatcgtctcgggcacctccaccatgcgcacgtagctgtagtcctcgtagtacacgtacccgccgcccagcttaaagtagcgccggtggttgccggtgcagggctcgatgaggtcgcgcgagatgaggagctcgttgtcgtcgccgagctggccctcgatcacgcccgtgccgttgtgctcgaaggtcaccagcgggcggctgtagcacgtgccgcgctcgccgggcacgcgcatggagttctgcacgtacacgccgccgcgcacctccacgcaccgcgagatggccatcacgtcgccgagcatgcgcgccgagacgcgctggcccagcgcggccgtggccacggcgctggggttcaggcgcgacatctcgccccacagggtgcggtccttgttctgcagctcgcaccaggcggccgcgatgcggctcagcatgtcgttcacgtgcgcctggatgtggtcgtaggtgaactgcaggcgcgcaaactcggccgagcccgtggtgatgcgcaggtgccccgtgccgttgacggccggcggctcgggcgtccccgccgggccgggggagcgccgggcccgacgggcggccgcgggggacgcggggcccacgacgccggcgaggccgaggcgctcgagctcgcgcgcgtacagctgcgccagctcgttcgagatcagcgggcggaaggccaccacgaagcccccgcgggcgaggtacacctcgggcttgtcgccggccagcacgtgcgtgttgttgtagcgccgccggtagatggcgtcgatggcctccgaggcctcgcggaggacgcagtcgcccaggtgcacgcgctgcaggtcgagctgcgtgacgtcgctgacgaaggaggcgcccagggcccgcgacgtgaagcggaaggacccgtcgcgcgtctcgtcgcggatcatctcctcggcctcgcgccacttggccaggctgcacacgcgccgcgtcttgggggcccagtcccaggccaccgtgaagtgcggcgtgcgcagaaagttgcgcgtcacgctctcggaggcgcggaggcgcgagtccaggtcgatggggtagtagtgctccacctgctggaagcgcccgggcgcgtagccgatgtgctccccgtgggccccctcgcgcaggccgtagaagggggacatgtacacgatgtcccccgtggacagggcgaaggagtcgtaggggtacacggagcgcgcctccacctcctcgacgatgcagttgacggaggtgcccgtgtggtagaagcccgcggcgccgatcttggtgtaggtgtcgttggtggtgtgccagccgcgggtgccgagcgcgttcaggcgcgaggggcgcaggtccacctcgacggggttctcgtcgcggtcgaaggcggtcaccttgtggttgttgcgcacgtactcggccttggagacgcacttgccgcggcggtcgatcacgtccgtgatctcctgcacggggacgggcacgcggtccgtgaagcggttcgtgatggccgcgtacgtgctcccggaccacacggtcgtgacgatgacgttcttgtagtagatgtgggccttgaacttgtgcggggcgatgttctccttgaagagcacggcgatcccctccgtgaagttgcgcccctgcgagtactcggggcaggcctgctcgggctccaggcgcaccaccgtggagccggacggcggcgggcagacgtagaagcggtcccgctcggtcgcggccgcgcgcacggccgtgcgcgcgtccaggtcgccgtactcgccgtcgggggcgtccgaggggccgggggagacggccccgtcgatctcctcgagggactcctccgcggagaagccgtctggggtggcgcccgtcccgggcgcgggcgaggccgaggcggcccgcgtcacggccgccgcgccgcacgtcggggtcgcggcgagcgccagcagcagcagcgctagcgcgacggcgccccgcgcagctgcagcgtggtgtggagcaggccaaagacgtccgaggccagcaccgccgtggtgcccgggccgatgcccgcggggcccgcgccaaagaccgccaccagcgggcatgacgcctcgtaggtcaggtacagcccgtcgcgtccggcgaagcgatccgcgcggaggacctcgacgtccccgcagtggaagacggacctgaagactgcaaccgccagcaccagctcgcggatatatcgccaggcctgccgctgggtggcggttacgccggacgtctggaagcggtagaagccgcggaactcgctgacgcaccaatcgcgggccaccatgaagcgcgccagctcctccttcaggtgcggcaggaggcccacgttctccaccgcaaagtacagcgccgtgttggggggctgggcgaaggcgtgcgtgtcgtgcgcgaagagcggcccgttgacgagctcgaagaacttgtgcgtgagcgccgggaggagcgcggcgtccacggggtggcgctgcaccgtggccttcatgaagctgtgcgcgtcgaagcagcccgtctcgcgctcgtccacgacggtgcccgcgcgcgcggcggcctcgcagaacgcgcgccgtgcgcggaacccggcggccaccgccacgtacgtgtgcagcagcgcgtcgccgtacacgttcacgcgcagggtcttctccagctcgcgccgctgctcgcgcacgcactgcgccaggctcgccgccgagcgccgcgagaggcggtccgcgtacaggcgccgccggcgctcggcgtcgcgcagcgccgcggccggcaggtccgcccacgccacggcgtccgccggcgggggcgcgccgggcccgtcttcgtcgccgacgcccccgtcgccggcgtccgcgccccccgcggcgccgccgtcggcgtccgcgtcgtcgccgccctcgaggaggcgctcgagggcgtcgcggttggcccggtccgggtccagcatgcgcagcatgggcgtggacatgtggtggtcgtagcacgcgcggatgagcgcctcgaggcgctcgtcgggcgaggccgcacagccgcccaccagcagcccgtcgatgatgtcgagctcgcgggccgcgcgggcgcggtcaaagtgctcggggcgccgcccgaagagcgcgagctccacggccgccgcgcgcaccgcggcgcggcgctcgcgccgctccaggtcgtccaggttgtgcgtgaacaggtcgagggcccgcaccgcctggttagtggacgccagccagaactgcagctcgctcacggcgtacaggcgccgcgaggcgggccggaacacgtcgtgcgcgtccaggagcgcgtccgcgcgccggcgcgccgtctcgcccgcgtccccggcctcggcgcccgccgccgccgcctccgcctcccgggcctcggccgcgtccacggcggcgagcgccgccagcgcgcgcccgcggcgcgcctcgtcgagcccgcgcacgtgcggcaggttcttggccacgtcctcggggtccacgcgcaccgccagctgccgcgtgaggtggtcgcacacgcagccgagcaggcgccggtgcgtggcctcgccctggttggccgtcacgcacagctcctcgaagcacaccgcgcacggctgcgccgggtcgtacagctccggcggcaccacgagcccgccgccgatcgtgcgcgcgaggaactcgtccacggccgacaggtccaccgcgtggggcgtgatcaggtggcagtagttgagctgcttgagcaggttctcggcgtcgtggaggaactgcagctccgtctccacgcgcccgccgtacgtgtcgagcgcgacgcgcgcgtggaagcggcacgcgccgcccatggtgcgctcaaagtaggcctcggcgtcgtcgccggcggcggccagcgcccccagcacgcggtccccctcggcccgcgcgtacgcgagcgccaggcgcagcgcgcacgtgagcggcgtcatctgcgcctccagcggcagccgccgcgcgaggtagcggcacatcagcgcgttcagcttgacgcgcggggccagctcgcgcaccaccgcggggtcgcagcgcttcagcatctccagctgaaacacgtacgtctgcacctgccccaggaccgccacgagccgccgctccgccatggccgagtccgcccgctcgctccctccctccgcccgcccgtccgccgcgagagaggccacgggacgcgcgtgtgtgtctgtcaaacgcggtcatctttattgatacagccgggcacccgcgcccgtcccctcctcctccccccatcacagcatgtcgaaggtcagccgcttctcgggcggggcgcccatgtcaaacaggtcgtcctccgggggcgggcgcttgccgcccagctccgcgggcgccagcgaggccggcgcgagccccgcgcccgcgtccccgcccgcgccgcaggccccaaagtcgaaggcggtctcctcgccgccgccgtccgcgcccgtctggccctcgaggtcgcgcaccagctcggcggccgcctccacgctccagcgcccgtcgcggtcggccacgcgcccgttgatctccgccagcgcggccgccaggaactcgtcgtccacgatcgcccgccagtcgtcgggctccaggtgctgcgtgcgcgccccgagcgcgtgcagcacggccgcgtacacggccgtctgcaccagcggcccgccgtcggccaggatggtcttgacgtgctccccgagcgagtggtcgtgcagcccggcgccgccgcccgtctgcgagcacgtgaagcccacgcgcgggcaggccagcacaaagtgccgcgtgcggtcaaacaccatcagggggcacacgtgcttgccgccgttgagcccgctccagttccccgcctggaagacgcggttgttgccggccatgccgtggtacttgctgacgcccaggcccagcaccaccagcgggcgcgaggccatgacggagcggagcaggtggctggacgtgtacgtggccacccacgtgtcgtcgcgcggcgcgtccagcacctcgcgcgcggcgcgctccgcgtcggccgcgtccgccagcgggcgccgcacccaggccatgacggcggccgggtcccgcgggcgcttcgactgcatcgtgatccccgtgaggctgttgatgaagaactgcttgtggtcgcagtagcgcaggatcaggttggccaggtagaactgggccagctccgccacggtgcccggggtcaggttcacgtagttgatggcgccgtagtccaccgagaagcgcttcacggccgcgatggtctcgatgtcgtccttggtcagcagccgcgccggcagctggttgcgctgcagcagcgtccagaaccactgcgggttggggctctgcccgccgggcggcttgccgcgcgggaacagcgtcgcgtggtgctgcttcagcaggaagcccagcgggccgttgagcacgtccacggccgtgtccgggcgctggtaggcgcccgcgagcccggccacgcgggcgcgcgcggcgtccgagaggctgcccgagcccgtcgagaacatgacccggctcttgacgcgcagctcccgcagcacctcgaagctcacgcgcgccaggtccccgtcgtggtcgcgcgccgggggctccgcgctgcccttggtggggtccggggccacgatgccgtcggcgagcgtcaccgtcaccgtcttggccgtgacgaagcccccgttgagcatgtccaccacgcggcggcgcagcaccggctggaactggttgcggaagttgcgcccctccacgggctgcccgtggagcacgccgtggcactggctcagggccaggtcctgcagcaccgccagcagcgtgcgccgcgccaggaagctcgtcgccgggcacagcgcgcccgagtacgggtccagcgagagggacatggtgtggttggcgtcgtagagcgcgtcgcggagcttgaagtcgcgcgtctccacgagcgtgcgcacgaagcggtccgccgcctgctcgaccgtgtcgcgcagggcgcgcagcgcgtgccggaagccggcgtggtccgtcaccgtctcgccgatgcgcgcgccggccacgtccgccagcagccggtccacggcggcgcggtacgtgtcctgcatgatcgccttcgggggctcgctgtcgttggggcgcttgagggccccgtacgaggcgtagttgccgagcacgtcgcagtcgctgtaggcgctgttcatggtcccgaagacgcccatggggctgcgcgtcggcgcgccgaagcgcgggaggcggtggcgcaggcggtgcagcgtcgtgtgcgcgcaggcggggcgcgaggcgcggtcgcagagcgagcaggggacctcggcgtcgaggctgccggccacgtagcgcacggcgtccacgtcgccgcggcccacgaacgtgcccgcgtcgcagcgctccaggtagaacagcacgcgcgccagcagctgcgggcagaagccgcacgccatcaccaggtggtccatggcgaagtcgtggttgcccggggccgtcgcgggctgcgaggccgtgtgcggcagcacgcgcccgtccttgtccgtctgcgggttcccggccaggtagggcgccgccacctggtagaagcggttaaacgaggggccggcgccctccttgccgccgccgtcggcggcgcccgcgtcgtccacctccgtcaggtacagcaccgagttggagctgaacaccagcgcccccacgaggccggccgcgcggctcacgtacgcgccgagcgccgcggcctgcgagacgggcgcgcccgcggaggcggccgcgccgccgccgccgtccttggcgcccgcctcggagagcacgggccagtcgtccagcgcggggggctcctcgtcgtacacgccggcggcgacgagcgcctccagcgacagcgccgcgtcggcgctcatgacggacgccatgcggcgctcgaggccgccggcggccgcgtcgccgccgcgccgccgctgctcaaagagcgtgaaggtcacgtcggcgggcagcacggcgctctcgtggttctcgtcgaaggccaggtgcgccgcgccgcgcgccaccgcgtccaggttccgcacgcgcgtggccacggccgccgggcccagcacgtagccgtgcagcaggcggcagagcgggccgttgaagaaggggcgcgggtacacaaactcctcgctgatggagcggttcttggcgttgaaggggtcggccacgagccggttgacgtccggcatgaagaggtgcacggggtagagcggcacgcgccacgcctcgtgcccgttgatggacaccgtgcccgcgcccccgtagtgcaggaaggcgttgcacaggtacaccgcctccttgaagccgtcggccaccacgaggtaggccgcgtgcgcgtccgcggccaggccgagccgcgcgcacacctccgcgcccgtcgtctcctcggcgttgtccaccggcgtccggtaggccgagaacccgaagcgggcgcgcgccgcctcgcaggcgcgcgagagggcgggcgcggcgctgctcgggggcacgcagtccccgttgtggaagaagaacacgttggggtggaagtggttgggcgtcagcttcagcgtgagcccgccgccgagcccggtggtccgggcgcccgccaccacggccacgtgcccggcgaagccggcctcgacggtcaggccgcgcaccagcggcgccaccgccgcggcgtcgtcggcgctgcgcgccacgagcagcgccagcaggtcgcggcgcagcgcctcgatcggcgtcacgtacacgtagcccagcggcgcggcgcgcacggtcacggtcttggccgcggcctccatcgtcgcgggtgagacgagacgagagcgggagcgggagcgggagcgggagagagagaaggcgcggcggacggggtcgagtctcggtcccctctccgggcggacgggtccggcggcggggtgaagagagagacggaggtgtcggcgccgaggggcgaacgcgacgtcggcggcggtggtagagcccggcccgccccggccgggtcttaaagcgtggggccccgccccgccggggcgtggcgccccgccccggagcctatacctcggccggcggcgttcgcacctccgcgtgggacgagcgatggcggcgcggcagggcagctacgtgacgcgcctgagcgagttcaagttcatcgcgccgcggtgcctggacgccccggaccagcgcggcgtgcacgtgggcacgctcgcgcgcgagcccacggtctactgcggcggcgccacgcggcccatcctccgcggggagcccttctggccgcggcgcgtcgccgcgtgggacggggcccccgagcggcccgtgagcccgcgcttcgagcgcttccacgtgtacgacatcgtcgagagcaccgagtacgccagcgcggacccgcgcttccccaacggcacggtggtcacgctgctggggctcagcgcctgcggcaagcgcgtggccgtgcacgtgtacggcgtgcgccactacttcttcctgggcaaggccgaggccgacgccgccctcggcgtgacgagcgccgagcagctcgcgcgcgcgctctcggcggcggcggcgcgcggcccgcgcctcgggcccgccgacgtggacgcgcgcgtcgtcgacgccgcgcccgtctactactacgacgccccccggcggcccttctaccgcgtctcgagcaacagcgggcgcctggtcgcgcacctccgcgagacggtctgcgccggcctggtgacgcacgaggccggcgtggacgcgaccacgcgcctgctgctcgaccacgacctgcccagcttcggctggtaccgcctgcggcccggccccgcgggggagcgcgtggtgctgcggcaccagcgcctcacgtccagcgacgtggaggtcaactgcacgcccctcaacctggcccgcgacgaggacggcccgtggcccgactacaagctcctgtgcttcgacatcgagtgcaaggccggcgacgacgcggccttccccgccgccgagaaccccgaggacctggtgatccagatctcgtgcctgctgtactcgctcgccacccagcggctggagcacacgctgctcttctcgctgggctcgtgcgactcggacgaccccgcggtgacggtcctcgagttcgacagcgagttcgagctgctgctcgccttcgtgaccttcctgaagcagtacgcgcccgagttcgccacggggtacaacatcatcaactttgactgggccttcgtgcacaccaagctcacggccgtctacgggctggccctcgacggctacgggcgcttcaaccgcggcggccagttccgcgtctacgacgcgggccagaacagcttccagaagcgcagcaaggtcaagatcaacggcctcgtctccctggacatgtacgccgtcgccgccgacaagctgaagctgccgagctacaagctcaacgccgtggccgaggaggcgctcggcgagcgcaagctggacctggactacaaggacatcccgcggtactacgccgcgggcccgcgcgagcgcggcgtcatcgggcgctactgcgtgcaggactcggcgctcgtgggcaagctcttcttcaagttcctgccgcacctcgagctctcggccgtggcgcggctcgccaacatcacgctcgcgcgcgccatctacgacggccagcagatccgcgtgttcacgtgcctgctgaagctcgcgggctcgcgcggcttcgtgctgcccgacaagcgccgcgacatcgcggacgaggacgacggcggcggctaccagggcgccaaggtgctcgagcccgactcgggcttccacgtggacccggtgctcgtgctggactttgccagcctgtacccgagcatcatccaggcgcacaacctgtgcttcacgacgctcgcgctcgcgcgcccggcgggcctgcgcgaggacgagttcagcgccttcgaggtcaacggggagcggctctactttgtgcacgcgggggtgcgcgagagcctgctctcgatcctgctgcgcgactggttggccatgcgcaaggccatccgcgcgcgcatcccgacgagcgcgcccgaggaggccgtgctgctggacaagcagcaggcggccatcaaggtggtgtgcaactcggtgtacgggttcacgggcgtggccaacgggctgctcccctgcctccccgtggcggccaccgtgacgaccatcggccgcgacatgctggtggccacgcgcgactacgtgcagacgcgctgggccacgcgcgagctgctcgagcgcgacctgcccgcgcgcccgcccgcgggcgagtacgccgtgcgcgtggtctacggggacacggactcggtcttcatccgcttctcgggcatcgcgtacgacgacgtgtgcgagctcggggagctcatggccgcgcgcatcacgcgcgacctcttccgcccccccatcaagctcgagtgcgagaagaccttccggcgcctgctgctcatcacgaagaagaagtacatcggcgtcatcaacggcggcaagatgctcatgaagggcgtcgacctggtgcgcaagaacaactgcgccttcatcaacgcgtacgcgcgccgcctggtggacctgctctttggcgacgaggccgtctcggcggccgcggccgcgatcgcgcgcgcgcccgcggcgcgctggctggagcgccccctgccgcgcggcttcgccgccttcgggcgcgtgctggccgaggcgcacgcgcgcgtcgccggcggcgccggcctggacgtggccgacttcgtcatgacggccgagctcagccgccccccggacgcgtacagcaacacgcgcctgccgcacctcaccgtctaccacaagctggccatgcgccacggggaggtgccgagcgtcaaggagcgcgtgtcctacgtcatcatcgcgcccacccccgaggccgagcgcgacgcgcgggccgtgcgcccgggcctcgccccggggaagctgctcgtcagcgacctggccgaggaccccgcctacgtggtcgcgcacggcgtgcccctcaacaccgagtactacttctcccacctgctgagcacgctcagcgtcaccttcaaggccctctttggaaatgacaccaagatcacggagaggttgctcaagcgctttattccggaaaccgccccgggggacgcgcccttcgagcacgaggccttcgcgccgctcacgggcgaggggggcgaaagtcttcaaacgctgcgtacaatcttttgtactccaacagcagctccccgtcgaagctgatgtcccgcatcttgcaataaatgtcgctcgcgctgacggcgggcacgtcgtccgcgggtctcccgtctcccttctttcgcaccaccaacacgaacatggtctgccacacgtgcgcgacgatgcggtggccggcgcaggccccgagcaggcggtccagcacgcggtggtgcacgtgcaccgacttctcggggaacaccacgtacatcatgagcgcgccctgcgcgtccgggtcctcgaagaagagcacgcgcacgtcgcgcaggcccccgtcgcgcagcacgtagtaggcgaaaaagagctccgggtgcgcgagcacctcctcgagcgcgcgctccgaggggtcccgcgcggccaccgaggccaggaacacgcggtactcgtagatgctgttgagctgctgcacgtaggccagcacgagcgccgcgcggtcgctgcgcccgagccgcggctcggcggcggcgcacgtcgggcagcagccgccgaggcccaggtagtagcccatgcccgagagcgacaggcagttgtcggccaccgcctggccgaggtcgaagggcagcgtcacgggggccgtcttgatgagcgggttcgagaggccgcgcacggtggccacctcgtccgagggctgcgcggccgcgtaggcaaagtagggcgagtagcgatcgcggaccgcgcgcgccagcgtcttgcgccgcgcggccgccgacttgcgccgcaggagtcgccgccgctcaaacatagctgccgagcgtgtgctcgagggagacgagcgcgaggccgtggcgccgcagcagggcgtgtgcgtcgcgcaggaaggcggcgcgggcagtgggcttcggcgggaacagctggtacgctttaaatacgtacgccagggtccacagctccccgcacacgcgcccctcgaagcggttctcggcggcgatctgcgccttgcgcaacgcgtcgccgcgctcaaagagcacgcgcgggtccgcctggcgctcggccacggcgcacagggggtcgcagaagaggtgcttgtagacggcgcgcgggcccgcggcgcgcatcagcgccaggaagcgcgcgtccgccgcgaagccctcgagcacgggctcgatgcagtcaaagaggcccgcgttgttctccgagtagctgagcacgcggcgccggaagcggcgcagcgccagccagtgcgcgcgcgtgagcagcatgttgcagagcaggcactcgccgtgcgtgcggttgcggccgcgcacgtgcttcagcgccagcgccagcacgggccccagcgtggcgtccaccgcgcggtagcgcgcgtacgccgcggcggtgttgcgggccaggaagccctcgtgctccgcccagtactcgtccacggcgcggtgccggcgctcctcggcggcgtccgtctgcgcgttgggctcccccgtggccacgttgcccagcagcagcatggcgaggtcggcgtagccgggcccgccggcgtccgcgtcctcggccccgcccccgcccccgcccccggcggcggcgacggcggcgcgctcgtacgcgtcgtcctcgcgctccgccgccgcgcagcacccggcgcacgcctcgtcgcccgtgtggtccttccagccccccaggctcggcggcgcgcgcgcgtccgccacggtgaaaaacatcgcgcgcgtcgcggcctggaccagaaaggactggttcgagaagcggatcttgtcgccgacggtgcggaagcagccgtgcaggaaaaagtgccgctgcacgtccacgagcgtgagcacgcgcgtggcgaaggcgcgctgcagcgacgcgcgcccgcgaaagtgcttgtccatcacgtacacgaactcgaaggcggccacgaagacggccgtggcgcacggggggcaggccagggccttggcgcacagcgccgcgtagtccgccagccagggcggggccagcgagcgctcgcggcggtacacgtcgatcgtgcgacacacgcggcagggctcgtccagcgccagcggcgccgcggcggcctccacgaagcccgcgggcgagtcctcctcgtccagcatcacgtgggcggcgaagatcagctcgcggaagagggcgtcgttcttggtcagcagcgcggggtcgaaggccgtgtacggcgcctcgaaggcgtcctcgctccagcccatggcgtgcgacggcggcggcagcggcggcggcgcggtcgatctgcgcgcggcgatcccggaggccgcgctgcgcgacttcgacgtggactttctcgaggccaactacctgcccccgcgggtgcgcgtctggttcgaggacgtgatgccgcgcgagctggaagtgatcctccccacgacggacgccaagctaaattacctggcccacacgcagcgcctggcggcggcgacgagcgagcgcgactgtgcccacggggaggcgctgcgggcgcggcgcgatcgcttcgccgcggccgtcaacaagtttctggacctgcaccaggtcctgcgcgacggggtggagctgggcgccctataaagtccgccgcccggccccgggagctccaccagccccaccaccaccgccactgccaccgcagcccgcccgcccgccgggaagaccgccgccgtcgccgcgtcgacgtcgacgtcttagccgcgccgctctccctcgccctcccggcccacggcgacgacaacgacgacgaccatgagcggcaccctgatccagcgcctgaagctcatcctctccggggggaacctgcgctgcagcgacggcgagacggcctgcgaccccgagcggcccccgacgcggtgcgtcttccaggtccacgggcaggacggctccaacgacaccttcccgctggagtacgtgctgcgcctcatgcgcagctgggcccacgtcccctgcgacccctacgtgcgcgtgcagaacacgggcgtgtcggtgctcttccagggcttcttcttccggcccgccgacgcccccctggccgcgatcacggccgagcacaacaacgtgatcttggcctcgacgcacagcaccggcatgagcctctcggcgctcgacgacatcaagcgcgccgggggcgtggacatgcggccgctgcgcgccatgatgtcggtgagctgcttcgtgcgcatgccgcgcgtgcagctctcgttccgcttcatgggtcccgacgacgcctcgcagatgcagcgcctcctggaccgcgccgagctgcggcagcgctcggccacccgccccggcggtggcgacgacggctgctcgggcgagggcccctcgccccgcgcgcccatccgcccacccgtcctctcgcccgtgcccggacacgcggccgccgccctcgtgggccaggccgcgtaccctccgcccacgcggttcccggcctcgctgctgcacgccctcctggggctgcggcgcctcgccggctacgccgtggcgtgcgtcaccggcgctctcgcgatagtcatcatcctgaacatgcgttaaaggcggccgcgcgcgcgggggcgcgcacagacgcgcgctccccgccgagccatcatgtccttcgacccgaacaatccccggacgatcaccgcgcagacgctcgagggcgccctgcccgtggacattctgctccggctcaaccgcgcgacggggctgcatatggatgcggccgaggcgcacgcgatcgtggaggacgcccgccggaccctgtttatcggcacctccctggccctcgtgaacctgcggcacgcgcacgacaagcacctcgtggagcgccagcccatgttcgccaccagcgactacagcagctgggccaggcccacggtcggcctgaagcggaccttctgccctcgcccctcgccctagccccgcgcgatcaataaagcgagccgcgttgcaccatcctctcgtctctcgtgtgttgtctgggtggggtttcccccgagatcccccgatctccccgccgggtctttgggtcccccaccgagttccccaccgagttccccaccgagttccccaccgagttccccaccgagttccccaaatgcccaccaccaccaagtctttgggtcccacaacccccaagttttggggtctcgggtccgagtcccccgagtcccccgagtcccccgagtcccccgagtcccccgagtcccccgagtcccccgagtcccccgagtcccccgagtcccccgagtcccccgagtcccccgagtcccccgagtccccgagtcccccgagtcccccgagtcccccgagtcccccgagtcccccgagtcccccgaacacaccaccgcagagacaaacaggttgggtaataaacaatttattaaccaagaatcaggcggacctgaaataggtccacgttgaaccggcgggcgctgcccttgaggttcgcccgcgtctcccgcaggcggcgcgcgatcaggcggcacgcggcgatgagcagggccagggccccaagtccggtggcgcgcatgtaccgcctgttgatgagagtgtccgactccaccacgcgcggggggagcagctcctgggccagagagacgtcctcgtcctcgtcgtcctcatcgtcctcgaactccgcgccccaggggcccggctcgggcgcctcctcggcgggctcgcgggtcaggtccgcccgggtgggccagacgcggccgaggccgcccaggggagagtccggcggtgggggtctctgctgctgccgcggcggcggaggagggtgggcgggagggacgagcgcggccaggtcgtcgaaccacggcgggggctggtggcggtccgccagcggggagcgcggggccggcggcgcaacctccggcgggagctcggggacggtggtgggcgcctcagacagcggcggccagggcagtcgctccagcggcgagcgcggcggggtcgccggggcctcggcctcttctccctcgctcgcccatagcggctgagggggcggcggcagcggtgatggcggcagcggcggcgcctctcctccggggaacgggaacgggaacggctggtccgagggcggcggggtgaagagcgcgggtgggccggcggggtccctcggctcgggccacgaccacggcgcctccggggggcgctgctgagacggctgcggcggcagcggcggaggtgtgaacaggagcctcgggggctccgcgggcttctcgggctccgccggcgccggtctcgtctgcgccggggcccgggcgggctcggccctcggcggcgccggggccggccgcgaagatggtggtggtggtggcggcggcggcggctggggtggtggtggagcggcggcggaggaggccgaggaggagatggtggtggcggcggtggaggaggaggtggtggtggcggtggcggtggtggtagaggccggtgctgttgttgctgttgttgttggagctgagacggcggcggggggccgcgcgctctcgggggcggcggccagcgacgtcggggccgtcagcggagaccgcggcggctgggtctgcgccggggccggagtctccggcgaggccggcagcggcaccgcgacgccctccagcggcacagactgcggcggcgggatgtggagacggaggtgcccggccggcgggcgcgcgacggcggcggctcgcctcactggtcctccgggggctgggaccacatctccgagcccgactccgagggcggggacggctgctgctgctgttgctgctgccgccgccggtgggcccgctggagcagtcgccggcgcttgggcttgggcttgaggcgggcgcggttcagctcccccgggaggacggccctcgggttgagccgcgggcggaagactcccggtctggcgcgaggctgatacgggccttcgcggaacaagagccatggattttcggcgactgtggtctctaatacccaccgacgttcgcgccttggtgcgtagaacagcggagggaaggggctgcgccactggggccgcggggagatgtcccgcaggttgatctccacggctcgcatccgaagccgggccaccgtctcctcggcgctggccaccactcccgggggctccaggaggtgctcgggccgctgctccggctgctgggggggaacctccgcgggagccatcgctcggtgttccggccggggaagaagaggaggacgaggcggcttctcgggacggagtcgaggcggtggaggaggacgcggacgagagctccgagtccgatgtcccgggaggagagcgggtccgacgctggtctggtccgaccgtgtgttcgcacttgtctgcgcaccccgctttatagcgttcgtcatcgagggggcggcccggggaggcggtccccgccaccctctcaccaaatttggtgtctcggacgtccggcggggcatcggccctcggcgctacgatgcggtcaccgtcgggtttctgggccgatggggcgggcccgggcgcggggggccggaaggtcgccgtcggggcagatgggggtggcggggcgggcggggcggcgggcccgggggtcgtcgtcggggcagatgggggtggcggggcgggcggggcggcgggcccgggggtcgtcgtcggggcagatgggggcggcggctgtgggggagcgggtggggcggggggtgccgggggcgcgggcggggcgccccggggcttggcggccggctgggacgatgggggctgagcggcggcggcggcgacggtggtggtcgcggcgggctgtggcgggggaccctgtgacggaggttggggccggggctggggctgggtctgggggatgggggccttggccgcggcgggctgcgacggtggggcctgggtcgggggagccggggccgacgggggagcctgagtctggggctggggttggggggtcggcttcgccgtcgcggcgggctgcggcgggggcgcctgggccgggggagccggggcctggggggtctgctccgccggcgcggcgggctgggtctggggggccttctcgggcttcgggggcgccgcggggtaaggcgggttgatgatcggcggcgggggctcgacgcgcgccttcagcgagggctggatctttcgcggcgacgagtgcggcttgggggtgggctcctggggccgctgcttgggggccgcggcggccttggcggccttcttggaggcggtggcggcggggggctcctgccgctgctgctgctgctgttgttgctgctgcggcggcggcggttgcgatggcgccttgggggcggcggggggctcctgctgccgctgcggcggcggctgctgcggctgcggcggcggcggcgccctggtgagcggggtcgcctccacgggcgcggcgggcggcggcgccggcggggccagcttgacgagcggggtcgtctccgcgggcgcggcgggcggcggcgccggcttggcggcgggggccgcctccgcggggggagccggtggcggcggcgccgggggggccagcggcgggagcgcgggcgggtcgaacggcggcgacgggggcagcggcagcgcgctggccaccaggtcgtcgccgagcaggtccagccacgggttggcgtcaaagtcgtcgtccgggacgaacccgtcgtcctcctcggccagctcgggcggcagcccggggatcggggtcgcgggccgggtgggcgtcgccgacacggcgtacgggaacgggtcgcccaggttgcggaagctcggccggccgtacagctcctccgcggtggccgggtccacgcgggcgtcgcccatgatgatggaggtgaacagcgggtcccgggggtccatccccggcagctggtcctccgtcgtctccaggaaggggatctcccgcagcagcgggtcgtcctcgagcgtgaagcccacgcgccgcggcggctcgggcggcgcccagagcacgcgcgacaccgccagctgcgagtccaccagcacgaggcaggccggcgtgccggccaggggccgcgccgcgatcagcgccgagagccgctccagctgcggcgtcaggcacgcgttctcgatggggttgtcgtcgagcgccagcaggcgctcgccccaggagctgaggtccgtggacacgccgcggtgcagcgcggggtcgcgcaggtcgcacagcgtcacggccagggtcaggcccgagccgggcgagtacacgtccaggttctccatggcgatcaccagcggggccccgacgagcaccgccgccgcggccaggtccatggcgctcacgcgcgtcgcgctgctcggcggcgagcccgtgacggtgaacgtcatgcccgtgcccacgggcgcgtacaggtcgccgggcgccggcgcgccgagcgccgcggcctcgcgcacgtgcgccgcggcgtccaggcgctccgtgaccgcggcgtcgtacgcggcgttcgcctccggctgcagggcggaccacagcgccgagagcagccgcggggggatgcacatgcgcgccagcacggtgaaggtggccagcgcgcccggcacggcgcgcgccagccgctggcccgtctccgtgtgcgcccagggcgcccacaggtccgtctccgagaagcggccctcggtccagtcggcgacgcgcagcgtgaacagcgcctgcccgtcgcgcagcgggcgccgcggcgggcgcgcgcccgcgtccagcgtgtccaccgcgtccagcgcgcggtccagcaccgcgtccagcgtcttggccatgtactcgtgctgcccggccaggtccacgcgcgtaaagttgaacaggtgcgccggctcgccggccaccgcggccaccatcaggtcggggagggcgagccgcaccgccggctccagcggcggctgcacgacgcggtccagcgccccgaaggtgcgcgagagcgtcgtggccagcacggccgcgtacgtctccgcggcggccttggccgccgagtcccagtcggcgcggcggtggcgcacgaagcgcgcgaaggtggcgaagcccgtgtcgcgggccttctcgtagttgagccgcaggtggatgatctcgttcatgaggcgcatgcccgccacggtcgccgtctccatgagcccctcggtgagcggcgggcgcagctcggcgccctcgtcggtgcacagcatggccgccagcttgtcgccgaccgcgcggtagcacacctggtactccacgggcacgttgttgtcgtccaggaagaccgtggggaacaccgcgtccgcggcgctgcgcgcctcgcgcaccgtgcagatgacgtcgcggtcgcccaggcgcgccgccacgcgctgcttcaggcgcgcgcacggcgccgtcgaggcgcgcggccgcgccggcgtcggccccgggccgccgtaggccgcgtagaactgcagccgcagcgtgagcagctcgcggtgcgcggcgtacttgggcgggacccagcgcggcagccgctccgcgcgcgcgtcgaaggcggccgccagcgcgcgcagggcgtcgtgctgcagctcgggcccgatgcgcgccgccatgcggtcgagctcgcgcatgtcctcctcgacggcgccggcgtcgcggaagaagtcgcccagggccccggcgcgcgccgagtacttgctctcgatgcgccccacgaacttggcgggcaggcgcgggtagacggcgtcgctgcgcagcgccagcacctccatggtcgccgtctgcagcgcgcgcgcgcgggcctcggccaggcgcgcctcgtcctcggaggcgccggcgcggccgcgcgcgcgcccaaagtcctcccaggcctcgtcccaggcgacctcggcggccgtcagccgctgccgcagcgtgtccgcctcctggcgcagcgtctgcagcgcgtcgatgcgctcggcgaactcgtccatgggcccgcggccgtcgacgcgcgtcgtcagcgggtgcgtgtcgatgatgagccgcgcctggtgcagccactcgacggcctgcacgtccagctcgggcgcctcctcgacggcgccgagcagcagcgccgcctgcgtcaccagctcctccgcggaggcggcccggtcgatgttgagcgaggccggcggcggcgtcacccgcagcaggttcttgaggttggccagctcgagggcgccgtcgcggtcgcgcgccgccgccgcgtccaccacctcgcgcagcaggcgcgtggcgcgctccgtggccgcggcctggtccgcctccgccgcggcgaggccgtcgcgcgccgccgccaggtcgcggcgcacgagcgcgcgcatggcctccgcgtacgccgtgcgctcgaagcgcgcaaagtcgaagcgctccaggtaggtcacggcgccgcgcagcagctcggtgctcggcacgtgcaggtgcacgccctcgtcgagcgcgcgcgccgcgtcggcggtcgcgcgcacgtacgtcacctcctcgagcgcgcggcagatctcggcgccgcggctgccgccggcctgcagcacgtccgcgcggcgcgcgcccagggcggccagctcggcctcgtactcggggaagccgcggcggtaaaagtccacgtacttggccaggcgcggcacgtgcgccatgtccgccccgacgaagcccacggcgtcgtagtagctgaagcgccccatgccggcggcggcgcgggccagcacgccgccggcgatgcgcgccagccgcgagagcggctccgtgtcggcgccgaagagcgacccgatgacgggcgccgcgacggcgtagtcgtcgagccaggtcaggcgctgcagcgcctgcagcggcgggcccgccgccgcgccggcgttctcgggcaggtgcgggttgaaggcgaacacggcgtccagcgccgtggtcacggtctcggcgttcgcggcgagcgcgcgctcggcggcggggcgcagctccgcggcgtcgtagccgcggtccgccgccacgtcgcgcaggcgcacgaactcggccgcgtcgaaggcgtgctgctcggcgcggcgcagcgccgcgtcggcggcgcgcgcccagagctggcgctgcgcgcgctccacctccgccgcgcggcgcgcctccacggccgccacggccgcctccagctcgccgacgcgctggcgcgccgcgagcgccatcttctgcagctgcggctcctccacggcggccgcgcgcgccacctccagcgccaggtgccgcgcctcgaccagcgtcccctcgtcgggctgcgcgcgcagccgcccgagcatctccacggccgccgccacggccgccgacagcgcggcctgcagcgccgccacgcgctcagactcgcgcagcgccgcggcgcgctcctcgcactgccgcgccagcggctccaggaagcccagctccgggtgctccacggcggcgcggagcacccccgcggcgcgcgccgccttctccgccgtgtccgcgtccgccagcgccgccgccaccgcgtcggcgtgcgcgaggaagaagcgcgtgatgggcgggttgtccgcgagcgcgttgaccagcgcgccgagcggctgcccggcgcccaccagcgcgccggggtcggaggtcagcagctcgcggtagcgcgccaccagccgccacagcgccgcgaccaccgtgccgggcacgggcgtggccgtgatgtgcgcgagcaggtccaccgccgggcccagcgtggcaaagtcgccgagcagcgcgcgcagccgcatgtagaggtcgttctgctcggcggcgcgctgctgcgccgcctgcaccagcgtgcgcagctcggcgtcgcgccgcgcgagccgcgcgcgcgtctcctggctcagcacgtggccgctcatgccgcggatgtgccgcagcaggtcctccatcgcgcgcacgcgcgtctcctcgtccggggcggccgccagggcggcgtccatggcgtcgaagcgcgccaggtcggcgtcggccgaggcgcgcttggccgcgcgctcgttgatctggttcacgtcgcgcgtgagcgtgtccagctcgcgccgctccagatgcccgtgcgcctgcgcctcggtcagcagcgcgaaccaggccccgaggcgctcgtccgtgctcacgtcctcgcccgcctccagcagctgcgccagcagcgcgccgtcgtgggcggcgggcgccgcgtccagcgcgcgcagccgctcctcggtggccgccagcgagtcgacgaggcggcgcaggcccgccaccgcggcgttggccacgcggaagcgctgcgcgccgttgcggtccatcagcatggccttggcgctgtactgcgcgccgcgcagaaagtactcgcggatggcgtcgccgaccatcagcgccagcttgcgcggcagcttggccgcgcgctccttgacgtcgcgcgtgttcggctcggccgcgccgggcggcgcccccgcgtccacggcgtcaaagttggcgtgcgcggcctcgagcgccgcctcgagcgcgctcacgtcgcgcgcgcgctcgagcgcgcgctgcgactcgcgggactcgcgcgcccgcgcgcgcgcaaacaggccccgcgcgcgcgacaccaccgtctggtcgtagacgccgccggggtcccgcgccgcggcctccatgaagcacgccgagagctgcgcgtaggagcccccggactcgagcgcgtccagcgcccggtgcatggcctcgttcgcgggccgcgcgacgcgcgccgccaccagcgccagcttggccacgggcgcggacgcctcgccgccggccgcgtccagcagcgcgcgcgcctccgtcagcaccatgtgcgtggcggccacaaagtccccggccgggccgcgctccggcagcgcggccaggacgggctcgaagagcgccgcgacggcgcacggcgcctccgcgtgggtgcgggcgccgttctccaccagaaagtcctgcaggtgcccgaagaggagctcggcgaagcgctccagcagcccggcgcgcccggggaacaggctccggcccgcgcacgcgaccacgtgctcctcgagctcgtctgcgtgggcctgcagcgggcccaggtcgagcgggaccagctccagctcccgcacggggaagcccatgggctcgcgggtgcgcgggcggtacttgcgcgacggccgcccgccgaccgtgtgcgtgctgctcaggtcctcctggctcgccggccacgggggccggcgccgcttgggcatgctcggccgcttctggacccgcggcggcgcggcgagggacgagggctggggctgggactggggcaccgccgtcgagaggggggcggcggggacgggggcgacggcgaggacggggatggtcccgacggcggcgacggcgggggcgcccccggggccgcgcggcggcggcgcggcggccgccgggtccgggggcagggccatcgggtgcacggagggcaccgcccgctccacgtacggctcgtccacgaggtacgtctcgcccacgccgtagagcgcgggcacggcggcgaactggtcgtggggcaggaagaacacgaaggccccggcccagcggagctcgctggcggcgtagcccgcgacgtgcgcgtacacgtccgccgggcgcaggcgcgccacgaaggcctgcgtgatctgcccgtggccgtgcgggtcgaagacgtagacgacgtcgccgcggcggtagaggcccaggcccacggcgccgaccacgaccagcgtgaaggactcggcgcgctgcgcccagaggccgtcgaagaaggcgcgcgccgtgatctgcgtgctctggaagccctcgccgggcggcgtgaagaagttgcactcgccgtgcacccgcgagaagacgcagtgcagcgcggcgccgccggcgccctcgtagacgatcttgttcgggagctcgctgatggcgcacatgcccccgtcgggccccgtccacgcctggccctcccgcaggcacgcgtccacggcgtccgccgtcagggcggcctccacgccgttcgtgaagaccaggcgcaggaaggagagggaggagcgcaggcacgagacgcccgacccgggccccaggtcggggtcatactgattacgatagccgacgaccaccgcgtcggccgtcatggctgaaataacacacgcgcgtgggggaaaaatctttttatttggacacggcgtcgacctcggccaggagccgatccacgggcacccacgtttgcgcgagcaggggcacgcccgcgcccgcgtcgtcgggcgcgcgcgcgccgcggcgcagcacctccaggttcacggcggcccagtcgcgcacgttgacgctcgccgagagcggcacggcgtcggggcccgcgtcgccgtcggtcgccacctcgtggtagtcgcccaggacgcggtccgcggcctcgcgcagctccccgggcgtgaaggcggccgcgggcggcgcctgctcctcgcgcgcctcggtccaggtctcgcggagcgtcgcggcgagcgccagcacgccgcgctcgtcgacggcgcggcgcagcgcggcgctgaccaggtcccagcgcgccaggaagcgctccacgggccgcaggacggtggcggcctgcaggcggccggccgtgagggcggcgctcgtctgcagcttgctcaggcccgcgtggtcggcgcacagcgtccggaacacgggcgcctgcaggcccagcgcctcgcactcgcgcagcgcctccgtcgtctgcgccatcacgccgcgcagcgcccccgcgtcggcgcgcatgtccgagcggtgcgtgttaacctcggcgtggaaggcgcccacggcgtccacgacgtccgccaggcgccacgtgcccaggcagggcccgccctcgccgggcgcgaagacggaggccgtgcgcccgcggaagacgaagcggcaggtgaagaggcgctcgagcgccaccacgcgctcggccacggcgcccgcggccgtggccatcgagtccagcaggtcgcccgtgtaggtggccaggtcctccaggcggcgccggagggcgcgcgccatcaggtccccgaagaaggcgctggtggccagcgcgggcggcgcgggcaccggcgcctcgagcacctcggcggccgcgcgcacgaccccgtgcagctcgtgcgcgagcgcctcgacgttgcgcgcgtgcgccaccgtcaggtcctcgcgcgccagcgccgccacgagcgcggcgagcttggtgcggtagccgctgacgttggggatgaggccggcgccgtagtcgcgcgcccacgtcagcacctgcgccagctccgcgcggcggtcgtaggaggccgtcaggaggcagttgtaggcgtcggccacggccgtcacgacgaagaagggcgcgaaggacgggtcggccatggcctcgaggtgcaccatggacgggtagttcacgaggtccgcggcggcggcgaggtcgacgagcgccgcgctcgtcggcgcctccatgggcgtctcgccggcgccgtcggcgcgcaggccccagcggctctgcgcgcggaagccgagccacgtgccgagggcctcgcgcgccgcgctgtgcgccagcccggcgcggcgctcggccaccgccacctcgcgcatcagcgcggcgcgcgcggcgcccacgtgctccgtgggcgggcgcggcgccatgcagtggcgcaccacgtgctccacgcccttctcggggagccccagcgccgccgtcacgtccccgtagaggggctccagcgtctccgcgacgtccgtgtaggcgcgcacgtagtccagcccgggcccgtagtcgtcgaggaggcggtgcacgacgccgccgacgaccgccgtgcagccgacggcgcgctgcgcgtccgtcaggcggggcgcgcggtccagcacggcgcgctcgaggaggtcgcagtggcggcgcgtgaagcccgcggtcacgagcgcctccacgacctgcagcggcgaggcgctggcctgcaccgcctcccacacgcccgtggcgtccacctgcgcgtggtcgaggtccgcggcggcggccacgtgcacggcctgcgccccggggtccagcagcgccagcacgccctggtccgtgcgcaggcgccgcgccacggtgcccgggcgcagctcgcgcacgtacgcgtccccgtgcgcgagggccgcggacacgcgcacctcgacggccgtgcgcggcgtggcgttgacctcgtccacgggcaccagcgccgcctcgtccacggcccgcgccgcctcggcgaactgctcgcgcagcgtcgggttctggcggtcgtggaagaactcgtcggcgatcgtgagggcaaagagcgcgcgcgtcagcggcttcaggccctcgttctgcatctggcgcaccgccagcgccagcggcagcgcccagttggtgcgccgcgccacgtggcgcaggccgtcctgcacaaagtccatgtcgtagagcgtggcgaagcggggctgcacgagcgcgcgcgccgtctgctccgggggcacgacgcggggcgcggccaggacgtcgtcggcggagcgcgcgcccgccagggcctcgtcgaaggcggcgcaccactcgctcacgaggcggaacatggcgtcggggccgaactcggccgaggtgcgcagcccggcctcggtgagcgcggccgtggtggcgtcggcgttgacgacccccaggagctccagcagcggggccgtgccctcgtcccagtcgtggcgcaggcgccacagcgccaggccggccaggttctcggcgagcacggccgcctcgcaggtgccggtcgcggcgtaggcgcggcaggccaggcgcagcgcgcgcgcccagagcccgcgcacccccgcgggcgtctcgcggcccggggccatgaagaactccaggatgtgcgactgcaccaccgtggcgacggcatggtcggcctcctcgagcgcgcgcacgagcgcctccattataagagggtccgcgcgcgggcgccgccgcgcactcgaccccgcgcggcgcgacgatgagcgtgcagatcggcaacgggctgctgatggtggtcgcgccgggcacactaaccgtgggctcggcgcgcgcgcgccttatacgccaggtgacgctggcggacttttgcgagccccaggccgagcgccccgggctggtggtgctggcgctgcggcaccccgcggacctggccggcgccgcctacgcggccacgccgcccggcaagaaccaccgcgacctggaggaggcgtggctggccctcgacgagggcgggcgcggcctcggcggcgacggcatccgcgcctccgtcgtctcgctcaactttttggtggcggccgccgagaacggggacgacgcgctgcgcgcccacgtgacgaccaactaccgcgaccggcgcacggccgcgcgcctcgagcgcttcgcggccgtgctgcgcgccatgatccggagccacgtgttcccgcaccgcgcgctgcacgtcctcggagggctcctgggccacgtgacgcaggaccggctggccagcgtcacgtgcgtggcgcgcggcgaccaggaggcggcgcgcaccaacgacatggccgcgcgccgctcgcaggtgcacgtgcccgcgtgcgcgctgatggacgtggaccgcgagctgcgcctcggcggcgacgacggcctccgcttcgcgtacctggtctttgtctacacgcagcgccaccgccgcgaggcgctgcgcgtccacgtggcggtgagccgcctgcccgagctcggcgacgccctcagcttcctcctggccggcacgcgcgtcgacaacgcgatccacggcacggacgaggccgacgcccccgccgcgcccgccgccgccgcggccttccccgcgtacctgttcaacgacccgcgcagcgcgcgctgcccgacgggccggctcaacacgcccgccgccgaggcgctgcccgtgtgggcccccgacatgcgcggccgcgccacccggaactcgtgcatgtacgccgcctacgtgcgcctcggcaccgtcgagcgcgtcgtgcgccgggccgagcgctgcggctcggtggacctgccgctggcccacatggagcgcttcacctgggacgtgggggcgtgggaggagtgtttcttctgaaaaaaaccgggggcggtgtgtgtgagacggatgtgatgttgctgacgaggctaataaaaagcgcgggctacacgcgcgcgtgtgtccccccgctgaccctccgtgccgcgtccgcgtcgtgtgtgactcacccccatcgtctctcccgcccgcgatcccggcccgtcccggcttgtcccgccccgcccagacacatcccatcatgcccccgcgcacgccccgcccgccgggcgcggttttcccccctttccccccattggcggcctccgccgctaaaagcccgcgcgccccgagctcgggactctccgcgcactccccgtttcgagcacacaccgtcatggcctcccccgtcgtgcccgccgccgccgccgccgctcccggcgcggactccttcctcgacgccgtctgccccgaggacgtcgcccgggccctggccgccgagctcgaggcgctccgcgccctcgggcacgacgtcggcgcgcccgcgcccggggcctcgcgtcgcgaggtggcgctgtttgtcacccgcgccgtggacgggctcaaggccttctcccgggtggacgagcgcgtgtacgtggcctgcggcaagctggtgcacctgcgcgtgcgcagccgcgaggcggacctggacgcctggctcgcctcgccggagctcgcgctgatccccgcggtggcgggcgccgtgcgccggcaccgcgcgcgcgtggaggcggccctccgctggttctggcgggaggcctacccggcgctctacgcgcgcggcctgcagtcggcgctcaagtacgaggagatgtacctggcccgcctcgagcacgggcgctgcgaggccatggaccagttctttgtgcgcctcgccgccgccgccgccacggcgacgcggcgcgccatggcgctcgtgctctgcggctcggacgcgtggccggaggtctttgacgcgtactttcgcgcgctggccacgcaggcgatcgtgcccgccaccccgctcatgctcttcgccggccgcgcgcgcggctcgctcgccagctgctacctgctgaaccccctgccgcgcaccaccgaggaggcggtgcgcgccatcacggacgaggtggcccccatcctgctgcgccgcggcggcgtcgggctctcgctgcagagcttcaaccgcacgccctcgggcgactgcacgcgcggcatcatggccgtgctcaaggcgctggactcgatgacggcggcgatcaacagcgacagcgagcgccccacgggcgtgtgcgtgtacgtcgagccgtggcacgcggacgtgcgcgccgtgctgaacatgcgcggcatgctcgccgccgacgagagcctccgctgcgacaacatcttcagctgcctctggacgccggacctgttcttccagcgctaccagcggcacctggacggcgagcgcgccgtcaagtggacgctctttgacgaccgcgcctcgcacctgacctcgctgcacgggcccgactttgcgcgcgagtacgagcgcctggagcgcgccggcctcggcgtcgagtccctgcccatccaggacatggccttcctcatcgtccgcagcgccgtcatgacgggcagccccttcctcatgatgaaggacgcgtgcaaccggcacttccacacggacacgcgcggcgccgccctcgccacgtccaacctctgcacggagatcgtgcagcgggccacgcccggcgagaacggcgtctgcaacctggccagcgtgaacctgcccgcgtgcctggcgggcggcgccttcgactttgccgcgctgcgccgggccgcgcgcgtcgccgccgtcttcgtcaacgccatgatgcgcatcgggaactacccgacggaggcctcggtcgagggcgtgcggcgcagccgctcgctgggcatcggcctgcagggcctgcacacgaccgtgctggcgctggacatggacatggccgacccggcggcgcggcgcctcaacgccgccatcgccgaggagctgctctacggcgtcatggacgccagcgtggagctctgcgagcgcggcctgcgccccttcgacggcttcgagcacagccgctacgcgcgcggggtcatgcccttcgacgcctacgagcgcgtctcgctccgcgagccgatgcgctgggacgcgctgcgcgtccgcatcgcggagcacggcgtgtacaacgcgcagtttgtggcgctgatgcccaccgtgtcctcgtcgcaggtcaccgagagcagcgagggcttctcgcccaccttcaccaacatgttcagcaaggtcaccatctcgggggagctcctgcggcccaacctgccgctcatggagacgctgcggcgcctgtttccgcgcgagtgcgcgcgccgggacgccgtggcgcggctggagcgcgcgcagtggtccgtggccgcggccttcggggagctgcccgccgggcacccgctggccaagttcaagacggccttcgagtacgaccaggagctgctcatcgacatgtgcgcggaccgcgccccctttgtggaccacagccagtccatgtccctcttcctgaccgagcccgccgacgggaagttacacgcctcccgcgtcatgggcctcctcatgcgggcatataacctgggcctcaagacgggcatgtactactgcaagatcaggaaggccaccaacaacggggtgttcaccggcggggacctggtctgcacgagctgccacctgtgagcgcgcgccatggagtacttttacacgtcccagtgccccgacatggaccacctccgctcgctgagcgtggccaaccgctggctcgagacggacctgccgctgggcgacgacgccaaggacgtggccgcgctcagcgaggccgagctcgagttctaccgcttcctcttcgccttcctctccgccgcggacgacctcgtcaacgtcaacctgggcagcctctcggagctcttcacgcagaaggacatcctgcactactacatcgagcaggagtgcatcgaggtggtgcactcgcgcgtgtacagcgccatccagctgatgctgttccgcggcgacgccgcagcgcgcgagcgctacgtgcgcgcggcgctgcgggacgaggccatccgccgcaaggtggagtggctcgactcgcgcgtcgccgagtgcgcctccgtcgcggaaaagtacctgctcatgatcctcatcgagggcatcttcttcgcctcctcgttcgcctccatctcgtacctccgcacgcacaacctgtttgtggtggcgtgccagtccaacgacttcatcagccgcgacgaggccatccacacctcggcctcgtgctgcatctacaacaactacctgggggacgccccgcgccccgacgaggcccgcatccaccagctcttcgccgaggcggtggagatcgagtgcgagtttctgcgggcgcgcgccccgcgcgacagcctcctgctggacctgccggccatcatctcgtacgtgcgctacagcgcggaccggctgctgcaggccatcggcgcgagcccgctctttggcgcgcccgcccccgcggcggactttcccatggcgctgatggtcgccgagaagcacaccaactttttcgagcggcgcagcaccaactacacggggaccgtcgtgaacgacctgtagcgcccagcggcggcgacccccgctctcccccctcctcctctctctgcgacaaaacactaataaagcgttgagacactagcgcgcgcctccagccgtcatccttggggcgagggtgggacgggaacggggatgggagagtggggtggggagagtgcggggatgatgacgggggaagggagatggggctgacgggggtggactgggactggggaaggatgatggcggccggcgcagagtgggaggcggtgaggtggacggcgggtgggacggcggggggaccgatgtggggcggcagaggggcggcagaggggcggcagaggggcggcagaggggcggcagaggggcggcagaggggcggctgaggggcggcagaggggcggcggcgaggtgagacggagcccgcggacggagtggccggcggggggaggcgacgacggagagagaaggtgggggagagggcgagcatcacacgggccgcacaaccgaggatttttttcaagttgttttttattttctcctatgggcgttacagtcgtcccagtacgtcatgaggacggcctggtagttcggaggcggaggtatgtgcttccaaaacaggcccgccagctccttcgcccgcgtctcgttcttcacgtgccgcagcagcgagccaaagaccttgttcacgtccgagggctcctgcacgatggggacgcgccgcagcacgctcaggtgcccgtgccgcgccggcgtgagcatggccaccacgtgctggatgaactcgcgctcggcgcggcgcgagcgcgcgtccccggccgccggcagcagcgcgagcgccttgccggcgtcgcgcatgatgtcggggcagcgggacgcgtacttgagcttgacgtcctcgggcgccacctcgcggccctccagcgactcggccacctgctgcaccgagtccacgtccggcgcccggtgcaggtccgtgtggcagcgcacgaacgcggccaggaactccgagtagtccacccccaggctcgcgaggacgtcgcggcagcgcagcaccagcgggaacatgggcgcgatgtcgaggatcatgtcgcacccggtgaggatcatgtccgtgtcggtcgtgtgcacctgcgcgaccgtgttggtgtggtagaggttggcgcagacgtcgtccgcctccatgtccgacacgtccacgtaggcgtagcccatgtggcggatgaggttgacgcagaggcggtggacgatgcgcggcgcgtgcagcatcgtgctccagcgcggccgcggcggcgcgtcggcggccccgtcgccgtcggcgcgcgccgccgcggccatgatggccttggcgccgtgcgcgacgcggccgttcccgaagatgccgcggtccgagacgaagatggggaagtaggtgcgcttgtgcagcatgcgcagcaggcgcagcaggcagcgggcggtcgtggtcgcgttgtcctcggtcgtctcctggtagtgcttctccatgagggtgtacatgacgttccacaggtcgatggcgatgggcgtgaggaccccgggcggcgtggagatggcctccgagcggaccagacgatggcgatgtgcgtactttaaaaggccaaagagccccatgtctccgctcgcacaccgaggcgccggtctaaaatacgcatgtggcggggcggggccacccgcgcatataagccgggcggtgattggtctggcgcacaccgcccgcccgagcccgctcgcccgccgcgatgtcgctgttcgacgacggcctcgaggacctggaccgccaccccacccacgcgcaccacccggcgcaggtgatccacgacgggcccttcgtgctggaggacggcgagcccctgcagcgcaccggcatgctggtgctcagcgacgagcacctggagcacgcgcgcgccgccatcgccccgctcgccgcgcacctcgcgcacgccttcctcgtcttcagcgaggccgggctgctggtgcacgccagcgtgcgcggcgagcaggtctacgtgaccctggccccggaccagttcagcacgttcgtgtggagcgggccccaggccgtgttcctgggcaacgtcgacggcagcggcggcgtgctcgacgcgctcaaggtcgaccggcggcggaccgtcttcaacgtcaccttcgaggtgtacggcgccttcccggcgcggctgctgacgcggcgcgcgtactttgcggacgcgggcctcctcgcggcggggcccggctccccgagcgtcgcctgcgtctacaagcacgagttcaacgactactgcatcatgctcccctcgcgggcgcccgacgtgagcctgacgctctcgcgcccccaggtggccaagctcgccgccgtggcgaagggcgccgcggccgggacgaccttcgcgctcgcccgcggcctcgacttctccgtctcctccagcgccggggtcgtgaccttcccggcgcgcgaccacgacgggaccgccgtgctggagcgcgccagccggcggcgccagggcgtcgacgcggtcggcgcgacggagcccttcgccatgacgctcgaggcggcgcacgggctgctgacgctgctgcagcggctgcgggccgggaacgccgagctcacgttcaactttttcacgacgccgcggcaggcgcccctgttcagcgtgaccacctgcggcccggtgcgggcgaccaccttcttcttctgcgcgcccgccgaccccgccaccgtgcccgccgcccccgagggcgccgccgccaccgtcgccgccgcctgcggggcgggcgcgtccgccgcctcccccgccgcgggggacaagcggcccgccgccccgcgcatgtacacgcccatcgccaagcgcccgcggaccgcctcgggggaagggggccacgcctacggagatttattctaataaagtgagcaggtgtataaaagagaggtcgcacctccgcgtctttactcgccgtcgcgatgatctcccgggggaacggagggggttgccgccccggcgagccatgctggcgctgcgcgctcgagtccacccgctgcatcacgctgatgggcgtgctcgtcgcgctcctcgccgcctgcatgctgtccgtcccgcccgcggcgtcgacgatgctgctcggcgtcgccagcctcatggccatgctgcgcctgcccatgcccctcgtggaccggttcatccccgcgtgcatgggcctccagctcgtcggcgccgccgtcttcgccgcgggctgggcgctggccagccgcgacgcgatctcggccggcgtcctgctgtgggccgtctgcgcgctcatctcgcacatgtacaacgtcgtctgcgtcgccagcgggcccgacgcgcactaccgccccgcctgcctcgtcatgggcgtcgccgcggcctgcggcgcggcgggggccctcgtgaacgtgcgcaccgaggcgcgcctcggcatcgccctggggctggccgtcacgtgcgccacgaacaacgtcgcccgcagcctgcgcggcacgtgcacctacgtcgccagccgcgcccggttcctggccgcgcccgcggacctggggcgcggctacagcgtggagaacgcggacgccgaccccaccgccgagcccgagcggcgcgtctacgaggcgaccgtcccgcccacccacgtctacgccggcagcatcgcgctcttcgccctcgtcttctcggccgcctcctccctccagtggatggtctcccagatggtgggccgcggcaaccagctcgtctcgcccaccaccgcggccgcggccggcgccgccgggttcctggacgcggcggcgctctcgctctttgtgcgcccgagcacgcgccacctctcggtggccgtcaagggcgcgcacgcgctcctcatcctcgcggccatcgtcctcacggccgccggcgagcccatgggcgtgcccatcagcctcgcggcctccaccggcctcgcgctgctcgaggcggaccacgtccgcctgcgccacacccgcgcgtaccggctcgccgccgcgcacgtgacgcgggccctgctcgtgcaggcgtacgtgaccgtcgccatgtgtgccactagcattaaatccgtttcctgattcacgcccacgctcgtgtcgtttttaaaaccgcgatggggggacggggggccattcgcacgcgccatggcctcgctcgcgcgtgcgatgctcgcgctgctggcgctctacacggcggccatcgccgcggcgccgtcgtccacgacggcgctcggcacgacgcccaacgggggcgggggcggcaacagcagcgcgggcgagctctcgccctcgccgccctcgacgcccgagcccgtctcggggacgacgggggccgcggcctccacgcccgccgccgtctcgacgccccgggtcccgccgccctcggtctcgcgccggaagccccagcggaacggcaacaggacgcgcgtccacggcgacaaggccacctcgcacgggcgcaagcgcatcgtgtgccgcgagcggctgttctcggcgagggtgggggacgcggtcagcttcgggtgcgccgtcgtcccgcgcgccggggagaccttcgaggtccgcttctgccgccgcgggcgcttccgctcgcccgacgccgaccccgagtactttgacgagcccccgcgcccggagctcccgcgggagcggctcctcttcagctccgccaacgcctccctcgcccacgcggacgcgctcgcctccgccgtcgtcgtcgagggcgagcgcgcgaccgtcgccaacgtctcgggcgaggtgtccgtgcgcgtggccgcggcggacgccgagaccgagggcgtctacacgtggcgcgtgctgtccgccaacggcaccgaggtccgcagcgccaacgtctcgctcgtcctgtaccaccagcccgagttcggcctgagcgcgccgcccgtcctcttcggcgagcccttccgggcggtgtgcgtcgtccgcgactactacccgcggcgcagcgtgcgcctgcgctggttcgcggacgagcacccggtggacgccgccttcgtgaccaacagcaccgtggccgacgagctcgggcgccacacgcgcgtctccgtggtgaacgtgacgcgcgcggacgtcccgggcctcgcggccgcggacgacgcggacgcgctcgcgccgagcctgcgctgcgaggccgtgtggtaccgcgacagcgtggcctcgcagcgcttctccgaggccctgcgcccccacgtctaccacccggcggcggtctcggtgcgcttcgtcgagggcttcgccgtctgcgacggcctctgcgtgcccccggaggcgcgcctcgcctggtccgaccacgccgccgacaccgtctaccacctcggcgcctgcgccgagcaccccggcctgctcaacgtgcggagcgcccgcccgctgtcggacctcgacgggcccgtcgactacacctgccgcctcgagggcatgccctcgcagctgcccatcttcgaggacacgcagcgctacgacgcctcccccacgtccgtgagctggcccgtcgtgaccagcatgatcaccgtcatcgccggcatcgccatcctagccatcgtgctggtcatcatggcgacgtgcgtctactaccgccggtccgcgctgtgacgccccgcgcgttccccccccacgtcgaatcaataaacgacagcgagtccgacccgagccctcgcgtcttgtgtgtgttcgcgcgcgccctctcccaccctctcccacccacactctctcccgccccaccacaccccgcaataaaccacacgagacgacggcgtgtacagcgcgaccgggcaatctttattggtcgtcggggcgggcgggaagggagcggggcggccgtcctcatcggtgggacatcatctgggagacgaagatgtcggcgccgcgctggcaggccacggcggccgagctggcgtcgatggtgccggcggagacgtctgccggggccgagaccgaggcctggacgggcggctggggggccgaggcgggcgcggccggggcggcggcctgaacgggggcggccgcggcggccgccgctgctggagccggagcgggggcggccgcggcggcggcggcggcgggcacggccaccggctgggcctgcaccaccaccggctgctgcgggaccacctgggcggggacgtgcgcgacgggggccgccagaccctggaggcccggcaggcccgggagcgccggcacggcgtgcgccacgggcgaggggggcggcgcgtgcagccccggctggtgggcgtacatcggcgggtacagcgccccggcggcgtggtgcgggtgctggtgcgcgtgtcccacgacggccccggggggcaggccgggcggcagcagctgcggcggcgtcgcgggcgcgtagtacggcgtcgggtagcggagctgctgggcgcgcaggtggctgatctcgcgctggagcgacgagaccacggcggcgaggtcgctcgcggggggcggcgcctccccggggtagtacgcgttgtcctgggcgtagtcgtcgtagcggcgcttcctggcggggggccgcggctcgggcgagggcgagcggcggtggcggcggtccatggcggccgagacgatggcgccgagctgggactccaggctgggcgccgggcgctggttcaccaccagctggttgtactgcgccgcgggcacgaagatgtagtcgccgggcgctgcgctcggcggggctgccatggccgcactgggcccgggcgccggggaggccccgcttttaggcagaagccccgcccacatcgtcgcctgcaggtaggtgtgtccgcgcacgccggcctcgcggcggcgcgccgcgaccagctcccagcggtcgcgcagcagcatgttgttcacggccgtcgacagcagcgcgcgcgtgagcgcctcctcgctcatgtgccacacgcgctcgcggtccggcgactcggcggcgcgcgccagcagctcggagcgcgcctgcgccgagagctgccggaacggcaccacggcggcctccggcgaggtgtcgtacagcacgatggtgcgcacgctctgtccgatcacgctccgagccacgtgcgcaaagatcatctcgtcgggggcctcgccgggcgccaggcgccgcgaggacagggacgccgagggcaggtagttgctcagcaggtacagcagccgctcctcgtccgagagccgcatgtccccgaagaagtccgggcccacggcgcgggcgagcacggcccccagctgcgggcagttgacgacgccgaggaagaaggggccgcggtcgtcgtcgaccacggccagcacggcgccgatgtcgcagcgcgggcggtggtcgatgttgatgggcagcgggcccgccgggggcagcgcggcggccacgacctcgcgcgtcagcgcgagctccccgccgtccctatcgtacagggccaggtagccggacacgtacacgggccccatggcggcggcggtggcgacggcggcggcgcgcgcgcggaggagactgcgggctcaggcggcggcgaactgcgggatatagcccaggcagagaaagtacaggaggtcgtagtcgctggagacgccaaactgcccgaggcgcttgtccgccgcgttgcgcaggtgcggctgctgcagcagcaggcccagcccctcctcgtactgcacggccacggcgtcgtgcgccgcgatgacctcgttgacgggggccgtcacgcgctggcggttctgcagctccagcgccaccaggcgcaccaggctgagctggtggcgctgcacgcgcgcgcggatcgagtgcgcgtccagcgccagcgcggccagcccggggaagagctgcgtgagctccgtgcgctcgtccgcgcggtaggccacgacgacgtagtgggccagcaggaagcgcaggttgccgtcgccgctgcgcgaggcgggcgcgtccccgctggcgccgcgcggcggcggcgcgttgggcaccagcgcgccgagctggaagttgttgcgcaggcggtccccgtacacgttgccgttgtgcagcaggcgccgcagcagcagcaggcccgtgatggtgttcaccgtggcgcgcagcagcggcgcgtcctcgccgaggaagaggttctggccgcggcccaggaaggcggccgcggcgcggttgacctcgtccacgaagcccgcgccgtcgtcgacctcgttggcgccgacgttcgcgccggggaccacgcgcaggcgcgccagcgcggcgagcacgctgtggccctccagcgcgtagcgcccgtggagctgctcgcgcggcacgcgcgcgtagttgtagcggtacgagatgcgcccgccctcggccagcgcctggctcagcgcgtccaggtactcggggatccgctccagcaggtccacgatgcccagcggcgcgggcgccgccgccgcggcggcggggccctgccgcgcgcggtgcaccaggtgcaggcacagcgcggcgctctcgagcgccgagtagtgccggttgcccacgtacaggcgcccgcaggcctgcagcgccgtggtcgtcacgcccatgaaggtgcgcgacatgcggccgtcgcgcgagtcgatggcgcgcgagacctgcgggcgctccgcgacgagcgtggcctggagcgtggcgtaccacgtgccgaagagcacgccggaggcggcgccgcgcgcggcgtacaccatggccaggaagtccagcgagaggttggcgtcgtaggcgagcgccacgtcgttcttggcgatctgcacctcgcggccctcgtccgcggccgcgaccgcctcgggcgcctcctcggcggcgcgcgccgcgtccgcctcctcggcggcgcgcgccgtctcctcgagcaccacgagcgcctcggccacgcgctccacctgccgctccagcggccgcagctgctcgtccacctcggcctcgaggcgcgcgcccgcggccatggcgttgtccagcgccgcggcggccgcgcggcggcgcgcgttcgcgtgcgccagcgcgaggcgcgcgtcgaggccctcgccgaagcccgggcgggcccagaagcccacggggaacggaagggcgatgaagtggcgcgcgctgcccgggatcgcagcggcctcgaaggcgaaccacgcgcggtccatggcgcggggggacatgggccgcgcggcggcgcgcgccgccttatcatccccgctccccgccgccgcccggcccggccccgcgcgcgccgcgatcgcgatcaccgccgcggcccggcgacgtactcggcgaggccgcgcacggtcgcggccatcgcgctcgcgttgccgcgcgtctgggtgcagggcaggcgcgtcacgtcgagcacgcgcatgctccgctgggccacaaacaccagcaggggcacgagcgtgatctcctcgccgcccgggggcacggcggcggcgaggaggcgcgccgagtcgcgcagctggcacagcccctcgtgccgctgcccgcgcttgctgggcgtgttgaggttccgggggaagcggcacgtcttgagctcgatgaggaagcacaggtgcgggcccgcccccagccgcaccacgcacacgcagtcggggcggcgcaccccgaggttgacttcaaaggccagggtcaaggacgccttcttaagcgtctctcggggaagcccgaagagactctcgccgtacgcggacgggtcgcgtcgcaggcgttcgtagaagcggttgtggcagcggatccccgcccggaagcgcgccgggatgcgcatcctccggatctacctcgacggcgcctacggcaccggcaagagcaccacggcccgggtgatggcgctcggcggggcgctgtacgtgcccgagccgatggcgtactggcgcactctgttcgacacggacacggtggccggtatttacgatgcgcagacccggaagcagaacggcagcctgagcgaggaggacgcggccctcgtcacggcgcagcaccaggccgccttcgcgacgccgtacctgctgctgcacacgcgcctggtcccgctcttcgggcccgcggtcgagggcccgcccgagatgacggtcgtctttgaccgccacccggtggccgcgacggtgtgcttcccgctggcgcgcttcatcgtcggggacatcagcgcggcggccttcgtgggcctggcggccacgctgcccggggagccccccggcggcaacctggtggtggcctcgctggacccggacgagcacctgcggcgcctgcgcgcccgcgcgcgcgccggggagcacgtggacgcgcgcctgctcacggccctgcgcaacgtctacgccatgctggtcaacacgtcgcgctacctgagctcggggcgccgctggcgcgacgactgggggcgcgcgccgcgcttcgaccagaccgtgcgcgactgcctcgcgctcaacgagctctgccgcccgcgcgacgaccccgagctccaggacaccctcttcggcgcgtacaaggcgcccgagctctgcgaccggcgcgggcgcccgctcgaggtgcacgcgtgggcgatggacgcgctcgtggccaagctgctgccgctgcgcgtctccaccgtcgacctggggccctcgccgcgcgtctgcgccgcggccgtggcggcgcaggcgcgcggcatggaggtgacggagtccgcgtacggcgaccacatccggcagtgcgtgtgcgccttcacgtcggagatgggggtgtgaccctcgcccctcccacccgcgccgcggccagatggagaccgcgacggaggcaacgacgacggcgtgggagggggctcggggcgcgtataaagccatgtgtatgtcatcccaataaagtttgccgtgcccgtcaccatgcccgcgtcgtccgtgcgcctcccgctgcgcctcctgaccctcgcgggcctcctggccctcgcgggggccgccgccctcgcccgcggcgcgccgcagggtgggccgccctcgccgcaggggggtcccgcgcccaccgcggcgcccgcgcgcgggcccaccctgttcgtcctggacggcgacggctccgcgtggttcgtcttccagctcggcgggctgggggcgctcaacgacacgcgcatccgcgggcacctgctcggccggtacctcgtctcgtaccaggtggtgcccccgcccgtctccgcgtggtactttgtgcagcgcccgcgcgagcgcccgcgcctctcggggccgccctcgggcgcggagctcgtggccttcgacgcgcccggcgtccggcgcacgtacaccacggcggcggtgtggcccgcggaggtggccgtcctcgcggacgcggaggcgcgctgccccgcggccgtcttcaacgtgacgctgggcgaggccttcctcggcctgcgcgtcgcgctgcgctccttcctgccgctggaggtcatcatctccgccgagcggatgcgcatgatcgcgcccccggcgctcggctcggacctggagccgccgggcccgcccgcgggccgcttccacgtgtacacgctcggcttcctctccgacggggccatgcaccagacgatgcgcgacgtggccgcctacgtgcacgagagcgacgactacctcgcccagctgtcggcggcgcacgcggccgccctggccgccgtggtgcagcccgggccgtactacttttaccgcgcggcggtgcgcctcggcgtggccgccttcgtcttctccgaggcggcgcgccgcgaccggcgcgcctcggcgccggcgctcctgcgcgtcgagagcgacgcgcgcctgctctcgcgcctgctcatgcgcgcggccggctgccccgcgggcttcgccgggctcttcgacgggcgcgccgagcgcgtcccggtggcgcccgcggaccagctccgcgccgcctggaccttcggcgaggacccggcgccccggctggacctcgcgcgggcgaccgtcgccgaggcgtaccgccgctccgtgcgggggaagcccttcgaccagcaggcgctcttcttcgccgtcgccctgctgctgcgcgccggcggccccggcgacgcgcgcgagaccctgctccgcaccacggccatgtgcaccgcggagcgcgccgccgcggccgccgagctcacgcgggccgcgctctcgccgacggccgcgtggaacgagcccttcagcctgcccgacgtcctctcgccgtgcgccgtctcgctgcgccgcgacctcggcggggacgccaccctggccaacctgggcgccgcggcgcggctcgcgctggcgcccgccggggccccgggcgcggcggcggcgacggacgagggggcggaggaggaggaggaccccgtcgcgcgcgccgcgcccgagatccccgccgaggcgctgctcgccctgcccctgcgcgggggcgccagcttcgtgttcacgcgccggcgcccggactgcggcccggcgtacacgctcggcggcgtggacatcgccaacccgctcgtgctcgccctcgtcagcaacgacagcgccgcgtgcgactacacggaccgcatgcccgagtcccagcacctgccggcgacggacaacccgtccgtgtgcgtgtactgcgactgcgtgttcgtgcgctactcctccgcgggcacgatcctggagaccgtcctcatcgagtccaaggacatggaggagcagctcatggccggcgccaactccaccatccccagcttcaacccgacgctgcacggcggcgacgccaaggccctgatgctcttccccaacggcaccgtggtcgacctgctgtcgttcacgtcgacgcggctcgcgcccgtgtccccggcctacgtggtggcctccgtcgtgggggcggccatcaccgtggggatcctgtacgccctgttcaagatgctctgcagcttctcctccgagggctattctcggttaataaacgccaggtcgtgaggcccgcgccgggccgcgaacccagactctctgcgtgcgcatgtttttccttgtcgggcgcaggggagacgggaggggggagagagacggcacgggcgccgtccgtcggggagacggcgggatgacattacgagagtcgggtggaggggatgcggggagcgccgtccacgggggaaggctgctggatgacataattagagccaggggtgaggatcccctttcccccgttccccgagtatacaccactctctcccccgcacacatttcccaccgggcgccgaacgtggattgcaccccggtccgcgctacccggttgcgtgtgagacgggcggagagccgccctcgccccggtcccccaaaaactcctgcggatgcgtggtccgacgaagcctccgcggatatgtccgccgtaccctcatccccctctctcccacagccctctctcccacagccctctctcccacagccctctctcccacagccccctctcccacagccctctctcccacagccctctctcccaccgccctctctcccaccgccctctctcccacagccctctctcccacagccctctctcccacagccctctctcccacagccctctctcccacagccagactcccgcctcccccgtccccgatcgccgaacgcgttcgagaattttttacccgctccgaggagggggtctctaaggggtctctaaggggtctctaaggggtctctaaggggtctctaaggggtctctaaggggtctctaaggggtctctaagggggaaccagagagtaaagaccggagagtaaagaccagagacacattttacaatttcgttgtacccgggtttattgaaaactttgttcaagagttaaagtgtctgccgcttgccggtgcgccgcgccgaggccgcgatccatccgccgtggaccgggggatccgtggggcccgcgggcctggattcgacgggacggcgggtccgagcgacggcggtccggcggcgtttcggctcgcggtcctcgcggcggcgacgatcctggtcagggggatccagcggcgacggcggcgctccgggttgtggcgccgggctccggctccccggtcttttatacccacacctcatgaatattcatgaatggttggtatgtgcgaatttttcggcgttcgcacttgttgtatataaaatattatatcctatatatattagcaattggtgcgaacgtgacgtggcccaatcattttcctcggatttgcccatgtgaccgagtttccaatagctccgcccggatcgatcaccgggccgatattttgccgataatccatcaatattttcttcggccgacgcgggttccacccgggccgtcccgccggggagcccggcccgggcgcgggactctgctctccgggcgccgcgcggcgtcgagaccgcgccgggacgatggcgtctgcgcatgcgcgggtggaggcgagtggccgggaatcccctctcgtgccgagccccgcccatcgtcgcgtgtcctctccctcacccccttctcccccgaccgggcgacgatgggccgcgtcctcgccacgatggaggagaggcgctcacgcacgcgcgggtacgcgggagttgcggggatcggggaccgggacgatcggggtgtgcggaccgtggcgcgtcggggggctgggtgcgagcgggggttgggcgcgcgcgtgtgtggggggggggaacgggaaacgggggacgggggaacccgccgaggcgcgcgtgcgcgcgcacgcgtgtgtgtgccgcggacagagacggacgggagaacggacgtgtgatttgtttaacggtttttattgaggacgatggagatgttggcgcgggccaggcgcgcccgaaacagctgaaacaggtactgggccccacggagggccgcgggcgagtcgccgaaggcggtcaggagcgccgcctcggcgcagacggcggacgtgagccgcagcgcgcggcccacgggcgcggtcgcggcgacgaggccccggccggcgtagagcccgtccagcacccgcgccccggccgcctccaccagctcgcacacgccgggcgccggcgcgttcgcctccgcgtcgctcatcatgaccgccagctccaggcccgtcgccgcgtcctcccgggcgtgcttcgtcgtcggcggtgaggcccccgccggggccgcggagacgatgggcgcgagcgcgggggcgtgcaccacggcggcgttcacgagcatccgcatcacctcgccgaagacggtcgcctcgcgcagcacgcggttgacggcgtgggccacggcgctcgcggcccgagaggcgccgccgtcgccgttgccgttgcctccgccgcctcccccctcgccgccgtcctcgtcgtcgcccgcgcccgccccggggcgctcgtccagcgtgtcgagcatgccctccagcgcggcgtagcagctcggcaggtgccagcgggccaggatgaactggtacagcgcgaaccgctgcgcgcccgtgagcgacgtggagcccgtcccgaagaaggggtccgccgcgacggcggacacggcctcgaccacggcggccgaggcgtcgtccgaggcgcacgcggcccgccgcaggatggcgtccgccccggccacgaggtcgcagagcatctgcagggacaggctcccgccgcgcgtcgcccagatgcgccgcgtcgccgggatgtagcgcacctgcacaaagtcgctgaccaccgtcgcgcgcagcgcgcgctcggcgtaccggcgcgggcgcacgctcagcgtgggggccgcgcgcccgcccttcaccaccacggccgtgggccgcgtctcgccgctgagcggggggcgcacgccggggatgccgtcaaagagcccctccgtcagcacctgcagcgagctgggcagctcggagaggtacgcctgcaccagggcgaagcaggcctcgttcacggcgtacacgaagcccgaggaggggtgcacgatggcgggctcggccaggagctccaggacacggtcctgctggcggtcgtgcacgagcgtcagcgagagcgccgacgccggcagcgagaccatgcactcgacgaggtaggggtcgtacacgtccagctccgcgcggccgcagaggtccagctccgtcagcggcagcagcagccccagggcgtccacgaagacgtcgtcgctcccctgcggggcggcccacgcgcggccgaggcgcgccagctccgtgcgcacgtagttggcgacgacgcggttccccggcgcgttcccgcgcagcgtgagcccgaacttgccgatctcccgcgagcgccggtgcacggacacgatgcagcccccgtgcacaaagtacgcgcggtccccgccgtcggcgatgtagaacagcaccccctggtgcacgatcgtgctctggtactcaaactccatggcggcggacacacgcggcgcggcaaatgggtgagcgaggcggcgcgaccactcctttaaacccgccggggggcgcctcccgccgcacacacgcggcggcgcggggatggaggacgcggcggcggtggacgtggacgcggcgtcggacgcgacgacgacggcggcggtgggggagaaggacgcgctgctgagctcggcctttgtgggcgcgcgcccgccgcgcccgcgcttcagctcgcacgtggtgagcctcctggtcctggcgctcgcgctccggcccgcgtgctgcctcgtcctggcgctccacggatcgcgggccaccctcgcggcgctcctcaccgcgctggccttctacgcgcgcgcggccgtctgcgccgccctcgtggcgcggaacgtggcccgggaccggatgccgctctcgccggcgcagcaggtggcgctggggctgctggccgcggcgcggctggccttcttgtacgtcgccctggacgcgggccgccactacgcgcccgccctcgccggggcgctgtacggcgccgactgcgtctgtgacgcgctcgccttcctgctgccgcgcgcgtacgcgcgctccatcatgcattaaaaggcggccgcgacgcgcccgcgccattcttcccccgccgcgccccagcgaccgcgcacacgcgcccgtccgcggtcgtcgctacacgatggagcgcccggccatcctgccgtccgggcagatcctgtccaacatcgaggtgcactcgcaccgcgcgctcttcgacctcttcaagcgcttccgctcggacgacaacaacctctacggcgccgagttcgacgcgctgctcggcacctactgcagcacgctctcgctcgtgcgcttcctggagctcgggctctcggtggcgtgcgtgtgcaccaagttcccggagctcagctacgtggccgagggcaccatccagtttgaggtgcagcagccgatgatcgcgcgcgacgggccgcacccggccgaccagcccgtgcacaactacatgatcaagcgcctcgaccgccgctccctcaacgccgccttctcgatcgccgtcgaggccctcgggctcatctcgggcgagaacctcgacggcacgcacatctcgtcggccatgcgcctgcgcgccatccagcagctcgcgcgcaacgtgcaggcggtgctggactcgttcgagcgcggcacggccgaccagatgctgcgcgtcctcatagagaaggcgccgcccctgtcgctgctggcgcccttcacgctctacgagggccggctcgcggaccgcgtggcctgcgccgcgctcgtgtcggagctgaagcgccgcgtgcgcgacgacaccttcttcctcacgaagcacgagcgcaacaaggacgcggtcctggaccggctctcggacctggtgaactgcaccgcgccctcggtggccgtcgcgcgcatgacccacgcggacacgcaggggcgccccgtcgacggggtgctggtgaccacggccggcgtgcgccagcggctcctgcaccacgtgctgaccctggcggacacgcacgcggacgtgcccgtgacctacggcgagatggtgatcgccaacaccaacctcgtgacggcgctggtgatgggcaaggccgtcagcaacatggacgacgtggcgcgctacctgctcggcggcgagccggcccccgacgacggcaagcccgtgggctcggcgcgcgtgcgcgcggacctcgtcgtcgtcggcgaccgcctggtgttcctcgaggccctcgagaagcgcgtgtaccaggccacgcaggtgccgtacccgctggtgggcaacctggacgtgaccttcgtcatgcccctgggggtcttcaagccggccgccgaccgctacgcgcgccacgccggcagcttcgcgcccacgccgggcctcccggacccgcgcacgcacccgccccgcgccgtgcacttcttcaacaaggacggcgtgccctgccacgtgaccttcgagcacgccatgggcaccctctgccacccctccttcctggacgtggacgccacgctggcggcgctgcgccaggagcccgcggaggtgcagtgcgccttcggcgcctacgtggccgacgcgcgccccgacgccctcgtcgggctcatgcagcgcttcctcgaggagtggcccggcatgatgcccgtgcgcccgcgctgggccgcgccgccggccgccgaccagctgctggcgcccggcaacgccgacctgcgcctggagctgcacccggcctttgacttcttcgtcgcgcccgaggtcgacgtccccgggcccttcgccgtgccccaggtgatggggcaggtgcgcgcgatgccgcgcatcatcaacggcaacatcccgctggccctgtgccccgtggactttagggacgcgcgcggcttcgagctcagcgtggaccggcaccggctggccccggccacggtggccgcggtgcgcggcgccttccgcgacgccaactaccccatggtgttctacatcatcgaggccgtgatccacgggagcgagcgcaccttctgcgcgctcgcgcggctcgtggcgcagtgcatccagagctactggcgcaacacacacaacgccgccttcgtgaacaacttctacatggtcatgtacatcaacacctacctgggcaacggggagctgcccgaggactgcgccgccgtgtacaaggacctgctggagcacgtgcacgccctgcggcgcctcatcggcgagttcacgctgcccggggacccgctgggcaaccagccccaggaggagctcaaccacgcgctggcggacgccgcgctgctgccgccgctcatctgggactgcgaccccatcctgtaccgcgacgggctcgccgagcgcctgccggagctgcgcgtcaacggcgcgcactttcagcacatcctgtgggtcgagatggcccaggtgaactttcgcaacgtcggcggcggcctggtccacaaccggcccgtgcggaacgagaaccagcccctgcacccgcaccacgacgccgagtggtcggtgctgtccaagatctactactacgccgtggtgcccgccttctcgcgcggcaactgctgcaccatgggcgtgcgctacgaccgcgtgtaccagctggtgcagacgatggtggtgcccgaggcggacgaggaggtgggcgcggacgacccccggcacccgctgcacccgcgcaacctcgtgcccaactcgctgaacgtgctcttccacaacgcctgcgtcgccgtggacgcggacgccatgctgatcctgcaggagacggtgacgaacatggccgagcgcacgacgccgctcctggcctcggcggcgcccgacgcgggcatggccacggtggccacgcgggacatgcgcacgcacgacggctcgctgcaccacggcctgctcatgatggcctaccagcccaacgacgccacgctgctcgagggggccttcttctacccggcgcccgtgaacgcgctctttgcctgcgccgaccacctcggcgccatgcgcgacgtgggcgccgaggtgcgcgccgccgcccagcacgtgccctgcgtgccccactttctcggggccaactactacgccacggtgcgccagcccgtggcccagcacgcggcgcagagccgcgccgacgagaacacgctctcgtacgcgctcatggcgggctacttcaagatgagccccgtggccttcacccaccagctgcggcgccagctgcaccccggcttcgccctgacggtggtgcgccaggaccgcttcgccacggagaacgtgctctttgcggagaaggccagcgagtcgtactttatgggccagatgcaggtggcgcgcaccgagagcggcggcgggctgcacatgcagctcacgcagccccgcgccaacgtggacctgggcgtgggcttcacggccgcgtacgcggcggccgcgctgcgtgcccccgtgacggacatgggcaacctgccgcagaacctcttcgccacgcgcggcgccccgcccatgctggacgcggacgcggacgactacctgcgccgcacggtcaacgccggcaaccgcctggcgcccgtgcccgtcttcggccagatgctgccccaggtgcccgtgggcctggcccgcgggcagcagtcggtgtgcgagttcatcgccacgcccgtctcggtcgacctggcctactttcggcgcgcgtgcaacccgcgcgggcgcgccgccggcgaggtgcacggcgaggaggggctgatgttcgaccacagccacgcggaccccgcgcacccccaccgcgccaccgccaacccctgggcctcgcagcggcactcgtacgcggaccggctctacaacggccagtacaacatgagcggcccggcctacagcccctgcttcaagttcttcacgcccgccgaggcggtcgccaagagccgggggctggcgcggctcatcgccgacacgggcgccgccgcctcgccgacgagcaacggcgagtaccagttcaagcggcccgtgggcgccggcgagctcgtcgaggacccgtgcgcgctcttccaggaggcgtacccgccgctgtgcgccagcgactcggcgctgctgcggacgcccctcggcgccgaggagcactttgcgcagtacctcatccgcgacgagtccccgctgaagggctgcttccagcacgcgagcgcctgagccgccggaccgataccggaccgccgccgaaccgtcgtctccgccctgccccctccccccccgactgctctgtcacccgcccccaccctcccgcgccggccgcgcgccgcgcgtgtatataaactgtgtacacggcacccgcccgcgtcagtgtcttctttcgctcgcgtcgccatggaggtggacattgcgctgccgaccctgtccccgggggatctgtcggccctgcagaggtgcgagggccgcgtggtgtttctggagacgctgcggcgtcacgcgacactgcgcgaggtcgcgctgccctgcggcggccatgtgctcgcggccatggccgcgtaccggcggcgcttcccggccgtcatcacgcgcgtgacgccgcaccgcatgctggccacgccgctcggcgtcagcggccgcgggcagtcgctggtgctgcagaacacggggcccttcgacctgaccaacggggaccacgtgtgcctcgtcccgccgctgctcggcgacgagtgcctgcggctcacgtccgccaacctggagctgcgcttccccatgacgctgccgctcgcccaggcccgcgagctgacggcgcgggtggtggcgcgcgccgtcgagacgctgcgcggcggcgcgcccgcgcggggcgccgacgtggtcttcaacaacggccggcgctaccagctgcccccgccgcaccgcgacaacgccgaggcggccacgcgctcgctggtgctgaacatgatcttcctgctcaacgagggcgccgtgatcctgctgtcgctcatcccgaacctgctgacgctgggggcgcaggacggctacgccaacgccgtgatccagctgggcagcgccacgcgcgagctcggccagctggtgcggcagcccccgccgccgctgccgcaggaccacgcgcggcgcttctgcgtgttcgaggcgctggaggcctggatcgcctcggcctcgcggctgggcgacacgctcggcacgcggcccgtggcgcgcgtgtgcatcttcgacggccccccgaccgtcccccccggcgagaaggccgcggtggtggaggtgtgacggcgcggcgccgagggggcccgtgtttcccccgccccaccccacccccctgcaataaaaaatccgcgaaggaaaacaaagaaacgagtcgtgcgcgtctggcgtttattttccgggtcggggaaaatgggggcgtcggggggtggagggcggacggtggacggtggatgggggaagccccctcaggcaaacagcccgtcgggcagcgtgctcaggtgcacggccatgacgagggccacggcgaggtcgtcggcgcagccgccgcgcttgccggtgaagacgcggtgctcgccggcccccgaggccgtctccacgaggttcccgagctgggcgttcaggtactcgacggggtcggcgcgcgcgcccacggtcacggacacgagctcctgcgaggccatgacggcgcccgagttgaactgcttcacgaaaaaggggaaggcgggggtcttttgcttctgcagcaggaagaaggggtagcgcacggcgcccgcgcccgcgcccggggcgccgtggtggtagaagaccacggccgggtcggcgacgacgcgcgcggcgcgcagctccgcgagctcgcggcgcatggcgcccacgatggccacggccgagtcctggctgctgttgccctcgacggccacgcgcaccgccgagaaggcgccgtcgtgcagcgccagcacgcgcgccaggcagcgcacgacgcagcgcgcgatctcgtcggcggaggcgccggtgagcgcgcgcaggaaaaagtgctccaggccgagcacgatccagctgcgccggtagctgcccacgacggccacgccggtgccggaggcgcgcgcgttggccgtgaaggccgggtccacgtacacgtgcagcgtgccggccagcagccggcggttggccaccgaggaggggcggtagagcaggaagcgctcgccggcggcgcgcgtgaggacgggcgcctcgcgcgcggccacggcgccgccgaggatctcctgcatgaaggagtccgggagcagccgctcggccgtggtgcgcgtggcggcgtccatggtgatgaagacgggcttgttgagcacgtagcacgagcaggcggtggcgtcggtgtgcgccaccacgcggcgcatgtgctcgtcgcagacgtacgtgaccacgttgagcatgtcggtgccgcggaggttggtgaggaagctggtgctggccttgccggtgttggtggaggagacgaagatgatcttgcagctggcctggttcatgaagccgaggatggtctgcacggcatcggggcgtataaagttggcctcgtcgacaaagagcaggttaaagtcctgtccgcggatcccctgtaacgacacgcggggagagagagggggggacggtgagcggcgccgcgatggacgcccacatcgccaacgagaccaagcagcagatgacgcgcttcgcgcccgcgctcgtgcacgtcatcgtgcccgaccccctcctgcggcgcgcgggcgtggacccgctcgcgcccttcgccgcccacgcgcagacgcgctaccacggctcgggcgtctgcgagccgtgggtctccgtgttcgcgggccacgtgcagacgggcgccgtcgagagcgtgctgacgctgccgccgctgcagcgcgcgcgcggccccggggggctgttcgtctcgctgcccctggcgctcggcgcccactttgacggcttcacggcggtggcgctgcgcgtgggcgcgcgcgagctgatcttcacctacgacgagctcctgccggcccgcacgcgctacaacgtggacggcgagcgcctggagcgcctgtgccgccagttcgccaactacgcgcgcgcgcggcgcgtggccccggaggtcgcggccgcggggggccacatcgacgcgctgctgcccccggcggcggccaccatcgacggcgaggcgcagctgacgcgcgggggcttcgacgacccggcggcgccgcacgcgcgcgacgccgaccgcgagatcctgtcgctggtgcgccgcgcggccgagctcgtcgcggcgcggcacccggtgcgcagccacgtggcgagcgggctgatgcagggcgccctcgcgcggcgcggcggcggaggaggcggcgcgggggcgctcgaggcggcggcgacggtgccggcgcccgcggcgcgcgaggacgcgaacggcggcggcgccgcctggcgcgacgagcccctgctcacgccccaggacccccggccgctgacggcgctcgactggctggacgcgggctacgccgcgctcgcgggcggcgacgcgccggcgcacgtgtggcgccggcggcccgtgtccctggtggcgcgccgccactaccagaccggcgagacgttcgtcgtcgtcgcctacgagcactccaccgcctggggcggccggcgcgggcccgcgagcgagcccctggcgcgcgtgctcgccgaggagtgcgagcgccacggcgtcgagcacccgcgcgccctgcccgcggaggcgcggctggagccggtgcggcggcacgccgagctggccgtgccgctgggggacgaggagccgccgctgccggtctttgacgccaccgcggagctggtgctgctggagcgcttccgcaacgcctgcgtgcgcgcgctgctggccggcgtccgcgagtccgtgcgccgcgagccgcgcatgcgccagatcatcgagttcgcgatccgcccgcgcgaccgcgaggccgtcctcgacgtggccgggcgcgccccggcgctgctggacgcgttcgcgcggcgcctcgagcagacgccggcgcgggagatggtcgactcgggcctgatgacggccgccgcggcgcacctcgccgcgcgcgccaccgccggctacgtgaccttcgagagcggcccgctgctgggcggcgttttcctcttcgactactacagcgccggcggggaggttataagggtgacgcgggccccgccggccgtggcagtggagccggcgacgcgcgggcagttcgcgtgccgcttccgcggcgcgtcgcatcgctgtctcccgggcgagagctacgcgtacctctgcgtcggcgtgtcgcgggacctgcgcgcgctggtggtgctgcccggcggcttcggcttcttcgcggccctgcggctggagtggcccgccgcgctggtggaccccgtgctcgagcgcctgtgccgccgcgtgtagcgagggtgggcgcgacgagcgccgccatggaggtggccgcggccctgacggaggactttgccgcctggcggctgctgcgctccgactcgcgcgtcaaggtctacgccgccctcggcgtggtgggcgcgcgcctggccgccgtggcgccgggcgccgcgaccgtcgacgcgcgcgtctacctgacgcgcccgcgggccctgcgcctggcccagggccgcttccacgtcgtggtcctgctcaacgacgccgcctacgcgctggtggcggccgtgaccacgaccacgctccgcgggagcggcggcgagctcgtgcgcctgacgctgggcgacgcgagcctcgaggccctgcccgccgacctgcccgtcgccgagcccgtgcccgccgcgccggtgagccgcctggacctggacgccgccgagcccgtgtccgcggcgcccgcgcgccgccgcgagtgcgtggtcctggcccccggcgcgtggtggtcgcgcgaccgcatctactttctgcagatggacccggcgctgctggcgctgtgccccgccggctggcgcgcgcgccacctcggcgccgtgctcgccgggctgctgagcccccgcgacgacgacggcggcagctgctgccgcgagtgccgcgtggaccacgtggacgcgctcaacgcgacgccgcaccccgacggggcgggcgcggcctgcctgtgcgccgcgccgtgcctgtggcgccaggcggacaagcgggagctgcgcgcgagcgactcgggcctcttccgcgtgctgttcctggacgccgtgcgcttcgtgcgcatgctgccccggcgcaagatcgtcgacgtcgcctccgagctcatcggcgggctggacgcccgcgggcgccacgtggtggtgaacgacgccggctggcgcctggtggccctggacccggacgcgagccacgcgctcgtgtgcggctgcccgctgctgcgcgccctctgcgaccccccctcccgcgccatccccgagctaataaacgactattgaaataaaaaaatgggtacttgcgtttgtgttgtggctggaggcgaacacgatggtgctgcgcgcgccgtcggggaaggtgacggtgatgttctcgcccttgacgtggtccacgcgcgcgtcgcggcaccagcggcgcaggcgcgcgtggatctcctcgaagacgggctccgtggccttgcggatgtgggccgtgtagccgacgcggatgccgcggaaggtggccagcgcgagggcgatgagcgggacgaggaaccaggtcttgccgtggcgccgcggcacgaggaagaccgtgacgcgctggcgaaagtggcgcaccgcgtcctcggagaagagcggcgtgtcgaaggccgccagcaggtaggcggtggcgcgctcggcgtggtccccgagcagcaccgtggccagaaagtacaccgcgtgcatgaggatcatcttctggaagagctccagcgtgccgcgccctccgccgccgccgccgcctctgccctttgccccgcttccagttccacctccaccgccgccgccgtctccgctctcgccgcggccctcctcggcggcgtcctccggctcggggcggccgggcgcgacccccgcgcccctcttctccgcggccccgtccccatcgtcccccgcctcctcgtcgccgtcgtcgttggccccggtcccggccccggccgtcccgagggcggccgtcgcggcgtcgcggaaggagcgccgcgacaggagcgcgaagcggttgacgaagcgccggagctggcggaaggccccggacgaccgcatggcgtccaccgccgccatggcgctcgtgtacgcgttgcggtggaaggccaggcgctcgccgtcgtaggcggcgaactcgagcgcctcggccgcgcgcgcgagcgccgggtccacggcggcgcggccgcgcgcgcccccgaagagcgcggcgcgcgcgagcccggcgaagatccgcgcgctctcgcagcagttgtgcagcgtgccgaccgccgggaccaccgtctggtggcgctgtggggcggcgatggcaaagttgaaaaagcgggcctcctcggcgcagtcgcgctcactggcctcgcgcttgcgcttgaggtccgcgtagtagcgccgcgtcgcgtcgcccaggccgaacatgttcgcctcggaccgccgggagcgccgcgtgcgcctcgcggaggcctttcagcgcgagagcgtcttcaaggcgcgcaccgtcgagcttctgcgcgggcgcgccgacaagaaaaaccccgagttcgtccgggcctttatggcggccaaacaggcccggcgggatgtagagcgccacctgcggctggccgcccgggtggagtctgtggaacaaaaagcgcgcgcgctgcaggcccgcgtggaagcccaggcggccgtccgcggggtcctggacaggcaccggcggttcacgcgggcggactttgccgaggcgctcgacgccgcggaggacgcgctcgtggccggcgaggaccggctcgacgacgcggccgcgctcgacgaggactgggccggcggtggggaccctgacgaggacgagggggaggaggcggacgaggccctgctgacccaatggctgctggaggaggcggcggaggcgtgagccggaccgcgctggcgcggccgcccatccaccgcggcacgtccgcgccctgcggcgacggcgacggcgacgaggcgtcccggctcctgggccgcgcgcagccgcgcgaggccccctacctgatcccgcgcccggacggggacctcgccgtgccggacgacctgcagtacgcgaccttcgacctcacgggcgaccccgtggccgtcggggccggctcgtacggcagcgtgctcgtgtacggctcggtggccgtgaagacgctccgcgccggcttcggccacgaggccgtcatgacgctgctggccgcggaggcgacgcgctccgccggcgtccgcggcgtggtgcgcctggtggggctctcggcgccgctgcgccagctcgtgttcccggcctacgagatggacatggacgcgtaccgccgctcgctcacggcgcgccccgggcacgtggtgcacgccctggggcgcgtcttcgccgagctcgggcgcgcgctcgtgttcctcaacagccgcgggctcagccacctggacgtcaagggcggcaacatctttgtgcgcacgtgcggcaacatggtcgtgacggccgtcatcggggactttagcctcatggccctcaactcgcgcagcacgctcgcggacccccgcttccgcctcgcgcgccgcaaggcgctgaagctcacgtcgctggcgcggagcccgccgacgggcgtcctcctggggcatgcgcgcgaccggcccacgggggtgctgatggactttattaacgggcgcccgccgccgccgggccccctgccgtacgaggtgggcctggcgatcgacctgtgcgcgctgggccacgtgctgctggacgtggcgctcggcctgcgcccgcagcgcggccagacgctgacgcgcgagtacgccgtggaggtgctcgcgcgccgctgcgtcctcttcgcggccctgctcccgccgggcagcggccccgccgccgaggccctcgccgaggacatcctcgaggaggagctggccgccagcttccgcgagtgcgtggccagctcgcgccccggcaaccagcccccgcgcacggtggccccgctgctcgagctcgtggcccggttctgcggcgaggatggtggcgctcgttttgccgaactcgctgcctgaggagctggccgcgcgcaccttcctgcggttcctgcgcgcggcgccgcgccccgcggccggcggcgcgccgctggcgtaccgcctggcgtacgtgcacgacctgctcgtcgagctggcgcgccacggcctcgccgcccccgacgcggccgcggcggcctttggcggcgcgcgcccgccgcccgcgcccgcggacgtccccgccgcggcggcgcgcgccgccatcctgacggtggaggcggccacgcgcgcgcagagcgagagcgacctgtggacgctgctgcgccgcgggctggccacggcctcgaccgtgcgctggggggcggacggcccgcgcttcccgccgacgtggtgcgaggccagcacggcgcgctgcggcacgcccgacaacgcggcgctcatctttggccgcgtcaacgagcgcgtggcccgcgccgccgtcgcggccctgtacgccgaggcgcccacgccggacctgcccgcggcggcgacgggcggcgacgagggcgacggggacggggcgaaggaggagatgttcaccttcgacgagacgggcgcgcccccgccgggtcacgacctcttctcgtgcgggctgctgctggacccgcgcaccggcatggtcggggcctcgctggacctgctggtctgcgaccgcgacgcgcgcgggcgcctggcgccgcaccgcacgcagacggagatgctcttcttcgagatcaagtgccgcgccaagtacctgttctcggcggacgacgcgagccccacggcgcgcgcgtacgcgcgcctgctcgggcgccccgacgcggacacgctgcgcggcttcctgaactccatcgcccggccgggcgtggagttcttcgagggcgccccgggccccggcgaggcgctggcgacggccgaccccgcgtggcggcgcgcgggcgccgaggacgcccccgcgacgcgtcggaggtgtggcgccttcgacgcgcgccacgtcgccgcgaacgcgcacgcccagtcggaggtctggctatttagcgacccggtggacggccgtcaggacattgtgccctgggcctcgggcgagcgcgcgctgagggtgcccgtgttcgcgaacccgcgccacgccaactttcggcagatcctggtgcagagctacgtggtcgccggcgtcttccccgaccgccccgcgcgcccccacctggccaccttcttcgggcgccgccgccgcccctgcgagcagaaccggacgctcgacctcgcctcgctgtgcgacgtgccgcccgcctgcgccgtgcccgtgctgctgatcgtcacgcccgtctccgtgtgcgaggaggcgttcgaggacctgcggacgcgcgccgaggaggcctttcgcgccaccgcgtcgcggacatgggacagtgctgctgccgattctccagcaaccgcgtcgtgacgagctccggcgacgtgctgaccttcgacgcggacgcgttcgaggactttgagctcgagcccatggtcggggagcccggccccgtgcgccccaaggccccgtaccgcgtgtcgcgcgggaacctccgtgaagcctcgcgggcgtactgactgcaataaacccgtttgtcatactatcgcatcgttcccgtctggtgtctcggggcgggagggacgatgggacgggggagacggaggacgggggagacggaggacgggggagacggaggacgggggagacggaggacgggggagacggaggacgggggagacggaggacgggggagacggaggacgggggagacggaggacgggggagacggaggacgggggagacggaggacgggggaggacggaggacgggggtggacggaggacggaggagacggaggacgggggagacggaggacgggggagacggaggacgggggatgtggcgggttgggtgtgagacgatggccgaggacgggggaagagggccgtggggcggggcgagatggaatggcgcaccggccgcaaagtgggacgatggggacgggcctcaacgcgggacgagggcggcggggcgggaccggggacaaggacaagggcggcgagagacggacacgagaggaccatgtgcgatcgaaacgaacgtttattcaaagccgaggttctcgtacacgacctcgtcgccgtcgtcaaactcgtcttcggtcatcccgggggccagcaggtactcttcgttgccccggccgtgcaggcgcttctgcaggtaccgccgggccgtcgtccgcgccttgtcgacgttggcgtagaaccggctgccccgggcgcggtgggcgatgcaggcgcggaccaggcgcacgacgatcatcgccagggcgatcagcgacaggaccgccaggacggcgcgcgtcccgatgacgacgccgtgcgtctccccgagggcctcgctgaacttggcgaagtagcggtgggcggacacgccgagggccgcgctggccacgagcacgcagaaggccggcgtggggagcacctgcacgtagccggacacgacgacctcgacgaacaccatcagcgccagcgccagcaccgtgaacacggccagggcgacctccgccgtcttccagaggctgatgtggaagctgttggccaggagcacgccgagcatcagcccgaccacggccgcgcccaggccgaccgcgccgaggacgacgttcgtcacgacggcgcgcgcgtgcaccgccacgcggcgcagcggggggctggccgccagggcggcgcgcacgtcacccgcgcccttgcaggcgtggcacgccccaaagagggcgagaaacacaaaatgggtgatgtaggcggcggcggccagacccgcctgcttgtgggccatcagcagcaccacggcctggagggtccaggcgcagagcgcgccgagcatcagggtccccggggcggccaccgtcgacgccgcgtagagcaccacgctgggctggaacgccagccggccggcctcgcgccgcagcaccacggcccccgcgaccgcgtacagggcgtgcgccagcagcacggtggccgtgaagccgaagaagctggtcacggtgggcgtttccagaaacagggcgggggccacgagcggctggcgcgtccacacgccgccggaggtgtcgttgagcgtgtcgtagtcggccacggtcgcgtaaaagcacgggaaccccatttccgggagcgaggcgaagataagggtgaggaccagcgtcagggcggccagggcgaacccgcagacctctatcagccaggagcgccagctcacggcctcggcgttgcgcggcccggacatggcggcggacgcgggcgagcgccgcggcgcgaccgaggaggacggcggggcgttcgcctcgagcgtgtcgctcgcgcggatgctatacggttgcgacctcccggccgtggtgcgcagccgctggccgggcgtctccctggacctgcagcgcgacgcgcccgtcgagcttccgtccccgcacgataccgcctgccgccgcgtcctcgtggcgcgcgctccgatgggctcgggcaagacgacggcgctgctgaaatggctctccgcggcgctggcggccactgatatgagcgcgctggtcctgtcctgccgacgcagcttcacccgcaccctggcgagacgaatggacgacgcgggcctgggatttgtgacgtacttcgactcggacgcatacgtcatgacgggccggccctaccgccggctgctggtgcagatcgagagcctgcaccgcgtggacgagcacctgatcaacaactacgacgtgctcgtcgtcgacgaggtcatgtccacgctggggcagctctactcgcccaccatggcgcggctggcccgcgtggacgccctgctcgcgcggctgctgcgcggctgcccgcgggtcctcgtcatggacgccacgatcaacgcgcagctcgtggagctgctcgtggagctgcgcggcgagcccagcgtgcacgtggtggtgagcgactacgccacggccgccttcgcgagccgccgctgcctcgtgctccggcacctcggggccgaggtcgcggcgggcgcggcgggcgcgcgggaggacggcggcggcgacgggagcgaggacgcggcgcgggccgggagccccgccccggtgacggcggcggcgacgacgacggcggtggaggcggcgggggcggcgggggacagcttcttcggcctcctgggcgcgcgcctcgccgcgggggacaacgtctgcgtcttctcgtcgacgctggccttctccgagctcgtggcgcggttctgcgcgcgcttcacgccctccgtgctggtgctcaactcgcagcggccccccgaggacgtgggccgctgggccgtgcgcgccctcgtctacaccaccgtggtcaccgtcgggctcagcttcgacgcgccgcactttcacagcatgttcgcctacgtgaagccgatggcgcacggcccggacatggcctccgtctaccagtccacggggcgcgtgcggcggctgctgcgcgacgagctcttcgtgtacgtggacggctccggcgcgcgcggcgagcccatcttcacgcccgtgctgctgaaccacgtcgtgggcgcgggctggccggcgcgcctgtcgcaggtgacgaacctcgtgtgcgcgcagttccagcgccgctgccgcccggcgttcgcggcggcgcgcgggatgcgcctcttctcccgcttcaagttcaagcacctgtttgagcgctgcacgctgacgagcgtcaacgacagcctcaacatcctgcacgcgctgctggagaacaaccgcctgcgggtggcgctcgagggctgcgagccgccgctgacggcgcgcgccttctgcgacttcctgcgcgacgcgcgcctggacgccttcgccagccagcaggtgctgcggcagctgcgccccccggaccggccggtggcggccgacatcgcggacagcggcgaggtggccaccttcgtggagaagtacctcgtggccgacgtgcccgaggacgagctgcaggagctgctgcgcgcgctcgccaaccccgtgacgcgggagcagtttgtgggcctggccgtgctcggcgcgtgcgcgcgcgtccccgaggcgctgcgcagcgagcgcgtcttcggggccgtgtacgggcactacgcctcgggggccgtgcccgtggtggccgacgggcgcctggagctggcggcgctggcccccgacttcaacgtgcccgcgcgctgggccctcacgcggcgctgcgcgcgcgtcgcggaggccgccgggctcttcgagggcgcctcgccggaggtggactcggccgcggtcgcggcggcggcggccgacgccgagctggcgccgctgctgctcgaggtgctgcggtgccacgtgctggacgcgacgacggcggcgcggcgccccgtgcgcgcggccctgagcgcgctgggggcgggcgggggcgcgggcccgctgagccgcgggcggcacgccgcgctcgtgttcaaggtgatgtgggaggaggccttcggcgtgcgcgtcggccgcagccggcagaccttcccggggcccacgcgcgtgaagaacctgcgcaaggccgagatcgccgcgctgctgcgcgacgccggcctggacccgcccgcgggcgccacccaccggcagctgtacgccctgctgatggaacgccgggaggactttgcgggggagaggtataaactgcgcctgcccgcctggagccggctcatgtacctgacccagggcggcttcgacgcgccgctggacgccgcgctctcgctcgtgcccgcggaggcgtggccgcgcatcgagggggccgtggactttgcggcgctatgacggccgcggcgggggagcgcgtgtgcgcggccgggatctacacggcctggacggagcccggcgcgccgggcgagctccacgcgctggcgcacctgctgtgccgcgacggggccggcgcgtacgcggcgcgcttcgtccacgtgacggcggagccgtggacgggctcggcggccgcggtggccgcggcgctgcgcgacgcgagcggcacgggggcgctggcggacccggcgctgtggcgcggcgcgcacggcgcggtcatggcggccctgcgccgcgccctcccggcgctggccttctacgagccgctgcgcttcgagacggacccggccacgaacctggtcacggccgcgacgccggtggccgggggtggcggcgaggagaggggcgagggggagaagagcgcggaggaggacggggacgacgaggacgaggccggcccgcgcgcgggcatcatccacgtggacgccgacgtcgtcgtggaccgcgacgccgcgcgcgcgcacgccaccgaggcgtggctgccgcacgcccagctgcgcgccatggccggctgcgccgcgctctccgtcgagatcacgacgcggcggaccagcttcgcgcgcgcctacgccgaggccggcgcgccgccgctgaagcgccggggcgagatgggcgacctctttgacgtgcgcgagacggtgctgcggccccggggccggcgcgtggcggcgcgcgtgctcgtcccgcgcggcttcgactgcctggtggcccacggcgtgagcaacgcggcgctggtggcgctctaccgccggtggcacgcggccgcgtacggcgcgggcgacgcgaccccgcccgtcttcgccttcctggggcccgagctcgcccgcgagggcgcggacgaggactactactgcgcgctgggcttccccctgtttgcgacgatcaaggtggccgtggccgcgccggaggcggtccgcggcgcgctcgccgcccaccggctcacggacgggctgtggcccgcgctcggcttccgcgccttccacgccctcgggcccgtgacctgcggcctgcgcctgcgctgcgacgtctggccggcggggcgcgtgaccagcgccgtggagcacccgtgcgccctccgcggggcctggctggccaagtttgacttcgcggccttcttccccaacctgtttctggccctgtgcccgggccacgcgcggctgcggcgcgccgtgcgggcgcgcgcggtcaagccggcgctcgtggccttcttcggcgggctcaagcacacgtgcccggaggcgtaccacagcgtcatcgcgctggccaacggcgtctcgcgcgccgtcgaggcggccgcggccgaggccggcctggtcacgtgcgcctacgtcaaggacggcttctggggcgtgctcggcggcgtgcccgcggacgcgccggaggcggccgccgccgccgcggccgagcgcgtgcgcgccgcctgcgaggccgccgcggcgcggcacctggccgacgcggggaccgccgcgacggacgtgacgctgcgctgcgagggcgtcttcgacgccgccctggcctggtcctgccacgcctactggctgcggcggcgcgagggccccgcggactttgtcggcttcccgtcgcgcagcggcttcggccgcgccgccaaggccgcgctggacgccctgctgcgcgaggccgtcgagggccgcgcggacgccggggccgcgcgcgccgcctgcgacgggctcgtcgagcgggccttcgcctcgcgccacgaccccgacttctgggccgccccgggggacgccctcgcccccgccgcgtgcgccggggcgaccgccgggcgcggcggcgcgcggtgctcgcggcgctgcgtgcgcctgcgcggcgccggcccggtgccctacgagcagctgcgccccccgctgatcctgccctggatagactgcctggcccacatggagcccgtcttcgcggccctcgtcggcatgtacaaccgcgcgctcgaggcgctgagcgacggcgacgccccaccgccctttgtgtttacatacacaccggacgcgtttctgtttggttaaacaataaagtctgtttattgtccgaacgagccacccggcgtcctgttgtcgtccgcggtcggggatggtgggggagagggaaggggagagggaaggggggagggcgagggggggagaggaggggggcagatggagaggggaaaggggaaaggggggtcggggcggggaggtggaggaggcggaggaggggggaggcgcgcttcactcgtagcgcacaaacatggtcttctcggtgagcgcccgcggcatcgtcgtccaccagcggtacacggcctcggccatctcgggcgtgcgcaggggcgcggtgagcgcgccgccggggcgcgtccgcaccagcccgtcgggcggcggcgcgtcgcccggcaggccgagcaggggcccgaacccggcggcgaagagggcgtccacgacgggcggggtccggggggccgacagcaggcgctgggcgatggggtagtggggcagcacgcgcgtctcgtcgagcgggctgaggtgggccgcgaacatgagcgtgttgagcgcgcgcgtggcgccgacgtgcagcaggcggcgcagcttctgcgccggcggggcgcgcgcgcgctccagccaccggcccaggcgcaggcaccgcgcggcgtccgcctcggccgcggggcagttctccaggaacatctgcagcacgcagagcgcgaactccgtcgggtcctcggggcgcaccagggccaggcggtgcggcagcgccaccggcggcacggccagcgagagcacgcggtcctcggcggccgtgaccaccgcgaaggcgaacccgcggaaggcctcgccggcgaggcagcggcggcggtactcgtcgcaggacaccgtctcgccgctctcgaggcgcagcgtcagcgcggcgccctcgacgcgcgcgtcagtcaccgaggcgctcgtgaagaccgggatcagcccgggcacctcgcgcacctcgcacaccagccgcgggagggcgtgccaggccagctcgtccatcacggcctccatcgcggacccgctcggtcgcccggcgcacgtccgccgcgaacagcgccgcgatctcggtcaggtaggcctcgaggcgcgtcttcttaaacaccacgccgcacaggtcctcctccgagcggtacacctcctggctggtgatgaggaagccgaggtggcgcacgcgcaggatggggaagaagtacggcgcgaggatgagcgagatggggctgttggagtacgagacggacacctcctggccctggttgttggccacgcgcgtcagcttgaagcagcggatcagctcctgctcccagagccgcgagaggcgctccacgtccgactcgtacggcggcacgtagcgcgactggaagctgttggccacgtaggcgtcgtcgcccatggcgcgcgtgatgtcgatgacctcgtggccgaggtcccgcggcggctggccgtccgcggcgccggcgcccgggccgggcgcgcgccccccggggccggcgccgcggtccgcgcccgcctgcgcggcgcgcagggcctgctcgcgcaggcgccgcacctcctgctcgcgctcgcgcacctgctcgcggaggcccccgttgtccgccttcaggctctcgatggtcttgaagaggttgttgacgtagccctcgagcatgccgttgatgctgttgaccacggaggtgcggaaggcgtcctgcaccggctgcgcgccggcgccgtggtgcccgaagcccggctgcgacgtgtccacgctgtccaggatgcgcgccccggtctcgtcgaggtacgagcgcaccgtctccgtgatgtcgccgatgtggcgcatgcccttcatgttcacgatgaggcgcacgaggcgcgccgcggccgagcccgccgggtcctcgccgagcatcttctccacggcgccggccgggtgcccctcggcgggcttgcgcccgacgagcaccttcaccggggccgtgttgagcagctggcagaggcgcgcgtgctcgcgcagcgcgtggcacgccagcacctcgccgtgcacgcgctgcgccggcgagtcaaagagcagctcgccgtcgcgccacagcggctgccagagcaccaggcactccccgcgctcgcccgtgagcgcgtcgcgcgccagcacgcgccggtgccggggctcgtagtagagctgcacgcggtcgtagtccatgatggtgacgccgcgcatggcctcggcgagctcggccacctcgtccaggccctggcccagcacgctgccggcgacgcgcaggtgcccggccaggtcgtgctcgtccacgcagcggtcgcgccgccgcttccccccggggcgcgcccggcgcaccgggaccacgccgaggcacacgatccagtcgatgtacttggcgaagctggccgtgccgttggactcgctggccgagaagcaggccgcgatgccgcgcacaaagtccaggagggccatctgcagcgtgcggtaccaggtgtcgaagaggcgctcggccacggcgggcgcctcgtcgccgaagcgccgcgccagcgcctcggcgccgaggccgcgcgcgcgcaggtgcgccatccagtcccgggccacgtcctcgtagcgcgccgcgttgagcgtgcgcgtgagcagcgtggactggatctgccgcacggcggcctcggtggagcgcaccgagttgtagatgccctggccctcggtgtagcccaggttgcccatgaggatctccttgaagaccatggtgcgcggcgtggggtggatgaggatccgcgagggggcgcccaacaggttattagcctcctccgcggcgccgccggggcagagcccgtccgcagccgccgccgccgtcgcagccgacatggccgccgccgcagagggtccgcgcgccaccaacgccacctatctgaactttacgtccatgcacggcgtcgagccgatcgtggagcgcgtccgggagctcgccgcgacgcccgcggaggcgccgccgccgctggcgtggttcaagtccctcgccgcggcggacaacccggtggatatagaggcgctggagctgcccttcgccgcgtacctgatcagcggcaatgccggctccggcaagagcacgtgcatccagaccctcaacgagaccatggactgcgtcatcacgggctcgacgcgcgtcgccgcccagaacgtctacgccaagctctcggcggcgtactcgagccgctacgtgaacaccatcttccaggagttcggcttccgcgggaaccacgtgcaggcgcagctcgggcgctacagctacgcgtgccccacgagcccgccgacggtgcgcgagctgcagaagcgcgacctggtgtactactgggaggtgctgcaggacatctcgcggcgcgtgctcgcggggagccacgaggagtttgcgcgcctgcgcgccctggagcggctcacggggcgcgcggcggagcacctggccttcgcgtgccacggctcgctgccggcgttcgcgcgcagcaacgtcatcgtgatcgacgaggccgggctgctcgggcggcacctgctcacggccgtcgtctactgctggtggctgatcaacgccgcgtacgacacgacgcagtacgcggcgcgcgcgcgccccgtgctcgtctgcgtgggctcgcccacgcagacggactcgctcgagtcgcgcttcgagcacgcgcggcagctgtgccgcgtgcgcgccagcgagaacctgctcacgtacctgatcaccaaccgcgcgctgcgcgagtacacggacctctcgcgcaactgggccatcttcatcaacaacaagcgctgccaggagtacgagttcggcgagctcatgaaggcgctcgagtacggcctcccgctgacggacgagcacctgcgcctcgtcgacagcttcgtggtgcccgaggcctacatcaacaacccggcgaacctgcagggctggacgcgcctgtactcatcgcaccgcgaggtcagcgcgtacatgagccgcctgcacgcgcacctcaaggtggcgggcgacgcgcagtttgtggtcttcacgctcccggcctacaccatcgtgcgcacggcggccttcgaccggtaccgcgaggccacgcagcagccgcacctgacgctggaccgctggctcgcggccaacgccggccgcatcaccaactactcgcagagccgcgaccaggacgccgcggcgctgcgctgcgaggcgcgcgcgcagcagggcgtcgtgctcgcccgctgcgaggtcacgtacgtgctcaacagccaggtggccgtcaccacgcgcctcaagaagctggtcatcggcttcagcggcaccttcgaggccttcgcggccgtcctgcgcgacgacgccttcgtccacgcgcagggcggcagcgccgagtacacgtaccgcttcctgtcgagcctgctgtttagcggcatgatcgccttctacaacttcctgcagcgccccgggctggcgcccgaggccgtcacggcggcctaccggcgcctggccgccgtgacggcggcggcgctgcgcgtgcccgaggagcactttgacttctcgggcgcggcggcgcccgccgcccccgcgggccccgcgggccccgggggccccgggggccccgggggcgcgcccgcgtccgcgccggcggacgacgacgacgacctctttgcggcgctcagcgagaacatgctggacatgctctactgccactacgactttgcgcgccccgagaccacgagcgaggtgtacgcgcagttcctgatgctcaagacgctcttcgccgaccgctacgcggcgctctccgagctcttcgggcccgcgttcgcgcgcgcgcccttcgagacgcacgtggacagcgtctcggtgcgcgggtgcgaggtgtttgtgggggggctgcgcggcgcgctgctctcgacggcgctgcagacggacagctacacgctcgtgggctacacgcacgcgacggcgccggccttcgccgaggagctggcgcggcgcaagctgcacgccggcaccgcggagctgctcgcctcgctggacacgccgcgcgtggtgctgcgcgaccagagcggcttcctgtccatcctcaacgtcaacctcagcgactttgtcgagtcgctggacgacctggagctggacatggccacctacgtcgactacggcatcagctccaagctggccatgaccatcgcgcggtcgcagggcctgagcctggagcgggtggccgtgtgcttcacgcgcgccaacctgcgcatgaacagcgtctacgtggccatgtcccgcgtgatctcgtcgcgcttcctgcgcatgaacatgaacccgctgcgcgaggagcccgagcgggacaacggcatcagcgagcatatattggccgcgctgcgcgacggcgcggtgcacatcgtgtactgacccgccgtccacccgcccgagggggccgagggaccgagggaccgagcgagaccgaccatgacggcgctggtggcctactcgttttacgaaataaagctacccggcggctgggcgcagccgggctgtgggcagaccgtgtgcgagtacgagcgcggcgtccgcgtcatggccacggacgggtgcacgcgctgcgacgcgctcgccccgggccgcgtcacgatccagcacgggccggtgctcacggtcctggccgtggacggggagccggagcggtgctcgtacgtgttcgcgcggacctggccggcggcccccgagggcgcgctcgtgatgcccttctcgacctggagctgcgccgagcgctcgcgccgcctgcgcgggcccgcgggcggcctgctggccaccctcgtcgcggagcgggccctgcacgtcaccatcaccgcgtaccggcccgacgtgctccgggacgccctgcgcgaggcccgcatcctggagtgagaggcggcgagggccgggcgagcggaggaggccgccgccatcaccatcgccaccaccgtcaccatccccatcccactcccatcccaccccgacaataaacgacgacggccgcgcgtgcgcggaaagagagaccgaaccggttgtgtcttttgtctggtccgtgggcatccgccctttattgatcgcggtagcaacaggaggcggggggcgcgcgccggccccgggacggggtccgggggcgcgggagggcggccgccgaggggacggcggcgtgggccgaggcgtggacgctgcgctggcggggtctcggcagctggggcgtcggggcggccgtcgccgaggcgttgatgctccgttgccggggccgcggcagctggaccgccggggcggccgtcgccgaggcgtggacgctgccgcggcggcgcgggggtgcggccgcgcacgactcgggtccgtccgcggccgtcgccgaggcgtggacgctgccgcggcggcgcggggcccgctgctgttgttgctgctgctgctgcggatgctgagagcggtggtggtgctgctgctggtggtggtggtgggtaggctggacgacggtggcgtagacggggtccggctcggtcgcgccgcgctggcgcgagcgcccgcgggcggtgcccaggccgccggcgcgcaggggcgcctcctcgtcggggggggcgcgcgaggaccgcggggcccgcgtctcgcccccgccggcggagacgcggcgggaggccgcggccaggcgccgggcggccgtctcgttggcctgcaggaaccggtggtagtcatcgaccgtggccacggacgcgcgtccgaaggtgctcatggcgccgcaaacacgttgtcccggtgcagccgcaccggctcgggggtgatgaactcgtggatgaacaccggcgcggccggccgcgttcgtccgctcgtcgggcgcgcgcggggctcgcgtgggcgtcgggcgcgcaccgcggaccggagctgatcgcgtacgcgcgccgcgtgggctctgcggcacaggataaacatctgcaggcttttatggccgcgcccgggggcgccgcgcgggccgcgcgcgccgcgcgcaaacgcggacgtcttggtgcaggcgacggggaggccggccagggccgcggccaggtcccggcggatcgtgtccgtcagccgccggcgcccgagctcgtccacggacgcgaccataaacagcgtgtcaaactccacgtgctgcgcctggcgcgggcccgccgccgccgtcgtcgtcgtcgtcgtcccttcgtcccccccatcttcctcgtcttccgtctcctccttctccgtcgccttcccctcctcgccgtcttccacgtccccctctccgtcttcgctcccgcgcaccccctcccggggagggggtgcgctctccgggggcgcgtccgcgtcctccgggcacggcccgggggactcgacctcctccacggcccatccccactcgcgcagcaccgccaggaccggcagcgcgctggccggggcgccggcgagcgcgggctccatcatccgctctcctccgtccatcccacccccctccccgcctcctcgtcctccgccggcggtgtgcgcgcacgcccgactcagtccacgctccagtcgacgggggcccggcccgtctggacgaggtacgcgttcgcctctccaaagtgcgggcaggtcctgaagggcgtgcgggccagcggcgacgggtggctgaaggtgagcaccttgtggcggcgcgggtccggcgcgcaggccttttgagcgtgggcgccccagagcatgaacaccagcttggggcgggtctcgcagagccgctggacgacggcgcgcacgagccgcgcccagccgagcggggcgtgggagccggggaccccgcgccgcacggtcagcgtcgtgttcagcagcagcaccccgcggcgcgcccaggcctccaggcagccgtgggcgggcgcgggcagcgtcgggtacgtcgcccggaccgccgcgaagatgttggccaggctcggggggatcggcaccccgcgccggacgctgaaggccagcccgtgggcctggcccggcccgtggtacgggtcctggccgatgatgaccaccttgacgtcctcgggcgccgtgaggcgcgtccaggcgaacacgtcctcgcgcgcgggcagcacctcctcggtcaggcagcggccgcggtactcggccagcagcaggcgcgcgtacggcttggcgagctcgggctccagcagcgcgcgccacgagggggccacgtcgaactcggcggcgaaggcgtcccacgtgggcgccccggggtcgggggccggggcggaggcgtcggcccccgtggctgttgtcgccgccgacgccgcgggcgtgcccttcgcgggccgggcggcggcggagggtcgggccggcttcgaggccccggcccctgcccctgcccctgcccctgcccctgccccggcgggggctgctgctgcagcggcggtggtggcgttagaggcagaagcggcggcggcaggcacgacgaggcgcaccccgggcggcaggccgcagggtctcttactcggcggggggccctccattatagcgcagcggcggcagggagtccttgtgcacggccacctctccgagctccttgagggccgcctcatttaagggctctccgacggccagggcctggatggcgacaaaggggttgaccaggtaggcggtgccagacgcccacaggatgacctccggcggcgagcagtagggcttcacgaccacgctcgtgacggcgttcatgtccggcggcagcgagtacgcgatgtagggatggcagcgcgatcccacgggcggctcgaggtccagcagcgggtgatcctcatcgtcccactccagctcctcgcgccgaggatggcgagtggggggcgcgggcgtgaccggggcctcgaagatggcatcgagcccacgggggtttcccgcgacgccggggccaggaatccccagcgccgccgaaaaaaacaccagcaccgccacgagcggcgacatcgcggctggtgtatcctttttcggcgcggggtcccggcttttatctccggaaagaggaaattgagaaataaactagacatccgcataatcccctttgatttacggaggggaaatacgtgccacgtggcggggggcggggcaactggagcgtggcgaagcggaacggggcgcgatggggcgcatcgaggcgcggacgcgtcccggggggagtctggggggagtctggggggagtctggggggagtctggggggagtctggggggagtttggggggagtttggggggagtctggggggagtctggggggaattccagctcggcgcgatcgccgcatcgcgcaaagtccggctgcagtaaatttactgcggatgcagttcccggggcggcccgccccggccagatggcgctgcagtatagctactgcggctgcagtttactacagttgcagtaccgcgcgccaccgctaaatactacagtagatttaccgcggccgcggtaaactgcagtttcctgcggccgcggtaaactgcagtatcgcgcggtaaattgtagtctggcagccgcgcgttactgcaattagcggtggctcccgacactctggccaattggtgctaatgggccgtgatggtccacgtgggggtgatgtaaccgccgggccccggttgggcactcagacagtggccgggcgccaggccaaagtgctgtctgagtgccactttatgactttgtttttctcaaacaacatcaattatggatgcacatcgtgtatataatccccggtccgcgctccgcccacccatcacagcagccgcggacgctgcgcgccggagcggtccatctcgccagccagccaaccagccgagccgcccggccgacccgagagccccgagagccagactccctcagccatagaagacaccgggcgggagagacggactgaaaaaatatatctttttttattttgtccgggcctggagacccgcagcaggagcggaggtgggtgtggggccgggagccggagcaggaccgggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctcagtcgtcgtcctgggtgaggtcgatgaagatggtagcggagcggggggatcccgacgagctagacgccggaggcccgccccggggggcggcggtctcgggggcagaggcagagggcgacgggcgccgcatcgaggaggagggtgaagacgagggggaggagcgagccgaagcggcggtgttcgccgaccccgggccggccccggcccccgaggcaccatgctgcgcagaggacccctcgccggacgatggcgcctccggagtctcgccctgggcctgtccgcccgtccggccgcgccgcaggcaccgagtccgtctctgcgcgcgcctcagcacggccgcccgtcgggccctgcgcggggagcgcctgggcgccggcctctggtcgtccgcggactcggaggcctccgtcagatcctccgtgtgcaccccgctgctcgaggcgcccgagtcttcctcgtcgggggaagacacctcagagtcagagtgcgcctcggactcggacgtgtcgatatagttcacaccctggtggctcatcggggctcgctgcatccgccgcatccactgcgccgatatgtcaaacagcgtatcgacgagggcgtgggtgtttgccccaaacatggggagcatggcctcggtcacgcgctggcggttcatcccgtgctcctggataatctcgacgatgttgtccacgacggcctcgcggatggggtcgctctcgatgaccgtcgagacctgcccataaagccagttgaagatggggactctggggcgggcgcgagacccagacccggagccctgcccttcggcctcctcgtggcgcacctcctcggtatagtcttcaccccagatgaccgcgaagcccccccctaccggctcatcctcttccccgtcgacatccgtcgccccctccacgggcgtctccacaaacgaagcgtcgctgtccacgtggtggaggatggaggtgacgcgggtcttgcacagcgggcaggcggtgctcgtcagggtccagcgctggatgcagtccagacagaacttgtgcatgcacggcagcgtctgcgcctcggtggccgcgacgtccaggcagatggggcagtccatgacggtgagtggcggtagcaggcgctgcggcggtggcgaggggcttctggggcggtagaagccaaagatcccccaggcgctggaagcctcggcggtggtcgtccgtctccgagagaccgtgcagcccatggtgtcgagggccccgttttttaaccgggtgcccggcccctttgtttgtcttcgcccggggcgtccacgcccccttccgcccgccccgtcggggctccccacgccccctcgggccgtcccgggacgatggccccggcgcgcgccgtcgtgggaagcgggccctcccgcggtgggtcgacggctcctggggctgaaagcggcgctacggatcccccgctctcacccctgggtccgtcgccgccccgcggtgcgtgcgacgcccgcgaacccccggcccacaacacaccggcaccaccgccaccatcatcatcccccctctccacagacacgctcgcactcgggccacacgctcccgtcctccgccgccaccctcccagcctcccagcctcgcgggtcctcgggacgaccgccgctcaccccgacggggggggggacggggacagggagcgaggggggaggaggacggggggggggaagaggggaacgcgagcgcggcgcaccccgtcgccgtccgtggtggcgtgcccgccaccccgtccgtccatccatccgtccatccaacccccttctcctttttccgcctcccgtcccacggcacggccccatcgacgccacaccgcgcggtgcgcgctctccagccgtcccatccccccacaccccccacgcttcccccattcgcttctcatcccgtcagacctgcgccatggagtttcgaggacgggcgcggggcaacggtggtgagaggcgagccgggggggtcgagtgggcctcgcactttacgcgcacacgcccctctcgcgcaccacacaccaccgcacatcgccgaggtcgggcgagagagggcgccctccccgcgaccccgctcgccccggtcccgacgccctcgcacccccacagcaccccctttcacccccccccccgcttctccccgctctctccgcgcgatcgcacacacaacacccgcgcgcatgctcggcttcttcacacggaggacaccatgggcagcagatggtacggtcagccgccggccgccgtgcgcatgtgtcgggctgtcgcggccaagagtatcagcgtggcttttttgttggtcagggtgtgaaaaaggcacgctgatgcgtccgccgctgcagcggagctcctcggcagcgagatcatcgaccaccaccaccacatcgctcttctcgcccgcgctcccccaccgccccccccccccgccatcgcccgcgccccttccccactcccccacccccgccccctctcaccccaccgccaccccacaccatcccccacccccccgtccaccctcaccccccatcgcgtcgcgagcgctctcacacatgcttacatgcacacgcgcccctccctcctcggcgcccccgcggcgggaccgtcgcaaccctctcccccgccgtcccacccgtccgcggccctcctctccttttccccctccccttccccttcccatccccgttcccccctgttccccatttccccgctccctccaccatcgactctcatcgacgggcttctccgccatcatcaccaccaaccaccaatcaccatctcccgccgccgcccggacggcgacccgcggtgctcccgggggcgcgttcccactcacaacccggtggtttcgatggccgcccccggggggttgatggggatgggcgctcgggggtgatcgtctgctaccaaaccgcaccaccgtccccctgtccctcatccacttctcgacggccggctccccgcggggcgggggctgcgggggggtgggtgggtgatagagcaggcgggcgggtctggacgggcggagggggggggtcccggcccctctccctcccctctcttcccccctcccctaccctctctccgtgccgactcgacgagcgggggcgaggcagggtgcgtaccgacccgcctaccaggcaggctagaagcctcacgctgctgcttggcagcgggtgggtacccaacccctcccctcaggacgcgaccgaccggtgtggagagagaaagatgcccgtcccccctgcccccaccatccccgatttttcggttcctctccccgtcgcgccgtcgggcccctcgtcgccaggggtttttttggtggaaggcccaccggggtgagggggggggggttggtggtgtgtgcgcgacggaacgggccgcaggaggatcggggtgtatgcacggtcgtccccgtctcaaacttcctcgtgtcccctgttccccatccttccacccatacattcatccgtgcggtggtggggatgagtggatggatggaggcgagggacgcggtggaccatgccaggcaagacctgcgtcactcgaagaaagggtgcctggattggccttcgcttctttgccggccacgagcgacggccaccaccgcccccccgacgggctctcttttcccccctcccgcttctcctctttcctctccggcgcgtaccccaccgttttcccgacgaggttcgagaccccccccaaagcaaagcgagagtaccccacacccgcacgcactccccggtgtcgctccttttcccccccggcagcgccggaaccgggtgcaggcggtgggggcgaagattgggttgggtgagagactagaaccggtgttctcaacccttctggagccctaccctctgtgcctggactttccagcgcgcgacacgggggggagcgggcacgcggcggtggcgatggtggcgacggcggtggctgggcgcgatcgaggagatgtggaggggtgccaagcgcccgccgccctcccccgcatctcttctctctcgcacacgcacacgacccgcggatggcgaggatgtggggggagaggtctcccggcgttagtgggtgctatttgattctcctctaaccccctctccctcgcggccccctcgaccatcgtcgtgcagcggcgacggcggctcccaccccgtctctctcccccacacgacctcgacgcgtcccgccaccccgcccagtcacgtcccaccccccggggagaggggagagggatgcacgggggtgggggtccccgccccctctctctgatcccaagacggtgtttgtccccgtctccggctcgcacgcacacatccccgccgggcgggccccggcgggatgcccggccagacaaaccgagggggaggagagggggaggaggaggaagtataggcctctcggggacgggcggcgcgtggcgccggagaggggggtggcggggaagagggaaacctagaaggcggcccatcccccgagatgacgagagcggagagaggcgctaaacggcgcctggggagtttgggggaggaggagacggcgcgcgaggagctgggggaggaggaggaggaggaggaggaggagacggcgcgcgaggaggaaaatggccggtccaacccttgggacgagaggtctcgaatcttgacaccatttcccctcccctccaccccactcccgtcgcggcttcgcgcggggaggaaaataatccgatccgacccccggggcgagaggctcgcgatcccacccccccgctccccgggggccgcgaaaaaagggggcggggcttaaagggggggggctaatttgcataaatttgcatgcatttgcatagccccgccccctcatttgcataaattagcatggcccctcccccccatttgcataaattagcatggcccctcccccaatttgcatggccccgccccctcatttacataacccctcccactcatttgcatgcatttgcataacccctccccctaatctgcataacccctccccctaatctgcataacgccctccccctaatctgcataacccctccccctaatctgcataacccctccccctaatctaccctaaactgcataacccctccccctaatctgcataacccctccccctaatctgcatatgacaacttcctccggaagtgacgctctggcccctcccccctcatttacatatgaccgcttcccccggacgtgacgccgggctcctcccacccccccccccatttgcatatgaccgcttcccggacgtgacgtttttgccgatgacgacttccgcctcatttgcgtatgaccgcttcccgcgcgcaacttccgccggacgtgacgccggtccccacggatctcatctgcatatcgcgccggccccttccgcttcccccggacgtgacgccggcttccggggcgcggacggggcgggctccgcggatcgcatcggcgcgccgagcctgccccttccgtcgcaccgggggtccgcgggcgggggcttccgctccgcggcacccgccccattggctcccctcgcgccacgcggtcgccgtcgtcatcgtcccgccggccaatcgggtggcagcgggggaggctgggagtggggacggaagacggaagccagatgaacctgttccgcccgctctcccaccgcctttccctcccctccacgacgaccacccgggaccaccaccaccaacaacaccaccaccccccccccacgcacacactttactactatcaccaccagggggcgatggttgcaatggcagttccctgtactgaccaccaccgtgtttttttctctctctctctttccttcccccccccctcgaccaccgcaggaccaccatcgtctaactcccacccgggaccaccgggaccctcgggaccatctacctcccaccaggacccgccgggaccaccaacaccgtccacctcccaccaccaccaccatcaaggacccccaacatccccaagaccctctacttcttcccacccagacccaccaggaggaggacccccatctgctgagacccaccaggacccaccgggaccctcatctgctgagacccaccaccaccaccaccaagaccctccaggaggaggacccccatccacttcttcccatcaccaccaccaagaccctccaggaggaggacccccatcacccccaccaagaccctccacctcttcttcttcctcccaccagggacccccatccacaagaccacctccgccccagagaccaccgccaagatggccgcctccatctccccaaaaaatctcagagactcgagctggttcagaaaatacagcacaaactttattttctcactctgaaaataaactcttttctcacccgatgggagaaggaggagaaggggaccgggggaccgcgggaggaggaggaggaggagaaggggaccgggacgatcctccgccgccgagccctccgccgcggccgccgccgccgcttccaccaccgccgccacctccgccgccgccgcagccgcctccggccgggggatccgcgcggaggagaaggagaggaggaggaggaggagggccgccgggccggggaggcaggcgccggggaggcaagcgccgccgggccgaggggaccgaggccgccgccgcggacgcggaggaggaggaggaggaggaggaggacggggacgaggacgaggaccgggccgggggcgaggggagagaagacggaggagaagggcctcgaggagccggtggaggggccggagagtcagagtcagagtccagccgggccgagggggcgccccgctcagcggagcagcaggtaggggttgccggcgtcctcggcctcctcgtcgtccgagatggcctccaccttgatgggcccgagcgggccgcggggccggccgtcgccgccgcggaagccgacgatctccaccgcggcagagtcctccccgtcctcgccgggcccccgggcgcccgagggccggtgggtctccacggcgccgccggcggcgcggacgctggtctcgaagggcgcaaagtcccagcccacggccggcggggcgcccgcggccgcgagggcgcccggggccagcaccagcggggcggcctcggcgtcgggctccagcagcgccgcggcgcagaaggcgcgcagctcggccggcaggccctcggggccgcggagctcggcgaggccccggcgaccgcaggagacgaagacgggccgcagcggggcgccgagcccccagcggttggccgcgcggtgcccgaaggcggcgcccgcgtcaaagtccgggtccccgagcccgagcgcggagcgctggcgggccatgtccttgcagccgtccacggtggggagcacgcgctggcggtaggcgcgcggcggcagcgggaccggggtccggggcccggcgcgggtgctcaccgtgtagcgcacgttgtcctggcggcagaggcgcagcggctcggccccggggtgcaggcgggcgaaggaggcctccacgcgggcgaagcaggccgggcccacgatggagctcgagtccaggacggccgcgcgcagctcgcggcactcgggccagcgcacggcgcactgggcggccgggtccaggcgggcgcggacgtagacgtggtagtcccccacggccgggccgtccgcgggccagtcctcgatggtgtccagcacgatgagccggcgccgcgccgcgccgagccgcgagcagaggtactcgacggcgccggcgaagccgaggtcccgcgccgagagcagcagcaccccctgggcgttgaggcggccgatgtcggggcgcccggtccagttcccggcccaggcgtgcgagtccggcgtgcagaggcggtgggcgaaggcggcgagcagcgccgagaggccgccgcggcgcgggtcccaggccgggcgcggggcgccctcggcgggctcggcgcagagctcctcgtggggcagcgggtcgtagagcaccaccacgcgcacgtcctcggggtcggctatctgccgcatccaggcggcgcggcggcggagcggggcgcccgcggccccgcggcgcgcggcgatgtgcgccagggcggccgggtcgaaggtgagcgccgggcgccagagttcggggaagacctcctggtccacgaggacgcgggccacctcgggcgggcagtaggcggcgagggccgcctcggagggccgcggcgtgtgggtctcgccggccgggacgcggcggaagccgccgtcgggcgcggggtgctcgggcatgggcccgagcgggcgccggagccggtcgtcctcggaggaggaggaggaggaggacagcagcgcgggagcggggtccggagcgggcccgagtccgagggagcggcgcttgcgccggggcccccggtcctcttcgtcgtcgcggtggccgtggccgtccccgcggagggccgagccggagagcccctcgtcctcctcgccgtccccggggcggcgggccccgggcgcgcggcgcttcttcttgcgccgctcgggcgctgggtccgggccggcggcgggggagctggcgtagccggaggagccggagaggccggacttggtgccggggctgccggacttggtgccggagctggacttggtgctggagctggacttggtgctggcggggctggagggcccggagccggggaggccggagggggcgcccgccgccgccggcgccggcgctgggacgacgaggccgggctgctcgggccagagcgggggcaggccgggcgcgggctccgcgggcccgggccgcgcggcggcctcggcgagccgggccgcggccacgttggccggggcgaagagggccgcggcgtaggtccaggcggcctcgcgggcgcgggccccgtccacgctgtagcgcaccagcggcgccacggtgcgggcgacgagggcgacagagtccgcggcctgctgccgctcggccgggccggccccggggatcgcgtcgcggagcgcgagcagcgcggcggtcacctcctcgaggcaggcgggcccgagggcggccggggcgcgggcgggcgcgggcagccggagcgggacgggcagcaggcgctcgaggacgccgcggcaggccaggacgcaggcgtccgccagctcgcggggcacgcggccgggctgcgcggcggcgaaggcggcgcggacgcgggcgcagagggcctcgacggtcgcctccccggcgcgggggtccgcggcgcggcccgggtaggccatgtcggcgtaggcccggcggaggctctgcaggatgaaggtcttctgggtgcgatcgtagcggcggctcatggccacggcgctcaccgcgtgcggcagggcccagagcgggtcctgggcggccatggcgtccccgatgtgcggcagcggcggggtcacgctgccggtgatgaaggagccgtggccgtggggcgcgtggacccggcgctggcagaactggttgaagcgctggtcgggggcctgcatccgcgggttctgcagccaggacatggcctcgccggcggccccgctgtagatgaggcgcacgagggcctcgtgctgcttcctcgagtcccccatctccgggatgaagacgggcacgggcccggccgcggcgcggtagcgggccgcggcctggcggacgtcgtcctcgtcccagagcccctcgcgggagtccccggcgccgccgtagcggacgcggccgtcggccggagggtcggagccgggccagggctccccgagcggggtgagcagcggcccgtcggtcggcgggggcccgtcggccatgagcgagaggtggttgttggtggggcggcgcttcctgcgcgggggccgggcgggctccggggccggggccggggaggccgcggcggaggaggaggaggaggaggcggcggcggaggaggccgagggccgcggggccgcggcgggcgccggcggagacggtggcggcccggcgcgggcgagtggggcgccggcccggactccttcgtcttcttctccctcggaggaggacgaggacgaggaggacgaggaggacgaggacgaggaggaggccgagcgccgcgcggcggcggcggcggaggcccggggggcggagggcgagcgggccggggagaggtccgagtcgctgccgctgctgctggagctgctgaagccgcggccgcggcggagggcgccctctccggcgcggcgccggcggggctgtctctgcgggggcgccccgccgtccccggcgaggccgagtccgtcctcgtccttctcggggccgcgggcgacgggctcgacggcgacggtggtggaggagctggagttggagttggagttggaggagacggggctccgggcgccaagcggccgaggatcgagccgcctcgcggcggcgggctcgtcgagcaggggctcgcggtgctggtgatggtgacgaccgcggtcccctccggcggagggggcgccgccgccgccgagcgccgagaccggcccggcggcgggggaggctggggaagcgggcccccgccgtgccggcgctgcggccaccgctgctggctgtgctggtggcgccggggtccgaggccgcgccgccggcccgggctcaccgaccgggtcccccctcgcgggggaccatctccgcggggctgccgaggggccgggggagccggaggaggaggccggggaggctgcggagggggacgggcgcccggggccgccgggggccccggcctctgccgctgcgagtgctgccggggtcggcggccggggcccggagccggcccgggaccggggcccgaggacgaggtgaccgtgctcggagccctgatggagagcccgaccgggggacccggcggccggggacccgggctcgtcctcctcctcgtcttcgtcgtctagcaccacgatctcgcccgagccccggcggggctgctgctgctgctgctgctgggccgaaggaggacggggcggcctcgtggctccggccgcggccgcgaggacggcggcctcggcctcggcctcgccggcatcgtcggagaagaggccgccggggccaaagaggagatcctcgccggaggggccgcggcgccgggagccctggctgccgccgtcggagccggccgcggtcccctcttcctcggccgccgcggccgcggccgccgccgccaggagctggctgaagttgccctcggtctcgatgaagtcaaagagatcgtcggccatggtctcgatcggggtctttctgcctgagcgaggccgggcgccgagcgcggagagcgggcggcggagaagaaggaggaaggcggccggaggaggagaagaagaagactcttctctggcgggccgagagcctcggtgggtcgggccgtccgtcgagggctgatagccgccggagagccggagtcttcagagtccgcgccggagccgagacgatcggatcccctcgggttggcagagaacgatgctgtccgtacctgcaccgcagtgaagtgctacgatggagaccgtgcctataaactccccgagaagagcccgcccccaggtaagcggaccaatggccgattttcgccgcggacttccccgacggccggccaatgggatttttctcgcccgcttcctctcgcgtctgctttgcatgcccggcccaagatggcggccgccggccaatgggatttcgcgaggaacttcctcgcgaggaccatttgcatgcccggcccccgcggcggccatcttgcccactcgacggccaatgggatttctctcgcccgcttcctctcgcgtctactttgcatgcccggccccgagggcgccatcttggcccctcgacggccaatgggatttctctccctacttcctctcgcgtctactttgcatgcccggcccccgcggcggccatctcggctcgcccgggccaatgggcgcgcggaggcgtctcccgcgcgcctctgatttgcatgcccggcccgctctgcggccatcttggccgcgggcggccaatgagattgtccgaaaatccctcgcgcgggcgcgaggcgcatgctcggcacgcgacccacccccgtggtgctagcgagccaatcagatgattttcggggaagcttccgtgtgcacgtcatttgcatgctcgccccacgtggccgccctcggccaatggggcctcacggtgcaagcttccgtgtgtctgcacgtggtccgcatgtgttgtggtggtctctcagtgttttgtggtggtctctgtgttgtgtggtggtctctcagtgttttgtggtggtctctgtgttgtgtggtggtctctcagtgttttgtggtggtctctgtgttgtgtggtggtctctcagtgttttgtggtggtctctgtgttgtgtggtggtctctcagtgttttgtggtggtctctgtgttgtgtggtggtctcaccgtctccccctgccactcgcgagacccccgagacccccgtttccccctccccgagacccttgaggccccccgcgacccccgcggtcgccccacccgcgcctgcgcgctcggcgcgcgctccgagggcgcccccagccggtcggagagacgagcggaaccgccgtcggaccggggaccggcgaccggacccgaaccgggaagcgacgccggggcgggagaaccggacccgaacctcgagcccggacccgcccggacccggaagggaggagccggacagccacgccttggatacttttgtcgcccacccaccccccctctacctctctctcccggtcccccctcccaccccacgagacacgccccagagtgaaaaaaataaaagttgttctcgttgcaccgtcttccggctcgtgtcgtccttccgcggtacctcgggcgggcgggagggggcgcgagaccggctcggcctcgtcgggagagagggagttgggggggaggggagccaagatggcggcggggcgtggcggggcgtggcagaggggaggggggggtcggagcgcggaccccgcccggtgggggggcgggtgcaaaggggcggggcccaaaatggacctcgggccgggaccccgggggcgctccgggagacgaagagggccgggcccccttcccggcggggagggccgggccggcgcgccgggacgcccctccgggggaaagagtgtccccgcgcgggcgccgcgtcccgccccgagcccccgggggcgcgcgggcctcggtcgcgcccgccggacgcggaggcgcgaggcccccgccccccggggggtccgggacggggggggtcaatttttgctgtgtgtgtgtgtgtgtgtagggaaggctcgctctctctcgtggagggttgggcggacggatggtcggtcggacggggcgggcggggagggtgtcgtccgtgtgtctgtgcgcggggagagtgtgatgtgggggtgtgcgagagagagagcgtgcgtgtgggtgggtgtgagggccggtgcgggtatgggggcccgtgcgggtgggagggtccgtgtgggtgtcggtggtgcgggtgttgctggtgataccggtaccgacggtggtggccgggcgggcggtctctccgtctccgtctccgccgtcgcctcggtccgaggaggggggcgcccgggcgtgtctcgcctttcccccagttcgcctcctccttctcctcctctctcgcctctcctctctctctccgcgtgtgtgcgcgcgcctctccccgtgcgtgtctcgctcgccctctccgcctctgtcctccgcccgactttccgactctcctccttctcctccttctcctcctctccccgcccccgccccctgtcccggtcccggtcccacgcgcgcgccccggggaagggtcgggcgatggccgccgccaccgccccacccttcccccggaccccctcccgcccgccggggcgccccgcgtgctccgggggcgccggccggccatccccccacacctcccctcctccccgtccccaacccgccccgcactcgcccgacactcggcccgcggcacgcgcgcccagaccttcctctcccccctccaccgcccgccctcgccccctccgtctcatgccacccgcggcagcatcccccctccccgtcgtcctcagccaacagccaacagccaacagccaacaacccacagccaacagcctcactctcgcgctctccccgtcgcgggaaaaaacacggcaccacaggcgggagcggggtcgctcgggggaaggtttcattcaaacgacaggcgacaggaggggggcccccgcgcgggtcgggccttcttccccatccccgccggcccccctctttctctctctctctcttcttccgcgtcttcttccgcgtccgcttcggccccccgcggccgcggcgtgggagagcggggcgggtggggaaggaagaggggacgggggggagaagaagaaacggggaagcggggggggaaggaacagacggggaagggggggggaaagggaaacggggaaacggggaagggcccgccggagggaagcgggggggagaggcgcggcgccccgcgcttccccttcccgcttccccctccgcccggccgcgggcgcccgggagacgggagcgaaaggaggagaggggaggaaaaaaagtctagagcggggaggagaagtcaccgtagtagtcatagtagtagtcatagtaatatagtaggagtaggagtagtaggagtagtagtagtaggagagtctgtgcttaccgagagaggagaagaagaagcagaagccggggcgcttcctccccggcggcccgggaagaaagccccggcgcctcgggccccggagatggggacgcggagaaggaaacacccaagggcggaggaggaggaagaaggagaggaagggaaaaaaaacctcgagagcggcggcggcggtcgccgcggccaggaggaaaaacggggggagcgggagcggctcccgagtccgggaaggaaaggcgggcggaggaaggcgggtgtggaggaggaagcgctcgcgcccctcgctctctctccccccgtccccctctccgcgtctcttccccgcgtcctctctctgcgttcgccggtcgcggcgcgggcggcggctgcagaggcggctgcggacgcggaggggggcgaggggacacagaactctttttgtcctcctcgccggctcccaggcggcgggcgcgtcccgcgtccccggcgtcatcacgcgggacccccggtccccgtgacgcaggacgcggggaggggaggggagggaggggagggggtcggggggaagggaggggggggaggagggggagagcgggtgacgccgcgggtgggccgaggccggcggggagggaggaggaggacgcggcggcggcggtgaaggaggagagccgcccggcgcgcgcccgccgccgcaggcgcgtccccggcgcgggcgcctctccacgccccgctccgcccggcgcccaatggcgcggccggctcggcggcccggcccttcctccgccctcctccttccccgccgccaccgccgcctcctcccttctccctcctccctcccctcccgccgcccgcgcgcccgcgtgcgctcgtgccggcgcggcatccccgcccaggcggcgggggagcgcggagcgcgccgcctccccgtgatcacgttcgcgcaccgcggcgccagcaggaaaaaaaaacaacaacacagagacacacctccctctccgtcctgcagaggcggctgcggacgcggaggggggcgaggggacacagaactctttttgtcctcctcgccggctcccaggcggcgggcgcgtcccgcgtccccggcgtcatcacgcgggacccccggtccccgtgacgcaggacgcggggaggggaggggagggaggggagggggtcggggggaagggaggggggggaggagggggagagcgggtgacgccgcgggtgggccgaggccggcggggagggaggaggaggacgcggcggcggcggtgaaggaggagagccgcccggcgcgcgcccgccgccgcaggcgcgtccccggcgcgggcgcctctccacgccccgctccgcccggcgcccaatggcgcggccggctcggcggcccggcccttcctccgccctcctccttccccgccgccaccgccgcctcctcccttctccctcctccctcccctcccgccgcccgcgcgcccgcgtgcgctcgtgccggcgcggcatccccgcccaggcggcgggggagcgcggagcgcgccgcctccccgtgatcacgttcgcgcaccgcggcgccagcaggaaaaaaaaacaacaacacagagacacacctccctctccgtctccctcgccttctccgttttggggggagaccgatgccggtgccacatacacatacacacacgcgcgcaccccgtcccccccccccttccccttcccctcgccccggccccccggctcggtgccggagaaaagggggtgccggcgcggaccggggtcgggcccgcggcgccggggggtgggcgcgcgtgtccgtgtgagagtttacggtgtgggtgggtgtcacggtggagcgagctggggggccccggctcgctcgctcgctcgctcgccaggccggcaggccgggggcccggcctggacgcgggccacactgtgcggcggaccgtgccaagtttagggcgctgcgacccagagagagcgccgagctaaaagctattttatcgccgctgcggtttcccctggcgcggcgcctagacaccgtttctgcagacagagccctctctaatggcgcgtccgggaaggcttctcccgggccgcgagggccgcggtggcgacccccttcctcccccctcctccccgtccccgtccccgtccccgtcccccgcccgctctccccgggccgccgcgataccgcgcgggcgataccgcgcgggccacccgcgggcgctaccgcgccgctccgctcgccgcctcttccccctcctccaccgccgccagtcctcctcgctcctctccgcccacgcaactcctcccgcccgcgtgcccccggggccatcggctggaacaccctgagccttcgctgagtgctgcccccgccccgcccgcggcactccgggcccgcgtgagagggtacagagcgaacccggccccgaccccctccgcccgccgggggggggcttcccggaaccagcgcggtcatcaaccccctcccccacccacccgcacagcgcgcacgccgaccgcccgcgtttgctacgatccctgcgcccgacggggccgcgggacggcgcgcgcggcacggaacgagaggggggcgggggtgcgggcgagtgagcgtgcgcgaaggggggagagggcgcgtgggcgagagggacggggcggggcggggtggggggagcggcggctcgccctccgtcctcccctccttcctctccccgtcgactcgcgtcgcggcgattcgggccgcatcgaggcgcgtcggggttttcgcgccctcgcgccgccgccgcctcctcatctttcgtggttaacacacaccacacatacacacaccctcccccgcgccgctgtctgtggcgcccgtcgtctctcccgccggcgcaaccccgggccctgagagctttctccgctttctatttcggccccggcctccccgacctctctcacccgccctctcgcgctctctttcccacacacacacacataacaagaggctcgccctcgttccatcatctcggcgtactcgccgcctcgtacactacatccgtctaccgccgccagcaaccctttttttcccccgctcactcctcttcccccctcctccttcccccctcccgcctctccattcatcaatctcgtccaggccgcgggcccattcaccatcgccgcggtccgagctcggccaggagagatggatggatggattgggggaaggaggggggcggggagcgcgagcgggagaaggttggcgaaggagagagcgagagcgggaactggagagggggctgggtggggtgaggcgagtgaccgccaccgccctctaccaccaccacccctcccctccctctcctctcctctctctccccggtcccgtgtgcacacacagagagtgtttttttttccttcttcttcttcccggttcctcttcgtcctccggaccctcggtgccgcgatcccccaccccgccccccttcctcctccccccatttcccccgcccccaattcccccactcctcccaccagcccccgccgccgcggccgcagccgccgcgatccgtcctccccattcatcctttttccagaggccattcatcgccccgctcatccgtcttcatcgccgcggagggctggctcgaggccagcgcgccctttcctctcccggcgcgcccgctccctccgcgtcccctctccccgccgcccccctcggccgcaccgagccttctccgcgcggtcccgtctctcccctccccccatcccgcccccactccccgcccccctctccccctccctctcccgccggtgttttcccacgcccacactcactttctccgctcccaccccaccctcctgactcacccgtcccgggggattccctgtcccccggggcgcggccgacccggcggtgacccgtccccgccccgccgcgcccctttttcctcgagagcgggcgtccgggaagggagaggtcgcggcgggtcacgcgcgctcccgcgacttcccggcccgcgatgcggtcccggaaatttccgggacggtcgcggcgaccgtctcggggcccgcggcgccgccaactcgagtcttggcacgcgtccagcgttcgcacttgttgtatataagatataatactagtatatattagcaattggtgcgaacgtgacgtggcccaatcaaatctctccacgggccccacgtgcccctattggcaatttatcgaccgctttccgaagggatcgaaccacgggggtgggtgccccggggacgcgccggccaatgggggagccggacccgcgtccccggagcccgcattggccggtcccggacgcccgtcccgcgggccggaccgccccccccgtctcgagtcttggcacgcgtccagcgttcgcacttgttgtatataagatataatactagtatatattagcaattggtgcgaacgtgacgtggcccaatcaaatctctccacgggccccacgtgcccctattggcaatttatcgaccgctttccgaagggatcgaaccacgggggtgggtgccccggggacgcgccggccaatgggggagccggacccgcgtccccggagcccgcattggccggtcccggacgcccgtcccgcgggccggaccgccccctcctcgagtcttggcacgcgtccagcgttcgcacttgcggtatataagatataatactagtatatattagcaattggtgcgaacgtgacgtggcccaatcaaatctctccacgggccccacgtgcccctattggcaatttatcgaccgctttccgaagggatcgaaccccgcggcacccgcctccggggacgcgccggccaatgggggagccggacccgcgtcccctgagcccacgtgtgaggccccggccaatgggcccgtcggcccggccccctcgacggggcgataaaggggtgcgtggcggcccggccgacggcactcgccgccccaacgacgccgacggggatcgaccgggagaggcgcggagcgcgccccgagccgacgaggagccgcgccccgctcgaggacgcccggacgacgacggtgacgtcggcatcggcctcgacgacgacgatcgcgggacccgacggtaccggatgcgggggatcccccggtcggggtgggccgggagtgggcgtggcacctcctcccccgtcccccctccctacccaccccgcgcgatcgcgctcatcgtatccgttctcgtttcaggtgccgcatcgttccggaagaaccgcctcgggggtcgcgcgtccgtcgcctaggtgagtcggggggcttcccgggtgcggctcccccctccccctccccctccccgggacgggtcgggtgggtaggtgggcgggggtcgggagggatcgggggaaggggagggggtcgtggctcacgctgacgtcatgcgttttctccccccccccacagggacgcgagcgggaccggctggtcgaccacccatccaatccatccaatccatccagccacccacccgtcggggcggccgggctcgggtccggttccgggtcggcgtcggggagcctcgcccacccacccggccccccgtcccagcccagccccccgcacacccccatcgcaccccctcccaccccaccccgttcccaccaccccgcccgacccaccgcgtccgccgcgcccatggaccgggtctgggccgactggtacgagcccgtgccctccccgccgttctcgcccgtcgacccgcccgggccccggcccgcgaccccggtcccggggagcagccccccgtcccccgcctcgacccccacgccccccaagcgcgggcgctacgtcgtcgagcaccccgagtacgggcccccgcccgaccccgaggaggtgcgcgtccacggcgcgcggggccccggcgccttctgcgcggccccctggcaccccgacacgcggcgcctcggggccgacgtgaaccgcctctttcgcggcatcgccgtctcggccgccgacgtgacgggcgacacgcgcgccctgcgccgcgccctctttgacttttacgccatgggctacacgcgccagcgcccctcggccccctgctggcaggccctgctccagctctcgcccgagcagagcgccccgctgcgcagcgcgctgcgcgagctcaacgagcgcgacgtctacgacccgcgcgtcctctccccgccggtcatcgagggcccgctctttggggaggagtgcgacgtcgacgaggacgacgccggctcggacaccaccgtcgcgtccgagtttagcttccgcggctcggtgtgcgaggacgacggggaggacgaagaggaagacggggaagaggaagacgaggacgaggagggggaagaggaagaagacgaggaggaagaggaaggggacgaggacggggagacggacgtgtacgaggaggacgaggacgaggacgacgaggacggggacgggctctgtgaggacgaggacgaggaggacggggacgactttgacggggccagcgtgggcgacgacgacgtgtttgagccccccgaggacggctcggacggagagggctcgggctcggacgacggcggggacgaggaagaggaagaggaagaggaagacggggacgaggacgaggaagaggaggacgaggaagaggaggacgaggaagaggaggacgaggaagaggaggacgaggaagaggaggacgaggaagaggaggacgaggaagaggaggacgaggaagaggaggacgaggaagaggaggacgaggaagaggaggacgaggaagaggaggacgaggaagaggaggacgaggaagaggaggacgggggggaagacggtgaagacggcgaagaagacgaagacggagagggcgaggagggcgggaaggacgccgcccgccgggggacgcgcgccccgacgcggcccgccgccgccccgtgaggcgggccgcgccccgctctgttcttcctccacccccaccgtccgccccgtcgtttgcccctttccccttccccctcccctctgttgtgccctcaataaacacggcggcccgccgctcgaacctcaactctctcgtctctcgggcgttttttccctcccggccctcgtggggaagggagatgggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagaggggagtgggatggggtggagacggtggagggagagaaaggacggaggcgtgtcatcgggtgccagagcgagagcgtttattgttaaagttgttggtggggggggagtccgggggagtccgggggagtccgggggagtccgggggagtccgggggagtccgggggagtccgggggagtccgggggagtcctcggcggctattggtgggaagggggagtgacgtcagggcagcgggggggggggaagggggggaaggggggagatgggggagggagagagaggacggaggcggcgcgagtctgcgggatggtgccgaggggagccggacggtcacggggagggaggatgtggcgcgcgtggggcgtgggccgcctctgccgcggtcgcgagccgccaacgccgccgccaccgcctcccgcccaaagtgatatcgcggcgtgccgggacggccggcgctcattcacaccgcacccgttcacccgcgtccccgcgggcaagcacgcacacacccggtcgcgcatcatgctggcgatgtggagatgggtcaccaagaggtcgcggctccgccgaggccacgcccatcttgggggaaataaaggagtccggggaatgtgttccttataccttgccgggctcagcagggggttgtcgcgcgtccacgcccagcgctcgcacgcagcaacaatggccgacgccggaatccccgacgagatcctgtactcggacatcagcgacgacgagatcatcatcgacggcgacggcgacagcagcggggacgaggacgacgatgacggggggctgacgcggcaggccgcgtcgcgcatcgccacggacctgggcttcgaggtgctgcagcccctgcagtcgggctcggagggccgcgtcttcgtggcccgccggcccggcgaggcggacacggtggtgctgaaggtgggccagaagccctcgacgctgatggagggcatgctgctgaagcgcctggcccacgataacgtcatgagcctgaagcagatgctcgcccggggcccggtgacgtgcctggtcctgccgcactttcggtgcgatctgtacagctacctgaccatgcgggacgggccgctggacatgcgcgacgccgggcgcgtgatccggtccgtgctccgcgggctcgcctacctgcacgggatgcgcatcatgcaccgcgacgtcaaggcggagaacatcttcctcgaggacgtggacacggtgtgcctgggggacctcggggccgcgcgctgcaacgtggcggcgcccaacttttacgggctcgccgggaccatcgagaccaacgcccccgaggtgctcgcgcgcgaccgctacgacaccaaggtcgacgtctggggtgcgggggtggtgctcttcgagacgctggcctaccccaagacgatcaccggcggggacgagcccgcgatcaacggggagatgcacctgatcgacctcatccgcgccctcggggtgcaccccgaggagttcccgcccgacacgcgcctccggagcgagttcgtccggtacgccgggacccaccgccagccgtacacgcagtacgcgcgcgtggctcgcctcgggctgcccgagacgggggctttcctgatttacaagatgttgacgtttgatcccgtccgccgcccttccgctgatgagatactcaactttggaatgtggaccgtataaaacggcccggctccgagcggtaggacacacacacctttgcgcatctccacagctcaacaatgaagtgggcaacgtggatcctcgccctcgggctcctcgtggtccgcaccgtcgtggccagagaggccccccgggagctctgctacggccaccccgtccacgacgaccggcggcccgtcgggcccgcgaccgacgcccagcccgtgaacccgctcgcccccgccaacgccaccgggacggactactctcgcggctgcgagatgcgcctcctggacccgcctctcgatgtctcgtcccgctccccggaccccgtcaacgtgaccgtcgcctggttctttgacggcggccactgcaaggtgcccctcgtccaccgcgagtactacggctgccccggggacgccatgccctccgtcgagacgtgcaccggcgggtactcgtacacccgcacgcgcatcgacaccctgatggagtacgccctcgtgaacgccagcctcgtgctgcagcccgggctgtacgacgccggcctgtacatcgtcgtgctcgtctttggcgacgacgcctacctcggcaccgtctccctgtcggtggaggccaacctggactacccctgcggcatgaagcacgggctcacgatcacccgccccggggccaccctcccgcccatcgcccccacggccggcgaccaccagcgctggcgcgggtgcttcccctcgaccgacgagggcgcctgggagaacgtgaccgccgccgagaagggcctgtccgacgactacgccgactactacgacgtgcacatcttccgcctggagtctgacgacgaggtcgtccacggcgatgcccccgaggcccccgagggcgaggaggtgaccgaggaggaggccgagctgacctccagcgacctcgacaacatcgagatcgaggtcgtgggctcgcccgccgctcccgtcgagggcgccggcgacggcgaggaggggcacggggacgaggaggacgaggagctgacctccagcgacctcgacaacatcgagatcgaggtcgtgggctcgcccgcggccgcccgcttcttcgccgcctccaccaccccccgcgcccccacccgcgcggccgagatcacgaccatgaccacggtcaccaccgtgcggacgaccgaggaccccagcggcatcaccgactgccgccggagcgacttcgtctcgccctctgacatcttcgtgacccccaccggcagccccgccctgctcctgggcttcctgggcagcgcgctcgcctcgcgccccctgcacctgacggccggggagacggcccagcacgtgcgcgaggcccagcagaagagccgccacgtccgctccctcggcggcctccagctctcggtcgagaccgagaccaccaacaccaccaccacccagacgggcctgtcgggcgacatccgcacctcgatctacatctgcgtcgccctcgccggcctggtcgtcgtgggcatcgtcatcatgtgcctccacatggcgatcatcagggcccgggcccggaacgacggctaccgccacgtggcctccgcctgacccggccccgcccgactcccccgcgattcccccctctctctcaccgggtgtccatcttcaataaagtatgtctcaaacacctaatttgcgtacggccttgcttacggggggtgcgatccacgcccagcggtccataaaattgggtcggcgccccaggttcccatacactcacctgccagcgccatgctgctcgcagcgctattggcggcgctggtcgcccggacgacgctcggcgcggacgtggacgccgtgcccgcgccgaccttccccccgcccgcgtacccgtacaccgagtcgtggcagctgacgctgacgacggtcccctcgcccttcgtcggccccgcggacgtctaccacacgcgcccgctggaggacccgtgcggggtggtggcgctgatctccgacccgcaggtggaccggctgctgaacgaggcggtggcccaccggcggcccacgtaccgcgcccacgtggcctggtaccgcatcgcggacgggtgcgcgcacctgctgtactttatcgagtacgccgactgcgaccccaggcagatctttgggcgctgccggcgccgcaccacgccgatgtggtggaccccgtccgcggactacatgttccccacggaggacgagctggggctgctcatggtggccccggggcggttcaacgagggccagtaccggcgcctggtgtccgtcgacggcgtgaacatcctcaccgacttcatggtggcgctccccgaggggcaagagtgcccgttcgcccgcgtggaccagcaccgcacgtacaagttcggcgcgtgctggagcgacgacagcttcaagcggggcgtggacgtgatgcgattcctgacgccgttctaccagcagcccccgcaccgggaggtggtgaactactggtaccgcaagaacggccggacgctcccgcgggcctacgccgccgccacgccgtacgccatcgaccccgcgcggccctcggcgggctcgccgaggcccaggccccggccccggcccaggccccggccgaagcccgagcccgccccggcgacgcccgcgccccccggccgcctgcccgagccggcgacgcgggaccacgccgccggggggcgccccacgccgcgacccccgaggcccgagacgccgcaccgccccttcgccccgccggccgtcgtgcccagcgggtggccgcagcccgcggagccgttcccgccccggaccaccgccgcgccgggcgtctcgcgccaccgctcggtgatcgtcggcacgggcaccgcgatgggcgcgctcctggtgggcgtgtgcgtctacatcttcttccgcctgaggggggcgaaggggtatcgcctcctgggcggtcccgcggacgccgacgagctaaaagcgcagcccggtccgtagcctccgcagtaccggcgtcgatgatgatggtggcgcgcgacgtgacccggctccccgcggggctcctcctcgccgccctgaccctggccgccctgaccccgcgcgtcgggggcgtcctcttcaggggcgccggcgtcagcgtgcacgtcgccggcagcgccgtcctcgtgcccggcgacgcgcccaacctgacgatagacgggacgctgctgtttctggaggggccctcgccgagcaactacagcgggcgcgtggagctgctgcgcctcgaccccaagcgcgcctgctacacgcgcgagtacgccgccgagtacgacctctgcccccgcgtgcaccacgaagccttccgcggctgcctgcgcaagcgcgagccgctcgcccggcgcgcgtccgccgcggtggaggcgcgccggctgctgttcgtctcgcgcccggcctcgggggacgcggggtcgtacgtgctgcgggtccgcgtgaacgggaccacggacctctttgtgctgacggccctggtgccgccgagggggcgccccgtccccacgtcgccgcccgcggacgagtgccggcccgtcgtcggatcgtggcacgacagcctgcgcgtcgtggaccccgccgaggacgccgtgttcaccacccagcccccgcccgagcccgagccgccgacgacccccgcgcccccccgggggaccggcgccacccccgagccccgatcggacgaggaggaggagggtgacgcggagacgacgacgccgacgctgaccccggcgcccgggaccctggacgcgaacggcacgatggtgctgaacgccagcgtcgtgtcgcgcgtcctgctcgccgccgccaacgccacggcgggcgcccggagccccgggaagatagccatggtgctggggcccacgatcgtcgtcctcctgatcttcctgggcgggatcgcctgcgtggcccggcgctgcgcgcggaatcgcatctaccggccgcgacccgggcgcggatcggcggtccatgcggcgcccccgcggcgcccgccccccaaccccgtcgccggggcgcccgtcccccagcccaagatgacgttggccgagctgcgccagaagctcgccaccatcgcagaagaacaataaaaaggtggtgtttgcataattttgtgggtggcgttttatctccgtccgcgccgttttaaacctgggcacccccgcgagtctcgcacacaccggggttgagaccatgcggccctttctgctgcgcgccgcgcagctcctggcgctgctggccctggcgctctccaccgaggccccgagcctctccgccgagacgaccccgggccccgtcaccgaggtcccgagtccctcggccgaggtctgggacgacctctccaccgaggccgacgacgatgacctcaacggcgacctcgacggcgacgaccgccgcgcgggcttcggctcggccctcgcatccctgagggaggcgcccccggcccatctggtgaacgtgtccgagggcgccaacttcaccctcgacgcgcgcggcgacggcgccgtgctggccgggatctggacgttcctgcccgtccgcggctgcgacgccgtgtcggtgaccacggtgtgcttcgagaccgcgtgccacccggacctggtgctgggccgcgcctgcgtccccgaggccccggagatgggcatcggcgactacctgccgcccgaggtgccgcggctccggcgcgagccgcccatcgtcaccccggagcggtggtcgccgcacctgagcgtcctgcgggccacgcccaacgacacgggcctctacacgctgcacgacgcctcggggccgcgggccgtgttctttgtggcggtgggcgaccggccgcccgcgccggcggacccggtgggccccgcgcgccacgagccccgcttccacgcgctcggcttccactcgcagctcttctcgcccggggacacgttcgacctgatgccgcgcgtggtctcggacatgggcgactcgcgcgagaactttaccgccacgctggactggtactacgcgcgcgcgcccccgcggtgcctgctgtactacgtgtacgagccctgcatctaccacccgcgcgcgcccgagtgcctgcgcccggtggacccggcgtgcagcttcacctcgccggcgcgcgcgcggctggtggcgcgccgcgcgtacgcctcgtgcagcccgctgctcggggaccggtggctgaccgcctgccccttcgacgccttcggcgaggaggtgcacacgaacgccaccgcggacgagtcggggctgtacgtgctcgtgatgacccacaacggccacgtcgccacctgggactacacgctcgtcgccaccgcggccgagtacgtcacggtcatcaaggagctgacggccccggcccgggccccgggcaccccgtggggccccggcggcggcgacgacgcgatctacgtggacggcgtcacgacgccggcgccgcccgcgcgcccgtggaacccgtacggccggacgacgcccgggcggctgtttgtgctggcgctgggctccttcgtgatgacgtgcgtcgtcgggggggccatctggctctgcgtgctgtgctcccggcgccgggcggcctcgcggccgttccgggtgccgacgcgggcgcggacgcacatgctctctccggtgtacaccagcctgcccacgcacgaggactactacgacggcgacgacgacgacgacgaggaggcgggcgtcatccgccggcggcccgcctcccccagcggagacagcggctacgaggggccgtacgcgagcctggaccccgaggacgagttcagcagcgacgaggacgacgggctgtacgtgcgccccgaggaggcgccccgctccggcttcgacgtctggttccgcgatccggagaaaccggaagtgacgaatggacccaactatggcgtgaccgccaaccgcctgttgatgtcccgccccgcttaaataccgggagaaccggtccgcccgcattccgacatgcccggcgccgcctccgtcgacatggacacgtttgaccccagcgcccccgtcccgacgagcgtctcgaacccggccgccgacgtcctgctggcccccaagggaccccgctccccgctgcgcccccaggacgactcggactgctactacagcgagagcgacaacgagacgcccagcgagttcctgcgccgcgtgggacgccggcaggcggcgcgtcggagacgccgccgctgcctgatgggcgtcgcgatcagcgccgccgcgctggtcatctgctcgctgtccgcgctactcgggggcatcgtcgccaggcacgtgtagcgagcgagcgagcgaacgggagcgggggcccgcccccatccgccgcgcccaggagaggggggagagagcggggggttgggcgcgccacgtggtgtgggcacggactcggacttgtcacaataaatgggccccggcgtgtccgggcgcacacagcagccttcctctcctccgcgtctctgttccgcccgtctctcgccggactcttcttctccaccgcctccaccgtcgcagttgtcgcgagcgcgttcgcaccatgggggtgacggccatcaccgtggtcacgctgatggacggggccgggcgcatccccgccttcgtgggcgaggcgcacccggacctgtggaaggtgctcaccgagtggtgctacgcgtcgatggtgcagcagcggcgcgccgccgacgagaactcgccgcggcagcacgtggtgctgcgctcctcggagatctcccccggctcgctggccctgctgccgcgcgccgtgcgccccgtcgtgcggacgcggtccgaccccacggcgccgttctacatcaccaccgagacgcacgagctgacgcggcgccccccggcggacggctcgaagcccggggagcccctcaggatcagcccacccccgcggctggacacggagtggtcgtccgtcctgaacgggatccagtacctgaactcgggggcccggggcacggcccccgtccacctgtggatcctgggcgccgccgacctctgcgaccaggtgctcctggccgcctcccgcagcaccgccgccggagcctcccacgcccagacgggcgcgcgcctgacccggcgccggcccgggctgacggacgccgacgccctggacgtgatcgtcgccgggatccaggcgacccgcgccatgttcgcgcgggtccacaaccgctcctggcgccacgccggcgagtggacggaggccctgcatgcccagatcgtgacccggggcgacgtgcgccggcgccgaggcgggcgcggcaacggacgcgagcgcgccccgcgatgtaccatctcctagacggcaggatctctccgcatcccccactccccccaaaaaaacaaacaataaacgctctcgctctggcacccgatgacacgcctccgtcctctctctccctcccccatctccccccttcccccccttcccccccccccgctgccctgacgtcactcccccttcccaccaatagccgccgaggactcccccggactcccccggactcccccggactcccccggactcccccggactcccccggactcccccggactcccccggactccccccccaccaacaactttaacaataaacgctctcgctctggcacccgatgacacgcctccgtcctttctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccaccccatcccactcccctctccctccaccgtctccacccccatctcccttccccacgagggccgggagggaaaaaacgcccgagagacgagagagttgaggttcgagcggcgggccgccgtgtttattgagggcacaacagaggggagggggaaggggaaaggggcaaacgacggggcggacggtgggggtggaggaagaacagagcggggcgcggcccgcctcacggggcggcggcgggccgcgtcggggcgcgcgtcccccggcgggcggcgtccttcccgccctcctcgccctctccgtcttcgtcttcttcgccgtcttcaccgtcttcccccccgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcgtccccgtcttcctcttcctcttcctcttcctcgtccccgccgtcgtccgagcccgagccctctccgtccgagccgtcctcggggggctcaaacacgtcgtcgtcgcccacgctggccccgtcaaagtcgtccccgtcctcctcgtcctcgtcctcacagagcccgtccccgtcctcgtcgtcctcgtcctcgtcctcctcgtacacgtccgtctccccgtcctcgtccccttcctcttcctcctcgtcttcttcctcttccccctcctcgtcctcgtcttcctcttccccgtcttcctcttcgtcctccccgtcgtcctcgcacaccgagccgcggaagctaaactcggacgcgacggtggtgtccgagccggcgtcgtcctcgtcgacgtcgcactcctccccaaagagcgggccctcgatgaccggcggggagaggacgcgcgggtcgtagacgtcgcgctcgttgagctcgcgcagcgcgctgcgcagcggggcgctctgctcgggcgagagctggagcagggcctgccagcagggggccgaggggcgctggcgcgtgtagcccatggcgtaaaagtcaaagagggcgcggcgcagggcgcgcgtgtcgcccgtcacgtcggcggccgagacggcgatgccgcgaaagaggcggttcacgtcggccccgaggcgccgcgtgtcggggtgccagggggccgcgcagaaggcgccggggccccgcgcgccgtggacgcgcacctcctcggggtcgggcgggggcccgtactcggggtgctcgacgacgtagcgcccgcgcttggggggcgtgggggtcgaggcgggggacggggggctgctccccgggaccggggtcgcgggccggggcccgggcgggtcgacgggcgagaacggcggggagggcacgggctcgtaccagtcggcccagacccggtccatgggcgcggcggacgcggtgggtcgggcggggtggtgggaacggggtggggtgggagggggtgcgatgggggtgtgcggggggctgggctgggacggggggccgggtgggtgggcgaggctccccgacgccgacccggaaccggacccgagcccggccgccccgacgggtgggtggctggatggattggatggattggatgggtggtcgaccagccggtcccgctcgcgtccctgtggggggggggagaaaacgcatgacgtcagcgtgagccacgaccccctccccttcccccgatccctcccgacccccgcccacctacccacccgacccgtcccggggagggggagggggaggggggagccgcacccgggaagccccccgactcacctaggcgacggacgcgcgacccccgaggcggttcttccggaacgatgcggcacctgaaacgagaacggatacgatgagcgcgatcgcgcggggtgggtagggaggggggacgggggaggaggtgccacgcccactcccggcccaccccgaccgggggatcccccgcatccggtaccgtcgggtcccgcgatcgtcgtcgtcgaggccgatgccgacgtcaccgtcgtcgtccgggcgtcctcgagcggggcgcggctcctcgtcggctcggggcgcgctccgcgcctctcccggtcgatccccgtcggcgtcgttggggcggcgagtgccgtcggccgggccgccacgcacccctttatcgccccgtcgagggggccgggccgacgggcccattggccggggcctcacacgtgggctcaggggacgcgggtccggctcccccattggccggcgcgtccccggaggcgggtgccgcggggttcgatcccttcggaaagcggtcgataaattgccaataggggcacgtggggcccgtggagagatttgattgggccacgtcacgttcgcaccaattgctaatatatactagtattatatcttatataccgcaagtgcgaacgctggacgcgtgccaagactcgaggagggggcggtccggcccgcgggacgggcgtccgggaccggccaatgcgggctccggggacgcgggtccggctcccccattggccggcgcgtccccggggcacccacccccgtggttcgatcccttcggaaagcggtcgataaattgccaataggggcacgtggggcccgtggagagatttgattgggccacgtcacgttcgcaccaattgctaatatatactagtattatatcttatatacaacaagtgcgaacgctggacgcgtgccaagactcgagacggcgggggcggtccggcccgcgggacgggcgtccgggaccggccaatgcgggctccggggacgcgggtccggctcccccattggccggcgcgtccccggggcacccacccccgtggttcgatcccttcggaaagcggtcgataaattgccaataggggcacgtggggcccgtggagagatttgattgggccacgtcacgttcgcaccaattgctaatatatactagtattatatcttatatacaacaagtgcgaacgctggacgcgtgccaagactcgagttggcggcgccgcgggccccgagacggtcgccgcgaccgtcccggaaatttccgggaccgcatcgcgggccgggaagtcgcgggagcgcgcgtgacccgccgcgacctctcccttcccggacgcccgctctcgaggaaaaaggggcgcggcggggcggggacgggtcaccgccgggtcggccgcgccccgggggacagggaatcccccgggacgggtgagtcaggagggtggggtgggagcggagaaagtgagtgtgggcgtgggaaaacaccggcgggagagggagggggagaggggggcggggagtgggggcgggatggggggaggggagagacgggaccgcgcggagaaggctcggtgcggccgaggggggcggcggggagaggggacgcggagggagcgggcgcgccgggagaggaaagggcgcgctggcctcgagccagccctccgcggcgatgaagacggatgagcggggcgatgaatggcctctggaaaaaggatgaatggggaggacggatcgcggcggctgcggccgcggcggcgggggctggtgggaggagtgggggaattgggggcgggggaaatggggggaggaggaaggggggcggggtgggggatcgcggcaccgagggtccggaggacgaagaggaaccgggaagaagaagaaggaaaaaaaaacactctctgtgtgtgcacacgggaccggggagagagaggagaggagagggaggggaggggtggtggtggtagagggcggtggcggtcactcgcctcaccccacccagccccctctccagttcccgctctcgctctctccttcgccaaccttctcccgctcgcgctccccgcccccctccttcccccaatccatccatccatctctcctggccgagctcggaccgcggcgatggtgaatgggcccgcggcctggacgagattgatgaatggagaggcgggaggggggaaggaggaggggggaagaggagtgagcgggggaaaaaaagggttgctggcggcggtagacggatgtagtgtacgaggcggcgagtacgccgagatgatggaacgagggcgagcctcttgttatgtgtgtgtgtgtgggaaagagagcgcgagagggcgggtgagagaggtcggggaggccggggccgaaatagaaagcggagaaagctctcagggcccggggttgcgccggcgggagagacgacgggcgccacagacagcggcgcgggggagggtgtgtgtatgtgtggtgtgtgttaaccacgaaagatgaggaggcggcggcggcgcgagggcgcgaaaaccccgacgcgcctcgatgcggcccgaatcgccgcgacgcgagtcgacggggagaggaaggaggggaggacggagggcgagccgccgctccccccaccccgccccgccccgtccctctcgcccacgcgccctctccccccttcgcgcacgctcactcgcccgcacccccgcccccctctcgttccgtgccgcgcgcgccgtcccgcggccccgtcgggcgcagggatcgtagcaaacgcgggcggtcggcgtgcgcgctgtgcgggtgggtgggggagggggttgatgaccgcgctggttccgggaagcccccccccggcgggcggagggggtcggggccgggttcgctctgtaccctctcacgcgggcccggagtgccgcgggcggggcgggggcagcactcagcgaaggctcagggtgttccagccgatggccccgggggcacgcgggcgggaggagttgcgtgggcggagaggagcgaggaggactggcggcggtggaggagggggaagaggcggcgagcggagcggcgcggtagcgcccgcgggtggcccgcgcggtatcgcccgcgcggtatcgcggcggcccggggagagcgggcgggggacggggacggggacggggacggggaggaggggggaggaagggggtcgccaccgcggccctcgcggcccgggagaagccttcccggacgcgccattagagagggctctgtctgcagaaacggtgtctaggcgccgcgccaggggaaaccgcagcggcgataaaatagcttttagctcggcgctctctctgggtcgcagcgccctaaacttggcacggtccgccgcacagtgtggcccgcgtccaggccgggcccccggcctgccggcctggcgagcgagcgagcgagcgagccggggccccccagctcgctccaccgtgacacccacccacaccgtaaactctcacacggacacgcgcgcccaccccccggcgccgcgggcccgaccccggtccgcgccggcacccccttttctccggcaccgagccggggggccggggcgaggggaaggggaagggggggggggacggggtgcgcgcgtgtgtgtatgtgtatgtggcaccggcatcggtctccccccaaaacggagaaggcgagggagacggagagggaggtgtgtctctgtgttgttgtttttttttcctgctggcgccgcggtgcgcgaacgtgatcacggggaggcggcgcgctccgcgctcccccgccgcctgggcggggatgccgcgccggcacgagcgcacgcgggcgcgcgggcggcgggaggggagggaggaggaagggccgggccgccgagccggccgcgccattgggcgccgggcggaacggggcgtggagaggcgcccgcgccggggacgcgcctgcggcggcgggcgcgcgccgggcggctctcctccttcaccgccgccgcgtcctcctcctcctccctccccgccggcctcggcccacccgcggcgtcaccccgctctccccccccctcccttccccccgaccccctcccctccctcccctcccctcccgcgtcctgcgtcacggggaccggggggtcccgcgtgatgacgccggggacgcgggacgcgcccgccgcctgggagccggcgaggaggacaaaaagagttctgtgtcccctcgcccccctccgcgtccgcagccgcctctgcagccgccgcccgcgccgcgaccggcgaacgcagagagaggacgcggggaagagacgcggagagggggacggggggagagagagcgaggggcgcgagcgcttcctcctccacacccgccttcctccgcccgcctttccttcccggactcgggagccgctcccgctccccccgtttttcctcctggccgcggcgaccgccgccgccgctctcgaggttttttttcccttcctctccttcttcctcctcctccgcccttgggtgtttccttctccgcgtccccatctccggggcccgaggcgccggggctttcttcccgggccgccggggaggaagcgccccggcttctgcttcttcttctcctctctcggtaagcacagactctcctactactactactcctactactcctactcctactatattactatgactactactatgactactacggtgacttctcctccccgctctagactttttttcctcccctctcctcctttcgctcccgtctcccgggcgcccgcggccgggcggagggggaagcgggaaggggaagcgcggggcgccgcgcctctcccccccgcttccctccggcgggcccttccccgtttccccgtttccctttcccccccccttccccgtctgttccttccccccccgcttccccgtttcttcttctcccccccgtcccctcttccttccccacccgccccgctctcccacgccgcggccgcggggggccgaagcggacgcggaagaagacgcggaagaagagagagagagagaaagaggggggccggcggggatggggaagaaggcccgacccgcgcgggggcccccctcctgtcgcctgtcgtttgaatgaaaccttcccccgagcgaccccgctcccgcctgtggtgccgtgttttttcccgcgacggggagagcgcgagagtgaggctgttggctgtgggttgttggctgttggctgttggctgttggctgaggacgacggggaggggggatgctgccgcgggtggcatgagacggagggggcgagggcgggcggtggaggggggagaggaaggtctgggcgcgcgtgccgcgggccgagtgtcgggcgagtgcggggcgggttggggacggggaggaggggaggtgtggggggatggccggccggcgcccccggagcacgcggggcgccccggcgggcgggagggggtccgggggaagggtggggcggtggcggcggccatcgcccgacccttccccggggcgcgcgcgtgggaccgggaccgggacagggggcgggggcggggagaggaggagaaggaggagaaggaggagagtcggaaagtcgggcggaggacagaggcggagagggcgagcgagacacgcacggggagaggcgcgcgcacacacgcggagagagagaggagaggcgagagaggaggagaaggaggaggcgaactgggggaaaggcgagacacgcccgggcgcccccctcctcggaccgaggcgacggcggagacggagacggagagaccgcccgcccggccaccaccgtcggtaccggtatcaccagcaacacccgcaccaccgacacccacacggaccctcccacccgcacgggcccccatacccgcaccggccctcacacccacccacacgcacgctctctctctcgcacacccccacatcacactctccccgcgcacagacacacggacgacaccctccccgcccgccccgtccgaccgaccatccgtccgcccaaccctccacgagagagagcgagccttccctacacacacacacacacacagcaaaaattgaccccccccgtcccggaccccccggggggcgggggcctcgcgcctccgcgtccggcgggcgcgaccgaggcccgcgcgcccccgggggctcggggcgggacgcggcgcccgcgcggggacactctttcccccggaggggcgtcccggcgcgccggcccggccctccccgccgggaagggggcccggccctcttcgtctcccggagcgcccccggggtcccggcccgaggtccattttgggccccgcccctttgcacccgcccccccaccgggcggggtccgcgctccgacccccccctcccctctgccacgccccgccacgccccgccgccatcttggctcccctcccccccaactccctctctcccgacgaggccgagccggtctcgcgccccctcccgcccgcccgaggtaccgcggaaggacgacacgagccggaagacggtgcaacgagaacaacttttatttttttcactctggggcgtgtctcgtggggtgggaggggggaccgggagagagaggtagagggggggtgggtgggcgacaaaagtatccaaggcgtggctgtccggctcctcccttccgggtccgggcgggtccgggctcgaggttcgggtccggttctcccgccccggcgtcgcttcccggttcgggtccggtcgccggtccccggtccgacggcggttccgctcgtctctccgaccggctgggggcgccctcggagcgcgcgccgagcgcgcaggcgcgggtggggcgaccgcgggggtcgcggggggcctcaagggtctcggggagggggaaacgggggtctcgggggtctcgcgagtggcagggggagacggtgagaccaccacacaacacagagaccaccacaaaacactgagagaccaccacacaacacagagaccaccacaaaacactgagagaccaccacacaacacagagaccaccacaaaacactgagagaccaccacacaacacagagaccaccacaaaacactgagagaccaccacacaacacagagaccaccacaaaacactgagagaccaccacaacacatgcggaccacgtgcagacacacggaagcttgcaccgtgaggccccattggccgagggcggccacgtggggcgagcatgcaaatgacgtgcacacggaagcttccccgaaaatcatctgattggctcgctagcaccacgggggtgggtcgcgtgccgagcatgcgcctcgcgcccgcgcgagggattttcggacaatctcattggccgcccgcggccaagatggccgcagagcgggccgggcatgcaaatcagaggcgcgcgggagacgcctccgcgcgcccattggcccgggcgagccgagatggccgccgcgggggccgggcatgcaaagtagacgcgagaggaagtagggagagaaatcccattggccgtcgaggggccaagatggcgccctcggggccgggcatgcaaagtagacgcgagaggaagcgggcgagagaaatcccattggccgtcgagtgggcaagatggccgccgcgggggccgggcatgcaaatggtcctcgcgaggaagttcctcgcgaaatcccattggccggcggccgccatcttgggccgggcatgcaaagcagacgcgagaggaagcgggcgagaaaaatcccattggccggccgtcggggaagtccgcggcgaaaatcggccattggtccgcttacctgggggcgggctcttctcggggagtttataggcacggtctccatcgtagcacttcactgcggtgcaggtacggacagcatcgttctctgccaacccgaggggatccgatcgtctcggctccggcgcggactctgaagactccggctctccggcggctatcagccctcgacggacggcccgacccaccgaggctctcggcccgccagagaagagtcttcttcttctcctcctccggccgccttcctccttcttctccgccgcccgctctccgcgctcggcgcccggcctcgctcaggcagaaagaccccgatcgagaccatggccgacgatctctttgacttcatcgagaccgagggcaacttcagccagctcctggcggcggcggccgcggccgcggcggccgaggaagaggggaccgcggccggctccgacggcggcagccagggctcccggcgccgcggcccctccggcgaggatctcctctttggccccggcggcctcttctccgacgatgccggcgaggccgaggccgaggccgccgtcctcgcggccgcggccggagccacgaggccgccccgtcctccttcggcccagcagcagcagcagcagcagccccgccggggctcgggcgagatcgtggtgctagacgacgaagacgaggaggaggacgagcccgggtccccggccgccgggtcccccggtcgggctctccatcagggctccgagcacggtcacctcgtcctcgggccccggtcccgggccggctccgggccccggccgccgaccccggcagcactcgcagcggcagaggccggggcccccggcggccccgggcgcccgtccccctccgcagcctccccggcctcctcctccggctcccccggcccctcggcagccccgcggagatggtcccccgcgaggggggacccggtcggtgagcccgggccggcggcgcggcctcggaccccggcgccaccagcacagccagcagcggtggccgcagcgccggcacggcgggggcccgcttccccagcctcccccgccgccgggccggtctcggcgctcggcggcggcggcgccccctccgccggaggggaccgcggtcgtcaccatcaccagcaccgcgagcccctgctcgacgagcccgccgccgcgaggcggctcgatcctcggccgcttggcgcccggagccccgtctcctccaactccaactccaactccagctcctccaccaccgtcgccgtcgagcccgtcgcccgcggccccgagaaggacgaggacggactcggcctcgccggggacggcggggcgcccccgcagagacagccccgccggcgccgcgccggagagggcgccctccgccgcggccgcggcttcagcagctccagcagcagcggcagcgactcggacctctccccggcccgctcgccctccgccccccgggcctccgccgccgccgccgcgcggcgctcggcctcctcctcgtcctcgtcctcctcgtcctcctcgtcctcgtcctcctccgagggagaagaagacgaaggagtccgggccggcgccccactcgcccgcgccgggccgccaccgtctccgccggcgcccgccgcggccccgcggccctcggcctcctccgccgccgcctcctcctcctcctcctccgccgcggcctccccggccccggccccggagcccgcccggcccccgcgcaggaagcgccgccccaccaacaaccacctctcgctcatggccgacgggcccccgccgaccgacgggccgctgctcaccccgctcggggagccctggcccggctccgaccctccggccgacggccgcgtccgctacggcggcgccggggactcccgcgaggggctctgggacgaggacgacgtccgccaggccgcggcccgctaccgcgccgcggccgggcccgtgcccgtcttcatcccggagatgggggactcgaggaagcagcacgaggccctcgtgcgcctcatctacagcggggccgccggcgaggccatgtcctggctgcagaacccgcggatgcaggcccccgaccagcgcttcaaccagttctgccagcgccgggtccacgcgccccacggccacggctccttcatcaccggcagcgtgaccccgccgctgccgcacatcggggacgccatggccgcccaggacccgctctgggccctgccgcacgcggtgagcgccgtggccatgagccgccgctacgatcgcacccagaagaccttcatcctgcagagcctccgccgggcctacgccgacatggcctacccgggccgcgccgcggacccccgcgccggggaggcgaccgtcgaggccctctgcgcccgcgtccgcgccgccttcgccgccgcgcagcccggccgcgtgccccgcgagctggcggacgcctgcgtcctggcctgccgcggcgtcctcgagcgcctgctgcccgtcccgctccggctgcccgcgcccgcccgcgccccggccgccctcgggcccgcctgcctcgaggaggtgaccgccgcgctgctcgcgctccgcgacgcgatccccggggccggcccggccgagcggcagcaggccgcggactctgtcgccctcgtcgcccgcaccgtggcgccgctggtgcgctacagcgtggacggggcccgcgcccgcgaggccgcctggacctacgccgcggccctcttcgccccggccaacgtggccgcggcccggctcgccgaggccgccgcgcggcccgggcccgcggagcccgcgcccggcctgcccccgctctggcccgagcagcccggcctcgtcgtcccagcgccggcgccggcggcggcgggcgccccctccggcctccccggctccgggccctccagccccgccagcaccaagtccagctccagcaccaagtccagctccggcaccaagtccggcagccccggcaccaagtccggcctctccggctcctccggctacgccagctcccccgccgccggcccggacccagcgcccgagcggcgcaagaagaagcgccgcgcgcccggggcccgccgccccggggacggcgaggaggacgaggggctctccggctcggccctccgcggggacggccacggccaccgcgacgacgaagaggaccgggggccccggcgcaagcgccgctccctcggactcgggcccgctccggaccccgctcccgcgctgctgtcctcctcctcctcctcctccgaggacgaccggctccggcgcccgctcgggcccatgcccgagcaccccgcgcccgacggcggcttccgccgcgtcccggccggcgagacccacacgccgcggccctccgaggcggccctcgccgcctactgcccgcccgaggtggcccgcgtcctcgtggaccaggaggtcttccccgaactctggcgcccggcgctcaccttcgacccggccgccctggcgcacatcgccgcgcgccgcggggccgcgggcgccccgctccgccgccgcgccgcctggatgcggcagatagccgaccccgaggacgtgcgcgtggtggtgctctacgacccgctgccccacgaggagctctgcgccgagcccgccgagggcgccccgcgcccggcctgggacccgcgccgcggcggcctctcggcgctgctcgccgccttcgcccaccgcctctgcacgccggactcgcacgcctgggccgggaactggaccgggcgccccgacatcggccgcctcaacgcccagggggtgctgctgctctcggcgcgggacctcggcttcgccggcgccgtcgagtacctctgctcgcggctcggcgcggcgcggcgccggctcatcgtgctggacaccatcgaggactggcccgcggacggcccggccgtgggggactaccacgtctacgtccgcgcccgcctggacccggccgcccagtgcgccgtgcgctggcccgagtgccgcgagctgcgcgcggccgtcctggactcgagctccatcgtgggcccggcctgcttcgcccgcgtggaggcctccttcgcccgcctgcaccccggggccgagccgctgcgcctctgccgccaggacaacgtgcgctacacggtgagcacccgcgccgggccccggaccccggtcccgctgccgccgcgcgcctaccgccagcgcgtgctccccaccgtggacggctgcaaggacatggcccgccagcgctccgcgctcgggctcggggacccggactttgacgcgggcgccgccttcgggcaccgcgcggccaaccgctgggggctcggcgccccgctgcggcccgtcttcgtctcctgcggtcgccggggcctcgccgagctccgcggccccgagggcctgccggccgagctgcgcgccttctgcgccgcggcgctgctggagcccgacgccgaggccgccccgctggtgctggccccgggcgccctcgcggccgcgggcgccccgccggccgtgggctgggactttgcgcccttcgagaccagcgtccgcgccgccggcggcgccgtggagacccaccggccctcgggcgcccgggggcccggcgaggacggggaggactctgccgcggtggagatcgtcggcttccgcggcggcgacggccggccccgcggcccgctcgggcccatcaaggtggaggccatctcggacgacgaggaggccgaggacgccggcaacccctacctgctgctccgctgagcggggcgccccctcggcccggctggactctgactctgactctccggcccctccaccggctcctcgaggcccttctcctccgtcttctctcccctcgcccccggcccggtcctcgtcctcgtccccgtcctcctcctcctcctcctcctcctccgcgtccgcggcggcggcctcggtcccctcggcccggcggcgcttgcctccccggcgcctgcctccccggcccggcggccctcctcctcctcctcctctccttctcctccgcgcggatcccccggccggaggcggctgcggcggcggcggaggtggcggcggtggtggaagcggcggcggcggccgcggcggagggctcggcggcggaggatcgtcccggtccccttctcctcctcctcctcctcccgcggtcccccggtccccttctcctccttctcccatcgggtgagaaaagagtttattttcagagtgagaaaataaagtttgtgctgtattttctgaaccagctcgagtctctgagattttttggggagatggaggcggccatcttggcggtggtctctggggcggaggtggtcttgtggatgggggtccctggtgggaggaagaagaagaggtggagggtcttggtgggggtgatgggggtcctcctcctggagggtcttggtggtggtgatgggaagaagtggatgggggtcctcctcctggagggtcttggtggtggtggtggtgggtctcagcagatgagggtcccggtgggtcctggtgggtctcagcagatgggggtcctcctcctggtgggtctgggtgggaagaagtagagggtcttggggatgttgggggtccttgatggtggtggtggtgggaggtggacggtgttggtggtcccggcgggtcctggtgggaggtagatggtcccgagggtcccggtggtcccgggtgggagttagacgatggtggtcctgcggtggtcgaggggggggggaaggaaagagagagagagaaaaaaacacggtggtggtcagtacagggaactgccattgcaaccatcgccccctggtggtgatagtagtaaagtgtgtgcgtggggggggggtggtggtgttgttggtggtggtggtcccgggtggtcgtcgtggaggggagggaaaggcggtgggagagcgggcggaacaggttcatctggcttccgtcttccgtccccactcccagcctcccccgctgccacccgattggccggcgggacgatgacgacggcgaccgcgtggcgcgaggggagccaatggggcgggtgccgcggagcggaagcccccgcccgcggacccccggtgcgacggaaggggcaggctcggcgcgccgatgcgatccgcggagcccgccccgtccgcgccccggaagccggcgtcacgtccgggggaagcggaaggggccggcgcgatatgcagatgagatccgtggggaccggcgtcacgtccggcggaagttgcgcgcgggaagcggtcatacgcaaatgaggcggaagtcgtcatcggcaaaaacgtcacgtccgggaagcggtcatatgcaaatgggggggggggtgggaggagcccggcgtcacgtccgggggaagcggtcatatgtaaatgaggggggaggggccagagcgtcacttccggaggaagttgtcatatgcagattagggggaggggttatgcagattagggggaggggttatgcagattagggtagattagggggaggggttatgcagattagggggaggggttatgcagattagggggagggcgttatgcagattagggggaggggttatgcagattagggggaggggttatgcaaatgcatgcaaatgagtgggaggggttatgtaaatgagggggcggggccatgcaaattgggggaggggccatgctaatttatgcaaatgggggggaggggccatgctaatttatgcaaatgagggggcggggctatgcaaatgcatgcaaatttatgcaaattagccccccccctttaagccccgcccccttttttcgcggcccccggggagcgggggggtggg

SEQ ID NO: 3 - DNA, 138015 bp, Pseudorabies virus
ccgggcgccacctcttttcccccaccccctccccccaaaaaaactgggcccagctctcccccgagcgcggatctgtgaaaaaaaattttccctcccccgcgttttccattggggtgaatggggagggggtgggggatgggcactcactgagaattggcaaaatgccactttataaccttgcactttgccaccaacccacaatccccctcctaccttggcaccctgccaaccccaatccccctcctaccttggcaccctgccaaccccaatccccgccccggtcctcccatcgcttttggcacggcgccaaccccgatcccccgccatcgccttttcccagtccttggctcgctgccaaccctagagggcgagacgcggtgctgcggtcgggcccacgggcggcgggtggttggagagggcgatgggcagggacgggcgcgcttgtggtggggacggtgtcggtgcccgcgacggtggcgcccgagagggttcgccttggatgcgatgggggccctccccattcaccccaatggaaaacgcgggggagggaaaattttttttcacagatccgcgctcgggggagagctgggcccagtttttttggggggagggggtgggggaaaagagctgggcccggctctgggtgggggacaacccccgagggagagtgtggccggtggccagactagaggagagccccgccgggaccggtgaggagagagggcctttccccccagccccctccgctcccccctttccccccgttctaagtggacgccgcgggcgcgcgggcccccacacggcgttcgctctcgtcccggccggtcgggccgccgggacggggcgctcggcgccgcgatgaccgacgcctcgtcgcggcccggcggggaagagccgatggaggtggacgagcacgtcgggccggggcccgagcccatggaggtggacgagccccccgcaccgccgtcggcaccggcaccgacggcggcgggcaccatcctcgcggtgggcttcacccagcccgcgcaggtcctgcgggtgtacccgatcggttgaggtgcgaacgatgggcgggggatgcccggggtcggacgcgctcacgtggtgccccacccaccccacgccgcgctctttttcccgggccgcccgaccggatcctcccgcagaggcggaggtgaggccggagagagacagggagagatgatggacgcggagaggggagcggccgccgcggggcgcgtggaggtggagctcgatgcccggccctccggcgccggagggcgcggcgcgagcccctcgcgccgggcgcgggacccccgaaccagcccgtgctcccggggccgagccgaccggcgttcccgggacagaaccggcgacgtcaccgggccgcgggcagcgcggcccgccaccctgggtattaacgtcccgggcaccgccgtccgccgccagccgccgtgggaggcagacatgcctccacaacgagcccgcggggctccgccgcgccgtcgcggcagcgacccgcccgatccaggcagcctcgccgggcggccctcgcccggggggagaggaggcggagggcagcgccgccccctctcgcgcagcagctcgctcacgtccgtcgcctcggcgcccgtggagacgcccgtcgtcgcggaggctccggggctcggcgcccccggctccaggccgccctcctacggggacgtcgtccgcgtcgggccgcgcccgcaccgatcgccggacacgccgctgtttgcccggggcccgcccccgtcctactcggagacgctcctgttcgacccgcccgcgtacgcggtgaccatcccggacccgccggcgtacgagcccaccgtcatcgggccgcacccgccgcgcccccgcgactggatctcctcgccctcggtggtgcagccgtcgctgctgggccccttcagccagtgcctcccgcgggtgacctgccccgactgccgctaccccgaagaccgcccgatggtgctcgtgggcttcctctgggggggactgctcctgctggtgggcctcgtgtttctgatcctgctcccggtgctccgggagtccgtcgtgtttccctgatcaataaactcggctcgcccgccccatcgatattcgcgtctcgtgtggtgctgcgacgaggggggaagaggtgggacgcgaagagatgaagggatcggggggaaagacggcgagaggaggagcgggagtgtgcggaagggacggtgaagcggcggtggtggagggagatggggagatggagggagatggggagatggagggagatggggagatggagggagatggggagatggagggagatggggagatggagggagatggagggagatggatgggggggggggggggggggggggggggggggggggggggaggggagaggggaggggggggagatggagggagatggagggagatggagggagatggagggagatggagggagatggagggagatggggagcgacgacgacagactcgcgcacgccagagaggtacggttcaacggttttattcaaaacaggtggttgcagtaaaagtacttcccgtgcatgtacacggggacgagggtgtagctgcacgtcaggcggcaggtgggggcgtcgcagccggtcttgtgggcgtccagcgcgtccatgatcccgccgaggcaggcgccgggcacgtactcctccagcggggcgggcgagtcccccaggtcctcgggcagcacgcagctcgagccgcgctcgacgacgcgggcgatgcgggccagcatcaccagggtgtaggtgacgatgcagcgcacgtcccgcaggcggcgccggcgcacgagctcgtcgacgggcgtgtcgcggaggtagcagcgcaggaacgggccgagcttcaggcgcaggttctggagcaccgcccccgcggtggccacgatggggtccagcgtgcgcagcgtgagccccttggtgaggatgagcttgacccacgtgagcgtctcgtccgccgaggcgagggactccgtcaggccctcgttcaggagcaccatctcgcggagcacgcgcgccgcctcggtggcgtgcggccgcacctcaaacaggcggtgcaggtcggccccgtggaacatgagcgtctcccacgtgacgcgccgcccgtccggggtgaactgctcggcgccgaactcgaggacggcggcccacggcgacgagcccgcggcgcggaagccgtcgttgaacacggggcgggagaggtcgcgccgcgccgaggcgaagagggcgttgacgcgcgtcgcgccgacccgcgcgtgcgtcgcggagacggtggcgcgcgcgccctccgacaggcggtcggggggcgcgtcgggcccgctgtcgggacgatccgcctccggccgccgccgctggtgctgctgctggcgctgctgctgctgttgctgtcgctgccgctgtcggagggcctgtcgggcggcgcgcccgccgcgacccccgcgccggcgccggacggccccgagccgcgcccggacgggcggccgctcggaggggcgcgcgcccaggcgctcgcggaccgggcgccgctcctcgctccccgaccgggacgcctcgctgccgctgctgttgccgctgtcctccatgaccgtggtgcgagcggggccgcggtgtgccgccggcgggcggcggggcgcctttaaccccgcgcccccgcgcgaccttcatccaaatatggtaatgtggcgctcgtagtgcagaaacagcaacacgccccctaccagcaggaggaggtacagcgccttggcaaagatcccggcgagcacggtcgagcagcaggcgccgcagacgccgagggcggcgcgcccgccgtcggcgcgcgcgggcggggccgtcgcgccggagacgcgcgcgatgtgcggcagcgtcgccgccaggagctccagcgccccgaccgccagcacaaagagccacacgagggcgcgggcgtacagcgggaagacgagggcgcacggggcgcgcgtgacgccgagggtcgtgaacagcgccatgcgcgcccccaggcggagcccgatctcgaggaggatgagcgcgatcagcgtggggtggcggtgcgccagcgtgatggggtcctcgcggaggtcgcggctgagcgcggcgcggcgcgtcgcgagctcggccatgtaccaggcgaacttggtgtagctgcagccgatgaccgtggcggcgagcacgctcgccgcgtagttgagcgtgtacttctccggggtcaggaactcggcggggtcgcggaaggggccgaacatgcgccgctcctggcgcgcgtacacgaaggccgcgtagaggagccacgcgaggaccgccaggcgaaagtgcacgtcccacaggtaggcgcggcagtcgcggcgcgcgtacacgacccgcgcgcggtcgcgcagctccgggctcagcgcgcccgtctcgttggggcccacgtagatggccgtggcgttgaaggcctcccagtccggcgcgccgccgccggcgtgctcgccgagcggcagcgccgcgtacacgacggggtggggcaggcgggcgacggtcgcgtagtacgcgcccaggccggcgtacgcgcccatcaccccgaggacgaggaggtgcagcgggcgtccgccgaggagcatgattgccccacctccgcggtgaaaatggtgcgcgtcgcgttgctgccgcactttgtagcgaagcactgctgactcagcgagatgcacagccggtcgtgggcgtcgacgctcagggccacgaaggtgcgggccggcgggctgcgcgcgtgccgggcccgcaggcagctgaagcgcgcgggcccgcagagctggaagagcacccagtcgggcttggccaccacccgctgcgcggtcgcgcccgcgtactcgtgcgcgcgcccgttgaagtgcgcgtcgaggtgccgcagcacgggcgccatggccacgtccgccacgaaggcctcgagcgcgcgccggtcctccgcgtccacggccaggccgcgcagcatgtccgccagcggcgtgcgccgggcgaagaaggcctcgcggttccgcgccgccttgcgctcgaagaagctctcgtactccccgccgaggctgtgcagcacgcgcgtgacggcgtgcgccgggcgggcgtggaagtgaaagttgcgcgggtcgtcgaagcccgggggcgcgtccccgaaggggagcaggcgcccgtggcagccgccgccgtcgaccttggcgaagaagggcagccgcaggctgcgcccgtgcgcgtacacgcccgtgtccacgaaggtgaagtcctggaggtacgggcccatgggctcgataaagtcccgctccagcagcacggcctgctggagcacacgcgccaggccgcggaccgtgtcggcgccggccaccgcgtacggcgacggcacgggcgtgcacacgcggaagcccatcttggcgcggcagccgcacgccgggcgctcgtccgccgcgggggcgcgcgcggggccgggttcggggtcgggggcggcggcggaggaggagccggggatcccggccccgacctcgtcgtcgtcgcacgtgtacacctcggcgggggcgcccgcgtcccagtccgcccagtcctcgccgtccggcgcggcgccaaagtcggcgggggcggcgacgtcgtcgtcccagggcgccccctcgaggccgtcgtcggaggaggccgcggcggcggaccccgcggcgccggcgcccccgggcggacaggcgctcttgtacacgtagcacgggtgcgcggcccagtcgccgtcggcctcggggaagagcagcgccagcgcgtcgagcaccgcgcggcggaacccgcgcatggccgcctcgagcgtgcccgcgggcaccgggcgcgcgaggcgaaagtcgacgtcgaggacgatgttggtcacggccagccgggcgttgaacacctcgtggcggcgcacgtacatctgcgccgccggctcgtgcgccgccatctccggcgccgcgtcgcgccgcgccgtcgcgcgcccccaggcgtccccggccagcaccgcgaaggcctggcgccggtgcgccagctccacgcggtacaccggcgccggggggcgcgcgtccggcaggccgagcagggcgcccagcgccgtgcgcgcgcgcccgccgcccggcgcggcggccagctccagcaggcggcgcaggacggtgccgtcggcgcccggctgctgctgctgcgagtgcgcctgctcctcgtcgggggcgcgcacccagccgtggggcgcgaggcgctcctcgacggcggtcacgtggcgcgcgaggccggcggccgcgtcgcagagcccgcgcggcgcgtcggccgcgagcgcggcgtacgtgcggtcctccacgtagccggcgcccacggccggggcgaggcgcgcgacgctcggcgtcacgttctgggcgacgtagtcgcggatgttgagctgcgcccgcacgtggcggaagaaggtggcggccgcgcgctccccgaggggcgagcggcccacgggcgccgcggggtccgtgacgttgacggcgtccaggtgctcgcggagccgcgcgcggttgaagctctcaaagtgggccaggtagacgtacgtcacgaactcgcggtcggggagcttgaggctgccgcggtcgtgggcgatgtagtcccggaggacgcccatctcggcgcggtcggcgcgcacgcgctcgtcgacgtagcgcgcggcgcgcgccgccgcgggcccccgcgcgtagccgctcaggcagcagaagcgcgagagcgccgcgaacgagagcagccccgcggggtccaggccgctggggttgggccccacgcgcgggttgtacgtgagcagcaggtccttgagggcccgcaggtcgtaggccggccgcgcgtggaagaggtggaggcgcgtggccagcaccagcgtcttctcctcgggcccgaagcgcgccacgtaccagaagggcgtgttgtggttcccgtagaggcgccggaaggcggccagcgtctgggtctcgtggtgcacaaagagcgaggtcagcccgcggcgcccgaccgcgatggagcgcagcgggcgctccggcgcgtactcggcgttgccggtgcgcgggcgcatctgctcgaggaccatggtcagggccgcgatgaggccgtcgtggagcgcgaaggtggccgcctcgtccatggcctcgaggacgtcccgcgccgcgaggggctccccgcggcagagcgcgcggacgaccgccggggtcgcggcggcgacgaacacgggccgcacgcggacgctggccacgtgcccggtgaggcagaacgtgacgccggggcggcgcgctaaaagctcggcctcggcgcgctcgcccggcagtgagaaggcctcgtccagcccggcgccgtcccacgcgtacgagacgacgtagaccgcccccgagggctcctggcccgtgagcagcatcagcgagtacgtgatcgcgcagccgtccgtggcgtacaggacgcggatcatgctgggcggcatcttctccgggtggtgcgggcgccgcggccgtgggagggcttatgagccgccggacgaactccgcctccgggagtcgctggccgtgctcaacgtgatcctccccgccccgctcgccgccgaggacgtgatggcctcggccgaggccgcgcgccgcctggcccgcgcggacacgctggcgcgcacctaccaggcctgccagcgcaacctcgagtgcctggcgcgccacgaggcctccggcgacgacggcctggacgccgtggtcgcggcgcacgcggccaacgcgcggcggctcgcggacacgtgcctggcggcgctcatgcccctgtacccctcggtcggcgccgtcgccgcggacacggacgacctcgtcgagcaggccctgcgcatgaccgccgagagcaacgtggtgatgtccgacgtggccgtcctggagcggacgctgggactggcccggcggcggcagccggcgcccgtccgggccgcgcccgccggggtgggcctgcccgcccccgtcccgcccgccccgcgcgtcgccgccccgccggccccgcagaccccgcccgaggaggaggcggatgcggaggcggaggaagacgaggctggcggggaccgccccgaggaggacgcggtcccgctgctgcccccgcgcgcccccgtcaagacgcgcccgcccgagaagaagaaggtccggaggctggccgaggcctgcaagatgtagacggagagcggggggagagagagggcacgcgcgaagagaagagacgaggggcgaccgagaagaaggggacggagaccaccgggtgtttttttttcttttcaataaacatctttattgaaaaaaagagaagcgtcctgcgtgtgttgtgtgcgcgcgcccgggtgccatcgccatcgtcccggcgcgggccaccgcggcctcacaggcccgtggagccgaagccgcgggtcccgcgcgcgctgctcggggcgacaaagtcccgcgtgaagagccacgcggggcggtgctgcatcggggtgcgcggggtggcggggaacggggaccgccccgtgatccagctcagcggctcgcgcgtcagcaccagctgcgccacgcgctggcccgcctccagggtcaccgggtgcgcgccgcggttctggatccggaagcggcacgggccgctctcccagggcgtggggaagacgacgatgccgcggaggttgagggacgagcgcccgaagacgtaggcccagtggcggccgtccgtgcggtgcaccggcagcgccacggtctccgcgccgccggggggcagcaccagctcgcgcgggcaggggatgtcgtatccggcgtcctcgtcgcgcttcggcgcaaagacctcgaagaagggcacgtccgtggcgatgcccggcgcgaggcgcagcggcgtgcaataaaactgcgcccgtgggcggcccggggctacaatcgcctgcacctcgccgcgaaagcccgcgtccacgatcccgttggtggtgcgaccgcccccgcgctgcgcgacgagcatcgcgtatccggtgggcagcgccaccttgaggcccaggggaaccttataaaatccctccggccccggactccggcacaccagcagccgccctccgtccactgtcacgggctcgctcgcagacaccaggatggtctcttcctccgcccggctctcgctgacgctcgtcgccgcgctctgcgcgctggtgacgcccgcgctctcttccatagtcttttccgaggggtcgctgccgctgctgcgcgaggagtcgcggaccagcttctggagcgccacctgcgccgccaggggagtcccggtggatcagccgacggccgccgccgtgacattttatatctgcctcctggcggtgctcgtcgtcgccctgggttacgccacccggacgtgcgcccggatgctgcacacctcccccgcggggaggcgcgtataaagatcgatccgcacgcgcccgatcccactcgctcgccatgtccagctcgagaaagacccgggtcgccgccgacgagaccgcctcgggggcgcgccgccgcgccggcagcgcctctcgcgcccgggccgccgccccggccgtcccgaccgccgcgacccccagacgcccctcggcctacgacgaggacgccggcttctcctaccggtctgccccgtcctacgacgacgatgactactacggctacgatggccacggctcctcccacgccccccgcgccgccaaggtgacgcccgcggccgcctcgcgggcctcgaccggggccaagagcgcctcggccgccaagacccccgcggccaagaccgcccgctcggcctcggccgccgccccggccgccgccgccaccaccaccgccgccgccgcggaaccggccgcccggcgcgcctcgacgcgggccgcccccggggagaacctcgacgtgggccgccgccggctcgccttcagcgacaggccgtgcgaggccaacgtgccctggcgcggcgcgacgcacgccttcaacaagcgcatcttctgcgcggccgtggggcgcgtggccgaggcccacgcccgcgccgccgccgagtccctctgggacatgaaccccccgacgacggacgcggcgctcgaccgcttcctgcaggccgcggtggtgcgcatcaccgtgtgcgaggggctggacctgatcgaggcggccaacgccgtcctggacgagagcaccccggggcggaagggaaaagtgtataaataaaggcgttgcaaatggcaccccgctcagtcgtccgctgtcttgcttgtgggggggtcggtgaggatgcgcgacgaggagtgcgtggtcgcgttcgacgaggccctgctggggggcgcggccccgggccggccgcccgtggtggccgtcccgcgggcggccagcccggacgcgctgtaccagcgcctcatcagcgacctggccttcgacgagggcccggcgctgctgagcgccatggagcgctggaacgaggacctgttctcgtgcctgcccggcaacgaggacctgtacgcgcgcatggtcatgctctcggcctccgcggacgaggtggcggcccaggtccaggcgccgacggcggacgcgagcgtcgacctcggggtgcccggggcggagcccatgccgcgggtcccggccgtggaggaggagctgcccgcgtacgtggcggccgtggagcgcttcttcgtgtccacgctgcgggcgcgcgaggagcggtacgcgcgcctgctggacgcctactgccgcgcggtgctgcgctacctgcacgcgcaggcgcgccgcgacgagggccggctccggcagctggtgcgcgggcgctactaccgcgacgtcgcgcgcctggcgcgcctgctctttctgcacctgtacgtggccacggcgcgcgagctctcgtggcgcctgcacgcggaccaggtggtggcccaggacgtgttcatgtccctgcgctacgagtgggaccagacgcggcagctgacgtgcctcttccacccggtgctgttcaaccacggcgtggtgctgctggcgggcgcgccggtgccggccgcgcagctgcgcgccgtcaaccgccgccggcgcgagctgggcctgccgctcgtgcgcgccggcctcatcgaggaggacggcgccgacctggtcgaggagccgccgttctccgccgcgctgccgcgcgccgccggctacctctcgcaccaggtgcggatcaagatggaggcctactcgcgcgagtaccgcgaccacacgtactgccgcccgccctcgcccgtggccagctacggcagcaccgcggaggccctgctgccgcccccgtcgccctcggccgtgctgccctgcgacccgacgccgccgccgcgcgtctcggccgccccgctcatcaccacggtgacgctggccgaggccgaggaggacggcgcgctgacgacggcggcgcccgcggccgcggtcgcgaccgcgacggtcgcctcgcccgggcccgccacccccgcctaccacctcatcccccgcgacgcgctcaaccgcatgtttgagatgtgacgccgcgccccgcgcgcgcggtcggatctgcggcgcccgaccacctgtgggcggggcgtataaagccgcgggccggcccggagggggggcatttgccgcagcgaccaccatgagcgacgcggggcccgtgcggcgcccgaggcgctcgcaagagcagcgcttccagccgttcccccgcgcggggctcctggtgcacctgcaggacgtgctcgtgggcgaggtgcggcgcccggacttccgccctccgcccgacgaggagagcagcgaggaggaggagcccgtctggaccgacgacgaggaggaggacgagggggaggaggggggagcccggcgcggggacggggagggggacgacgacgtggacgtggacgacgaggaggaggagagcgaggggggcgcgtggtccgacggggagctgtacgtggcggccgacggcgacgcgtgggacgcggaagaggacgaggacggggacgaggatgaggatgatgactatgacgatgacgacgacggctatgacggccgcggcgccgcggcctcccgcgccgcggtcgggccccccgccgccgtggactacctgtcgcgcgccctccgcggcatggagacggcctccggccccgacgagcgggaggccttcacccgctacgccggcacgctctaccgccgccagttccagcccgggcggccggggtaccggcccccgcggccgcgcgacccgccgcctcccccgcccccgcgggcgcggccgccgctgacgacggcgacgacggccgcgcccgccgtcggcaccgccgccccctgcggcggcgacgactccgcgccgcgcctcgagcgggacccggacgccgcgtacgccagctggacgcgcgacatgcccgacgggacctttctggccatgccgtcgtgggtggcgctgcgccgcgagccccgcgggcccccgggccgccccgtcgggcccccggacatcctgcgccgcgccccgcgggccctgaccttcacgccgcaggcggcctcggcggcgctcgtgagccgcgaccaggacgcctgggaggcgctggtgccgctgcaccacctgcagatgcactcgtggggcggcagcgcgccgccctcgcgcgggggcgtgtacgcgacgcccgcgcccgagacgcgcggcgtgtggcgccgcgcgctgcggcaggccatcgccctgcaccacgccatgttcctggcgccgctgcagtcctcgacggtcggcgcgcccgcgctcgcgggcgaggcgctggccttcgcgctggacgccgcggcgcgcgcggccgtcaaccacgacgccgcggcgcgcgagctccccgagcgcgcggcgcgcgcgctgcgcgccgtcggccggcgcgcgcccgcggcctcggggccgcggcccggcctcttccgcgccttctgcgggtccctggcctactggccggagctgcgcgtcctcagcgggcaccccgaggacgtccgcttcgcggccttccacctggcggtggccgaggtgtacctgctggcgcgcgcccacggccagaacccgggcttcacggccgaggagcgcgagctgctcgcgcccatgttcacgctgaccgtgctcgcggtgcaccacgcgctgcgctggctcacgacggccgtggcgcgcgccgtctcggacatccccgacgacgaggccttccgcgccgtgcgctcgcgcgtgccggcgtcgctggtgccgctgggcagcatcgcgctctcggacgccgagtacgccgtgctgaacgcgaccgaggtggcggcggcgcgcgacgccacgggcctgggccaggccgtgtcgctgggctacgccgccgcgcgctccgcgctcacgggcctcatgcgggcccacgccggcgggagcgacgccctcgtcgccttcgcgctggtgctgcagcgcctggcgggccacgcgaacctgctgctcaactgcctgctcggcgccgccgtccacggcggccggccggtgcgcgtgtacgaggcggcgctggacgactacgcggagctcatggacgcgctcgacccgctggtgcgcgcgtgcccgctcgcggagttctgggagcagcgcgacgccgtcatgcgccagctgcggctgacgccggccccgggcccccccacgggcggcaagcggctcgtcatcgcgcccgcgctgccctcggaggacctggacgggctcgtcccgcccacggccgtgcgccccgcccacttggggccggacgtcgacctcgccgaatacgccgcgcgccacccggagctggtgggcgtgccggaaccccgagcccatttatcggcgcgccggccgccccggggccgctaacccgtccctgcctccgccatgatccgccgcgcccgaggaacgcgccgcgcttcgtggaaggatgcctcgcgccgcgtcaccgaggggcgcacccgcgccagctgcctgctggcggcccccggggaggtgctgacggcggccgtggcggccctgcgcgaggcggccgagcgccagtgcgcgcccgcgctcttcggcgcccagcgcgcctcggccctggcgctcgtgcgcagcaactacgcccccgagtccgtgatcacggcgtgctcggcggagccgcaccgcgaggcgtacgcgcgcgcggccgaggccgcgctgaagggcatctcggcggacgtggcctggcgcgcggtgctggccacctactggcgctacctgaaggccagctcgggcgtggaggtgcgctcggacgacgagcgcgacgcccccaccgtcatgctgctgtggtcgacgttcggcaagacgctgtcgaagcacccgttcaagcacaaggcccagagcgccgcgtacggcgccacgcgcgccgcgctcgccgaggcgaccgaggccgtggagcggtacgcctacttcatgcgcccgctcgaccccatggtctccagcccccagacgagcgtgcgcctgcacgagatgctggcgtacgcggccacgctgtaccgctggctgctgtggatgatggacacggtcgacgcgcgcgtgctccgccacctggggcgcacgccgcgggtggtgcgcggcccgcgcgagacgatgtcgcccgaggcgctctttgggcggcaccgcgccggcgggcccgccgtggcctcgggctcgggcgaggtgctcgtgctgacgagccacatggcgacggtgtttgacgccctggagatcctcggcgccacgtgggccgagacgccctggaagagcggcgtgtgcggcctcacggccgccgtcgtggccgccgtggacctcgtgaccttcgtgcaccaccacgcgcagaccctcatcaacctgacgctcgccggctacgtgtgctggctcgacgacgggatggaggacccgtacctgcgcgcggcgctgcgcgcgcagtgccgcttccaccacctgatcggcgacctggcccccacgagctcgtcgcacgcctggggcgccatggagcgcggcacgctcgcctggttcaactacgccatcgcgcgcagcctggcctcgtacggcgggcccacggagcgcttcgcgcgcgtgctggcgacgacgggccagcgggacatgcgcttcctggccgtcgcccccgcgcagccgccgctgccgcccctgccgcgggcgtcgcggacgccctcgccgccgccccgcgaccgcgacgcggagccgccccacgactacgtggaccccctcgccgagtacctgaactggcccgtgcccgcctcgccgcccccgctccccgaggggcccccgagctcggacgacgagctggaggtggacggcggcgggcggcgcccgctccggcgcagccgcgacgccgccacgtacgtgaaccgcaaggacattgcccgcgcgatggcgggggcggaggacgaggacgaggatgaggaggaggacggccggtacccgcgccgcgacggggacgccacggcctcgacgagctcgtcgcaggagagcgccgccgacgaggagttcccgcccgggatcggcgtgcgcgacggcgaggaggacgacgaggacgagggggtggacgaggaggaggaggaggaggacgtgtacgtcggccccgacggtaccggcgaacggggcgccgccggcggcggcaacgccgcggacgacgacgacggcgccccctgggccccgctgacgcgccacgggagcatgcgcaccagcttccggcgcggggtccgcgcggcccagcgcttcgtgcgccgccggctctcgcgcacgagcgccgaggcggccccgcgggcctccggcgactcagcctccgcggcggcccccgccgccgcctccgcccgcggcgagaccgaccacgtgtaccagcacccccgcccgcggacccgcgcggacgacggcctgtaccagcaaccccgacccgtcatcgacctcaccggccaccgcgcgtcgcgccgcaagagctggcgcgtgtgacggctcccacggactcaccgttgtattgttttcccccgttgcccccctgtgtggaaataaagtttttttctaattctgtacacaaggcgtgtgtgtcgtggcggtcgtggtcgcgggacgggagggcggcggggagaggggagacgagaggggagaggggagacgagaggggagagggggggacgcggcgggggggggggggagaggagagggacgcgagacgggcgatcaacggcatctttatttttttccatctgcggggagggggcgagggggcgtcggggtcctcgttctcgaggcgctggtagtgccggcggcgcgtggccatcgccccgacgcggctggccagcagcgcgggcccgctgttcttcttgcgcgccttgtgctcctgctgctcgagggccgacacgatggacatgtaccggatcatgtcccgggcctggtccagcttggcctcgtccacgtcgtcctcttcgacgccgtcctccttgagcgccttcgtcgtgacggggtacagggccttcatggggttgcggcgcaggcgcgagatgtgccggtaggccaggaaggccgcgaccaggccggccagcaccagcagcccgatggcgagcgccccgaaggggttggacaggaaggacaccatgccgccgacggccgagatcacggcccccgtggcgcccaggaccaccttgccgacggcggcgcccacgtcgccgaggccctggaagaagttggcgatgccgcgcagcagcaccacgttgtggtccaccttgaccacgcggtcaatgtcgtagaacttgagcgcgtgcagctggttgcggcgctggatctcgctgtagtccaggaggcccgtgtcggcgagctcctcgcgcgtgtacacctcgaggggcaggaactcgcggtcctcgagcagcgtcaggttcagggtcacccgcgtgctgatcgtctcgggcacctccaccatgcgcacgtagctgtagtcctcgtagtacacgtacccgccgcccagcttaaagtagcgccggtggttgccggtgcagggctcgatgaggtcgcgcgagatgaggagctcgttgtcgtcgccgagctggccctcgatcacgcccgtgccgttgtgctcgaaggtcaccagcgggcggctgtagcacgtgccgcgctcgccgggcacgcgcatggagttctgcacgtacacgccgccgcgcacctccacgcaccgcgagatggccatcacgtcgccgagcatgcgcgccgagacgcgctggcccagcgcggccgtggccacggcgctggggttcaggcgcgacatctcgccccacagggtgcggtccttgttctgcagctcgcaccaggcggccgcgatgcggctcagcatgtcgttcacgtgcgcctggatgtggtcgtaggtgaactgcaggcgcgcaaactcggccgagcccgtggtgatgcgcaggtgccccgtgccgttgacggccggcggctcgggcgtccccgccgggccgggggagcgccgggcccgacgggcggccgcgggggacgcggggcccacgacgccggcgaggccgaggcgctcgagctcgcgcgcgtacagctgcgccagctcgttcgagatcagcgggcggaaggccaccacgaagcccccgcgggcgaggtacacctcgggcttgtcgccggccagcacgtgcgtgttgttgtagcgccgccggtagatggcgtcgatggcctccgaggcctcgcggaggacgcagtcgcccaggtgcacgcgctgcaggtcgagctgcgtgacgtcgctgacgaaggaggcgcccagggcccgcgacgtgaagcggaaggacccgtcgcgcgtctcgtcgcggatcatctcctcggcctcgcgccacttggccaggctgcacacgcgccgcgtcttgggggcccagtcccaggccaccgtgaagtgcggcgtgcgcagaaagttgcgcgtcacgctctcggaggcgcggaggcgcgagtccaggtcgatggggtagtagtgctccacctgctggaagcgcccgggcgcgtagccgatgtgctccccgtgggccccctcgcgcaggccgtagaagggggacatgtacacgatgtcccccgtggacagggcgaaggagtcgtaggggtacacggagcgcgcctccacctcctcgacgatgcagttgacggaggtgcccgtgtggtagaagcccgcggcgccgatcttggtgtaggtgtcgttggtggtgtgccagccgcgggtgccgagcgcgttcaggcgcgaggggcgcaggtccacctcgacggggttctcgtcgcggtcgaaggcggtcaccttgtggttgttgcgcacgtactcggccttggagacgcacttgccgcggcggtcgatcacgtccgtgatctcctgcacggggacgggcacgcggtccgtgaagcggttcgtgatggccgcgtacgtgctcccggaccacacggtcgtgacgatgacgttcttgtagtagatgtgggccttgaacttgtgcggggcgatgttctccttgaagagcacggcgatcccctccgtgaagttgcgcccctgcgagtactcggggcaggcctgctcgggctccaggcgcaccaccgtggagccggacggcggcgggcagacgtagaagcggtcccgctcggtcgcggccgcgcgcacggccgtgcgcgcgtccaggtcgccgtactcgccgtcgggggcgtccgaggggccgggggagacggccccgtcgatctcctcgagggactcctccgcggagaagccgtctggggtggcgcccgtcccgggcgcgggcgaggccgaggcggcccgcgtcacggccgccgcgccgcacgtcggggtcgcggcgagcgccagcagcagcagcgctagcgcgacggcgccccgcgcagctgcagcgtggtgtggagcaggccaaagacgtccgaggccagcaccgccgtggtgcccgggccgatgcccgcggggcccgcgccaaagaccgccaccagcgggcatgacgcctcgtaggtcaggtacagcccgtcgcgtccggcgaagcgatccgcgcggaggacctcgacgtccccgcagtggaagacggacctgaagactgcaaccgccagcaccagctcgcggatatatcgccaggcctgccgctgggtggcggttacgccggacgtctggaagcggtagaagccgcggaactcgctgacgcaccaatcgcgggccaccatgaagcgcgccagctcctccttcaggtgcggcaggaggcccacgttctccaccgcaaagtacagcgccgtgttggggggctgggcgaaggcgtgcgtgtcgtgcgcgaagagcggcccgttgacgagctcgaagaacttgtgcgtgagcgccgggaggagcgcggcgtccacggggtggcgctgcaccgtggccttcatgaagctgtgcgcgtcgaagcagcccgtctcgcgctcgtccacgacggtgcccgcgcgcgccacggcctcgcagaacgcgcgccgcgcgcggaacccggcggccaccgccacgtacgtgtgcagcagcgcgtcgccgtacacgttcacgcgcagggtcttctccagctcgcgccgctgctcgcgcacgcactgcgccaggctcgccgccgagcgccgcgagaggcggtccgcgtacaggcgccgccggcgctcggcgtcgcgcagggccgcggccggcaggtccgcccacgccacggcgtccgccggcgggggcgcgccgggcccgtcctcgccgccaccgccccccgcgccgccgccggcgtccgcgccccccgcggcgccgccctcggggtccgcgtcgtcgccgccctcgaggaggcgctcgagggcgtcgcggttggcccggtccgggtccagcatgcgcagcatgggcgtggacatgtggtggtcgtagcacgcgcggatgagcgcctcgaggcgcgcgtcgggcgaggccgcacagccgcccaccagcagcccgtcgatgatgtcgagctcgcgggccgcgcgggcgcggtcaaagtgctcggggcgccgcccgaagagcgcgagctccacggccgccgcgcgcaccgcggcgcggcgctcgcgccgctccaggtcgtccaggttgtgcgtgaacaggtcgagggcccgcaccgcctggttagtggacgccagccagaactgcagctcgctcacggcgtacaggcgccgcgaggcgggccggaacacgtcgtgcgcgtccaggagcgcgtccgcgcgccggcgcgccgtctcgcccgcgtccccggcctcggcgcccgccgccgccgcctccgcctcccgggcctcggccgcgtccacggcggcgagcgccgccagcgcgcgcccgcggcgcgcctcgtcgagcccgcgcacgtgcggcaggttcttggccacgtcctcggggtccacgcgcaccgccagctgccgcgtgaggtggtcgcacacgcagccgagcaggcgccggtgcgtggcctcgccctggttggccgtcacgcacagctcctcgaagcacaccgcgcacggctgcgccgggtcgtacagctccggcggcaccacgagcccgccgccgatcgtgcgcgcgaggaactcgtccacggccgacaggtccaccgcgtggggcgtgatcaggtggcagtagttgagctgcttgagcaggttctcggcgtcgtggaggaactgcagctccgtctccacgcgcccgccgtacgtgtcgagcgcgacgcgcgcgtggaagcggcacgcgccgcccatggtgcgctcaaagtaggcctcggcgtcgtcgccggcggcggccagcgcccccagcacgcggtccccctcggcccgcgcgtacgcgagcgccaggcgcagcgcgcacgtgagcggcgtcatctgcgcctccagcggcagccgccgcgcgaggtagcggcacatcagcgcgttcagcttgacgcgcggggccagctcgcgcaccaccgcggggtcgcagcgcttcagcatctccagctgaaacacgtacgtctgcacctgccccaggaccgccacgagccgccgctccgccatggccgagtccgcccgctcgctccctccctccgcccgcccgtccgccgcgagagaggccacgggacgcgcgtgtgtgtctgtcaaacgcggtcatctttattgatacagccgggcacccgcgcccgtcccctcctcctcctccccccatcacagcatgtcgaaggtcagccgcttctcgggcggggcgcccatgtcaaacaggtcgtcctccgggggcgggcgcttgccgcccagctccgcgggcgccagcgaggccggcgcgagccccgcgcccgcgtccccgcccgcgccgcaggccccaaagtcgaaggcggtctcctcgccgccgccgtccgcgcccgtctggccctcgaggtcgcgcaccagctcggcggccgcctccacgctccagcgcccgtcgcggtcggccacgcgcccgttgatctccgccagcgcggccgccaggaactcgtcgtccacgatcgcccgccagtcgtcgggctccaggtgctgcgtgcgcgccccgagcgcgtgcagcacggccgcgtacacggccgtctgcaccagcggcccgccgtcggccaggatggtcttgacgtgctccccgagcgagtggtcgtgcagcccggcgccgccgcccgtctgcgagcacgtgaagcccacgcgcgggcaggccagcacaaagtgccgcgtgcggtcaaacaccatcagggggcacacgtgcttgccgccgttgagcccgctccagttccccgcctggaagacgcggttgttgccggccatgccgtggtacttgctgacgcccaggcccagcaccaccagcgggcgcgaggccatgacggagcggagcaggtggctggacgtgtacgtggccacccacgtgtcgtcgcgcggcgcgtccagcacctcgcgcgcggcgcgctccgcgtcggccgcgtccgccagcgggcgccgcacccaggccatgacggcggccgggtcccgcgggcgcttcgactgcatcgtgatccccgtgaggctgttgatgaagaactgcttgtggtcgcagtagcgcaggatcaggttggccaggtagaactgggccagctccgccacggtgcccggggtcaggttcacgtagttgatggcgccgtagtccaccgagaagcgcttcacggccgcgatggtctcgatgtcgtccttggtcagcagccgcgccggcagctggttgcgctgcagcagcgtccagaaccactgcgggttggggctctgcccgccgggcggcttgccgcgcgggaacagcgtcgcgtggtgctgcttcagcaggaagcccagcgggccgttgagcacgtccacggccgtgtccgggcgctggtaggcgcccgcgagcccggccacgcgggcgcgcgcggcgtccgagaggctgcccgagcccgtcgagaacatgacccggctcttgacgcgcagctcccgcagcacctcgaagctcacgcgcgccaggtccccgtcgtggtcgcgcgccgggggctccgcgctgcccttggtggggtccggggccacgatgccgtcggcgagcgtcaccgtcaccgtcttggccgtgacgaagcccccgttgagcatgtccaccacgcggcggcgcagcaccggctggaactggttgcggaagttgcgcccctccacgggctgcccgtggagcacgccgtggcactggctcagggccaggtcctgcagcaccgccagcagcgtgcgccgcgccaggaagctcgtcgccgggcacagcgcgcccgagtacgggtccagcgagagggacatggtgtggttggcgtcgtagagcgcgtcgcggagcttgaagtcgcgcgtctccacgagcgtgcgcacgaagcggtccgccgcctgctcgaccgtgtcgcgcagggcgcgcagcgcgtgccggaagccggcgtggtccgtcaccgtctcgccgatgcgcgcgccggccacgtccgccagcagccggtccacggcggcgcggtacgtgtcctgcatgatcgccttcgggggctcgctgtcgttggggcgcttgagggccccgtacgaggcgtagttgccgagcacgtcgcagtcgctgtaggcgctgttcatggtcccgaagacgcccatggggctgcgcgtcggcgcgccgaagcgcgggaggcggtggcgcaggcggtgcagcgtcgtgtgcgcgcaggcggggcgcgaggcgcggtcgcagagcgagcaggggacctcggcgtcgaggctgccggccacgtagcgcacggcgtccacgtcgccgcggcccacgaacgtgcccgcgtcgcagcgctccaggtagaacagcacgcgcgccagcagctgcgggcagaagccgcacgccatcaccaggtggtccatggcgaagtcgtggttgcccggggccgtcgcgggctgcgaggccgtgtgcggcagcacgcgcccgtccttgtccgtctgcgggttcccggccaggtagggcgccgccacctggtagaagcggttaaacgaggggccggcgccctccttgccgccgccgtcggcggcgcccgcgtcgtccacctccgtcaggtacagcaccgagttggagctgaacaccagcgcccccacgaggccggccgcgcggctcacgtacgcgccgagcgccgcggcctgcgagacgggcgcgcccgcggaggcggccgcgccgccgccgccgtccttggcgcccgcctcggagagcacgggccagtcgtccagcgcggggggctcctcgtcgtacacgccggcggcgacgagcgcctccagcgacagcgccgcgtcggcgctcatgacggacgccatgcggcgctcgaggccgccggcggccgcgtcgccgccgcgccgccgctgctcaaagagcgtgaaggtcacgtcggcgggcagcacggcgctctcgtggttctcgtcgaaggccaggtgcgccgcgccgcgcgccaccgcgtccaggttccgcacgcgcgtggccacggccgccgggcccagcacgtagccgtgcagcaggcggcagagcgggccgttgaagaaggggcgcgggtacacaaactcctcgctgatggagcggttcttggcgttgaaggggtcggccacgagccggttgacgtccggcatgaagaggtgcacggggtagagcggcacgcgccacgcctcgtgcccgttgatggacaccgtgcccgcgcccccgtagtgcaggaaggcgttgcacaggtacaccgcctccttgaagccgtcggccaccacgaggtaggccgcgtgcgcgtccgcggccaggccgagccgcgcgcacacctccgcgcccgtcgtctcctcggcgttgtccaccggcgtccggtaggccgagaacccgaagcgggcgcgcgccgcctcgcaggcgcgcgagagggcgggcgcggcgctgctcgggggcacgcagtccccgttgtggaagaagaacacgttggggtggaagtggttgggcgtcagcttcagcgtgagcccgccgccgagcccggtggtccgggcgcccgccaccacggccacgtgcccggcgaagccggcctcgacggtcaggccgcgcaccagcggcgccaccgccgcggcgtcgtcggcgctgcgcgccacgagcagcgccagcaggtcgcggcgcagcgcctcgatcggcgtcacgtacacgtagcccagcggcgcggcgcgcacggtcacggtcttggccgcggcctccatcgtcgcgggtgagacgagacgagagcgggagcgggagcgggagcgggagcgggagagagagaaggcgcggcggacggggtcgagtctcggtcccctctccgggcggacgggtccggcggcggggtgaagagagagacggaggtgtcggcgccgaggggcgaacgcgacgtcggcggcggtggtagagcccggcccgccccggccgggtcttaaagcgtggggccccgccccgccggggcgtggcgccccgccccggagcctatacctcggccggcggcgttcgcacctccgcgtgggacgagcgatggcggcgcggcagggcagctacgtgacgcgcctgagcgagttcaagttcatcgcgccgcggtgcctggacgccccggaccagcgcggcgtgcacgtgggcacgctcgcgcgcgagcccacggtctactgcggcggcgccacgcggcccatcctccgcggggagcccttctggccgcggcgcgtcgccgcgtgggacggggcccccgagcggcccgtgagcccgcgcttcgagcgcttccacgtgtacgacatcgtcgagagcaccgagtacgccagcgcggacccgcgcttccccaacggcacggtggtcacgctgctggggctcagcgcctgcggcaagcgcgtggccgtgcacgtgtacggcgtgcgccactacttcttcctgggcaaggccgaggccgacgccgccctcggcgtgacgagcgccgagcagctcgcgcgcgcgctctcggcggcggcggcgcgcggcccgcgcctcgggcccgccgacgtggacgcgcgcgtcgtcgacgccgcgcccgtctactactacgacgccccccggcggcccttctcccgcgtctcgagcaacagcgggcgcctggtcgcgcccctccgcgagacggtctgcgccggcctggtgacgcacgaggccggcgtggacgcgaccacgcgcctgctgctcgaccacgacctgcccagcttcggctggtaccgcctgcggcccggccccgcgggggagcgcgtggtgctgcggcaccagcgcctcacgtccagcgacgtggaggtcaactgcacgcccctcaacctggcccgcgacgaggacggcccgtggcccgactacaagctcctgtgcttcgacatcgagtgcaaggccggcgacgacgcggccttccccgccgccgagaaccccgaggacctggtgatccagatctcgtgcctgctgtactcgctcgccacccagcggctggagcacacgctgctcttctcgctgggctcgtgcgactcggacgaccccgcggtgacggtcctcgagttcgacagcgagttcgagctgctgctcgccttcgtgaccttcctgaagcagtacgcgcccgagttcgccacggggtacaacatcatcaactttgactgggccttcgtgcacaccaagctcacggccgtctacgggctggccctcgacggctacgggcgcttcaaccgcggcggccagttccgcgtctacgacgcgggccagaacagcttccagaagcgcagcaaggtcaagatcaacggcctcgtctccctggacatgtacgccgtcgccgccgacaagctgaagctgccgagctacaagctcaacgccgtggccgaggaggcgctcggcgagcgcaagctggacctggactacaaggacatcccgcggtactacgccgcgggcccgcgcgagcgcggcgtcatcgggcgctactgcgtgcaggactcggcgctcgtgggcaagctcttcttcaagttcctgccgcacctcgagctctcggccgtggcgcggctcgccaacatcacgctcgcgcgcgccatctacgacggccagcagatccgcgtgttcacgtgcctgctgaagctcgcgggctcgcgcggcttcgtgctgcccgacaagcgccgcgacatcgcggacgaggacgacggcggcggctaccagggcgccaaggtgctcgagcccgactcgggcttccacgtggacccggtgctcgtgctggactttgccagcctgtacccgagcatcatccaggcgcacaacctgtgcttcacgacgctcgcgctcgcgcgcccggcgggcctgcgcgaggacgagttcagcgccttcgaggtcaacggggagcggctctactttgtgcacgcgggggtgcgcgagagcctgctctcgatcctgctgcgcgactggttggccatgcgcaaggccatccgcgcgcgcatcccgacgagcgcgcccgaggaggccgtgctgctggacaagcagcaggcggccatcaaggtggtgtgcaactcggtgtacgggttcacgggcgtggccaacgggctgctcccctgcctccccgtggcggccaccgtgacgaccatcggccgcgacatgctggtggccacgcgcgactacgtgcagacgcgctgggccacgcgcgagctgctcgagcgcgacctgcccgcgcgcccgcccgcgggcgagtacgccgtgcgcgtggtctacggggacacggactcggtcttcatccgcttctcgggcatcgcgtacgacgacgtgtgcgagctcggggagctcatggccgcgcgcatcacgcgcgacctcttccgcccccccatcaagctcgagtgcgagaagaccttccggcgcctgctgctcatcacgaagaagaagtacatcggcgtcatcaacggcggcaagatgctcatgaagggcgtcgacctggtgcgcaagaacaactgcgccttcatcaacgcgtacgcgcgccgcctggtggacctgctctttggcgacgaggccgtctcggcggccgcggccgcgatcgcgcgcgcgcccgcggcgcgctggctggagcgccccctgccgcgcggcttcgccgccttcgggcgcgtgctggccgaggcgcacgcgcgcgtcgccggcggcgccggcctggacgtggccgacttcgtcatgacggccgagctcagccgccccccggacgcgtacagcaacacgcgcctgccgcacctcaccgtctaccacaagctggccatgcgccacggggaggtgccgcgcgtcaaggagcgcgtgtcctacgtcatcatcgcgcccacccccgaggccgagcgcgacgcgcgggccgtgcgcccgggcctcgccccggggaagctgctcgtcagcgacctggccgaggaccccgcctacgtggtcgcgcacggcgtgcccctcaacaccgagtactacttctcccacctgctgagcacgctcagcgtcaccttcaaggccctctttggaaatgacaccaagatcacggagaggttgctcaagcgctttattccggaaaccgccccgggggacgcgcccttcgagcacgaggccttcgcgccgctcacgggcgaggggggcgaaagtcttcaaacgctgcgtacaatcttttgtactccaacagcagctccccgtcgaagctgatgtcccgcatcttgcaataaatgtcgctcgcgctgacggcgggcacgtcgtccgcgggtctcccgtctcccttctttcgcaccaccaacacgaacatggtctgccacacgtgcgcgacgatgcggtggccggcgcaggccccgagcaggcggtccagcacgcggtggtgcacgtgcaccgacttctcggggaacaccacgtacatcatgagcgcgccctgcgcgtccgggtcctcgaagaagagcacgcgcacgtcgcgcaggcccccgtcgcgcagcacgtagtaggcgaaaaagagctccgggtgcgcgagcacctcctcgagcgcgcgctccgaggggtcccgcgcggccaccgaggccaggaacacgcggtactcgtagatgctgttgagctgctgcacgtaggccagcacgagcgccgcgcggtcgctgcgcccgagccgcggctcggcggcggcgcacgtcgggcagcagccgccgaggcccaggtagtagcccatgcccgagagcgacaggcagttgtcggccaccgcctggccgaggtcgaagggcagcgtcacgggggccgtcttgatgagcgggttcgagaggccgcgcacggtggccacctcgtccgagggctgcgcggccgcgtaggcaaagtagggcgagtagcgatcgcggaccgcgcgcgccagcgtcttgcgccgcgcggccgccgacttgcgccgcaggagtcgccgccgctcaaacatagctgccgagcgtgtgctcgagggagacgagcgcgaggccgtggcgccgcagcagggcgtgtgcgtcgcgcaggaaggcggcgcgggcagtgggcttcggcgggaacagctggtacgctttaaatacgtacgccagggtccacagctccccgcacacgcgcccctcgaagcggttctcggcggcgatctgcgccttgcgcaacgcgtcgccgcgctcaaagagcacgcgcgggtccgcctggcgctcggccacggcgcacagggggtcgcagaagaggtgcttgtagacggcgcgcgggcccgcggcgcgcatcagcgccaggaagcgcgcgtccgccgcgaagccctcgagcacgggctcgatgcagtcaaagaggcccgcgttgttctccgagtagctgagcacgcggcgccggaagcggcgcagcgccagccagtgcgcgcgcgtgagcagcatgttgcagagcaggcactcgccgtgcgtgcggttgcggccgcgcacgtgcttcagcgccagcgccagcacgggccccagcgtggcgtccaccgcgcggtagcgcgcgtacgccgcggcggtgttgcgggccaggaagccctcgtgctccgcccagtactcgtccacggcgcggtgccggcgcgcctcggcggcgtccgtctgcgcgttgggctcccccgtggccacgttgcccagcagcagcatggcgaggtcggcgtagccgggcccgccggcgtccgcgtcctcggccccgcccccgcccccggcggcggcgacggcggcgcgctcgtacgcgtcgtcctcgcgctccgccgccgcgcagcacccggcgcacgcctcgtcgcccgtgtggtccttccagccccccaggctcggcggcgcgcgcgcgtccgccacggtgaaaaacatcgcgcgcggcgcggcctggaccagaaaggactggttcgagaagcggatcttgtcgccgacggtgcggaagcagccgtgcaggaaaaagtgccgctgcacgtccacgagcgtgagcacgcgcgtggcgaaggcgcgctgcagcgacgcgcgcccgcgaaagtgcttgtccatcacgtacacgaactcgaaggcggccacgaagacggccgtggcgcacggggggcaggccagggccttggcgcacagcgccgcgtagtccgccagccagggcggggccagcgagcgctcgcggcggtacacgtcgatcgtgcgacacacgcggcagggctcgtccagcgccagcggcgccgcggcggcctccacgaagcccgcgggcgagtcctcctcgtccagcatcacgtgggcggcgaagatcagctcgcggaagagggcgtcgttcttggtcagcagcgcggggtcgaaggccgtgtacggcgcctcgaaggcgtcctcgctccagcccatggcgtgcgacggcggcggcagcggcggcggcgcggtcgatctgcgcgcggcgatcccggaggccgcgctgcgcgacttcgacgtggactttctcgaggccaactacctgcccgcgcgggtgcgcgtctggttcgaggacgtgatgccgcgcgagctggaagtgatcctccccacgacggacgccaagctaaattacctggcccacacgcagcgcctggcggcggcgacgagcgagcgcgactgtgcccacggggaggcgctgcgggcgcggcgcgatcgcttcgccgcggccgtcaacaagtttctggacctgcaccaggtcctgcgcgacggggtggagctgggcgccctataaagtccgccgcccggccccgggagctccaccagccccaccaccaccgccactgccaccgcagcccgcccgcccgccgggaagaccgccgccgtcgccgcgtcgacgtcgacgtcttagccgcgccgctctccctcgccctcccggcccacggcgacgacaacgacgacgaccatgagcggcaccctgatccagcgcctgaagctcatcctctccggggggaacctgcgctgcagcgacggcgagacggcctgcgaccccgagcggcccccgacgcggtgcgtcttccaggtccacgggcaggacggctccaacgacaccttcccgctggagtacgtgctgcgcctcatgcgcagctgggcccacgtcccctgcgacccctacgtgcgcgtgcagaacacgggcgtgtcggtgctcttccagggcttcttcttccggcccgccgacgcccccctggccgcgatcacggccgagcacaacaacgtgatcttggcctcgacgcacagcaccggcatgagcctctcggcgctcgacgacatcaagcgcgccgggggcgtggacatgcggccgctgcgcgccatgatgtcggtgagctgcttcgtgcgcatgccgcgcgtgcagctctcgttccgcttcatgggccccgacgacgcctcgcagatgcagcgcctcctggaccgcgccgagctgcggcagcgctcggtctcccgccccggcggtggcgacgacggctgctcgggcgagggcccctcgccccgcgcgcccatccgcccacccgtcctctcgcccgtgcccggacacgcggccgccgccctcgtgggccaggccgcgtaccctccgcccacgcggttcccggcctcgctgctgcacgccctcctggggctgcggcgcctcgccggctacgccgtggcgtgcgtcaccggcgctctcgcgatagtcatcatcctgaacatgcgttaaaggcggccgcgcgcgcgggggcgcgcacagacgcgcgctccccgccgagccatcatgtccttcgacccgaacaatccccggacgatcaccgcgcagacgctcgagggcgccctgcccgtggacattctgctccggctcaaccgcgcgacggggctgcatatggatgcggccgaggcgcacgcgatcgtggaggacgcccgccggaccctgtttatcggcacctccctggccctcgtgaacctgcggcacgcgcacgacaagcacctcgtggagcgccagcccatgttcgccaccagcgactacagcagctgggccaggcccacggtcggcctgaagcggaccttctgccctcgcccctcgccctagccccgcgcgatcaataaagcgagccgcgttgcaccgtcctctcgtctctcgtgtgttgtctgggtggggtttcccccgagatcccccgatctccccgccgggtctttgggtcccccaccgagttccccaccgagttccccaccgagttccccaccgagttccccaaatgcccaccaccaccaagtctttgggtcccacaacccccaagttttggggtctcgggtccgagtcccccgagtcccccgagtcccccgcgccccccgagtcccccgagtcccccgcggcccccccgtcccccgagtcccccgagtcccccgagtcccccgagtcccccgagtcccccgaacacaccaccgcagagacaaacaggttgggtaataaacaatttattaaccaagaatcaggcggacctgaaataggtccacgttgaaccggcgggcgctgcccttgaggttcgcccgcgtctcccgcaggcggcgcgcgatcaggcggcacgcggcgatgagcagggccagggccccaagtccggtggcgcgcatgtaccgcctgttgatgagagtgtccgactccaccacgcgcggggggagcagctcctgggccagagagacgtcctcgtcctcgtcgtcctcatcgtcctcgaactccgcgccccaggggcccggctcgggcgcctcctcggcgggctcgcgggtcaggtccgcccgggtgggccagacgcggccgaggccgcccaggggagagtccggcggtgggggtctctgctgctgccgcggcggcggcggagggtgggcgggagggacgagcgcggccaggtcgtcgaaccacggcgggggctggtggcggtccgccagcggggagcgcggggccggcggcgcaacctccggcgggagctcggggacggtggtgggcgcctcagacagcggcggccagggcagtcgctccagcggcgagcgcggcggggtcgccggggcctcggcctcttctccctcgctcgcccatagcggctgagggggcggcggcagcggtgatggcggcagcggcggcgcctctcctccggggaacgggaacgggaacggctggtccgagggcggcggggtgaagagcgcgggtgggccggcggggtccctcggctcgggccacgaccacggcgcctccggggggcgctgctgagacggctgcggcggcagcggcggaggtgtgaacaggagcctcgggggctccgcgggcttctcgggctccgccggcgccggtctcgtctgcgccggggcccgggcgggctcggccctcggcggcgccggggccggccgcgaagatggtggtggtggtggcggcggcggctggggtggtggtggagcggcggcggaggaggccgaggaggagatggtggtggcggtggaggaggaggtggtggtggcggtggcggtggtggtagaggccggtgctgttgttgctgttgttgttggagctgagacggcggcggggggccgcgcgctctcgggggcggcggccagcgacgtcggggccgccagcggagaccgcggcggctgggtctgcgccggggccggagtctccggcgaggccggcagcggcaccgcgacgccctccagcggcacagactgcggcggcgggatgtggagacggaggtgcccggccggcgggcgcgcgacggcggcggctcgcctcactggtcctccgggggctgggaccacatctccgagcccgactccgagggcggggacggctgctgctgttgctgctgccgccgccggtgggcccgctggagcagtcgccggcgcttgggcttgggcttgaggcgggcgcggttcagctcccccgggaggacggccctcgggttgagccgcgggcggaagactcccggtctggcgcgaggctgatacgggccttcgcggaacaagagccatggattttcggcgactgtggtctctaatacccaccgacgttcgcgccttggtgcgtagaacagcggagggaaggggctgcgccactggggccgcggggagatgtcccgcaggttgatctccacggctcgcatccgaagccgggccaccgtctcctcggcgctggccaccactcccgggggctccaggaggtgctcgggccgctgctccggctgctgggggggaacctccgcgggagccatcgctcggtgttccggccggggaagaagaggaggacgaggcggcttctcgggacggagtcgaggcggtggaggaggacgcggacgagagctccgagtccgatgtcccgggaggagagcgggtccgacgctggtctggtccgaccgtgtgttcgcacttgtctgcgcaccccgctttatagcgttcgtcatcgagggggcggcccggggaggcggtccccgccaccctctcaccaaatttggtgtctcggacgtccggcggggcagcggccctcggcgctacgatgcggtcaccgtcgggtttctgggccgatggggcgggcccgggcgcggggggccggaaggtcgccgtcggggcagatgggggtggcggggcgggcggggcggcgggcccgggggtcgtcgtcggggcagatgggggcggcggctgtgggggagcgggcggggcggggggtgccgggggcgcgggcggggcgccccggggcttggcggccggctgggacgatgggggctgagcggcggcggcggcgacggtggtggtcgcggcgggctgtggcgggggaccctgtgacggaggttggggccggggctggggctgggtctgggggatgggggccttggccgcggcgggctgcggcgggggggccggggtggggggggccggggccgacgggggggcctgggtcgggggcgggggttggggggtcggcttcgccgtcgcggcgggcggcggcgggggcgccggggcggggggggccggggccgggggggtcggcggcggcggcgcggcgggcggggtctgggggggccttctcgggcttcgggggcgccgcggggtaaggcgggttgatgatcggcggcgggggctcgacgcgcgccttcagcgagggctggatctttcgcggcgacgagtgcggcttgggggtgggctcctggggccgctgcttgggggccgcggcggccttggcggccttcttggaggcggtggcggcggggggctcctgccgctgctgctgctgctgttgttgctgctgcggcggcggcggttgcgatggcgccttgggggcggcggggggctcctgctgccgctgcggcggcggctgctgcggctgcggcggcggcggcgccctggtgagcggggtcgcctccacgggcgcggcgggcggcggcgccggcggagccagcttgacgagcggggtcgtctccgcgggcgcggcgggcggcggcgccggcttggcggcgggggccgcctccgcggggggagccggtggcggcggcgccgggggggccagcggcgggagcgcgggcgggtcgaacggcggcgacgggggcagcggcagcgcgctggccaccaggtcgtcgccgagcaggtccagccacgggttggcgtcaaagtcgtcgtccgggacgaacccgtcgtcctcctcggccagctcgggcggcagcccggggcgcgggggcgcgggccgggtgggcgtcgccgacacggcgtacgggaacgggtcgcccaggttgcggaagctcggccggccgtacagctcctccgcggtggccgggtccacgcgggcgtcgcccatgatgatggaggtgaacagcgggtcccgggggtccatccccggcagctggtcctccgtcgtctccaggaaggggatctcccgcagcagcgggtcgtcctcgagcgtgaagcccacgcgccgcggcggctcgggcggcgcccagagcacgcgcgacaccgccagctgcgagtccaccagcacgaggcaggccggcgtgccggccaggggccgcgccgcgatcagcgccgagagccgctccagctgcggcgtcaggcacgcgttctcgatggggttgtcgtcgagcgccagcaggcgctcgccccaggagctgaggtccgtggacacgccgcggtgcagcgcggggtcgcgcaggtcgcacagcgtcacggccagggtcaggcccgagccgggcgagtacacgtccaggttctccatggcgatcaccagcggggccccgacgagcaccgccgccgcggccaggtccatggcgctcacgcgcgtcgcgctgctcggcggcgagcccgtgacggtgaacgtcatgcccgtgcccacgggcgcgtacaggtcgccgggcgccggcgcgccgagcgccgcggcctcgcgcacgtgcgccgcggcgtccaggcgctccgtgaccgcggcgtcgtacgcggcgttcgcctccggctgcagggcggaccacagcgccgagagcagccgcggggggatgcacatgcgcgccagcacggtgaaggtggccagcgcgcccggcacggcgcgcgccagccgctggcccgtctccgtgtgcgcccagggcgcccacaggtccgtctccgagaagcggccctcggtccagtcggcgacgcgcagcgtgaacagcgcctgcccgtcgcgcagcgggcgccgcggcgggcgcgcgcccgcgtccagcgtgtccaccgcgtccagcgcgcggtccagcaccgcgtccagcgtcttggccatgtactcgtgctgcccggccaggtccacgcgcgtaaagttgaacaggtgcgccggctcgccggccaccgcggccaccatcaggtcggggagggcgagccgcaccgccggctccagcggcggctgcacgacgcggtccagcgccccgaaggtgcgcgagagcgtcgtggccagcacggccgcgtacgtctccgcggcggccttggccgccgagtcccagtcggcgcggcggtggcgcacgaagcgcgcgaaggtggcgaagcccgtgtcgcgggccttctcgtagttgagccgcaggtggatgatctcgttcatgaggcgcatgcccgccacggtcgccgtctccatgagcccctcggtgagcggcgggcgcagctcggcgccctcgtcggtgcacagcatggccgccagcttgtcgccgcccgcgcggtagcacacctggtactccacgggcacgttgttgtcgtccaggaagaccgtggggaacaccgcgtccgcggcgctgcgcgcctcgcgcaccgtgcagatgacgtcgcggtcgcccaggcgcgccgccacgcgctgcttcaggcgcgcgcacggcgccgtcgaggcgcgcggccgcgccggcgtcggccccgggccgccgtaggccgcgtagaactgcagccgcagcgtgagcagctcgcggtgcgcggcgtacttgggcgggacccagcgcggcagccgctccgcgcgcgcgcgtcgcgcgcggcgaaggcggccgccagcgcgcgcagggcgtcgtgctgcagctcgggcccgatgcgcgccgccatgcggtcgagctcgcgcatgtcctcctcgacggcgccggcgtcgcggaagaagtcgcccagggccccggcgcgcgccgagtacttgctctcgatgcgccccacgaacttggcgggcaggcgcgggtagacggcgtcgctgcgcagcgccagcacctccatggtcgccgtctgcagcgcgcgcgcgcgggcctcggccaggcgcgcctcgtcctcggaggcgccggcgcggccgcgcgcgcgcccaaagtcctcccaggcctcgtcccaggcgacctcggcggccgtcagccgctgccgcagcgtgtccgcctcctggcgcagcgtctgcagcgcgtcgatgcgctcggcgaactcgtccatgggcccgcggccgtcgacgcgcgtcgtcagcgggtgcgtgtcgatgatgagccgcgcctggtgcagccactcgacggcctgcacgtccagctcgggcgcctcctcgacggcgccgagcagcagcgccgcctgcgtcaccagctcctccgcggcggcggcccggtcgatgttgagcgaggccggcggcggcgtcacccgcagcaggttcttgaggttggccagctcgagggcgccgtcgcggtcgcgcgccgccgccgcgtccaccacctcgcgcagcaggcgcgtggcgcgctccgtggccgcggcctggtccgcctccgccgcggcgaggccgtcgcgcgccgccgccaggtcgcggcgcacgagcgcgcgcatggcctccgcgtacgccgtgcgctcgaagcgcgcaaagtcgaagcgctccaggtaggtcacggcgccgcgcagcagctcggtgctcggcacgtgcaggtgcacgccctcgtcgagcgcgcgcgccgcgtcggcgggcgcgcgcacgtacgtcccctcctcgagcgcgcggcagatctcggcgccgcggctgccgccggcctgcagcacgtccgcgcggcgcgcgcccagggcggccagctcggcctcgtactcggggaagccgcggcggtaaaagtccacgtacttggccaggcgcggcacgtgcgccatgtccgccccgacgaagcccacggcgtcgtagtagctgaagcgccccatgccggcggcggcgcgggccagcacgccgccggcgatgcgcgccagccgcgagagcggctccgtgtcggcgccgaagagcgagccgatgacgggcgccgcgacggcgtagtcgtcgagccaggtcaggcgctgcagcgcctgcagcggcgggcccgccgccgcgccggcgttctcgggcaggtgcgggttgaaggcgaacacggcgtccagcgccgtggtcacggtctcggcgttcgcggcgagcgcgcgctcggcggcggggcgcagctccgcggcgtcgtagccgcggtccgccgccacgtcgcgcaggcgcacgaactcggccgcgtcgaaggcgtgctgctcggcgcggcgcagcgccgcgtcggcggcgcgcgcccagagctggcgctgcgcgcgctccacctccgccgcgcggcgcgcctccacggccgccacggccgcctccagctcgccgacgcgctggcgcgccgcgagcgccatcttctgcagctgcggctcctccacggcggccgcgcgcgccacctccagcgccaggtgccgcgcctcgaccagcgtcccctcgtcgggctgcgcgcgcagccgcccgagcatctccacggccgccgccacggccgccgacagcgcggcctgcagcgccgccacgcgctcagactcgcgcagcgccgcggcgcgctcctcgcactgccgcgccagcggctccaggaagcccagctccgggtgctccacggcggcgcggagcacccccgcggcgcgcgccgccttctccgccgtgtccgcgtccgccagcgccgccgccaccgcgtcggcgtgcgcgaggaagaagcgcgtgatcggcgggttgtccgcgagcgcgttgaccagcgcgccgagcggctgcccggcgcccaccagcgcgccggggtcggaggtcagcagctcgcggtagcgcgccaccagccgccacagcgccgcgaccaccgtgccgggcacgggcgtggccgtgatgtgcgcgagcaggtccaccgccgggcccagcgtggcaaagtcgccgagcagcgcgcgcagccgcatgtagaggtcgttctgctcggcggcgcgctgctgcgccgcctgcaccagcgtgcgcagctcggcgtcgcgccgcgcgagccgcgcgcgcgtctcctggctcagcacgtggccgctcatgccgcggatgtgccgcagcaggtcctccatcgcgcgcacgcgcgtctcctcgtccggggcggccgccagggcggcgtccatggcgtcgaagcgcgccaggtcggcgtcggccgaggcgcgcttggccgcgcgctcgttgatctggttcacgtcgcgcgtgagcgtgtccagctcgcgccgctccagatgcccgtgcgcctgcgcctcggtcagcagcgcgaaccaggccccgaggcgctcgtccgtgctcacgtcctcgcccgcctccagcagctgcgccagcagcgcgccgtcgtgggcggcgggcgccgcgtccagcgcgcgcagccgctcctcggtggccgccagcgagtcgacgaggcggcgcaggcccgccaccgcggcgttggccacgcggaagcgctgcgcgccgttgcggtccatcagcatggccttggcgctgtactgcgcgccgcgcagaaagtactcgcggatggcgtcgccgaccatcagcgccagcttgcgcggcagcttggccgcgcgctccttgacgtcgcgcgtgttcggctcggccgcgccgggcggcgcccccgcgtccacggcgtcaaagttggcgtgcgcggcctcgagcgccgcctcgagcgcgctcacgtcgcgcgcgcgctcgagcgcgcgctgcgactcgcgggactcgcgcgcccgcgcgcgcgcaaacaggccccgcgcgcgcgacaccaccgtctggtcgtagacgccgccggggtcccgcgccgcggcctccatgaagcatgccgagagctgcgcgtaggagcccccggactcgagcgcgtccagcgcccggtgcatggcctcgttcgcgggccgcgcgacgcgcgccgccaccagcgccagcttggccacgggcgcggacgcctcgccgccggccgcgtccagcagcgcgcgcgcctccgtcagcaccatgtgcgtggcggccacaaagtccccggccgggccgcgctccggcagcgcggccaggacgggctcgaagagcgccgcgacggcgcacggcgcctccgcgtgggtgcgggcgccgttctccaccagaaagtcctgcaggtgcccgaagaggagctcggcgaagcgctccagcagcccggcgcgcccggggaacaggctccggcccgcgcacgcgaccacgtgctcctcgagctcgtcggcgtgggcctgcagcgggcccaggtcgagcgggaccagctccagctcccgcacggggaagcccatgggctcgcgggtgcgcgggcggtacttgcgcgacggccgcccgccgaccgtgtgcgtgctgctcaggtcctcctggctcgccggccccgggggccggcgccgcttgggcatgctcggccgcttctggacccgcggcggcgcggcgagggacgagggctggggctgggactggggcaccgccgtcgagaggggggcggcggggacgggggcgacggcgaggacggggatggtcccgacggcggcgacggcgggggcgcccccggggccgcgcggcggcggcgcggcggccgccgggtccgggggcagggccatcgggtgcacggagggcaccgcccgctccacgtacggctcgtccacgaggtacgtctcgcccacgccgtagagcgcgggcacggcggcgaactggtcgtggggcaggaagaacacgaaggccccggcccagcggagctcgctggcggcgtagcccgcgacgtgcgcgtacacgtccgccgggcgcaggcgcgccacgaaggcctgcgtgatctgcccgtggccgtgcgggtcgaagacgtagacgacgtcgccgcggcggtagaggcccaggcccacggcgccgaccacgaccagcgtgaaggactcggcgcgctgcgcccagaggccgtcgaagaaggcgcgcgccgtgatctgcgtgctctggaagccctcgccgggcggcgtgaagaagttgcactcgccgtgcacccgcgagaagacgcagtgcagcgcggcgccgccggcgccctcgtagacgatcttgttcgggagctcgctgatggcgcacatgcccccgtcgggccccgtccacgcctggccctcccgcaggcacgcgtccacggcgtccgccgtcagggcggcctccacgccgttcgtgaagaccaggcgcaggaaggagagggaggagcgcaggcacgagacgcccgacccgggccccaggtcggggtcatactgattacgatagccgacgaccaccgcgtcggccgtcatggctgaaataacacacgcgcgtgggggaaaaatctttttatttggacacggcgtcgacctcggccaggagccgatccacgggcacccacgtttgcgcgagcaggggcacgcccgcgcccgcgtcgtcgggcgcgcgcgcgccgcggcgcagcacctccaggttcacggcggcccagtcgcgcacgttgacgctcgccgagagcggcacggcgtcggggcccgcgtcgccgtcggtcgccacctcgtggtagtcgcccaggacgcggtccgcggcctcgcgcagctccccgggcgtgaaggcggccgcgggcggcgcctgctcctcgcgcgcctcggtccaggtctcgcggagcgtcgcggcgagcgccagcacgccgcgctcgtcgacggcgcggcgcagcgcggcgctgaccaggtcccagcgcgccaggaagcgcgccacgggccgcagcacggtggcggcctgcaggcggccggccgtgagggcggcgctcgtctgcagcttgctcaggcgcgcgtggtcggcgcacagcgtccggaacacgggcgcctgcaggcccagcgcctcgcactcgcgcagcgcctccgtcgtctgcgccatcacgccgcgcagcgcccccgcgtcggcgcgcatgtccgagcggtgcgtgttaacctcggcgtggaaggcgcccacggcgtccacgacgtccgccaggcgccacgtgcccaggcagggcccgccctcgccgggcgcgaagacggaggccgtgcgcccgcggaagacgaagcggcaggtgaagaggcgctcgagcgccaccacgcgctcggccacggcgcccgcggccgtggccatcgagtccagcaggtcgcccgtgtaggtggccaggtcctccaggcggcgccggagggcgcgcgccatcaggtccccgaggaaggcgctggtggccagcgcgggcggcgcgggcaccggcgcctcgagcacctcggcggccgcgcgcacgaccccgtgcagctcgtgcgcgagcgcctcgacgttgcgcgcgtgcgccaccgtcaggtcctcgcgcgccagcgccgccacgagcgcggcgagcttggtgcggtagccgctgacgttggggatgaggccggcgccgtagtcgcgcgcccacgtcagcacctgcgccagctccgcgcggcggtcgtaggaggccgtcaggaggcagttgtaggcgtcggccacggccgtcacgacgaagaagggcgcgaaggacgggtcggccatggcctcgaggtgcaccatggacgggtagttcacgaggtccgcggcggcggcgaggtcgacgagcgccgcgctcgtcggcgcctccatgggcgtctcgccggcgccgtcggcgcgcaggccccagcggctctgcgcgcggaagccgagccacgtgccgagggcctcgcgcgccgcgctgtgcgccagcccggcgcggcgctcggccaccgccacctcgcgcatcagcgcggcgcgcgcggcgcccacgtgctccgtgggcgggcgcggcgccatgcagtggcgcaccacgtgctccacgcccttctcggggagccccagcgccgccgtcacgtccccgtagaggggctccagcgtctccgcgacgtccgtgtaggcgcgcacgtagtccagcccgggcccgtagtcgtcgaggaggcggtgcacgacgccgccgacgaccgccgtgcagccgacggcgcgctgcgcgtccgtcaggcggggcgcgcggtccagcacggcgcgctcgaggaggtcgcagtggcggcgcgtgaagcccgcggtcacgagcgcctccacgacctgcagcggcgaggcgctggcctgcaccgcctcccacacgcccgtggcgtccacctgcgcgtggtcgaggtccgcggcggcggccacgtgcacggcctgcgccccggggtccagcagcgccagcacgccctggtccgtgcgcaggcgccgcgccacggtgcccgggcgcagctcgcgcacgtacgcgtccccgtgcgcgagggccgcggacacgcgcacctcgacggccgtgcgcggcgtggcgttgacctcgtccacgggcaccagcgccgcgtcgtccacggcccgcgccgcctcggcgaactgctcgcgcagcgtcgggttctggcggtcgtggaagaactcgtcggcgatcgtgagggcaaagagcgcgcgcgtcagcggcttcaggccctcgttctgcatctggcgcaccgccagcgccagcggcagcgcccagttggtgcgccgcgccacgtggcgcaggccgtcctgcacaaagtccatgtcgtagagcgtggcgaagcggggctgcacgagcgcgcgcgccgtctgctccgggggcacgacgcggggcgcggccaggacgtcgtcggcggagcgcgcgcccgccagggcctcgtcgaaggcggcgcaccactcgctcacgaggcggaacatggcgtcggggccgaactcggccgaggtgcgcagcccggcctcggtgagcgcggccgtggtggcgtcggcgttgacgacccccaggagctccagcagcggggccgtgccctcgtcccagtcgtggcgcaggcgccacagcgccaggccggccaggttctcggcgagcacggccgcctcgcaggtgccggtcgcggcgtaggcgcggcaggccaggcgcagcgcgcgcgcccagagcccgcgcacccccgcgggcgtctcgcggcccggggccatgaagaactccaggatgtgcgactgcaccaccgtggcgacggcatggtcggcctcctcgagcgcgcgcacgagcgcctccattataagagggtccgcgcgcgggcgccgccgcgcactcgaccccgcgcggcgcgacgatgagcgtgcagatcggcaacgggctgctgatggtggtcgcgccgggcacactaaccgtgggctcggcgcgcgcgcgccttatacgccaggtgacgctggcggacttttgcgagccccaggccgagcgccccgggctggtggtgctggcgctgcggcaccccgcggacctggccggcgccgcctacgcggccacgccgcccggcaagaaccaccgcgacctggaggaggcgtggctggccctcgacgagggcgggcgcggcctcggcggcgacggcatccgcgcctccgtcgtctcgctcaactttttggtggcggccgccgagaacggggacgacgcgctgcgcgcccacgtgacgaccaactaccgcgaccggcgcacggccgcgcgcctcgagcgcttcgcggccgtgctgcgcgccatgatccggagccacgtgttcccgcaccgcgcgctgcacgtcctcggagggctcctgggccacgtgacgcaggaccggctggccagcgtcacgtgcgtggcgcgcggcgaccaggaggcggcgcgcaccaacgacatggccgcgcgccgctcgcaggtgcacgtgcccgcgtgcgcgctgatggacgtggaccgcgagctgcgcctcggcggcgacgacgggctccgcttcgtgtacctggtcttcgtctacacgcagcgccaccgccgcgaggcgctgcgcgtccacgtggcggtgagccgcctgcccgagctcggcgacgccctcagcttcctcctggccggcacgcgcgtcgacaacgcgatccacggcacggacgaggccgacgcccccgccgcgcccgccgccgccgcggccttccccgcgtacctgttcaacgacccgcgcagcgcgcgctgcccgacgggccggctcaacacgcccgccgccgaggcgctgcccgtgtgggcccccgacatgcgcggccgcgccacccggaactcgtgcatgtacgccgcctacgtgcgcctcggcaccgtcgagcgcgtcgtgcgccgggccgagcgctgcggctcggtggacctgccgctggcccacatggagcgcttcacctgggacgtgggggcgtgggaggagtgtttcttctgaaaaaaacggggggaggtgtgcgtgagacgggtctgatgttgctgacgagactaataaaaagcgggggatacgcgcgcgcgtgtgtccccccgttgacccttcatgccgcgtccgcgtcgtgtgtgactcacccccatcgtctctcccgcccgcgatcccggcccgtcccgactcgtcccgcccgcccagacgcgtcccgtcacgcccccgcgcacgccccgcccgccgggcgcggttttcccccctttccccccattggcggcctccgccgctaaaagcccgcgcgccccgagctcgggactctccgcgcactccccgtttcgagcacacaccgtcatggcctcccccgtcgtgcccgccgccgccgccgccgctcccggcgcggactccttcctcgacgccgtctgccccgaggacgtcgcccgggccctggccgccgagctcgaggcgctccgcgccctcgggcacgacgtcggcgcgcccgcgcccggggcctcgcgtcgcgaggtggcgctgtttgtcacccgcgccgtggacgggctcaaggccttctcccgggtggacgagcgcgtgtacgtggcctgcggcaagctggtgcacctgcgcgtgcgcagccgcgaggcggacctggacgcctggctcgcctcgccggagctcgcgctgatccccgcggtggcgggcgccgtgcgccggcaccgcgcgcgcgtggaggcggccctccgctggttctggcgggaggcctacccggcgctctacgcgcgcggcctgcagtcggcgctcaagtacgaggagatgtacctggcccgcctcgagcacgggcgctgcgaggccatggaccagttctttgtgcgcctcgccgccgccgccgccacggcgacgcggcgcgccatggcgctcgtgctctgcggctcggacgcgtggccggaggtctttgacgcgtactttcgcgcgctggccacgcaggcgatcgtgcccgccaccccgctcatgctcttcgccggccgcgcgcgcggctcgctcgccagctgctacctgctgaaccccctgccgcgcaccaccgaggaggcggtgcgcgccatcacggacgaggtggcccccatcctgctgcgccgcggcggcgtcgggctctcgctgcagagcttcaaccgcacgccctcgggcgactgcacgcgcggcatcatggccgtgctcaaggcgctggactcgatgacggcggcgatcaacagcgacagcgagcgccccacgggcgtgtgcgtgtacgtcgagccgtggcacgcggacgtgcgcgccgtgctgaacatgcgcggcatgctcgccgccgacgagagcctccgctgcgacaacatcttcagctgcctctggacgccggacctgttcttccagcgctaccagcggcacctggacggcgagcgcgccgtcaagtggacgctctttgacgaccgcgcctcgcacctgacctcgctgcacgggcccgactttgcgcgcgagtacgagcgcctggagcgcgccggcctcggcgtcgagtccctgcccatccaggacatggccttcctcatcgtccgcagcgccgtcatgacgggcagccccttcctcatgatgaaggacgcgtgcaaccggcacttccacacggacacgcgcggcgccgccctcgccacgtccaacctctgcacggagatcgtgcagcgggccacgcccggcgagaacggcgtctgcaacctggccagcgtgaacctgcccgcgtgcctggcgggcggcgccttcgactttgccgcgctgcgccgggccgcgcgcgtcgccgccgtcttcgtcaacgccatgatgcgcatcgggaactacccgacggaggcctcggtcgagggcgtgcggcgcagccgctcgctgggcatcggcctgcagggcctgcacacgaccgtgctggcgctggacatggacatggccgacccggcggcgcggcgcctcaacgccgccatcgccgaggagctgctctacggcgtcatggacgccagcgtggagctctgcgagcgcggcctgcgccccttcgacggcttcgagcacagccgctacgcgcgcggggtcatgcccttcgacgcctacgagcgcgtctcgctccgcgagccgatgcgctgggacgcgctgcgcgtccgcatcgcggagcacggcgtgtacaacgcgcagtttgtggcgctgatgcccaccgtgtcctcgtcgcaggtcaccgagagcagcgagggcttctcgcccaccttcaccaacatgttcagcaaggtcaccatctcgggggagctcctgcggcccaacctgccgctcatggagacgctgcggcgcctgtttccgcgcgagtgcgcgcgccgggacgccgtggcgcggctggagcgcgcgcagtggtccgtggccgcggccttcggggagctgcccgccgggcacccgctggccaagttcaagacggccttcgagtacgaccaggagctgctcatcgacatgtgcgcggaccgcgccccctttgtggaccacagccagtccatgtccctcttcctgaccgagcccgccgacgggaagttacacgcctcccgcgtcatgggcctcctcatgcgggcatataacctgggcctcaagacgggcatgtactactgcaagatcaggaaggccaccaacaacggggtgttcaccggcggggacctggtctgcacgagctgccacctgtgagcgcgcgccatggagtacttttacacgtcccagtgccccgacatggaccacctccgctcgctgagcgtggccaaccgctggctcgagacggacctgccgctgggcgacgacgccaaggacgtggccgcgctcagcgaggccgagctcgagttctaccgcttcctcttcgccttcctctccgccgcggacgacctcgtcaacgtcaacctgggcagcctctcggagctcttcacgcagaaggacatcctgcactactacatcgagcaggagtgcatcgaggtggtgcactcgcgcgtgtacagcgccatccagctgatgctgttccgcggcgacgccgcggcgcgcgagcgctacgtgcgcgcggcgctgcgggacgaggccatccgccgcaaggtggagtggctcgactcgcgcgtcgccgagtgcgcctccgtcgcggaaaagtacctgctcatgatcctcatcgagggcatcttcttcgcctcctcgttcgcctccatctcgtacctccgcacgcacaacctgtttgtggtgacgtgccagtccaacgacttcatcagccgcgacgaggccatccacacctcggcctcgtgctgcatctacaacaactacctgggggacgccccgcgccccgacgaggcccgcatccaccagctcttcgccgaggcggtggagatcgagtgcgagtttctgcgggcgcgcgccccgcgcgacagcctcctgctggacctgccggccatcatctcgtacgtgcgctacagcgcggaccggctgctgcaggccatcggcgcgagcccgctctttggcgcgcccgcccccgcggcggactttcccatggcgctgatggtcgccgagaagcacaccaactttttcgagcggcgcagcaccaactacacggggaccgtcgtgaacgacctgtagcgcccagcggcggcgacccccgctctcccccctcctcctctctctgcgacaaaacactaataaagcgttgagacactagcgcgcgcctccggccgtcatccttggggcgagggtgggatggggaggtggaggcgggatgggagagtgtggggagagtgtggggagagtggggtggggagagtgcggggatgatgacgggggaagggagatggggctgacgggggtggactgggactggggaaggatgacggcggccggcgcagagtgggaggcggtgaggtggacgggggacggaggggaggaccggagtgggggggaagaggaggggcggcggagggggcggcgggggggcggcgggggggcggcgggggggcggcagaggtggacgggcggggggcgggagcggggcggcgggggagggggggggggggggggggcgggcaggggggcggcggcgaggtgagacggagcccgcggacggagtggccggcggggggaggcgacgacggagagagaaggtgggggagagggcgagcatcacacgggccgcacaaccgaggatttttttcaagttgttttttattttctcctatgggcgttacagtcgtcccagtacgtcatgaggacggcctggtagttcggaggcggaggtatgtgcttccaaaacaggcccgccagctccttcgcccgcgtctcgttcttcacgtgccgcagcagcgagccaaagaccttgttcacgtccgagggctcctgcacgatggggacgcgccgcagcacgctcaggtgcccgtgccgcgccggcgtgagcatggccaccacgtgctggatgaactcgcgctcggcgcggcgcgagcgcgcgtccccggccgccggcagcagcgcgagcgccttgccggcgtcgcgcatgatgtcggggcagcgggacgcgtacttgagcttgacgtcctcgggcgccacctcgcggccctccagcgactcggccacctgctgcaccgagtccacgtccggcgcccggtgcaggtccgtgtggcagcgcacgaacgcggccaggaactccgagtagtccacccccaggctcgcgaggacgtcgcggcagcgcagcaccagcgggaacatgggcgcgatgtcgaggatcatgtcgcacccggtgaggatcatgtccgtgtcggtcgtgtgcacctgcgcgaccgtgttggtgtggtagaggttggcgcagacgtcgtccgcctccatgtccgacacgtccacgtaggcgtagcccatgtggcggatgaggttgacgcagaggcggtggacgatgcgcggcgcgtgcagcatcgtgctccagcgcggccgcggcggcgcgtcggcggccccgtcgccgtcggcgcgcgccgccgcggccatgatggccttggcgccgtgcgcgacgcggccgttcccgaagatgccgcggtccgagacgaagatggggaagtaggtgcgcttgtgcagcatgcgcagcaggcgcagcaggcagcgggcggtcgtggtcgcgttgtcctcggtcgtctcctggtagtgcttctccatgagggtgtacatgacgttccacaggtcgatggcgatgggcgtgaggaccccgggcggcgtggagatggcctccgagcggaccagacgatggcgatgtgcgtactttaaaaggccaaagagccccatgtctccgctcgcacaccgaggcgccggtctaaaatacgcaggtggcggggcggggccacccgcgcatataagccgggcggtgattggtctggcgcacaccgcccgcccgagcccgctcgcccgccgcgatgtcgctgttcgacgacggcctcgaggacctggaccgccaccccacccacgcgcaccacccggcgcaggtgatccacgacgggcccttcgtgctggaggacggcgagcccctgcagcgcaccggcatgctggtgctcagcgacgagcacctggagcacgcgcgcgccgccatcgccccgctcgccgcgcacctcgcgcacgccttcctcgtcttcagcgaggccgggctgctggtgcacgccagcgtgcgcggcgagcaggtctacgtgaccctggccccggaccagttcagcacgttcgtgtggagcgggccccaggccgtgttcctgggcaacgtcgacggcagcggcggcgtgctcgacgcgctcaaggtcgaccggcggcggaccgtcttcaacgtcaccttcgaggtgtacggcgccttcccggcgcggctgctgacgcggcgcgcgtactttgcggacgcgggcctcctcgcggcggggcccggctccccgagcgtcgcctgcgtctacaagcacgagttcaacgactactgcatcatgctcccctcgcgggcgcccgacgtgagcctgacgctctcgcgcccccaggtggccaagctcgccgccgtggcgaagggcgccgcggccgggacgaccttcgcgctcgcccgcggcctcgacttctccgtctcctccagcgccggggtcgtgaccttcccggcgcgcgaccacgacgggaccgccgtgctggagcgcgccagccggcggcgccagggcgtcgacgcggtcggcgcgacggagcccttcgccatgacgctcgaggcggcgcacgggctgctgacgctgctgcagcggctgcgggccgggaacgccgagctcacgttcaactttttcacgacgccgcggcaggcgcccctgttcagcgtgaccacctgcggcccggtgcgggcgaccaccttcttcttctgcgcgcccgccgaccccgccaccgtgcccgccgcccccgagggcgccgccgccaccgtcgccgccgcctgcggggcgggcgcgtccgccgcctcccccgccgcgggggacaagcggcccgccgccccgcgcatgtacacgcccatcgccaagcgcccgcggaccgcctcgggggaagggggccacgcctacggagatttattctaataaagtgagcaggtgtataaaagagaggtcgcacctccgcgtctttactcgccgtcgcgatgatctcccgggggaacggagggggttgccgccccggcgagccatgctggcgctgcgcgctcgagtccacccgctgcatcacgctgatgggcgtgctcgtcgcgctcctcgccgcctgcatgctgtccgtcccgcccgcggcgtcgacgatgctgctcggcgtcgccagcctcatggccatgctgcgcctgcccatgcccctcgtggaccggttcatccccgcgtgcatgggcctccagctcgtcggcgccgccgtcttcgccgcgggctgggcgctggccagccgcgacgcgatctcggccggcgtcctgctgtgggccgtctgcgcgctcatctcgcacatgtacaacgtcgtctgcgtcgccagcgggcccgacgcgcactaccgccccgcctgcctcgtcatgggcgtcgccgcggcctgcggcgcggcgggggccctcgtgaacgtgcgcaccgaggcgcgcctcggcatcgccctggggctggccgtcacgtgcgccacgaacaacgtcgcccgcagcctgcgcggcacgtgcacctacgtcgccagccgcgcccggttcctggccgcgcccgcggacctggggcgcggctacagcgtggagaacgcggacgccgaccccaccgccgagcccgagcggcgcgtctacgaggcgaccgtcccgcccacccacgtctacgccggcagcatcgcgctcttcgccctcgtcttctcggccgcctcctccctccagtggatggtctcccagatggtgggccgcggcaaccagctcgtctcgcccaccaccgcggccgcggccggcgccgccgggttcctggacgcggcggcgctctcgctctttgtgcgcccgagcacgcgccacctctcggtggccgtcaagggcgcgcacgcgctcctcatcctcgcggccatcgtcctcacggccgccggcgagcccatgggcgtgcccatcagcctcgcggcctccaccggcctcgcgctgctcgaggcggaccacgtccgcctgcgccacacccgcgcgtaccggctcgccgccgcgcacgtgacgcgggccctgctcgtgcaggcgtacgtgaccgtcgccatgtgtgccactagcattaaatccgtttcctgattcacgcccacgcccgtgtcgtttttaaaaccgcgatggggggacggggggccattcgcacgcgccatggcatcgctcgcgcgtgcgatgctcgcgctgctggcgctctacacggcggccatcgccgcggcgccgtcgtccacgacggcgctcggcacgacgcccaacgggggcgggggcggcaacagcagcgcgggcgagctctcgccctcgccgccctcgacgcccgagcccgtctcggggacgacgggggccgcggcctccacgcccgccgccgtctcgacgccccgggtcccgccgccctcggtctcgcgccggaagccccagcggaacggcaacaggacgcgcgtccacggcgacaaggccacctcgcacgggcgcaagcgcatcgtgtgccgcgagcggctgttctcggcgagggtgggggacgcggtcagcttcgggtgcgccgtcgtcccgcgcgccggggagaccttcgaggtccgcttctgccgccgcgggcgcttccgctcgcccgacgccgaccccgagtactttgacgagcccccgcgcccggagctcccgcgggagcggctcctcttcagctccgccaacgcctccctcgcccacgcggacgcgctcgcctccgccgtcgtcgtcgagggcgagcgcgcgaccgtcgccaacgtctcgggcgaggtgtccgtgcgcgtggccgcggcggacgccgagaccgagggcgtctacacgtggcgcgtgctgtccgccaacggcaccgaggtccgcagcgccaacgtctcgctcgtcctgtaccaccagcccgagttcggcctgagcgcgccgcccgtcctcttcggcgagcccttccgggcggtgtgcgtcgtccgcgactactacccgcggcgcagcgtgcgcctgcgctggttcgcggacgagcacccggtggacgccgccttcgtgaccaacagcaccgtggccgacgagctcgggcgccgcacgcgcgtctccgtggtgaacgtgacgcgcgcggacgtcccgggcctcgcggccgcggacgacgcggacgcgctcgcgccgagcctgcgctgcgaggccgtgtggtaccgcgacagcgtggcctcgcagcgcttctccgaggccctgcgcccccacgtctaccacccggcggcggtctcggtgcgcttcgtcgagggcttcgccgtctgcgacggcctctgcgtgcccccggaggcgcgcctcgcctggtccgaccacgccgccgacaccgtctaccacctcggcgcctgcgccgagcaccccggcctgctcaacgtgcggagcgcccgcccgctgtcggacctcgacgggcccgtcgactacacctgccgcctcgagggcatgccctcgcagctgcccatcttcgaggacacgcagcgctacgacgcctcccccacgtccgtgagctggcccgtcgtgaccagcatgatcaccgtcatcgccggcatcgccatcctagccatcgtgctggtcatcatggcgacgtgcgtctactaccgccggtccgcgctgtgacgcccccgcccgccccgaatcaataaacgaccgcgcgttgaaccgagccatcgcgtccggttgtttgtttgtgcgtgtgtgtgtgagctctcgcgcgtccccgggccccgctctctcccgtcccactccactctcctctcccgcaccctccccacacacacgcccggcccgcaataaaccacacgagacgacggcgtgttgtaccggcgcgaccgggccatctctttattggtcgtcggggaacagggacctggccgggggggggggggtgggcggcgggcgagcgacgccgtcctcatcggtgggacatcatctgggagacgaagatgtcggcgccgcgctggcaggccacggcggccgagctggcgtcgatggtgccggcggagacgtctgccggggccgagaccgaggcctggacgggcggctggggggccgaggcgggcgcggccggggcggcggcctgaacgggggcggccgcggcggccgccgctgctggagccggagcgggggcggccgcggcggcggcggcggcgggcacggccaccggctgggcctgcaccaccaccggctgctgcgggaccacctgggcggggacgtgcgcgacgggggccgccagaccctggaggcccggcaggcccgggagcgccggcacggcgtgcgccacgggcgaggggggcggcgcgtgcagccccggctggtgggcgtacatcggcgggtacagcgccccggcggcgtggggcgggtgctggtgcgcgtgtcccacgacggccccggggggcaggccgggcggcagcagctgcggcggcgtcgcgggcgcgtagtacggcgtcgggtagcggagctgctgggcgcgcaggtggctgatctcgcgctggagcgacgagaccacggcggcgaggtcgctcgcggggggcggcgcctccccggggtagtacgcgttgtcctgcgcgtagtcgtcgtagcggcgcttccgggcggggggccgcggctcgggcgagggcgagcggcggtggcggcggtccatggcggccgagacgatggcgccgagctgggactccaggctgggcgccgggcgctggttcaccaccagctggttgtactgcgccgcgggcacgaagatgtagtcgccgggcgctgcgctcggcggggctgccatggccgcactgggcccgggcgccggggaggccccgcttttaggcagaagccccgcccacatcgtcgcctgcaggtaggtgtgtccgcgcacgccggcctcgcggcggcgcgccgcgaccagctcccagcggtcgcgcagcagcatgttgttcacggccgtcgacagcagcgcgcgcgtgagcgcctcctcgctcatgtgccacacgcgctcgcggtccggcgactcggcggcgcgcgccagcagctcggagcgcgcccgcgccgagagctgccggaacggcgccacggcggcctcgggcgaggcgtcgtacaccacgatggtgcccacgcgccgcccgatcacgcagagcgccacgtgcgcaaagagcgtctcgtcgggggcctcgccgggcgccaggcgccgcgaggacagggacgccgagggcaggtagttgctcagcaggtacagcagccgctcctcgtccgagagccgcatgtccccgaagaagtccgggcccacggcgcgggcgagcacggcccccagctgcgggcagttgacgacgccgaggaagaaggggccgcggtcgtcgtcgaccacggccagcacggcgccgatgtcgcagcgcgggcggtggtcgatgttgatgggcagcgggcccgccgggggcagcgcggcggccacgacctcgcgcgtcagcgcgagctccccgccgtccctatcgtacagggccaggtagccggacacgtacacgggccccatggcggcggcggtggcgacggcggcggcgcgcgcgcggaggagactgcgggctcaggcggcggcgaactgcgggatatagcccaggcagagaaagtacaggaggtcgtagtcgctggagacgccaaactgcccgaggcgcttgtccgccgcgttgcgcaggtgcggctgctgcagcagcaggcccagcccctcctcgtactgcacggccacggcgtcgtgcgccgcgatgacctcgttgacgggggccgtcacgcgctggcggttctgcagctccagcgccaccaggcgcaccaggctgagctggtggcgctgcacgcgcgcgcggatcgagtgcgcgtccagcgccagcgcggccagcccggggaagagctgcgtgagctccgtgcgctcgtccgcgcggtaggccacgacgacgtagtgggccagcaggaagcgcaggttgccgtcgccgctgcgcgaggcgggcgcgtccccgctggcgccgcgcggcggcggcgcgttgggcaccagcgcgccgagctggaagttgttgcgcaggcggtccccgtacacgttgccgttgtgcagcaggcgccgcagcagcagcaggcccgtgatggtgttcaccgtggcgcgcagcagcggcgcgtcctcgccgaggaagaggttctggccgcggcccaggaaggcggccgcggcgcggttgacctcgtccacgaagcccgcgccgtcgtcgacctcgttggcgccgacgttcgcgccggggaccacgcgcaggcgcgccagcgcggcgagcacgctgtggccctccagcgcgtagcgcccgtggagctgctcgcgcggcacgcgcgcgtagttgtagcggtacgagatgcgcccgccctcggccagcgcctggctcagcgcgtccaggtactcggggatccgctccagcaggtccacgatgcccagcggcgcgggcgccgccgccgcggcggcggggccctgccgcgcgcggtgcaccaggtgcaggcacagcgcggcgctctcgagcgccgagtagtgccggttgcccacgtacaggcgcccgcaggcctgcagcgccgtggtcgtcacgcccatgaaggtgcgcgacatgcggccgtcgcgcgagtcgatggcgcgcgagacctgcgggcgctccgcgacgagcgtggcctggagcgtggcgtaccacgtgccgaagagcacgccggaggcggcgccgcgcgcggcgtacaccatggccaggaagtccagcgagaggttggcgtcgtaggcgagcgccacgtcgttcttggcgatctgcacctcgcggccctcgtccgcggccgcgaccgcctcgggcgcctcctcggcggcgcgcgccgcgtccgcctcctcggcggcgcgcgccgtctcctcgagcaccacgagcgcctcggccacgcgctccacctgccgctccagcggccgcagctgctcgtccacctcggcctcgaggcgcgcgcccgcggccatggcgttgtccagcgccgcggcggccgcgcggcggcgcgcgttcgcgtgcgccagcgcgaggcgcgcgtcgaggccctcgccgaagcccgggcgggcccagaagcccacggggaacgggggcgcgatgaagtggcgcgcgctgcccgggatcgcagcggcctcgaaggcgaaccacgcgcggtccatggcgcggggggacatgggccgcgcggcggcgcgcgccgccttatcatccccgctccccgccgccgcccggcccggccccgcgcgcgccgcgatcgcgatcaccgccgcggcccggcgacgtactcggcgaggccgcgcacggtcgcggccatcgcgctcgcgttgccgcgcgtctgggtgcagggcaggcgcgtcacgtcgagcacgcgcatgctccgctgggccacaaacaccagcaggggcacgagcgtgatctcctcgccgcccgggggcacggcggcggcgaggaggcgcgccgagtcgcgcagctggcacagcccctcgtgccgctgcccgcgcttgctgggcgtgttgaggttccgggggaagcggcacgtcttgagctcgatgaggaagcacaggtgcgggcccgcccccagccgcaccacgcacacgcagtcggggcggcgcaccccgaggttgacttcaaaggccagggtcaaggacgccttcttaagcgtctctcggggaagcccgaagagactctcgccgtacgcggacgggtcgcgtcgcaggcgttcgtagaagcggttgtggcagcggatccccgcccggaagcgcgccgggatgcgcatcctccggatctacctcgacggcgcctacggcaccggcaagagcaccacggcccgggtgatggcgctcggcggggcgctgtacgtgcccgagccgatggcgtactggcgcactctgttcgacacggacacggtggccggtatttacgatgcgcagacccggaagcagaacggcagcctgagcgaggaggacgcggccctcgtcacggcgcagcaccaggccgccttcgcgacgccgtacctgctgctgcacacgcgcctggtcccgctcttcgggcccgcggtcgagggcccgcccgagatgacggtcgtctttgaccgccacccggtggccgcgacggtgtgcttcccgctggcgcgcttcatcgtcggggacatcagcgcggcggccttcgtgggcctggcggccacgctgcccggggagccccccggcggcaacctggtggtggcctcgctggacccggacgagcacctcgcgtctgcgccgcggccgtggcggcgcaggcgcgcggcatggaggtgacggagtccgcgtacggcgaccacatccggcagtgcgtgtgcgccttcacgtcggagatgggggtgtgaccctcgcccctcccacccgcgccgcggccggatggagaccgcgacggaggcaacgacgacggcgtgggagggggctcggggcgcgtataaagccatgtgtatgtcatcccaataaagtttgccgtgcccgtcaccatgcccgcgtcgtccgtgcgcctcccgctgcgcctcctgaccctcgcgggcctcctgaccctcgcgggcctcctggccctcgcgggggccgccgccctcgcccgcggcgcgccgcagggtgggccgccctcgccgcaggggggtcccgcgcccaccgcggcgcccgcgcgcgggcccaccctgttcgtcctggacggcgacggctccgcgtggttcgtcttccagctcggcgggctgggggcgctcaacgacacgcgcatccgcgggcacctgctcggccggtacctcgtctcgtaccaggtggtgcccccgcccgtctccgcgtggtactttgtgcagcgcccgcgcgcgcgcccgcgcctctcggggccgccctcgggcgcggagctcgtggccttcgacgcgcccggcgtccggcgcacgtacaccacggcggcggtgtggcccgcggaggtggccgtcctcgcggacgcggaggcgcgctgccccgcggccgtcttcaacgtgacgctgggcgaggccttcctcggcctgcgcgtcgcgctgcgctccttcctgccgctggaggtcatcatctccgccgagcggatgcgcatgatcgcgcccccggcgctcggctcggacctggagccgccgggcccgcccgcgggccgcttccacgtgtacacgctcggcttcctctccgacggggccatgcaccagacgatgcgcgacgtggccgcctacgtgcacgagagcgacgactacctcgcccagctgtcggcggcgcacgcggccgccctggccgccgtggtgcagcccgggccgtactacttttaccgcgcggcggtgcgcctcggcgtggccgccttcgtcttctccgaggcggcgcgccgcgaccggcgcgcctcggcgccggcgctcctgcgcgtcgagagcgacgcgcgcctgctctcgcgcctgctcatgcgcgcggccggctgccccgcgggcttcgccgggctcttcgacgggcgcgccgagcgcgtcccggtggcgcccgcggaccagctccgcgccgcctggaccttcggcgaggacccggcgccccggctggacctcgcgcgggcgaccgtcgccgaggcgtaccgccgctccgtgcgggggaagcccttcgaccagcaggcgctcttcttcgccgtcgccctgctgctgcgcgccggcggccccggcgacgcgcgcgagaccctgctccgcaccacggccatgtgcaccgcggggcgcgccgccgcggccgccgagctcacgcgggccgcgctctcgccgacggccgcgtggaacgagcccttcagcctgctcgacgtcctctcgccgtgcgccgtctcgctgcgccgcgacctcggcggggacgccaccctggccaacctgggcgccgcggcgcggctcgcgctggcgcccgccggggccccgggcgcggcggcggcgacggacgagggggcggaggaggaggaggaccccgtcgcgcgcgccgcgcccgagatccccgccgaggcgctgctcgccctgcccctgcgcgggggcgccagcttcgtgttcacgcgccggcgcccggactgcggcccggcgtacacgctcggcggcgtggacatcgccaacccgcgcgtgctcgccctcgtcagcaacgacagcgccgcgtgcgactacacggaccgcatgcccgagtcccagcacctgccggcgacggacaacccgtccgtgtgcgtgtactgcgactgcgtgttcgtgcgctactcctccgcgggcacgatcctggagaccgtcctcatcgagtccaaggacatggaggagcagctcatggccggcgccaactccaccatccccagcttcaacccgacgctgcacggcggcgacgtcaaggccctgatgctcttccccaacggcaccgtggtcgacctgctgtcgttcacgtcgacgcggctcgcgcccgtgtccccggcctacgtggtggcctccgtcgtgggggcggccatcaccgtggggatcctgtacgccctgttcaagatgctctgcagcttctcctccgagggctattctcggttaataaacgccaggtcgtgaggcccgcgccgggccgcgaacccagactctctgcgtgcgcgtgtttttccttgtcgggcgcaggggagacgggaggggggagagagacggcacgggcgccgtccgtcggggagacggcgggatgacattacgagagtcgggtggaggggatgcggggagcgccgtccacgggggaaggctgctggatgacataattagagccaggggtgaggatcccctttcccccgttccccgagtatacaccactctctcccccgcacacatttcccaccgggcgccgaacgtggattgcaccccggtccgcgctacccggttgcgtgtgagacgggcggagagccgccctcgccccggtcccccaaaaactcctgcggatgcgtggtccgacgaagcctccgcggatatgtccgccgtaccctcatccccctctctcccacagccctctctcccacagccctctctcccacagccctctctcccacagccctctctcccacagccctctctcccacagccctctctcccacagccctctctcccacagccctctctcccacagccctctctcccacagccctctctcccacagccagactcccgcctcccccgtccccgatcgccgaacgcgttcaagaattttttacccgctccgaggaaggggtctctaaggggtctctaaggggtctctaaggggtctctaaggggtctctaaggggtctctaaggggtctctaaggggtctctaaggggtctctaaggggtctctaaggggtctctaaggggtctctaagggggaaccagagagtaaagaccggagagtaaagaccagagacacattttacaatttcgttgtacccgggtttattgaaaactttgttcaagagttaaagtgtctgccgcttgccggtgcgccgcgccgaggccgcgatccgtccgccgtggaccgggggatccgtggggcccgcgggcctggattcgacgggacggcgggtccgagcgacggcggtccggcggcgtttcggctcgcggtcctcgcggcggcgacgatcctggtccgggggatacagcggcgacggcggcgctccgggttgtggcgccgggctccggctccccggtcttttatacccacacctcgtgaatattcatgaatggttggtatgtgcgaatttttcggcgttcgcacttgttgtatataaaatattatatcctatatatattagcaattggtgcgaacgtgacgtggcccaatcattttcctcggatttgcccatgtgaccgagtttccaatagctccgcccggatcgatcaccgggccgatattttgccgataatccatcaatattttcttcggccgacgcgggttccacccgggccgtcccgccggggagcccggcccgggcgcgggactctgctctccgggcgccgcgcggcgtcgagaccgcgccgggacgatggcgtctgcgcatgcgcgggtggaggcgagtggccgggaatcccctctcgtgccgagccccgcccatcgtcgcgtgtcctctccctcacccccttctcccccgaccgggcgacgatgggccgcgtcctcgccacgatggaggagaggcgctcacgcacgcgcgggtacgcgggagttgcggggatcggggaccgggacgatcggggtgtgcggaccgtggcgcgtcggggggctgggtgcgggcggggggtgggcgcgcgcggggggtgggggggggggaacgggaaactggggacgggggaacccgccgaggcgcgcgtgcgcgcgcacgcgtgtgtgtgccgcggacagagacggacgggagaacggacgtgtgatttgtttaacggtttttattgaggacgatggagatgttggcgcgggccaggcgcgcccgaaacagctgaaacaggtactgggccccacggagggccgcgggcgagtcgccgaaggcggtcaggagcgccgcctcggcgcagacggcggacgtgagccgcagcgcgcggcccacgggcgcggtcgcggcgacgaggccccggccggcgtagagcccgtccagcacccgcgccccggccgcctccaccagctcgcacacgccgggcgccggcgcgttcgcctccgcgtcgctcatcatgaccgccagctccaggcccgtcgccgcgtcctcccgggcgtgcttcgtcgtcggcggtgaggcccccgccggggccgcggagacgatgggcgcgagcgcgggggcgtgcaccacggcggcgttcacgagcatccgcatcacctcgccgaagacggtcgcctcgcgcagcacgcggttgacggcgtgggccacggcgctcgcggcccgagaggcgccgccgtcgccgttgccgttgcctccgccgcctcccccctcgccgccgtcctcgtcgtcgcccgcgcccgccccggggcgctcgtccagcgtgtcgagcatgccctccagcgcggcgtagcagctcggcaggtgccagcgggccaggatgaactggtacagcgcgaaccgctgcgcgcccgtgagcgacgtggagcccgtcccgaagaaggggtccgccgcgacggcggacacggcctcgaccacggcggccgaggcgtcgtccgaggcgcacgcggcccgccgcaggatggcgtccgccccggccacgaggtcgcagagcatctgcagggacaggctcccgccgcgcgtcgcccagatgcgccgcgtcgccgggatgtagcgcacctgcacaaagtcgctgaccaccgtcgcgcgcagcgcgcgctcggcgtaccggcgcgggcgcacgctcagcgtgggggccgcgcgcccgcccttcaccaccacggccgtgggccgcgtctcgccgctgagcggggggcgcacgccggggatgccgtcaaagagccccgccgtcagcacctgcagcgagctgggcagctcggagaggtacgcctgcaccagggcgaagcaggcctcgttcacggcgtacacgaagcccgaggaggggtgcacgatggcgggctcggccaggagctccaggacacggtcctgctggcggtcgtgcacgagcgtcagcgagagcgccgacgccggcagcgagaccatgcactcgacgaggtaggggtcgtacacgtccagctccgcgcggccgcagaggtccagctccgtcagcggcagcagcagccccagggcgtccacgaagacgtcgtcgctcccctgcggggcggcccacgcgcggccgaggcgcgccagctccgtgcgcacgtagttggcgacgacgcggttccccggcgcgttcccgcgcagcgtgagcccgaacttgccgatctcccgcgagcgccggtgcacggacacgatgcagcccccgtgcacaaagtacgcgcggtccccgccgtcggcgatgtagaacagcaccccctggtgcacgatcgtgctctggtactcaaactccatggcggcggacacacgcggcgcggcaaatgggtgagcgaggcggcgcgaccactcctttaaacccgccggggggcgcctcccgccgcacacacgcggcggcgcggggatggaggacgcggcggcggtggacgtggacgcggcgtcggacgcgacgacgacggcggcggtgggggagaaggacgcgctgctgagctcggcctttgtgggcgcgcgcccgccgcgcccgcgcttcagctcgcacgtggtgagcctcctggtcctggcgctcgcgctccggcccgcgtgctgcctcgtcctggcgctccacggatcgcgggccaccctcgcggcgctcctcaccgcgctggccttctacgcgcgcgcggccgtctgcgccgccctcgtggcgcggaacgtggcccgggaccggatgccgctctcgccggcgcagcaggtggcgctggggctgctggccgcggcgcggctggccttcttgtacgtcgccctggacgcgggccgccattacgcgcccgccctcgccggggcgctgtacggcgccgactgcgtctgtgacgcgctcgccttcctgctgccgcgcgcgtacgcgcgctccatcatgcattaaaaggcggccgcgacgcgcccgcgccattcttcccccgccgcgccccagcgaccgcgcacacgcgcccgtccgcggtcgtcgctacacgatggagcgcccggccatcctgccgtccgggcagatcctgtccaacatcgaggtgcactcgcaccgcgcgctcttcgacctcttcaagcgcttccgctcggacgacaacaacctctacggcgccgagttcgacgcgctgctcggcacctactgcagcacgctctcgctcgtgcgcttcctggagctcgggctctcggtggcgtgcgtgtgcaccaagttcccggagctcagctacgtggccgagggcaccatccagtttgaggtgcagcagccgatgatcgcgcgcgacgggccgcacccggccgaccagcccgtgcacaactacatgatcaagcgcctcgaccgccgctccctcaacgccgccttctcgatcgccgtcgaggccctcgggctcatctcgggcgagaacctcgacggcacgcacatctcgtcggccatgcgcctgcgcgccatccagcagctcgcgcgcaacgtgcaggcggtgctggactcgttcgagcgcggcacggccgaccagatgctgcgcgtcctcatggagaaggcgccgcccctgtcgctgctggcgcccttcacgctctacgagggccggctcgcggaccgcgtggcctgcgccgcgctcgtgtcggagctgaagcgccgcgtgcgcgacgacaccttcttcctcacgaagcacgagcgcaacaaggacgcggtcctggaccggctctcggacctggtgaactgcaccgcgccctcggtggccgtcgcgcgcatgacccacgcggacacgcaggggcgccccgtcgacggggtgctggtgaccacggccggcgtgcgccagcggctcctgcaccacgtgctgaccctggcggacacgcacgcggacgtgcccgtgacctacggcgagatggtgatcgccaacaccaacctcgtgacggcgctggtgatgggcaaggccgtcagcaacatggacgacgtggcgcgctacctgctcggcggcgagccggcccccgacgacggcaagcccgtgggctcggcgcgcgtgcgcgcggacctcgtcgtcgtcggcgaccgcctggtgttcctcgaggccctcgagaagcgcgtgtaccaggccacgcaggtgccgtacccgctggtgggcaacctggacgtgaccttcgtcatgcccctgggggtcttcaagccggccgccgaccgctacgcgcgccacgccggcagcttcgcgcccacgccgggcctcccggacccgcgcacgcacccgccccgcgccgtgcacttcttcaacaaggacggcgtgccctgccacgtgaccttcgagcacgccatgggcaccctctgccacccctccttcctggacgtggacgccacgctggcggcgctgcgccaggagcccgcggaggtgcagtgcgccttcggcgcctacgtggccgacgcgcgccccgacgccctcgtcgggctcatgcagcgcttcctcgaggagtggcccggcatgatgcccgtgcgcccgcgctgggccgcgccgccggccgccgaccagctgctggcgcccggcaacgccgacctgcgcctggagctgcacccggcctttgacttcttcgtcgcgcccgaggtcgacgtccccgggcccttcgccgtgccccaggtgatggggcaggtgcgcgcgatgccgcgcatcatcaacggcaacatcccgctggccctgtgccccgtggactttagggacgcgcgcggcttcgagctcagcgtggaccggcaccggctggccccggccacggtggccgcggtgcgcggcgccttccgcgacgccaactaccccatggtgttctacatcatcgaggccgtgatccacgggagcgagcgcaccttctgcgcgctcgcgcggctcgtggcgcagtgcatccagagctactggcgcaacacacacaacgccgccttcgtgaacaacttctacatggtcatgtacatcaacacctacctgggcaacggggagctgcccgaggactgcgccgccgtgtacaaggacctgctggagcacgtgcacgccctgcggcgcctcatcggcgagttcacgctgcccggggacccgctgggcaaccagccccaggaggagctcaaccacgcgctggcggacgccgcgctgctgccgccgctcatctgggactgcgaccccatcctgtaccgcgacgggctcgccgagcgcctgccggagctgcgcgtcaacggcgcgcactttcagcacatcctgtgggtcgagatggcccaggtgaactttcgcaacgtcggcggcggcctggtccacaaccggcccgtgcggaacgagaaccagcccctgcacccgcaccacgacgccgagtggtcggtgctgtccaagatctactactacgccgtggtgcccgccttctcgcgcggcaactgctgcaccatgggcgtgcgctacgaccgcgtgtaccagctggtgcagacgatggtggtgcccgaggcggacgaggaggtgggcgcggacgacccccggcacccgctgcacccgcgcaacctcgtgcccaactcgctgaacgtgctcttccacaacgcctgcgtcgccgtggacgcggacgccatgctgatcctgcaggagacggtgacgaacatggccgagcgcacgacgccgctcctggcctcggcggcgcccgacgcgggcatggccacggtggccacgcgggacatgcgcacgcacgacggctcgctgcaccacggcctgctcatgatggcctaccagcccaacgacgccacgctgctcgagggggccttcttctacccggcgcccgtgaacgcgctctttgcctgcgccgaccacctcggcgccatgcgcgacgtgggcgccgaggtgcgcgccgccgcccagcacgtgccctgcgtgccccactttctcggggccaactactacgccacggtgcgccagcccgtggcccagcacgcggcgcagagccgcgccgacgagaacacgctctcgtacgcgctcatggcgggctacttcaagatgagccccgtggccttcacccaccagctgcggcgccagctgcaccccggcttcgccctgacggtggtgcgccaggaccgcttcgccacggagaacgtgctctttgcggagaaggccagcgagtcgtactttatgggccagatgcaggtggcgcgcaccgagagcggcggcgggctgcacatgcagctcacgcagccccgcgccaacgtggacctgggcgtgggcttcacggccgcgtacgcggcggccgcgctgcgtgcccccgtgacggacatgggcaacctgccgcagaacctcttcgccacgcgcggcgccccgcccatgctggacgcggacgcggacgactacctgcgccgcacggtcaacgccggcaaccgcctggcgcccgtgcccgtcttcggccagatgctgccccaggtgcccgtgggcctggcccgcgggcagcagtcggtgtgcgagttcatcgccacgcccgtctcggtcgacctggcctactttcggcgcgcgtgcaacccgcgcgggcgcgccgccggcgaggtgcacggcgaggaggggctgatgttcgaccacagccacgcggaccccgcgcacccccaccgcgccaccgccaacccctgggcctcgcagcggcactcgtacgcggaccggctctacaacggccagtacaacatgagcggcccggcctacagcccctgcttcaagttcttcacgcccgccgaggcggtcgccaagagccgggggctggcgcggctcatcgccgacacgggcgccgccgcctcgccgacgagcaacggcgagtaccagttcaagcggcccgtgggcgccggcgagctcgtcgaggacccgtgcgcgctcttccaggaggcgtacccgccgctgtgcgccagcgactcggcgctgctgcggacgcccctcggcgccgaggagcactttgcgcagtacctcatccgcgacgagtccccgctgaagggctgcttccagcacgcgagcgcctgagccgccggaccgataccggaccgccgccgaaccgtcgtctccgccctgccccctccccccccgactgctctgtcacccgcccccaccctcccgcgccggccgcccgccgcgcgtgtatataaactgtgtacacggcacccgcccgcgtcagtgtcttctttcgctcgcgtcgccatggaggtggacattgcgctgccgacgctgtccccgggggatctgtcggccctgcagaggtgcgagggccgcgtggtgtttctggagacgctgcggcgtcacgcgacgctgcgcgaggtcgcgctgccctgcggcggcgatgtgctcgcggccatggccgcgtaccggcggcgcttcgcggccgtcatcacgcgcgtgacgccgcaccgcatgctggccacgccgctcggcgtcggcggccgcgggcagtcgctggtgctgcagaacacggggcccttcgacctgaccaacggggaccacgtgtgcctcgtcccgccgctgctcggcgacgagtgcctgcggctcacgtccgccaacctggagctgcgcttccccatgacgctgccgctcgcccaggcccgcgagctgacggcgcgggtggtggcgcgcgccgtcgagacgctgcgcggcggcgcgcccgcgcggggcgccgacgtggtcttcaacaacggccggcgctaccagctgcccccgccgcaccgcgacaacgccgaggcggccacgcgctcgctggtgctgaacatgatcttcctgctcaacgagggcgccgtgatcctgctgtcgctcatcccgaacctgctgacgctgggggcgcaggacggctacgccaacgccgtgatccagctgggcagcgccacgcgcgagctcggccagctggtgcggcagcccccgccgccgctgccgcaggaccacgcgcggcgcttctgcgtgttcgaggcgctggaggcctggatcgcctcggcctcgcggctgggcgacacgctcggcacgcggcccgtggcgcgcgtgtgcatcttcgacggccccccgaccgtcccccccggcgagaaggccgcggtggtggaggtgtgacggcgcggcgccgagggggcccgtgtttcccccgccccaccccacccccctgcaataaaaaatccgcgaaggaaaacaaagaaacgagtcgtgcgcgtctggcgtttattttccgggtcggggaaaatgggggcgtcggggggtggagggcggacggtggacggtggatgggggaagccccctcaggcaaacagcccgtcgggcagcgtgctcaggtgcacggccatgacgagggccacggcgaggtcgtcggcgcagccgccgcgcttgccggtgaagacgcggtgctcgccggcccccgaggccgtctccacgaggttcccgagctgggcgttcaggtactcgacggggtcggcgcgcgcgcccacggtcacggacacgagctcctgcgaggccatgacggcgcccgagttgaactgcttcacgaaaaagtcgaaggcgggggtcttttgcttctgcagcaggaagaaggggtagcgcacggcgcccgcgcccgcgcccggggcgccgtggtggtagaagaccacggccgggtcggcgacgacgcgcgcggcgcgcagctccgcgagctcgcggcgcatggcgcccacgatggccacggccgagtcctggctgctgttgccctcgacggccacgcgcaccgccgagaaggcgccgtcgtgcagcgccagcacgcgcgccaggcagcgcacgacgcagcgcgcgatctcgtcggcggaggcgccggtgagcgcgcgcaggaaaaagtgctccaggccgagcacgatccagctgcgccggtagctgcccacgacggccacgccggtgccggaggcgcgcgcgttggccgtgaaggccgggtccacgtacacgtgcagcgtgccggccagcagccggcggttggccaccgaggaggggcggtagagcaggaagcgctcgccggcggcgcgcgtgaggacgggcgcctcgcgcgcggccacggcgccgccgaggatctcctgcatgaaggagtccgggagcagccgctcggccgtggtgcgcgtggcggcgtccatggtgatgaagacgggcttgttgagcacgtagcacgagcaggcggtggcgtcggtgtgcgccaccacgcggcgcatgtgctcgtcgcagacgtacgtgaccacgttgagcatgtcggtgccgcggaggttggtgaggaagctggtgctggccttgccggtgttggtggaggagacgaagatgatcttgcagctggcctggttcatgaagccgaggatggtctgcacggcatcggggcgtataaagttggcctcgtcgacaaagagcaggttaaagtcctgtccgcggatcccctgtaacgacacgcggggagagagagagggggggacggtgagcggcgccgcgatggacgcccacatcgccaacgagaccaagcagcagatgacgcgcttcgcgcccgcgctcgtgcacgtcatcgtgcccgaccccctcctgcggcgcgcgggcgtggacccgctcgcgcccttcgccgcccacgcgcagacgcgctaccacggctcgggcgtctgcgagccgtgggtctccgtgttcgcgggccacgtgcagacgggcgccgtcgagagcgtgctgacgctgccgccgctgcagcgcgcgcgcggccccggggggctgttcgtctcgctgcccctggcgctcggcgcccactttgacggcttcacggcggtggcgctgcgcgtgggcgcgcgcgagctgatcttcacctacgacgagctcctgccggcccgcacgcgctacaacggggccggcgagcgcctggagcgcctgtgccgccagttcgccaactacgcgcgcgcgcggcgcgtggccccggaggtcgcggccgcggggggccacatcgacgcgctgctgcccccggcggcggccaccatcgacggcgaggcgcagctgacgcgcgggggcttcgacgacccggcggcgccgcacgcgcgcgacgccgaccgcgagatcctgtcgctggtgcgccgcgcggccgagctcgtcgcggcgcggcacccggtgcgcagccacgtggcgagcgggctgatgcagggcgccctcgcgcggcgcggcggggacggcggcggctccggggcgctcgaggcggccgcggcggtgcccgccgcgccgcgcgaggccgcgggcggcggcgacggcggcggcgccgcctggcgcgacgagctcctgctcacgccccaggacccccggccgctgacggcgctcgactggctggacgcgggctacgccgcgctcgcgggcggcgacgcgccggcgcacgtgtggcgccggcggcccgtgtccctggtggcgcgccgccactaccagaccggcgagacgttcgtcgtcgtcgcctacgagcactccaccgcctggggcggccggcgcgggcccgcgagcgagcccctggcgcgcgtgctcgccgaggagtgcgagcgccacggcgtcgagcacccgcgcgccctgcccgcggaggcgcggctggagctggtgcggcggcacgccgagctggccgtgccgctgggggacgaggagccgccgctgccggtctttgacgccaccgcggagctggtgctgctggagcgcttccgcaacgcctgcgtgcgcgcgctgctggccggcgtccgcgagtccgtgcgccgcgagccgcgcatgcgccagatcatcgagttcgcgatccgcccgcgcgaccgcgaggccgtcctcgacgtggccgggcgcgccccggcgctgctggacgcgttcgcgcggcgcctcgagcagacgccggcgcgggagatggtcgactcgggcctgatgacggccgccgcggcgcacctcgccgcgcgcgccaccgccggctacgtgaccttcgagagcggcccgctgctgggcggcgttttcctcttcgactactacagcgccggcggggaggttataagggtgacgcgggccccgccggccgtggcagtggagccggcgacgcgcgggcagttcgcgtgccgcttccgcggcgcgtcgcatcgctgtctcccgggcgagagctacgcgtacctctgcgtcggcgtgtcgcgggacctgcgcgcgctggtggtgctgcccggcggcttcggcttcttcgcggccctgcggctggagtggcccgccgcgctggtggaccccgtgctcgagcgcctgtgccgccgcgtgtagcgagggtgggcgcgacgagcgccgccatggaggtggccgcggccctgacggaggactttgccgcctggcggctgctgcgctccgactcgcgcgtcaaggtctacgccgccctcggcgtggtgggcgcgcgcctggccgccgtggcgccgggcgccgcgaccgtcgacgcgcgcgtctacctgacgcgcccgcgggccctgcgcctggcccagggccgcttccacgtcgtggtcctgctcaacgacgccgcctacgcgctggtggcggccgtgaccacgaccacgctccgcgggagcggcggcgagctcgtgcgcctgacgctgggcgacgcgagcctcgaggccctgcccgccgacctgcccgtcgccgagcccgtgcccgccgcgccggtgagccgcctggacctggacgccgccgagcccgtgtccgcggcgcccgcgcgccgccgcgagtgcgtggtcctggcccccggcgcgtggtggtcgcgcgaccgcatctactttctgcagatggacccggcgctgctggcgctgtgccccgccggctggcgcgcgcgccacctcggcgccgtgctcgccgggctgctgagcccccgcgacgacgacggcggcagctgctgccgcgagtgccgcgtggaccacgtggacgcgctcaacgcgacgccgcaccccgacggggcgggcgcggcctgcctgtgcgccgcgccgtgcctgtggcgccaggcggacaagcgggagctgcgcgcgagcgactcgggcctcttccgcgtgctgttcctggacgccgtgcgcttcgtgcgcatgctgccccggcgcaagatcgtcgacgtcgcctccgagctcatcggcgggctggacgcccgcgggcgccacgtggtggtgaacgacgccggctggcgcctggtggccctggacccggacgcgagccacgcgctcgtgtgcggctgcccgctgctgcgcgccctctgcgaccccccctcccgcgccatccccgagctaataaacgactattgaaataaaaaaatgggtacttgcgtttgtgttgtggctggaggcgaacacgatggtgctgcgcgcgccgtcggggaaggtgacggtgatgttctcgcccttgacgtggtccacgcgcgcgtcgcggcaccagcggcgcaggcgcgcgtggatctcctcgaagacgggctccgtggccttgcggatgtgggccgtgtagccgacgcggatgccgcggaaggtggccagcgcgagggcgatgagcgggacgaggaaccaggtcttgccgtggcgccgcggcacgaggaagaccgtgacgcgctggcgaaagtggcgcaccgcgtcctcggagaagagcggcgtgtcgaaggccgccagcaggtaggcggtggcgcgctcggcgtggtccccgagcagcaccgtggccagaaagtacaccgcgtgcatgaggatcatcttctggaagagctccagcgtgccgcgccctccgccgccgccgccgcctctgccctttgccccgcttccagttccacctccaccgccgccgccgtctccgctctcgccgcggccctcctcggcggcgtcctccggctcggggcggccgggcgcgacccccgcgcccctcttctccgcggccccgtccccatcgtcctccgcctcctcgtcgccgtcgtcgttggccccggtcccggccccggccgtcccgagggcggccgtcgcggcgtcgcggaaggagcgccgcgacaggagcgcgaagcggttgacgaagcgccggggctggcggaaggccccggacgaccgcatggcgtccaccgccgccatggcgctcgtgtacgcgttgcggtggaaggccaggcgctcgccgtcgtaggcggcgaactcgagcgcctcggccgcgcgcgcgagcgccgggtccacggcggcgcggccgcgcgcgcccccgaagagcgcggcgcgcgcgagcccggcgaagatccgcgcgctctcgcagcagttgtgcagcgtgccgaccgccgggaccaccgtctggtggcgctgtggggcggcgatggcaaagttgaaaaagcgggcctcctcggcgcagtcgcgctcactggcctcgcgcttgcgcttgaggtccgcgtagtagcgccgcgtcgcgtcgcccaggccgaacatgttcgcctcggaccgccgggagcgccgcgtgcgcctcgcggaggcctttcagcgcgagagcgtcttcaaggcgcgcaccgtcgagcttctgcgcgggcgcgccgacaagaaaaaccccgagttcgtccgggcctttatggcggccaaacaggcccggcgggatgtagagcgccacctgcggctggccgcccgggtggagtctgtggaacaaaaagcgcgcgcgctgcaggcccgcgtggaagcccaggcggccgtccgcggggtcctggacaggcaccggcggttcacgcgggcggactttgccgaggcgctcgacgccgcggaggacgcgctcgtggccggcgaggaccggctcgacgacgcggccgcgctcgacgaggactgggccggcggtggggaccctgacgaggacgagggggaggaggcggacgaggccctgctgacccaatggctgctggaggaggcggcggaggcgtgagccggaccgcgctggcgcggccgcccatccaccgcggcacgtccgcgccctgcggcgacggcgacggcgacgaggcgtcccggctcctgggccgcgcgcagccgcgcgaggccccctacctgatcccgcgcccggacggggacctcgccgtgccggacgacctgcagtacgcgaccttcgacctcacgggcgaccccgtggccgtcggggccggctcgtacggcagcgtgctcgtgtacggctcggtggccgtgaagacgctccgcgccggcttcggccacgaggccgtcatgacgctgctggccgcggaggcgacgcgctccgccggcgtccgcggcgtggtgcgcctggtggggctctcggcgccgctgcgccagctcgtgttcccggcctacgagatggacatggacgcgtaccgccgctcgctcacggcgcgccccgggcacgtggtgcacgccctggggcgcgtcttcgccgagctcgggcgcgcgctcgtgttcctcaacagccgcgggctcagccacctggacgtcaagggcggcaacatctttgtgcgcacgtgcggcaacatggtcgtgacggccgtcatcggggactttagcctcatggccctcaactcgcgcagcacgctcgcggacccccgcttccgcctcgcgcgccgcaaggcgctgaagctcacgtcgctggcgcggagcccgccgacgggcgtcctcctggggcatgcgcgcgaccggcccacgggggtgctgatggactttattaacgggcgcccgccgccgccgggccccctgccgtacgaggtgggcctggcgatcgacctgtgcgcgctgggccacgtgctgctggacgtggcgctcggcctgcgcccgcagcgcggccagacgctgacgcgcgagtacgccgtggaggtgctcgcgcgccgctgcgtcctcttcgcggccctgctcccgccgggcagcggccccgccgccgaggccctcgccgaggacatcctcgaggaggagctggccgccagcttccgcgagtgcgtggccagctcgcgccccggcaaccagcccccgcgcacggtggccccgctgctcgagctcgtggcccggttctgcggcgaggatggcggcgctcgttttgccgaactcgctgcctgaggagctggccgcgcgcaccttcctgcggttcctgcgcgcggcgccgcgccccgcggccggcggcgcgccgctggcgtaccgcctggcgtacgtgcacgacctgctcgtcgagctggcgcgccacggcctcgccgcccccgacgcggccgcggcggcctttggcggcgcgcgcccgccgcccgcgcccgcggacgtccccgccgcggcggcgcgcgccgccatcctgacggtggaggcggccacgcgcgcgcagagcgagagcgacctgtggacgctgctgcgccgcgggctggccacggcctcgaccgtgcgctggggggcggacggcccgcgcttcccgccgacgtggtgcgaggccagcacggcgcgctgcggcacgcccgacaacgcggcgctcatctttggccgcgtcaacgcgcgcgtggcccgcgccgccgtcgcggccctgtacgccgaggcgcccacgccggacctgcccgcggcggcgacgggcggcgacgagggcgacggggacggggcgaaggaggagatgttcaccttcgacgagacgggcgcgcccccgccgggtcacgacctcttctcgtgcgggctgctgctggacccgcgcaccggcatggtcggggcctcgctggacctgctggtctgcgaccgcgacgcgcgcgggcgcctggcgccgcaccgcacgcagacggagatgctcttcttcgagatcaagtgccgcgccaagtacctgttctcggcggacgacgcgagccccacggcgcgcgcgtacgcgcgcctgctcgggcgccccgacgcggacacgctgcgcggcttcctgaactccatcgcccggccgggcgtggagttcttcgagggcgccccgggccccggcgaggcgctggcgacggccgaccccgcgtggcggcgcgcgggcgccgaggacgcccccgcgacgcgtcggaggtgtggcgccttcgacgcgcgccacgtcgccgcgaacgcgcacgcccagtcggaggtctggctatttagcgacccggtggacggccgtcaggacattgtgccctgggcctcgggcgagcgcgcgctgagggtgcccgtgttcgcgaacccgcgccacgccaactttcggcagatcctggtgcagagctacgtggtcgccggcgtcttccccgaccgccccgcgcgcccccacctggccaccttcttcgggcgccgccgccgcccctgcgagcagaaccggacgctcgacctcgcctcgctgtgcgacgtgccgcccgcctgcgccgtgcccgtgctgctgatcgtcacgcccgtctccgtgtgcgaggaggcgttcgaggacctgcggacgcgcgccgaggaggcctttcgcgccaccgcgtcgcggacatgggacagtgctgctgccgattctccagcaaccgcgtcgtgacgagctccggcgacgtgctgaccttcgacgcggacgcgttcgaggactttgagctcgagcccatggtcggggagcccggccccgtgcgccccaaggccccgtaccgcgtgtcgcgcgggaacctccgtgaagcctcgcgggcgtactgactgcaataaacccgtttgtcatactatcgcatcgttcccgtctggtgtctcggggcgggagggacgatgggacgggggagacggaggacgggggagacggaggacgggggagacggaggacgggggagacggaggacgggggagacggaggacgggggagacggaggacgggggagacggaggacgggggagacggaggacgggggatgtggcgggttgggtgtgagacgatggccgaggacgggggaagagggccgtggggcggggcgagatggaatggcgcaccggccgcaaagtgggacgatggggacgggcctcaacgcgggacgagggcggcggggcgggaccggggacaaggacaagggcggcgagagacggacacgagaggaccatgtgcgatcgaaacgaacgtttattcaaagccgaggttctcgtacacgacctcgtcgccgtcgtcaaactcgtcttcggtcatcccgggggccagcaggtactcttcgttgccccggccgtgcaggcgcttctgcaggtaccgccgggccgtcgtccgcgccttgtcgacgttggcgtagaaccggctgccccgggcgcggtgggcgatgcaggcgcggaccaggcgcacgacgatcatcgccagggcgatcagcgacaggaccgccaggacggcgcgcgtcccgatgacgacgccgtgcgtctccccgagggcctcgctgaacttggcgaagtagcggtgggcggacacgccgagggccgcgctggccacgagcacgcagaaggccggcgtggggagcacctgcacgtagccggacacgacgacctcgacgaacgccatcagcgccagcgccagcaccgtgaacacggccagggcgacctccgccgtcttccagaggctgatgtggaagctgttggccaggagcacgccgagcatcagcccgaccacggccgcgcccaggccgaccgcgccgaggacgacgttcgtcacgacggcgcgcgcgtgcaccgccacgcggcgcagcggggggctggccgccagggcggcgcgcacgtcacccgcgcccttgcaggcgtggcacgccccaaagagggcgagaaacacaaaatgggtgatgtaggcggcggcggccagacccgcctgcttgtgggccatcagcagcaccacggcctggagggtccaggcgcagagcgcgccgagcatcagggtccccggggcggccaccgtcgacgccgcgtagagcaccacgctgggctggaacgccagccggccggcctcgcgccgcagcaccacggcccccgcgaccgcgtacagggcgtgcgccagcagcacggtggccgtgaagccgaagaagctggtcacggtgggcgtttccagaaacagggcgggggccacgagcggctggcgcgtccacacgccgccggaggtgtcgttgagcgtgtcgtagtcggccacggtcgcgtaaaagcacgggaaccccatttccgggagcgaggcgaagataagggtgaggaccagcgtcagggcggccagggcgaacccgcagacctctatcagccaggagcgccagctcacggcctcggcgttgcgcggcccggacatggcggcggacgcgggcgagcgccgcggcgcgaccgaggaggacggcggggcgttcgcctcgagcgtgtcgctcgcgcggatgctatacggttgcgacctcccggccgtggtgcgcagccgctggccgggcgtctccctggacctgcagcgcgacgcgcccgtcgagcttccgtccccgcacgataccgcctaccgccgcgtcctcgtggcgcgcgctccgatgggctcgggcaagacgacggcgctgctgaaatggctctccgcggcgctggcggccactgatatgagcgcgctggtcctgtcctgccgacgcagcttcacccgcaccctggcgagacgaatggacgacgcgggcctgggatttgtgacgtacttcgactcggacgcatacgtcatgacgggccggccctaccgccggctgctggtgcagatcgagagcctgcaccgcgtggacgagcacctgatcaacaactacgacgtgctcgtcgtcgacgaggtcatgtccacgctggggcagctctactcgcccaccatggcgcggctggcccgcgtggacgccctgctcgcgcggctgctgcgcggctgcccgcgggtcctcgtcatggacgccacgatcaacgcgcagctcgtggagctgctcgtggagctgcgcggcgagcccagcgtgcacgtggtggtgagcgactacgccacggccgccttcgcgagccgccgctgcctcgtgctccggcacctcggggccgaggtcgcggcgggcgcggcgggcgcgcgggaggacggcggcggcgacgggagcgaggacgcggcgcgggccgggagccccgccccggtgacaggcggcggcgacgacgacggcggtggaggcggcgggggcggcgggggacagcttcttcggcctcctgggcgcgcgcctcgccgcgggggacaacgtctgcgtcttctcgtcgacgctggccttctccgagctcgtggcgcggttctgcgcgcgcttcacgccctccgtgctggtgctcaactcgcagcggccccccgaggacgtgggccgctgggccgtgcgcgccctcgtctacaccaccgtggtcaccgtcgggctcagcttcgacgcgccgcactttcacagcatgttcgcctacgtgaagccgatggcgcacggcccggacatggcctccgtctaccagtccacggggcgcgtgcggcggctgctgcgcgacgagctcttcgtgtacgtggacggctccggcgcgcgcggcgagcccatcttcacgcccgtgctgctgaaccacgtcgtgggcgcgggctggccggcgcgcctgtcgcaggtgacgaacctcgtgtgcgcgcagttccagcgccgctgccgcccggcgttcgcggcggcgcgcgggatgcgcctcttctcccgcttcaagttcaagcacctgtttgagcgctgcacgctgacgagcgtcaacgacagcctcaacatcctgcacgcgctgctggagaacaaccgcctgcgggtggcgctcgagggctgcgagccgccgctgacggcgcgcgccttctgcgacttcctgcgcgacgcgcgcctggacgccttcgccagccagcaggtgctgcggcagctgcgccccccggaccggccggtggcggccgacatcgcggacagcggcgaggtggccaccttcgtggagaagtacctcgtggccgacgtgcccgaggacgagctgcaggagctgctgcgcgcgctcgccaaccccgtgacgcgggagcagtttgtgggcctggccgtgctcggcgcgtgcgcgcgcgtccccgaggcgctgcgcagcgagcgcgtcttcggggccgtgtacgggcactacgcctcgggggccgtgcccgtggtggccgacgggcgcctggagctggcggcgctggcccccgacttcaacgtgcccgcgcgctgggccctcacgcggcgctgcgcgcgcgtcgcggaggccgccgggctcttcgagggcgcctcgccggaggtggactcggccgcggtcgcggcggcggcggccgacgccgagctggcgccgctgctgctcgaggtgctgcggtgccacgtgctggacgcgacgacggcggcgcggcgccccgtgcgcgcggccctgagcgcgctgggggcgggcgggggcgcgggcccgctgagccgcgggcggcacgccgcgctcgtgttcaaggtgatgtgggaggaggccttcggcgtgcgcgtcggccgcagccggcagaccttcccggggcccacgcgcgtgaagaacctgcgcaaggccgagatcgccgcgctgctgcgcgacgccggcctggacccgcccgcgggcgccacccaccggcagctgtacgccctgctgatggaacgccgggaggactttgcgggggagaggtataaactgcgcctgcccgcctggagccggctcatgtacctgacccagggcggcttcgacgcgccgctggacgccgcgctctcgctcgtgcccgcggaggcgtggccgcgcatcgagggggccgtggactttgcggcgctatgacggccgcggcgggggagcgcgtgtgcgcggccgggatctacacggcctggacggggcccggcgcgccgggcgagctccacgcgctggcgcacctgctgtgccgcgacggggccggcgcgtacgcggcgcgcttcgtccacgtgacggcggagccgtggacgggctcggcggccgcggtggccgcggcgctgcgcgacgcgagcggcacgggggcgctggcggacccggcgctgtggcgcggcgcgcacggcgcggtcatggcggccctgcgccgcgccctcccggcgctggccttctacgagccgctgcgcttcgagacggacccggccacgaacctggtcacggccgcgacgccggtggccgggggtggcggcgaggagaggggcgagggggggaagagcgcggaggaggacggggacgacgaggacgaggccggcccgcgcgcgggcatcatccacgtggacgccgacgtcgtcgtggaccgcgacgccgcgcgcgcgcacgccaccgaggcgtggctgccgcacgcccagctgcgcgccatggccggctgcgccgcgctctccgtcgagatcacgacgcggcggaccagcttcgcgcgcgcctacgccgaggccggcgcgccgccgctgaagcgccggggcgagatgggcgacctctttgacgtgcgcgagacggtgctgcggccccggggccggcgcgtggcggcgcgcgtgctcgtcccgcgcggcttcgactgcctggtggcccacggcgtgagcaacgcggcgctggtggcgctctaccgccggtggcacgcggccgcgtacggcgcgggcgacgcgaccccgcccgtcttcgccttcctggggcccgagctcgcccgcgagggcgcggacgaggactactactgcgcgctgggcttccccctgtttgcgacgatcaaggtggccgtggccgcgccggaggcggtccgcggcgcgctcgccgcccaccggctcacggacgggctgtggcccgcgctcggcttccgcgccttccacgccctcgggcccgtgacctgcggcctgcgcctgcgctgcgacgtctggccggcggggcgcgtgaccagcgccgtggagcacccgtgcgccctccgcggggcctggctggccaagtttgacttcgcggccttcttccccaacctgtttctggccctgtgcccgggccacgcgcggctgcggcgcgccgtgcgggcgcgcgcggtcaagccggcgctcgtggccttcttcggcgggctcaagcacacgtgcccggaggcgtaccacagcgtcatcgcgctggccaacggcgtctcgcgcgccgtcgaggcggccgcggccgaggccggcctggtcacgtgcgcctacgtcaaggacggcttctggggcgtgctcggcggcgtgcccgcggacgcgccggaggcggccgccgccgccgcggccgagcgcgtgcgcgccgcctgcgaggccgccgcggcgcggcacctggccgacgcggggaccgccgcgacggacgtgacgctgcgctgcgagggcgtcttcgacgccgccctggcctggtcctgccacgcctactggctgcggcggcgcgagggccccgcggactttgtcggcttcccggcgcgcagcggcttcggccgcgccgccaaggccgcgctggacgccctgctgcgcgaggccgtcgagggccgcgcggacgccggggccgcgcgcgccgcctgcgacgggctcgtcgagcgggccttcgcctcgcgccacgaccccgacttctgggccgccccgggggacgccctcgcccccgccgcgtgcgccggggcgaccgccgggcgcggcggcgcgcggtgctcgcggcgctgcgtgcgcctgcgcggcgccggcccggtgccctacgagcagctgcgccccccgctgatcctgccctggatagactgcctggcccacatggagcccgtcttcgcggccctcgtcggcatgtacaaccgcgcgctcgaggcgctgagcgacggcgacgccccaccgccctttgtgtttacatacacaccggacgcgtttctgtttggttaaacaataaagtctgtttattgtccgaacgagccacccggcgtcctgttgtcgtccgcggtcggggatggtgggggagcgggaaggggggagggaggggggggggggggagggggaagggggggggcggggcggggaggtggaggaggcggaggaggggggaggcgcgcttcactcgtagcgcacaaacatggtcttctcggtgagcgcccgcggcatcgtcgtccaccagcggtacacggcctcggccatctcgggcgtgcgcaggggcgcggtgagcgcgccgccggggcgcgtccgcaccagcccgtcgggcggcggcgcgtcgcccggcaggccgagcaggggcccgaacccggcggcgaagagggcgtccacgacgggcggggtccggggggccgacagcaggcgctgggcgatggggtagtggggcagcacgcgcgtctcgtcgagcgggctgaggtgggccgcgaacatgagcgtgttgagcgcgcgcgtggcgccgacgtgcagcaggcggcgcagcttctgcgccggcggggcgcgcgcgcgctccagccaccggcccaggcgcaggcaccgcgcggcgtccgcctcggccgcggggcagttctccaggaacatctgcagcacgcagagcgcgaactccgtcgggtcctcggggcgcaccagggccaggcggtgcggcagcgccaccggcggcacggccagcgagagcacgcggtcctcggcggccgtgaccaccgcgaaggcgaacccgcggaaggcctcgccggcgaggcagcggcggcggtactcgtcgcaggacaccgtctcgccgctctcgaggcgcagcgtcagcgcggcgccctcgacgcgcgcgtcagtcaccgaggcgctcgtgaagaccgggatcagcccgggcacctcgcgcacctcgcacaccagccgcgggagggcgtgccaggccagctcgtccatcacggcctccatcgcggacccgctcggtcgcccggcgcacgtccgccgcgaacagcgccgcgatctcggtcaggtaggcctcgaggcgcgtcttcttaaacaccacgccgcacaggtcctcctccgagcggtacacctcctggctggtgatgaggaagccgaggtggcgcacgcgcaggatggagaagaagtacggcgcgaggatgagcgagatggagctgttggagtacgagacggacacctcctggccctggttgttggccacgcgcgtcagcttgaagcagcggatcagctcctgctcccagagccgcgagaggcgctccacgtccgactcgtacggcggcacgtagcgcgactggaagctgttggccacgtaggcgtcgtcgcccatggcgcgcgtgatgtcgatgacctcgtggccgaggtcccgcggcggctggccgtccgcggcgccggcgcccgggccgggcgcgcgccccccggggccggcgccgcggtccgcgcccgcctgcgcggcgcgcagggcctgctcgcgcaggcgccgcacctcctgctcgcgctcgcgcacctgctcgcggaggcccccgttgtccgccttcaggctctcgatggtcttgaagaggttgttgacgtagccctcgagcatgccgttgatgctgttgaccacggaggtgcggaaggcgtcctgcaccggctgcgcgccggcgccgtggtgcccgaagcccggctgcgacgtgtccacgctgtccaggatgcgcgccccggtctcgtcgaggtacgagcgcaccgtctccgtgatgtcgccgatgtggcgcatgcccttcatgttcacgatgaggcgcacgaggcgcgccgcggccgagcccgccgggtcctcgccgagcatcttctccacggcgccggccgggtgcccctcggcgggcttgcgcccgacgagcaccttcaccggcgccgtgttgagcagctggcagaggcgcgcgtgctcgcgcagcgcgtggcacgccagcacctcgccgtgcacgcgctgcgccggcgagtcaaagagcagctcgccgtcgcgccacagcggctgccagagcaccaggcactccccgcgctcgcccgtgagcgcgtcgcgcgccagcacgcgccggtgccggggctcgtagtagagctgcacgcggtcgtagtccatgatggtgacgccgcgcatggcctcggcgagctcggccacctcgtccaggccctggcccagcacgctgccggcgacgcgcaggtgcccggccaggtcgtgctcgtccacgcagcggtcgcgccgccgcttccccccggggcgcgcccggcgcaccgggaccacgccgaggcacacgatccagtcgatgtacttggcgaagctggccgtgccgttggactcgctggccgagaagcaggccgcgatgccgcgcacaaagtccaggagggccatctgcagcgtgcggtaccaggtgtcgaagaggcgctcggccacggcgggcgcctcgtcgccgaagcgccgcgccagcgcctcggcgccgaggccgcgcgcgcgcaggtgcgccatccagtcccgggccacgtcctcgtagcgcgccgcgttgagcgtgcgcgtgagcagcgtggactggatctgccgcacggcggcctcggtggagcgcaccgagttgtagatgccctggccctcggtgtagcccaggttgcccatgaggatctccttgaagaccatggtgcgcggcgtggggtggatgaggatccgcgagggggcgcccaacaggttattagcctcctccgcggcgccgccggggcagagcccgtccgcagccgccgccgccgtcgcagccgacatggccgccgccgcagagggtccgcgcgccaccaacgccacctatctgaactttacgtccatgcacggcgtcgagccgatcgtggagcgcgtccgggagctcgccgcgacgcccgcggaggcgccgccgccgctggcgtggttcaagtccctcgccgcggcggacaacccggtggatatagaggcgctggagctgcccttcgccgcgtacctgatcagcggcaatgccggctccggcaagagcacgtgcatccagaccctcaacgagaccatggactgcgtcatcacgggctcgacgcgcgtcgccgcccagaacgtctacgccaagctctcggcggcgtactcgagccgctacgtgaacaccatcttccaggagttcggcttccgcgggaaccacgtgcaggcgcagctcgggcgctacagctacgcgtgccccacgagcccgccgacggtgcgcgagctgcagaagcgcgacctggtgtactactgggaggtgctgcaggacatctcgcggcgcgtgctcgcggggagccacgaggagtttgcgcgcctgcgcgccctggagcggctcacggggcgcgcggcggagcacctggccttcgcgtgccacggctcgctgccggcgttcgcgcgcagcaacgtcatcgtgatcgacgaggccgggctgctcgggcggcacctgctcacggccgtcgtctactgctggtggctgatcaacgccgcgtacgacacgacgcagtacgcggcgcgcgcgcgccccgtgctcgtctgcgtgggctcgcccacgcagacggactcgctcgagtcgcgcttcgagcacgcgcggcagctgtgccgcgtgcgcgccagcgagaacctgctcacgtacctgatcaccaaccgcgcgctgcgcgagtacacggacctctcgcgcaactgggccatcttcatcaacaacaagcgctgccaggagtacgagttcggcgagctcatgaaggcgctcgagtacggcctcccgctgacggacgagcacctgcgcctcgtcgacagcttcgtggtgcccgaggcctacatcaacaacccggcgaacctgcagggctggacgcgcctgtactcatcgcaccgcgaggtcagcgcgtacatgagccgcctgcacgcgcacctcaaggtggcgggcgacgcgcagtttgtggtcttcacgctcccggcctacaccatcgtgcgcacggcggccttcgaccggtaccgcgaggccacgcagcagccgcacctgacgctggaccgctggctcgcggccaacgccggccgcatcaccaactactcgcagagccgcgaccaggacgccgcggcgctgcgctgcgaggcgcgcgcgcagcagggcgtcgtgctcgcccgctgcgaggtcacgtacgtgctcaacagccaggtggccgtcaccacgcgcctcaagaagctggtcatcggcttcagcggcaccttcgaggccttcgcggccgtcctgcgcgacgacgccttcgtccacgcgcagggcggcagcgccgagtacacgtaccgcttcctgtcgagcctgctgtttagcggcatgatcgccttctacaacttcctgcagcgccccgggctggcgcccgaggccgtcacggcggcctaccggcgcctggccgccgtgacggcggcggcgctgcgcgtgcccgaggagcactttgacttctcgggcgcggcggcgcccgccgcccccgcgggccccgcgggccccgggggccccgggggccccgggggcgcgcccgcgtccgcgccggcggacgacgacgacgacctctttgcggcgctcagcgagaacatgctggacatgctctactgccactacgactttgcgcgccccgagaccacgagcgaggtgtacgcgcagttcctgatgctcaagccgctcttcgccgaccgctacgcggcgctctccgagctcttcgggcccgcgttcgcgcgcgcgcccgtcgagacgcacgtggacagcgtctcggtgcgcgggtgcgaggtgtttgtgggggggctgcgcggcgcgctgctctcgacggcgctgcagacggacagctacacgctcgtgggctacacgcacgcgacggcgccggccttcgccgaggagctggcgcggcgcaagctgcacgccggcaccgcggagctgctcgcctcgctggacacgccgcgcgtggtgctgcgcgaccagagcggcttcctgtccatcctcaacgtcaacctcagcgactttgtcgagtcgctggacgacctggagctggacatggccacctacgtcgactacggcatcagctccaagctggccatgaccatcgcgcggtcgcagggcctgagcctggagcgggtggccgtgtgcttcacgcgcgccaacctgcgcatgaacagcgtctacgtggccatgtcccgcgtgatctcgtcgcgcttcctgcgcatgaacatgaacccgctgcgcgaggagcccgagcgggacaacggcatcagcgagcatatattggccgcgctgcgcgacggcgcggtgcacatcgtgtactgacccgccgtccacccgcccgagggggccgagggaccgagggaccgagcgagaccgaccatgacggcgctggtggcctactcgttttacgaaataaagctacccggcggctgggcgcagccgggctgtgggcagaccgtgtgcgagtacgagcgcggcgtccgcgtcatggccacggacgggtgcacgcgctgcgacgcgctcgccccgggccgcgtcacgatccagcacgggccggtgctcacggtcctggccgtggacggggagccggagcggtgctcgtacgtgttcgcgcggacctggccggcggcccccgagggcgcgctcgtgatgcccttctcgacctggagctgcgccgagcgctcgcgccgcctgcgcgggcccgcgggcggcctgctggccaccctcgtcgcggagcgggccctgcacgtcaccatcaccgcgtaccggcccgacgtgctccgggacgccctgcgcgaggcccgcatcctggagtgagaggcggcgagggccgggcgagcggaggaggccgccgccatcaccatcgccgccaccgtcaccatccccatcccactcccatcccaccccgacaataaacgacgacggccgcgcgtgcgcggaaagagagaccgaaccggttgtgtcttttgtctggtccgtgggcatccgccctttattgatcgcggtagcaacaggaggcggggggcgcgcgccggccccgggacggggtccgggggcgcgggagggcggccgccgaggggacggcggcgtgggccgaggcgtggacgctgcgctggcggggtctcggcagctggggcgtcggggcggccgtcgccgaggcgttgatgctccgttgccggggccgcggcagctggaccgccggggcggccgtcgccgaggcgtggacgctgccgcggcggcgcgggggtgcggccgcgcacgactcgggtccgtccgcggccgtcgccgaggcgtggacgctgccgcggcggcgcggggcccgctgctgttgttgctgctgctgcggatgctgagagcggtggtggtgctgctgctggtggtggtggtgggtaggctggacgacggtggcgtagacggggtccggctcggtcgcgccgcgctggcgcgagcgcccgcgggcggtgcccaggccgccggcgcgcaggggcgcctcctcgtcggggggggcgcgcgaggaccgcggggcccgcgtctcgcccccgccggcggagacgcggcgggaggccgcggccaggcgccgggcggccgtctcgttggcctgcaggaaccggtggtagtcatcgaccgtggccacggacgcgcgtccgaaggtgctcatggcgccgcaaacacgttgtcccggtgcagccgcaccggctcgggggtgatgaactcgtggatgaacaccggcgcggccggccgcgttcgtccgctcgtcgggcgcgcgcggggctcgcgtgggcgtcgggcgcgcaccgcggaccggagctgatcgcgtacgcgcgccgcgtgggctctgcggcacaggataaacatctgcaggcttttatggccgcgcccgggggcgccgcgcgggccgcgcgcgccgcgcgcaaacgcggacgtcttggtgcaggcgacggggaggccggccagggccgcggccaggtcccggcggatcgtgtccgtcagccgccggcgcccgagctcgtccacggacgcgaccataaacagcgtgtcaaactccacgtgctgcgcctggcgcgggcccgccgccgccgtcgtcgtcgtcgtcgtcccttcgtcccccccatcttcctcgtcttccgtctcctccttctccgtcgccttcccctcctcgccgtcttccacgtccccctctccgtcttcgctcccgcgcaccccctcccggggagggggtgcgctctccgggggcgcgtccgcgtcctccgggcacggcccgggggactcgacctcctccacggcccatccccactcgcgcagcaccgccaggaccggcagcgcgctggccggggcgccggcgagcgcgggctccatcatccgctctcctccgtccatcccaccccccctccccgcctcctcgtcctccgccggcggtgtgcgcgcacgcccgactcagtccacgctccagtcgacgggggcccggcccgtctggacgaggtacgcgttcgcctctccaaagtgcgggcaggtcctgaagggcgtgcgggccagcggcgacgggtggctgaaggtgagcaccttgtggcggcgcgggtccggcgcgcaggccttttgagcgtgggcgccccagagcatgaacaccagcttggggcgggtctcgcagagccgctggacgacggcgcgcacgagccgcgcccagccgagcggggcgtgggagccggggaccccgcgccgcacggtcagcgtcgtgttcagcagcagcaccccgcggcgcgcccaggcctccaggcagccgtgggcgggcgcgggcagcgtcgggtacgtcgcccggaccgccgcgaagatgttggccaggctcggggggatcggcaccccgcgccggacgctgaaggccagcccgtgggcctggcccggcccgtggtacgggtcctggccgatgatgaccaccttgacgtcctcgggcgccgtgaggcgcgtccaggcgaacacgtcctcgcgcgcgggcagcacctcctcggtcaggcagcggccgcggtactcggccagcagcaggcgcgcgtacggcttggcgagctcgggctccagcagcgcgcgccacgagggggccacgtcgaactcggcggcgaaggcgtcccacgtgggcgccccggggtcgggggccggggcggaggcgtcggcccccgtggctgttgtcgccgccgacgccgcgggcgtgcccttcgcgggccgggcggcggcggagggtcgggccggcttcgaggccccggcccctgcccctgcccctgcccctgcccctgccccggcgggggctgctgctgcagcggcggtggtggcgttagaggcagaagcggcggcggcaggcacgacgaggcgcaccccgggcggcaggccgcagggtctcttactcggcggggggccctccattatagcgcagcggcggcagggagtccttgtgcacggccacctctccgagctccttgagggccgcctcatttaagggctctccgacggccagggcctggatggcgacaaaggggttgaccaggtaggcggtgccagacgcccacaggatgacctccggcggcgagcagtagggcttcacgaccacgctcgtgacggcgttcatgtccggcggcagcgagtacgcgatgtagggatggcagcgcgatcccacgggcggctcgaggtccagcagcgggtgatcctcatcgtcccactccagctcctcgcgccgaggatggcgagtggggggcgcgggcgtgaccggggcctcgaagaaggcatcgagcccacgggggtttcccgcgacgccggggccaggaatccccagcgccgccgaaaaaaacaccagcaccgccacgagcggcgacatcgcggctggtgtatcctttttcggcgcggggtcccggcttttatctccggaaagaggaaattgagaaataaactagacatccgcataatcccctttgatttacggaggggaaatacgtgccacgtggcggggggcggggcaactggagcgtggcgaagcggaacggggtgcgatggggcgcatcgaggcgcggacgcgtctgggggagtctggggggagtctggggggagtcggggggggggcggggggggggcgggggggagggcggggggaattccagctcggcgcgatcgccgcatcgcgcaaagtccggctgcagtaaatttactgcggatgcagttcccggggcggccgccccggccagatggcgctgcagtatagctactgcggctgcagtttactacagttgcagtaccgcgcgccaccgctaaatactacagtagatttaccgcggccgcggtaaactgcagtttcctgcggccgcggtaaactgcagtatcgcgcggtaaattgtagtctggcagccgcgcgttactgcaattagcggtggctcccgacactctggccaattggtgctaatgggccgtgatggtccacgtgggggtgatgtaaccgccgggccccggttgggcactcagacagtggccgggcgccaggccaaagtgctgtctgagtgccactttatgactttgtttttctcaaacaacatcaattatggatgcacatcgtgtatataatccccggtccgcgctccgcccacccatcacagcagccgcggacgctgcgcgccggagcggtccatctcgccagccagccaaccagccgagccgcccggccgacccgagagccccgagagccagactccctcagccatagaagacaccgggcgggagagacggactgaaaaaatatatctttttttattttgtccgggcctggagacccgcagcaggagcggaggtgggtgtggggccgggagccggagcaggaccgggaacaggaacaggaacaggaacaggaacaggagtggggccgggagcaggagcaggagcaggagcgggagccgaagtgggggcaggagcggcggcggccgcagcagcaacagggtcgccccagtccgcggcgaggaagagggagctcagtcgtcgtcctgggtgaggtcgatgaagatggtagcggagcggggggatcccgacgagctagacgccggaggcccgccccggggggcggcggtctcgggggcagaggcagagggcgacgggcgccgcatcgaggaggagggtgaagacgagggggaggagcgagccgaagcggcggtgttcgccgaccccgggccggccccggcccccgaggcaccatgctgcgcagaggacccctcgccggacgatggcgcctccggagtctcgccctgggcctgtccgcccgtccggccgcgccgcaggcaccgagtccgtctctgcgcgcgcctcagcacggccgcccgtcgggccctgcgcggggagcgcctgggcgccggcctctggtcgtccgcggactcggaggcctccgtcagatcctccgtgtgcaccccgctgctcgaggcgcccgagtcttcctcgtcgggggaagacacctcagagtcagagtgcgcctcggactcggacgtgtcgatatagttcacaccctggtggctcatcggggctcgctgcatccgccgcatccactgcgccgatatgtcaaacagcgtatcgacgagggcgtgggtgtttgccccaaacatggggagcatggcctcggtcacgcgctggcggttcatcccgtgctcctggataatctcgacgatgttgtccacgacggcctcgcggatggggtcgctctcgatgaccgtcgagacctgcccataaagccagttgaagatggggactctggggcgggcgcgagacccagacccggagccctgcccttcggcctcctcgtggcgcacctcctcggtatagtcttcaccccagatgaccgcgaagcccccccctaccggctcatcctcttccccgtcgacatccgtcgccccctccacgggcgtctccacaaacgaagcgtcgctgtccacgtggtggaggatggaggtgacgcgggtcttgcacagcgggcaggcggtgctcgtcagggtccagcgctggatgcagtccagacagaacttgtgcatgcacggcagcgtctgcgcctcggtggccgcgacgtccaggcagatggggcagtccatgacggtgagtggcggtagcaggcgctgcggcggtggcgaggggcttctggggcggtagaagccaaagatcccccaggcgctggaagcctcggcggtggtcgtccgtctccgagagaccgtgcagcccatggtgtcgagggccccgttttttaaccgggtgcccggcccctttgtttgtcttcgcccggggcgtccacgcccccttccgcccgccccgtcggggctccccacgccccctcgggccgtcccgggacgatggccccggcgcgcgccgtcgtgggaagcgggccctcccgcggtgggtcgacggctcctgggtctgaaagcggcgctacggatcccccgctctcacccctgggtccgtcgccgccccgcggtgcgtgcgacgcccgcgaacccccggcccacaacacaccggcaccaccgccaccatcatcatcccccctctccacagacacgctcgcactcgggccacacgctcccgtcctccgccgccaccctcccagcctcccagcctcgcgggtcctcgggacgaccgccgctcaccccgacgggggggggacggggacagggagcgaggggggaggaggacgggggggggaagaggggaacgcgagcgcggcgcaccccgtcgccgtccgtggtggcgtgcccgccaccccgtccgtccatccatccgtccatccaacccccttctcctttttccgcctcccgtcccacggcacggccccatcgacgccacaccgcgcggtgcgcgctctccagccgtcccatccccccacaccccccacgctgcccccattcgcttctccccccgtcagacctgcgccatggagtttcgaggacgggcgcggggcaacggtggtgagaggcgagccgggggggtcgagtgggcctcgcactttacgcgcacacgcccctctcgcgcaccacacaccaccgcacatcgccgaggtcgggcgagagagggcgccctccccgcgaccccgctcgccccggtcccgacgccctcgcacccccacagccccccccccctcccccccccccccccccccgctcccccccgctctctccgcgcgatcgcacacacaacacccgcgcgcatgctcggcttcttcacacggaggacaccatgggcagcagatggtacggtcagccgccggccgccgtgcgcatgtgtcgggctgtcgcggccaagagtatcagcgtggcttttttgttggtcagggtgtgaaaaaggcacgctgatgcgtccgccgctgcagcggagctcctcggcagcgagatcatcgaccaccaccaccacatcgctcttctcgcccgcgctcccccaccgccccccccgccatcgcccgcgccccttcccccctcccccccccccgcccccccccccccccccgccccccccccccatcccccacccccccgtccaccctcaccccccatcgcgtcgcgagcgctctcacacatgcttacatgcacacgcgcccctccctcctcggcgcccccgcggcgggaccgtcgcaaccctctcccccgccgtcccacccgtccgcggccctcctctccttttccccctccccttccccttcccatccccgttcccccctgttccccatttccccgctccctccaccatcgactctcatcgacgggcttctccgccatcatcaccaccaaccaccaatcaccatctcccgccgccgcccggacggcgacccgcggtgctcccgggggcgcgttcccactcacaacccggtggtttcgatggccgcccccggggggttgatggggatgggcgctcgggggtgatcgtctgctaccaaaccgcaccaccgtccccctgtccctcatccacttctcgacggccggctccccgcggggcgggggctgcgggggggtgggtgggtgatagagcaggcgggcgggtctggacgggcggaggggggggtcccggcccctctccctcccctctcttcccccctcccctaccctctctccgtgccgactcgacgagcgggggcgaggcagggtgcgtaccgacccgcctaccaggcaggctagaagcctcacgctgctgcttggcagcgggtgggtacccaacccctcccctcaggacgcgaccgaccggtgtggagagagaaagatgcccgtcccccctgcccccaccatccccgatttttcggttcctctccccgtcgcgccgtcgggcccctcgtcgccaggggtttttttggtggaaggcccaccggggtgagggggggggggggtggtggtgtgtgcgcgacggaacgggccgcaggaggatcggggtgtatgcacggtcgtccccgtctcaaacttcctcgtgtcccctgttccccatccttccacccatacattcatccgtgcggtggtggggatgagtggatggatggaggcgagggacgcggtggaccatgccaggcaagacctgcgtcactcgaagaaagggtgcctggattggccttcgcttctttgccggccacgagcgacggccaccaccgcccccccgacgggctctcttttcccccctcccgcttctcctctttcctctccggcgcgtaccccaccgttttcccgacgaggttcgagaccccccccaaagcaaagcgagagtaccccacacccgcacgcactccccggtgtcgctccttttcccccccggcagcgccggaaccgggtgcaggcggtgggggcgaagattgggttgggtgagagactagaaccggtgttctcaacccttctggagccctaccctctgtgcctggactttccagcgcgcgacacgggggggagcgggcacgcggcggtggcgatggtggcgacggcggtggctgggcgcgatcgaggagatgtggaggggtgccaagcgcccgccgccctcccccgcatctcttctctctcgcacacgcacacgacccgcggatggcgaggatgtggggggagaggtctcccggcgttagtgggtgctatttgattctcctctaaccccctctccctcgcggccccctcgaccatcgtcgtgcagcggcgacggcggctcccaccccgtctctctcccccacacgacctcgacgcgtcccgccaccccgcccagtcacgtcccaccccccggggagaggggagagggatgcacgggggtgggggtccccgccccctctctctgatcccaagacggtgtttgtccccgtctccggctcgcacgcacacatccccgccgggcgggccccggcgggatgcccggccagacaaaccgagggggaggagagggggaggaggaggaagtataggcctctcggggacgggcggcgcgtggcgccggagaggggggtggcggggaagagggaaacctagaaggcggcccatcccccgagatgacgagagcggagagaggcgctaaacggcgcctggggagtttgggggaggaggagacggcgcgcgaggagctgggggaggaggaggaggaggaggaggagacggcgcgcgaggaggaaaatggccggtccaacccttgggacgagaggtctcgaatcttgacaccatttcccctcccctccaccccactcccgtcgcggcttcgcgcggggaggaaaataatccgatccgacccccggggcgagaggctcgcgatcccacccccccgctccccgggggccgcgaaaaaagggggcggggcttaaagggggggggctaatttgcataaatttgcatgcatttgcatagccccgccccctcatttgcataaattagcctggccccccccccccatttgcataaattagcatggcccctcccccaatttgcatggccccaccccctcatttacataacccctcccactcatttgcatgcatttacataacccctccccctaatctgcataacccctccccctaatccgcataacccctccccctaatctgcataacccctccccctaatctgcataacccctccccctaatctgcatatgacaacttcctccggaagtgacgctctggcccctcccccctcatttacatatgaccgcttcccccggacgtgacgccgggctcctcccacccccccccccatttgcatatgaccgcttcccggacgtgacgtttttgccgatgacgacttccgcctcatttgcgtatgaccgcttcccgcgcgcaacttccgccggacgtgcgtatgaccgcttcccgcgcgcaacttccgccggacgtgacgccggtccccacggatctcatctgcatatcgcgccggccccttccgcttcccccggacgtgacgccggcttccggggcgcggacggggcgggctccgcgggtcgcatcggcgcgccgagcctgccccttccgtcgcaccgggggtccgcgggcgggggcttccgctccgcggcacccgccccattggctcccctcgcgccacgcggtcgccgtcgtcatcgtcccgccggccaatcgggtggcagcgggggaggctgggagtggggacggaagacggaagccagatgaacctgttccgcccgctctcccaccgcctttcccgcccctccacgacgaccacccgggaccaccaccaccaacaacaccaccacccccccccacgcacacactttactactatcaccaccagggggcgatggttgcaatggcagttccctgtactgaccacccccgtgtttttttctctctctctctttcctccccccccccccccctcgaccaccgcaggaccaccatcgtctaactcccacccgggaccaccgggaccctcgggaccatctacctcccaccaggacccgccgggaccaccaacaccgtccacctcccaccaccaccaccatcaaggacccccaacatccccaagaccctctacttcttcccacccagacccaccaggaggaggacccccatctgctgagacccaccaggacccaccgggaccctcatctgctgagacccaccaccaccaccaagaccctccaggaggaggacccccatctgctgagacccaccaggacccaccaggacccccatctgctgagacccaccaccaccaagacccaccaggaggaggacccccatccacttcttcccatcaccaccaccaagaccctccaggaggaggacccccatcacccccaccaagaccctccacctcttcttcttcctcccaccagggacccccatccacaagaccacctccgccccagagaccaccgccaagatggccgcctccatctccccaaaaaatctcagagactcgagctggttcagaaaatacagcacaaactttattttctcactctgaaaataaactcttttctcacccgatgggagaaggaggagaaggggaccgggggaccgcgggaggaggaggaggaggagaaggggaccgggacgatcctccgccgccgagccctccgccgcggccgccgccgccgcttccaccaccgccgccaccaccgccgccaccaccgccgccacctccgccgccgccgcagccgcctccggccgggggatccgcgcggaggagaaggagaggaggaggaggaggagggccgccgggccggggaggcaggcgccggggaggcaagcgccgccgggccgaggggaccgaggccgccgccgcggacgcggaggaggaggaggaggaggaggacggggacgaggacgaggaccgggccgggggcgaggggagagaagacggaggagaagggcctcgaggagccggtggaggggccggagagtcagagtcagagtccagccgggccgagggggcgccccgctcagcggagcagcaggtaggggttgccggcgtcctcggcctcctcgtcgtccgagatggcctccaccttgatgggcccgagcgggccgcggggccggccgtcgccgccgcggaagtcgacgatctccaccgcggcagagtcctccccgtcctcgccgggcccccgggcgcccgagggccggtgggtctccacggcgccgccggcggcgcggacgctggtctcgaagggcgcaaagtcccagcgcacggccggcggggcgcccgcggccgcgagggcgcccggggccagcaccagcggggcggcctcggcgtcgggctccagcagcgccgcggcgcagaaggcgcgcagctcggccggcaggccctcggggccgcggagctcggcgaggccccggcgaccgcaggagacgaagacgggccgcagcggggcgccgagcccccagcggttggccgcgcggtgcccgaaggcggcgcccgcgtcaaagtccgggtccccgagcccgagcgcggagcgctggcgggccatgtccttgcagccgtccacggtggggagcacgcgctggcggtaggcgcgcggcggcagcgggaccggggtccggggcccggcgcgggtgctcaccgtgtagcgcacgttgtcctggcggcagaggcgcagcggctcggccccggggtgcaggcgggcgaaggaggcctccacgcgggcgaagcaggccgggcccacgatggagctcgagtccaggacggccgcgcgcagctcgcggcactcgggccagcgcacggcgcactgggcggccgggtccaggcgggcgcggacgtagacgtggtagtcccccacggccgggccgtccgcgggccagtcctcgatggtgtccagcacgatgagccggcgccgcgccgcgccgagccgcgagcagaggtactcgacggcgccggcgaagccgaggtcccgcgccgagagcagcagcaccccctgggcgttgaggcggccgatgtcggggcgcccggtccagttcccggcccaggcgtgcgagtccggcgtgcagaggcggtgggcgaaggcggcgagcagcgccgagaggccgccgcggcgcgggtcccaggccgggcgcggggcgccctcggcgggctcggcgcagagctcctcgtggggcagcgggtcgtagagcaccaccacgcgcacgtcctcggggtcggctatctgccgcatccaggcggcgcggcggcggagcggggcgcccgcggccccgcggcgcgcggcgatgtgcgccagggcggccgggtcgaaggtgagcgccgggcgccagagttcggggaagacctcctggtccacgaggacgcgggccacctcgggcgggcagtaggcggcgagggccgcctcggagggccgcggcgtgtgggtctcgccggccgggacgcggcggaagccgccgtcgggcgcggggtgctcgggcatgggcccgagcgggcgccggagccggtcgtcctcggaggaggaggaggaggaggacagcagcgcgggagcggggtccggagcgggcccgagtccgagggagcggcgcttgcgccggggcccccggtcctcttcgtcgtcgcggtggccgtggccgtccccgcggagggccgagccggagagcccctcgtcctcctcgccgtccccggggcggcgggccccgggcgcgcggcgcttcttcttgcgccgctcgggcgctgggtccgggccggcggcgggggagctggcgtagccggaggagccggagaggccggacttggtgccggggctgccggacttggtgccggagctggacttggtgctggagctggacttggtgctggcggggctggagggcccggagccggggaggccggagggggcgcccgccgccgccggcgccggcgctgggacgacgaggccgggctgctcgggccagagcgggggcaggccgggcgcgggctccgcgggcccgggccgcgcggcggcctcggcgagccgggccgcggccacgttggccggggcgaagagggccgcggcgtaggtccaggcggcctcgcgggcgcgggccccgtccacgctgtagcgcaccagcggcgccacggtgcgggcgacgagggcgacagagtccgcggcctgctgccgctcggccgggccggccccggggatcgcgtcgcggagcgcgagcagcgcggcggtcacctcctcgaggcaggcgggcccgagggcggccggggcgcgggcgggcgcgggcagccggagcgggacgggcagcaggcgctcgaggacgccgcggcaggccaggacgcaggcgtccgccagctcgcggggcacgcggccgggctgcgcggcggcgaaggcggcgcggacgcgggcgcagagggcctcgacggtcgcctccccggcgcgggggtccgcggcgcggcccgggtaggccatgtcggcgtaggcccggcggaggctctgcaggatgaaggtcttctgggtgcgatcgtagcggcggctcatggccacggcgctcaccgcgggcggcagggcccagagcgggtcctgggcggccatggcgtccccgatgtgcggcagcggcggggtcacgctgccggtgatgaaggagccgtggccgtggggcgcgtggacccggcgctggcagaactggttgaagcgctggtcgggggcctgcatccgcgggttctgcagccaggacatggcctcgccggcggccccgctgtagatgaggcgcacgagggcctcgtgctgcttcctcgagtcccccatctccgggatgaagacgggcacgggcccggccgcggcgcggtagcgggccgcggcctggcggacgtcgtcctcgtcccagagcccctcgcgggagtccccggcgccgccgtagcggacgcggccgtcggccggagggtcggagccgggccagggccccccgagcggggtgagcagcggcccgtcggtcggcgggggcccgtcggccatgagcgagaggtggttgttggtggagcggcgcttcctgcgcgggggccgggcgggctccggggccggggccggggcggccgcggcggcggaggcggaggaggcggcggcggcggaggaggccgagggccgcggggccgcggcgggcgccggcggagacggtggcggcccggcgcgggcgagtggggcgccggcccggactccttcgtcttcttctccctcggaggacgtcccctcggaggaggacgaggacgaggaggacgaggaggacgaggacgaggaggaggccgagcgccgcgcggcggcggcggcggaggcccggggggcggagggcgagcgggccggggagaggtccgagtcgctgccgctgctgctggagctgctgaagccgcggccgcggcggagggcgccctctccggcgcggcgccggcggggctgtctctgcgggggcgccccgccgtccccggcgaggccgagtccgtcctcgtccttctcggggtcgcgggcgacgggctcgacggcgacggtggtggaggagctggagttggagttggagttggaggagacggggctccgggcgccaagcggccgaggatcgagccgcctcgcggcggcgggctcgtcgagcaggggctcgcggtgctggtgatggtgacgaccgcggtcccctccggcggagggggcgccgccgccgccgagcgccgagaccggcccggcggcgggggaggctggggaagcgggcccccgccgtgccggcgctgcggccaccgctgctggctgtgctggtggcgccggggtccgaggccgcgccgccggcccgggctcaccgaccgggtcccccctcgcgggggaccatctccgcggggctgccgaggggccgggggagccggaggaggaggccggggaggctgcggagggggacgggcgcccggggccgccgggggccccggcctctgccgcggcgagtgctgccggggtcggcggccggggcccggagccggcccgggaccggggcccgaggacgaggtgaccgtgctcggagccctgatggagagcccgaccgggggacccggcggccggggacccgggctcgtcctcctcctcgtcttcgtcgtctagcaccacgatctcgcccgagccccggcggggctgctgctgctgctgctgctgggccgaaggaggacggggcggcctcgtggctccggccgcggccgcgaggacggcggcctcggcctcggcctcgccggcatcgtcggagaagaggccgccggggccaaagaggagatcctcgccggaggggccgcggcgccgggagccctggctgccgccgtcggagccggccgcggtcccctcttcctcggccgccgcggccgcggccgccgccgccaggagctggctgaagttgccctcggtctcgatgaagtcaaagagatcgtcggccatggtctcgatcggttctcggggtgtctctctctctctcgcgcgccgggagccgagctcggagagcgggtgctggagggtgagagggagccggaggagcgggcgcggcagaagaagaagaaggagagggcgaagaaggagaagaagaagagggctctcctctctcttccccccggtgggtcgggccgtccgtcgagggctgatagccgccggagagccggagtcttcagagtccgcgccggagccgagacgatcggatcccctcgggttggcagagaacgatgctgtccgtacctgcaccgcagtgaagtgctacgatggagaccgtgcctataaactccccgagaagagcccgcccccaggtaagcggaccaatggccgattttcgccgcggacttccccgacggccggccaatgggatttttctcgcccgcttcctctcgcgtctgctttgcatgcccgcggcccggcccaagatggcggccgccggccaatgggatttcgcgaggaacttcctcgcgaggaccatttgcatgcccggcccccgcggcggccatcttgcccactcgacggccaatgggatttctctcgcccgcttcctctcgcgtctactttgcatgtccgtcccccgcggcggccatcttggtcgacggcggccaatgggatttctctcgcccgcttcctctcgcgtctactttgcatgcccggccccgagggcgccatcttggcccctcgacggccaatgggatttctctccctacttcctctcgcgtctactttgcatgcccggcccccgcggcggccatctcggctcgcccgggccaatgggcgcgcggaggcgtctcccgcgcgcctctgatttgcatgcccggcccgctctgcggccatcttggccgcgggcggccaatgagattgtccgaaaatccctcgcgcgggcgcgaggcgcatgctcggcacgcgacccacccccgtggtgctagcgagccaatcagatgattttcggggaagcttccgtgtgcacgtcatttgcatgctcgccccacgtggccgccctcggccaatggggcctcacggtgcaagcttccgtgtgtctgcacgtggtccgcatgtgttgtggtggtctctcagtgttttgtggtggtctctgtgttgtgtggtggtctctcagtgttttgtggtggtctctcagtgttttgtggtggtctctgtgttgtgtggtggtctcaccgcctccccctgccactcgcgagacccccgagacccccgtttccccctccccgagacccttgaggccccccgcgacccccgcggtcgccccccccgcgcctgcgcgctcggcgcgcgctccgagggcgcccccagccggtcggagagacgagcggaaccgccgtcggaccggggaccggcgaccggacccgaaccgggaagcgacgccggggcgggagaaccggacccgaacctcgagcccggacccgcccggacccggaagggaggagccggacagccacgccttggatacttttgtcgcccacccacccccccccctctacctctctctcccggtcccccctcccaccccacgagacacgccccagagtgaaaaaaataaaagttgttctcgttgcaccgtcttccggctcgtgtcgtccttccgcggtacctcgggcgggcgggagggggcgcgagaccggctcggcctcgtcgggagagagggagttgggggggaggggagccaagatggcggcggggcgtggcggggcgtggcagaggggaggggggggggtcggagcgcggaccccgcccggtgggggggcgggtgcaaaggggcggggcccaaaatggacctcgggccgggaccccgggggcgctccgggagacgaagcgggccgggcccccttcccggcggggagggccgggccggcgcgccgggccgcccctccgggggaaagagtgtccccgcgcgggcgccgcgtcccgccccgagaccccgggggcgcgcgggcctcggtcgcgcccgccggacgcggaggcgcgaggcccccgccccccggggggtccgggacgggggggcccaaaatcgggctgggggacggcgggggggtttgctccggaggtctgcgggtgtgagtgtgtgtgtgtgtgcggggagagaaggctcgctctctctctcggggggccgggcgggtggggggtgggggggggggaggggcgggcgggcggggcgggcggggggggtgtcgtccgggggtcggtgcgcggggggaggggggggtgggggtgtgcgagagagagagcgtgcgtgtgggtgggtgtgagggccggtgcgggtatgggggcccgtgcgggtgggagggtccgtgtgggtgtcggtggtgcgggtgttgctggtgataccggtaccgacggtggtggccgggcgggcggtctctccgtctccgtctccgccgtcgcctcggtccgaggaggggggcgcccgggcgtgtctcgcctttcccccagttcgccgcctccttctcctccttctcctccttctcctccttctcctccttctcctccttctcctccttctcctcctccccctccctcccctcccctcctccccctccccccgccccctgtcccggtcccggtcccacgcgcgcgccccggggaagggtcgggcgatggccgccgccaccgccccacccttcccccggaccccctcccgcccgccggggcgccccgcgtgctccgggggcgccggccggccatccccccccacctcccctcctccccgtccccaacccgccccgcactcgcccgacactcggcccgcggcacgcgcgcccagaccttcctctcccccctccaccgcccgccctcgccccctccgtctcatgccacccgcggcagcaccccccctccccgtcgtcctcagccaacagccaacagccaacagccaacaacccacagccaacagcctcactctcgcgctctccccgtcgcgggaaaaaaacacggcaccacaggcgggagcggggtcgctcgggggaaggtttcattcaaacgacaggcgacaggaggggggcccccgcgcgggtcgggccttcttccccatccccgccggcccccctctttctctctctctcttcttccgcgtcttcttccgcgtccgcttcggccccccgcggccgcggcgtgggagagcggggcgggtggggaaggaagaggggacgggggggagaagaagaaacggggaagcgggggggaaggaacagacggggaaggggggggaaagggaaacggggaaacggggaaggggccggcggaggggaagcgggagggagaggcgcggcgccccgcgcttccccttcccgcttccccctccgcccggccgcgggcgcccgggagacgggagcgaaaggaggagaggggaggaaaaaaagtctagagcggggaggagaagtcaccgtagtagtcatagtagtagtcatagtaatatagtaggagtaggagtagtaggagtagtagtagtaggagagtctgtgcttaccgagagaggagaagaagaagcagaagccggggcgcttcctccccggcggcccgggaagaaagccccggcgcctcgggccccggagatggggacgcggagaaggaaacacccaagggcggaggaggaggaagaaggagaggaagggaaaaaaaaacctcgagagcggcggcggcggtcgccgcggccaggaggaaaaacggggggagcgggagcggctcccgagtccgggaaggaaaggcgggcggaggaaggcgggtgtggaggaggaagcgctcgcgcccctcgctctctctccccccgtccccctctccgcgtctcttccccgcgtcctctctctgcgttcgccggtcgcggcgcgggcggcggctgcagaggcggctgcggacgcggaggggggcgaggggacacagaactctttttgtcctcctcgccggctcccaggcggcgggcgcgtcccgcgtccccggcgtcatcacgcgggaccccccggtccccgtgacgcaggacgcggggaggggaggggagggaggggagggggtcggggggaagggaggggggggaggagggggagagcgggtgacgccgcgggtgggccgaggccggcggggaggggggaggaggacgcggcggcggcggtgaaggaggagagccgcccggcgcgcgcccgccgccgcaggcgcgtccccggcgcgggcgcctctccacgccccgctccgcccggcgcccaatggcgcggccggctcggcggcccggcccttcctccgccctcctccttccccgccgccaccgccgcctcctcccttctccctcctccctcccctcccgccgcccgcgcgcccgcgtgcgctcgtgccggcgcggcatccccgcccaggcggcgggggagcgcggagcgcgccgcctccccgtgatcacgttcgcgcaccgcggcgccagcaggaaaaaaaaacaacaacacagagacacacctccctctccgtctccctcgccttctccgttttggggggagaccgatgccggtgccacatacacatacacacgcgcgcgcaccccgtcccccccccttccccttcccctcgccccggccccccggctcggtgccggagaaaagggggtgccggcgcggaccggggtcgggcccgcggcgccggggggtgggcgcgcgtgtccgtgtgagagtttacggtgtgggtgggtgtcacggtggagcgagctggggggccccggctcgctcgctcgctcgctcgccaggccggcaggccgggggcccggcctggacgcgggccacactgtgcggcggaccgtgccaagtttagggcgctgcgacccagagagagcgccgagctaaaagctattttatcgccgctgcggtttcccctggcgcggcgcctagacaccgtttctgcagacagagccctctctaatggcgcgtccgggaaggcttctcccgggccgcgagggccgcggtggcgacccccttccgcccccctcctccccgtccccgtccccgtcccccgcccgctctccccgggccgccgcgataccgcgcgggcgataccgcgcgggccacccgcgggcgctaccgcgccgctccgctcgccgcctcttccccctcctccaccgccgccagtcctcctcgctcctctccgcccacgcaactcctcccgcccgcgtgcccccggggccatcggctggaacaccctgagccttcgctgagtgctgcccccgccccgcccgcggcactccgggcccgcgtgagagggtacagagcgaacccggccccgaccccctccgcccgccgggggaggcttcccggaaccagcgcggtcatcaaccccctcccccacccacccgcacagcgcgcacgccgaccgcccgcgtttgctacgatccctgcgcccgacggggccgcgggacggcgcgcgcggcacggaacgagaggggggcgggggtgcgggcgagtgagcgtgcgcgaaggggggagagggcgcgtgggcgagagggacggggcggggcggggtggggggagcggcggctcgccctccgtcctcccctccttcctctccccgtcgactcgcgtcgcggcgattcgggccgcatcgaggcgcgtcggggttttcgcgccctcgcgccgccgccgcctcctcatctttcgtggttaacacacaccacacatacacacaccctcccccgcgccgctgtctgtggcgcccgtcgtctctcccgccggcgcaaccccgggccctgagagctttctccgctttctatttcggccccggcctccccgacctctctcacccgccctctcgcgctctctttcccacacacacacacataacaagaggctcgccctcgttccatcatctcggcgtactcgccgcctcgtacactacatccgtctaccgccgccagcaaccctttttttcccccgctcactcctcttcccccctcctccttcccccctcccgcctctccattcatcaatctcgtccaggccgcgggcccattcaccatcgccgcggtccgagctcggccaggagagatggatggatggattgggggaaggaggggggcggggagcgcgagcgggagaaggttggcgaaggagagagcgagagcgggaactggagagggggctgggtggggtgaggcgagtgaccgccaccgccctctaccaccaccacccctcccctcccgccctctcctctctctccccggtcccgtgtgcacacacagcgcgtgtttttttttccttcttcttcttcccggttcctcttcgtcctccggggccgcagccgccgcgctgggtgcagtccgtccccattcatcctttttccagaggccattcatcgccccgctcatccgtcttcatcgccgcggagggctggctcgaggcccgcgcgccctttcctcccccggcgcgcccgctccctccgcgtcccctctccccgccgccccctcggccgcaccgagccttctccgcgcggtcccgtctctcccctccccccatcccgccccactccccgcccccctctccccctccctctcccgccggtgttttcccacgcccacactcactttctccgctcccaccccaccctcctgactcacccgtcccgggggattccctgtcccccggggcgcggccgacccggcggtgacccgtccccgccccgccgcgcccctttttcctcgagagcgggcgtccgggaagggagaggtcgcggcgggtcacgcgcgctcccgcgacttcccggcccgcgatgcggtcccggaaatttccgggacggtcgcggcgaccgtctcggggcccgcggcgccgccaactcgagtcttggcacgcgcccagcgttcgcacttgttgtatataagatataatactagtatatattagcaattggtgcgaacgtgacgtggcccaatcaaatctctccacgggccccacgtgcccctattggcaatttatcgaccgctttccgaagggatcgaaccccgggggtgggtgccccggggacgcgccggccaatgggggagccggacccgcgtcccctgagcccacgtgtgaggccccggccaatgggcccgtcggcccggccccctcgacggggcgataaaggggtgcgcggcggcccggccgacggcactcgccgccccaacgacgccgacggggatcgaccgggagaggcgcggagcgcgccccgagccgacgaggagccgcgccccgctcgaggacgcccggacgacgacggtgacgtcggcatcggcctcgacgacgacgatcgcgggacccgacggtaccggatgcgggggatcccccggtcggggtgggccgggagtgggcgtggcacctcctcccccgtcccccctccctacccaccccgcgcgatcgcgctcatcgtatccgttctcgtttcaggtgccgcatcgttccggaagaaccgcctcgggggtcgcgcgtccgtcgcctaggtgagtcggggggcttcccgggtgcggctcccccctccccctccccctccccgggacgggtcgggtgggtaggtgggcgggggtcgggagggatcgggggaaggggagggggtcgtggctcacgctgacgtcatgcgttttcttccccccccccacagggacgcgggcgggaccggctggtcgcccacccacccaatccctcccatccctcccgccccccacccggcggggcggccgggctcgggtccggttccgggtcggcgtcggggagcctcgcccacccccccggccccccgtcccagcccagccccccgcacacccccatcgcaccccctcccaccccaccccgttcccacccccccgcccgccccaccgcgtccgccgcgcccatggaccgggtctgggccgactggtacgagcccgtgccctccccgccgttctcgcccgtcgacccgcccgggccccggcccgcgaccccggtcccggggagcagccccccgtcccccgcctcgacccccacgccccccaagcgcgggcgctacgtcgtcgagcaccccgagtacgggcccccgcccgaccccgaggaggtgcgcgtccacggcgcgcggggccccggcgccttctgcgcggccccctggcaccccgacacgcggcgcctcggggccgacgtgaaccgcctctttcgcggcatcgccgtctcggccgccgacgtgacgggcgacacgcgcgccctgcgccgcgccctctttgacttttacgccatgggctacacgcgccagcgcccctcggccccctgctggcaggccctgctccagctctcgcccgagcagagcgccccgctgcgcagcgcgctgcgcgagctcaacgagcgcgacgtctacgacccgcgcgtcctctccccgccggtcatcgagggcccgctctttggggaggagtgcgacgtcgacgaggacgacgccggctcggacaccaccgtcgcgtccgagtttagcttccgcggctcggtgtgcgaggacgacggggaggacgaagaggaagacggggaagaggaagacgaggacgaggagggggaagaggaagaagacgaggaggaagaggaaggggacgaggacggggagacggacgtgtacgaggaggacgaggacgaggacgacgaggacggggacgggctctgtgaggacgaggacgaggaggacggggacgactttgacggggccagcgtgggcgacgacgacgtgtttgagccccccgaggacggctcggacggagagggctcgggctcggacgacggcggggaggacgaggaagaggaggacgaggaagaggaggacgaggaagaggaggacgaggaagaggaggacgaggaagaggaggacgaggaagaggaggacgaggaagaggaggacgggggggaagacggtgaagacggcgaagaagacgaagacggagagggcgaggagggcgggaaggatgccgcccgccgggggacgcgcgccccgacgcggcccgccgccgccccgtgaggcgggccgcgccccgctctgttcttcctccacccccaccgtccgccccgtcgtttgcccctttccccttccccccccctctgttgtgccctcaataaacacggcggcccgccgctcgaacctcaactctctcgtctctcgggcgttttttcccctcccggccctcgtggggaagggagatggggggatgagacggggtggaggggggggggggggagggggacggggggatgagacggggggatgagacggggagcgagccgcggaaccctcccgcggggggcgagagtcggacgaagcgggggcaggggggggggggggacgggggggggggggggaccgccggtccggccgacgccgggcgccggacgccggacgacgcggtcgcgcacccagcgcaccacacgcgccgcgcaccatcgtcggcggcgcgcggggggggggatgggggggggggggggggggggaagtggcgtcaggggtgacgtcggtgaagcgggagggggaaaggggtgagagtgggtgggtggtggcggcgcgagtctgcgggatggtgccgaggggagccggacggtcacggggagggaggatgtggcgcgcgtggggcgtgggccgcctctgccgcggtcgcgagccgccaacgccgccgccaccgcctcccgcccaaagtgatatcgcggcgtgccgggacggccggcgctcattcacaccgcacccgttcacccgcgtccccgcgggcaagcacgcacacacccggtcgcgcatcatgctggcgatgtggagatgggtcaccaagaggtcgcggctccgccgaggccacgcccatcttgggggaaataaaggagtccggggaatgtgttccttataccttgccgggctcagcagggggttgtcgcgcgtccacgcccagcgctcgcacgcagcaacaatggccgacgccggaatccccgacgagatcctgtactcggacatcagcgacgacgagatcatcatcgacggcgacggcgacagcagcggggacgaggacgacgatgacggggggctgacgcggcaggccgcgtcgcgcatcgccacggacctgggcttcgaggtgctgcagcccctgcagtcgggctcggagggccgcgtcttcgtggcccgccggcccggcgaggcggacacggtggtgctgaaggtgggccagaagccctcgacgctgatggagggcatgctgctgaagcgcctggcccacgataacgtcatgagcctgaagcagatgctcgcccggggcccggtgacgtgcctggtcctgccgcactttcggtgcgatctgtacagctacctgaccatgcgggacgggccgctggacatgcgcgacgccgggcgcgtgatccggtccgtgctccgcgggctcgcctacctgcacgggatgcgcatcatgcaccgcgacgtcaaggcggagaacatcttcctcgaggacgtggacacggtgtgcctgggggacctcggggccgcgcgctgcaacgtggcggcgcccaacttttacgggctcgccgggaccatcgagaccaacgcccccgaggtgctcgcgcgcgaccgctacgacaccaaggtcgacgtctggggtgcgggggtggtgctcttcgagacgctggcctaccccaagacgatcaccggcggggacgagcccgcgatcaacggggagatgcacctgatcgacctcatccgcgccctcggggtgcaccccgaggagttcccgcccgacacgcgcctccggagcgagttcgtccggtacgccgggacccatcgccagccgtacacgcagtacgcgcgcgtggctcgcctcgggctgcccgagacgggggctttcctgatttacaagatgttgacgtttgatcccgtccgccgcccttccgctgatgagatactcaactttggaatgtggaccgtataaaacggcccggctccgagcggtaggacacacacacctttgcgcatctccacagctcaacaatgaagtgggcaacgtggatcctcgccctcgggctcctcgtggtccgcaccgtcgtggccagagaggcccctcgggagctctgctacggccaccccgtccacgacgaccggcggcccgtcgggcccgcgaccgacgcccagcccgtgaacccgctcgcccccgccaacgccaccgggacggactactctcgcggctgcgagatgcgcctcctggacccgcctctcgatgtctcgtcccgctccccggaccccgtcaacgtgaccgtcgcctggttctttgacggcggccactgcaaggtgcccctcgtccaccgcgagtactacggctgccccggggacgccatgccctccgtcgagacgtgcaccggcgggtactcgtacacccgcacgcgcatcgacaccctgatggagtacgccctcgtgaacgccagcctcgtgctgcagcccgggctgtacgacgccggcctgtacatcgtcgtgctcgtctttggcgacgacgcctacctcggcaccgtctccctgtcggtggaggccaacctggactacccctgcggcatgaagcacgggctcacgatcacccgccccggggccaccctcccgcccatcgcccccacggccggcgaccaccagcgctggcgcgggtgcttcccctcgaccgacgagggcgcctgggagaacgtgaccgccgccgagaagggcctgtccgacgactacgccgactactacgacgtgcacatcttccgcctggagtctgacgacgaggtcgtccacggcgatgcccccgaggcccccgagggcgaggaggtgaccgaggaggaggccgagctgacctccagcgacctcgacaacatcgagatcgaggtcgtgggctcgcccgccgctcccgtcgagggcgccggcgacggcgaggaggggcacggggacgaggaggacgaggagctgacctccagcgacctcgacaacatcgagatcgaggtcgtgggctcgcccgcggccgcccgcttcttcgccgcctccaccaccccccgcgcccccacccgcgcggccgagatcacgaccatgaccacggtcaccaccgtgcggacgaccgaggaccccagcggcatcaccgactgccgccggagcgacttcgtctcgccctctgacatcttcgtgacccccaccggcagccccgccctgctcctgggcttcctgggcagcgcgctcgcctcgcgccccctgcacctgacggccggggagacggcccagcacgtgcgcgaggcccagcagaagagccgccacgtccgctccctcggcggcctccagctctcggtcgagaccgagaccaccaacaccaccaccacccagacgggcctgtcgggcgacatccgcacctcgatctacatctgcgtcgccctcgccggcctggtcgtcgtgggcatcgtcatcatgtgcctccacatggcgatcatcagggcccgggcccggaacgacggctaccgccacgtggcctccgcctgacccggccccgcccgactcccccgcgattcccccctctctctcaccgggtgtccatcttcaataaagtatgtctcaaacacctaatttgcgtacggccttgcttacggggggtgcgatccacgcccagcggtccataaaattgggtcggcgccccaggttcccatacactcacccgccagcgccatgctgctcgcagcgctattggcggcgctggtcgcccggacgacgctcggcgcggacgtggacgccgtgcccgcgccgaccttccccccgcccgcgtacccgtacaccgagtcgtggcagctgacgctgacgacggtcccctcgcccttcgtcggccccgcggacgtctaccacacgcgcccgctggaggacccgtgcggggtggtggcgctgatctccgacccgcaggtggaccggctgctgaacgaggcggtggcccaccggcggcccacgtaccgcgcccacgtggcctggtaccgcatcgcggacgggtgcgcgcacctgctgtactttatcgagtacgccgactgcgaccccaggcagatctttgggcgctgccggcgccgcaccacgccgatgtggtggaccccgtccgcggactacatgttccccacggaggacgagctggggctgctcatggtggccccggggcggttcaacgagggccagtaccggcgcctggtgtccgtcgacggcgtgaacatcctcaccgacttcatggtggcgctccccgaggggcaagagtgcccgttcgcccgcgtggaccagcaccgcacgtacaagttcggcgcgtgctggagcgacgacagcttcaagcggggcgtggacgtgatgcgattcctgacgccgttctaccagccgcccccgcaccgggaggtggtgaactactggtaccgcaagaacggccggacgctcccgcgggcctacgccgccgccacgccgtacgccatcgaccccgcgcggccctcggcgggctcgccgaggcccaggccccggccccggcccaggccccggccgaagcccgagcccgccccggcgacgcccgcgccccccggccgcctgcccgagccggcgacgcgggaccacgccgccggggggcgccccacgccgcgacccccgaggcccgagacgccgcaccgccccttcgccccgccggccgtcgtgcccagcgggtggccgcagcccgcggagccgttcccgccccggaccaccgccgcgccgggcgtctcgcgccaccgctcggggatcgtcggcacgggcaccgcgatgggcgcgctcctggtgggcgtgtgcgtctacatcttcttccgcctgaggggggcgaaggggtatcgcctcctgggcggtcccgcggacgccgacgagctaaaagcgcagcccggtccgtagcctccgcagtaccggcgtcgatgatgatggtggcgcgcgacgtgacccggctccccgcggggctcctcctcgccgccctgaccctggccgccctgaccccgcgcgtcgggggcgtcctcttcaggggcgccggcgtcagcgtgcacgtcgccggcagcgccgtcctcgtgcccggcgacgcgcccaacctgacgatagacgggacgctgctgtttctggaggggccctcgccgagcaactacagcgggcgcgtggagctgctgcgcctcgaccccaagcgcgcctgctacacgcgcgagtacgccgccgagtacgacctctgcccccgcgtgcaccacgaagccttccgcggctgcctgcgcaagcgcgagccgctcgcccggcgcgcgtccgccgcggtggaggcgcgccggctgctgttcgtctcgcgcccggcctcgggggacgcggggtcgtacgtgctgcgggtccgcgtgaacgggaccacggacctctttgtgctgacggccctggtgccgccgagggggcgccccgtccccacgtcgccgcccgcggacgagtgccggcccgtcgtcggatcgtggcacgacagcctgcgcgtcgtggaccccgccgaggacgccgtgttcaccacccagcccccgcccgagcccgagccgccgacgacccccgcgcccccccgggggaccggcgccacccccgagccccgatcggacgaggaggaggagggtgacgcggagacgacgacgccgacgctgaccctccctccaccccgtctcatccccccatctcccttccccacgagggccgggaggggaaaaaacgcccgagagacgagagagttgaggttcgagcggcgggccgccgtgtttattgagggcacaacagagggggggggggaaggggaaaggggcaaacgacggggcggacggtgggggtggaggaagaacagagcggggcgcggcccgcctcacggggcggcggcgggccgcgtcggggcgcgcgtcccccggcgggcggcatccttcccgccctcctcgccctctccgtcttcgtcttcttcgccgtcttcaccgtcttcccccccgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcctcttcctcgtcctcgtccccgtcttcctcttcctcttcctcttccccgtccccgccgtcgtccgagcccgagccctctccgtccgagccgtcctcggggggctcaaacacgtcgtcgtcgcccacgctggccccgtcaaagtcgtccccgtcctcctcgtcctcgtcctcacagagcccgtccccgtcctcgtcgtcctcgtcctcgtcctcctcgtacacgtccgtctccccgtcctcgtccccttcctcttcctcctcgtcttcttcctcttccccctcctcgtcctcgtcttcctcttccccgtcttcctcttcgtcctccccgtcgtcctcgcacaccgagccgcggaagctaaactcggacgcgacggtggtgtccgagccggcgtcgtcctcgtcgacgtcgcactcctccccaaagagcgggccctcgatgaccggcggggagaggacgcgcgggtcgtagacgtcgcgctcgttgagctcgcgcagcgcgctgcgcagcggggcgctctgctcgggcgagagctggagcagggcctgccagcagggggccgaggggcgctggcgcgtgtagcccatggcgtaaaagtcaaagagggcgcggcgcagggcgcgcgtgtcgcccgtcacgtcggcggccgagacggcgatgccgcgaaagaggcggttcacgtcggccccgaggcgccgcgtgtcggggtgccagggggccgcgcagaaggcgccggggccccgcgcgccgtggacgcgcacctcctcggggtcgggcgggggcccgtactcggggtgctcgacgacgtagcgcccgcgcttggggggcgtgggggtcgaggcgggggacggggggctgctccccgggaccggggtcgcgggccggggcccgggcgggtcgacgggcgagaacggcggggagggcacgggctcgtaccagtcggcccagacccggtccatgggcgcggcggacgcggtgggtcgggcggggtggtgggaacggggtggggggggggggggggcggggggggggtgcggggggctgggctgggacggggggccgggggtgggtggcgaggctccccgacgccgacccggaaccggacccgagcccggccgccccgacgggtgggtggctggatggattggatggattggatgggtggtcgaccagccggtcccgctcgcgtccctgtggggggggggaagaaaacgcatgacgtcagcgtgagccacgaccccctccccttcccccgatccctcccgacccccgcccacctacccacccgacccgtcccggggagggggagggggaggggggagccgcacccgggaagccccccgactcacctaggcgacggacgcgcgacccccgaggcggttcttccggaacgatgcggcacctgaaacgagaacggatacgatgagcgcgatcgcgcggggtgggtagggaggggggacgggggaggaggtgccacgcccactcccggcccaccccgaccgggggatcccccgcatccggtaccgtcgggtcccgcgatcgtcgtcgtcgaggccgatgccgacgtcaccgtcgtcgtccgggcgtcctcgagcggggcgcggctcctcgtcggctcggggcgcgctccgcgcctctcccggtcgatccccgtcggcgtcgttggggcggcgagtgccgtcggccgggccgccgcgcacccctttatcgccccgtcgagggggccgggccgacgggcccattggccggggcctcacacgtgggctcaggggacgcgggtccggctcccccattggccggcgcgtccccggaggcgggtgcgcgcggggttcgatcccttcggaaagcggtcgataaattgccaataggggcacgtggggcccgtggagagatttgattgggccacgtcacgttcgcaccaattgctaatatatactagtattatatcttatatacaacaagtgcgaacgctgggcgcgtgccaagactcgagttggcggcgccgcgggccccgagacggtcgccgcgaccgtcccggaaatttccgggaccgcatcgcgggccgggaagtcgcgggagcgcgcgtgacccgccgcgacctctcccttcccggacgcccgctctcgaggaaaaaggggcgcggcggggcggggacgggtcaccgccgggtcggccgcgccccgggggacagggaatcccccgggacgggtgagtcaggagggtggggtgggagcggagaaagtgagtgtgggcgtgggaaaacaccggcgggagagggagggggagaggggggcggggagtggggcggggtggggggaggggggagacgggaccgcgcggagaaggcgcggtgcggccgagggggcggcggggagaggggacgcggagggagcgggcgcgccgggagaggaaagggcgcgctggcctcgagccagccctccgcggcgatgaagacggatgagcggggcgatgaatggcctctggaaaaaggatgaatggggaggacggatcgcggcggctgcggccgcggcggcgggggctggtgggaggagtgggggaattgggggcggggggaattcgggggagcacacgggaccggggagagagaggagaggagagggaggggagggggggtggtggtagagggcggtggcggtcactcgcctcaccccacccagccccctctccagttcccgctctcgctctctccttcgccaaccttctcccgctcgcgctccccgcccccctccttcccccaatccatccatccatctctcctggccgagctcggaccgcggcgatggtgaatgggcccgcggcctggacgagattgatgaatggagaggcgggaggggggaaggaggaggggggaagaggagtgagcgggggaaaaaaagggttgctggcggcggtagacggatgtagtgtacgaggcggcgagtacgccgagatgatggaacgagggcgagcctcttgttatgtgtgtgtgtgtgggaaagagagcgcgagagggcgggtgagagaggtcggggaggccggggccgaaatagaaagcggagaaagctctcagggcccggggttgcgccggcgggagagacgacgggcgccacagacagcggcgcgggggagggtgtgtgtatgtgtggtgtgtgttaaccacgaaagatgaggaggcggcggcggcgcgagggcgcgaaaaccccgacgcgcctcgatgcggcccgaatcgccgcgacgcgagtcgacggggagaggaaggaggggaggacggagggcgagccgccgctccccccaccccgccccgccccgtccctctcgcccacgcgccctctccccccttcgcgcacgctcactcgcccgcacccccgcccccctctcgttccgtgccgcgcgcgccgtcccgcggccccgtcgggcgcagggatcgtagcaaacgcgggcggtcggcgtgcgcgctgtgcgggtgggtgggggagggggttgatgaccgcgctggttccgggaagcctcccccggcgggcggagggggtcggggccgggttcgctctgtaccctctcacgcgggcccggagtgccgcgggcggggcgggggcagcactcagcgaaggctcagggtgttccagccgatggccccgggggcacgcgggcgggaggagttgcgtgggcggagaggagcgaggaggactggcggcggtggaggagggggaagaggcggcgagcggagcggcgcggtagcgcccgcgggtggcccgcgcggtatcgcccgcgcggtatcgcggcggcccggggggggcgggcggggggcggggacggggacggggcggaggggggaggaagggggtcgccaccgcggccctcgcggcccgggagaagccttcccggacgcgccattagagagggctctgtctgcagaaacggtgtctaggcgccgcgccaggggaaaccgcagcggcgataaaatagcttttagctcggcgctctctctgggtcgcagcgccctaaacttggcacggtccgccgcacagtgtggcccgcgtccaggccgggcccccggcctgccggcctggcgagcgagcgagcgagcgagccggggccccccagctcgctccaccgtgacacccacccacaccgtaaactctcacacggacacgcgcgcccaccccccggcgccgcgggcccgaccccggtccgcgccggcacccccttttctccggcaccgagccggggggccggggcgaggggaaggggaagggggggggacggggtgcgcgcgcgtgtgtatgtgtatgtggcaccggcatcggtctccccccaaaacggagaaggcgagggagacggagagggaggtgtgtctctgtgttgttgtttttttttcctgctggcgccgcggtgcgcgaacgtgatcacggggaggcggcgcgctccgcgctcccccgccgcctgggcggggatgccgcgccggcacgagcgcccgcgggcgcgcgggcggcgggaggggagggaggagggagaagggaggaggcggcggtggcggcggggaaggaggagggcggaggaagggccgggccgccgagccggccgcgccattgggcgccgggcggagcggggcgtggagaggcgcccgcgccggggacgcgcctgcggcggcgggcgcgcgccgggcggctctcctccttcaccgccgccgccgcgtcctcctcctccctccccgccggcctcggcccacccgcggcgtcacccgctctccccctcctcccccccctcccttccccccgaccccctcccctccctcccctcccctccccgcgtcctgcgtcacggggaccggggggtcccgcgtgatgacgccggggacgcgggacgcgcccgccgcctgggagccggcgaggaggacaaaaagagttctgtgtcccctcgcccccctccgcgtccgcagccgcctctgcagccgccgcccgcgccgcgaccggcgaacgcagagagaggacgcggggaagagacgcggagagggggacggggggagagagagcgaggggcgcgagcgcttcctcctccacacccgccttcctccgcccgcctttccttcccggactcgggagccgctcccgctccccccgtttttcctcctggccgcggcgaccgccgccgccgctctcgaggtttttttttcccttcctctccttcttcctcctcctccgcccttgggtgtttccttctccgcgtccccatctccggggcccgaggcgccggggctttcttcccgggccgccggggaggaagcgccccggcttctgcttcttcttctcctctctcggtaagcacagactctcctactactactactcctactactcctactcctactatattactatgactactactatgactactacggtgacttctcctccccgctctagactttttttcctcccctctcctcctttcgctcccgtctcccgggcgcccgcggccgggcggagggggaagcgggaaggggaagcgcggggcgccgcgcctctccctcccgcttcccctccgccggccccttccccgtttccccgtttccctttccccccccttccccgtctgttccttcccccccgcttccccgtttcttcttctcccccccgtcccctcttccttccccacccgccccgctctcccacgccgcggccgcggggggccgaagcggacgcggaagaagacgcggaagaagagagagagagaaagaggggggccggcggggatggggaagaaggcccgacccgcgcgggggcccccctcctgtcgcctgtcgtttgaatgaaaccttcccccgagcgaccccgctcccgcctgtggtgccgtgtttttttcccgcgacggggagagcgcgagagtgaggctgttggctgtgggttgttggctgttggctgttggctgttggctgaggacgacggggaggggggatgctgccgcgggtggcatgagacggagggggcgagggcgggcggtggaggggggagaggaaggtctgggcgcgcgtgccgcgggccgagtgtcgggcgagtgcggggcgggttggggacggggaggcggggagggggggggggatggccggccggcgcccccggagcacgcggggcgcgccggcgggcgggagggggtccgggggaagggtggggcggtggcggcggccatcgcccgacccttccccggggcgcgcgcgtgggaccgggaccgggacagggggcgggggcggggagaggaggagaaggaggagaaggaggagaaggaggagaaggaggagaaggaggaggcgaactgggggaaaggcgagacacgcccgggcgcccccctcctcggaccgaggcgacggcggagacggagacggagagaccgcccgcccggccaccaccgtcggtaccggtatcaccagcaacacccgcaccaccgacacccacacgccccctcccacccgcacgggcccccatacccgcaccggccctcacccccccccccccgcacgctctcctctctcgcacccccccacccccccccccccccgcgcacagacacacggccgaccccccccccgcccgccccgtccggccgcccccccgcccgcccacccctccccccgcccggccctccgagagagagagcgagccttctctccccgcacacacacacacacacccacacccgcagacctccggagcaaacccccccgccgtcccccagcccgattttgggcccccccgtcccggaccccccggggggcgggggcctcgcgcctccgcgtccggcgggcgcgaccgaggcccgcgcgcccccggggtctcggggcgggacgcggcgcccgcgcggggacactctttcccccggaggggcgtcccggcgcgccggcccggccccccccgccgggaagggggcccggccctcttcgtctcccggagcgcccccggggtcccggcccgaggtccattttgggccccgcccctttgcacccgcccccccaccgggcggggtccgcgctccgacccccccccctcccctctgccacgccccgccacgccccgccgccatcttggctcccctcccccccaactccctctctcccgacgaggccgagccggtctcgcgccccctcccgcccgcccgaggtaccgcggaaggacgacacgagccggaagacggtgcaacgagaacaacttttatttttttcactctggggcgtgtctcgtggggtgggaggggggaccgggagagagaggtagaggggggggggtgggtgggcgacaaaagtatccaaggcgtggctgtccggctcctcccttccgggtccgggcgggtccgggctcgaggttcgggtccggttctcccgccccggcgtcgcttcccggttcgggtccggtcgccggtccccggtccgacggcggttccgctcgtctctccgaccggctgggggcgccctcggagcgcgcgccgagcgcgcaggcgcgggtggggcgaccgcgggggtcgcggggggccgcaaggggctcggggggggggaacgggggtctcgggggtctcgcgagtggcagggggaggcggtgagaccaccacacaacacagagaccaccacaaaacactgagagaccaccacacaacacagagaccaccacaaaacactgagagaccaccacaacacatgcggaccacgtgcagacacacggaagcttgcaccgtgaggccccattggccgagggcggccacgtggggcgagcatgcaaatgacgtgcacacggaagcttccccgaaaatcatctgattggctcgctagcaccacgggggtgggtcgcgtgccgagcatgcgcctcgcgcccgcgcgagggattttcggacaatctcattggccgcccgcggccaagatggccgcagagcgggccgggcatgcaaatcagaggcgcgcgggagacgcctccgcgcgcccattggcccgggcgagccgagatggccgccgcgggggccgggcatgcaaagtagacgcgagaggaagtagggagagaaatcccattggccgtcgaggggccaagatggcgccctcggggccgggcatgcaaagtagacgcgagaggaagcgggcgagagaaatcccattggccgccgtcgaccaagatggccgccgcgggggacggacatgcaaagtagacgcgagaggaagcgggcgagagaaatcccattggccgtcgagtgggcaagatggccgccgcgggggccgggcatgcaaatggtcctcgcgaggaagttcctcgcgaaatcccattggccggcggccgccatcttgggccgggcatgcaaagcagacgcgagaggaagcgggcgagaaaaatcccattggccggccgtcggggaagtccgcggcgaaaatcggccattggtccgcttacctgggggcgggctcttctcggggagtttataggcacggtctccatcgtagcacttcactgcggtgcaggtacggacagcatcgttctctgccaacccgaggggatccgatcgtctcggctccggcgcggactctgaagactccggctctccggcggctatcagccctcgacggacggcccgacccaccggggggaagagagaggagagccctcttcttcttctccttcttcgccctctccttcttcttcttctgccgcgcccgctcctccggctccctctcaccctccagcacccgctctccgagctcggctcccggcgcgcgagagagagagagacaccccgagaaccgatcgagaccatggccgacgatctctttgacttcatcgagaccgagggcaacttcagccagctcctggcggcggcggccgcggccgcggcggccgaggaagaggggaccgcggccggctccgacggcggcagccagggctcccggcgccgcggcccctccggcgaggatctcctctttggccccggcggcctcttctccgacgatgccggcgaggccgaggccgaggccgccgtcctcgcggccgcggccggagccacgaggccgccccgtcctccttcggcccagcagcagcagcagcagcagccccgccggggctcgggcgagatcgtggtgctagacgacgaagacgaggaggaggacgagcccgggtccccggccgccgggtcccccggtcgggctctccatcagggctccgagcacggtcacctcgtcctcgggccccggtcccgggccggctccgggccccggccgccgaccccggcagcactcgcagcggcagaggccggggcccccggcggccccgggcgcccgtccccctccgcagcctccccggcctcctcctccggctcccccggcccctcggcagccccgcggagatgggcccccgcgaggggggacccggtcggtgagcccgggccggcggcgcggcctcggaccccggcgccaccagcacagccagcagcggtggccgcagcgccggcacggcgggggcccgcttccccagccccccccgccgccgggccggtctcggcgctcggcggcggcggcgccccctccgccggaggggaccgcggtcgtcaccatcaccagccccgcgcgcccctgctcgacgagcccgccgccgcgaggcggctcgatcctcggccgcttggcgcccggagccccgtctcctccaactccaactccaactccagctcctccaccaccgtcgccgtcgagcccgtcgcccgcgaccccgagaaggacgaggacggactcggcctcgccggggacggcggggcgcccccgcagagacagccccgccggcgccgcgccggagagggcgccctccgccgcggccgcggcttcagcagctccagcagcagcggcagcgactcggacctctccccggcccgcccgccctccgccccccgggcctccgccgccgccgcgcggcgctcggcctcctcctcctcctcgtcctcgtcctcctcgtcctcctcgtcctcgtcctcccccgagggagaagaagacgaaggagtccgggccggcgccccactcgcccgcgccgggccgccaccgtctccgccggcgcccgccgcggccccgcggccctcggcctcctccgccgccgcctcctcctcctcctcctccgccgcggcctccccggccccggccccggagcccgcccggcccccgcgcaggaagcgccgctccaccaacaaccacctctcgctcatggccgacgggcccccgccgaccgacgggccgctgctcaccccgctcggggagccctggcccggctccgaccctccggccgacggccgcgtccgctacggcggcgccggggactcccgcgaggggctctgggacgaggacgacgtccgccaggccgcggcccgctaccgcgccgcggccgggcccgtgcccgtcttcatcccggagatgggggactcgaggaagcagcacgaggccctcgtgcgcctcatctacagcggggccgccggcgaggccatgtcctggctgcagaacccgcggatgcaggcccccgaccagcgcttcaaccagttctgccagcgccgggtccacgcgccccacggccacggctccttcatcaccggcagcgtgaccccgccgctgccgcacatcggggacgccatggccgcccaggacccgctctgggccctgccgcacgcggtgagcgccgtggccatgagccgccgctacgatcgcacccagaagaccttcatcctgcagagcctccgccgggcctacgccgacatggcctacccgggccgcgccgcggacccccgcgccggggaggcgaccgtcgaggccctctgcgcccgcgtccgcgccgccttcgccgccgcgcagcccggccgcgtgccccgcgagctggcggacgcctgcgtcctggcctgccgcggcgtcctcgagcgcctgctgcccgtcccgctccggctgcccgcgcccgcccgcgccccggccgccctcgggcccgcctgcctcgaggaggtgaccgccgcgctgctcgcgctccgcgacgcgatccccggggccggcccggccgagcggcagcaggccgcggactctgtcgccctcgtcgcccgcaccgtggcgccgctggtgcgctacagcgtggacggggcccgcgcccgcgaggccgcctggacctacgccgcggccctcttcgccccggccaacgtggccgcggcccggctcgccgaggccgccgcgcggcccgggcccgcggagcccgcgcccggcctgcccccgctctggcccgagcagcccggcctcgtcgtcccagcgccggcgccggcggcggcgggcgccccctccggcctccccggctccgggccctccagccccgccagcaccaagtccagctccagcaccaagtccagctccggcaccaagtccggcagccccggcaccaagtccggcctctccggctccgccggctacgccagctcccccgccgccggcccggacccagcgcccgagcggcgcaagaagaagcgccgcgcgcccggggcccgccgccccggggacggcgaggaggacgaggggctctccggctcggccctccgcggggacggccacggccaccgcgacgacgaagaggaccgggggccccggcgcaagcgccgctccctcggactcgggcccgctccggaccccgctcccgcgctgctgtcctcctcctcctcctcctccgaggacgaccggctccggcgcccgctcgggcccatgcccgagcaccccgcgcccgacggcggcttccgccgcgtcccggccggcgagacccacacgccgcggccctccgaggcggccctcgccgcctactgcccgcccgaggtggcccgcgtcctcgtggaccaggaggtcttccccgaactctggcgcccggcgctcaccttcgacccggccgccctggcgcacatcgccgcgcgccgcggggccgcgggcgccccgctccgccgccgcgccgcctggatgcggcagatagccgaccccgaggacgtgcgcgtggtggtgctctacgacccgctgccccacgaggagctctgcgccgagcccgccgagggcgccccgcgcccggcctgggacccgcgccgcggcggcctctcggcgctgctcgccgccttcgcccaccgcctctgcacgccggactcgcacgcctgggccgggaactggaccgggcgccccgacatcggccgcctcaacgcccagggggtgctgctgctctcggcgcgggacctcggcttcgccggcgccgtcgagtacctctgctcgcggctcggcgcggcgcggcgccggctcatcgtgctggacaccatcgaggactggcccgcggacggcccggccgtgggggactaccacgtctacgtccgcgcccgcctggacccggccgcccagtgcgccgtgcgctggcccgagtgccgcgagctgcgcgcggccgtcctggactcgagctccatcgtgggcccggcctgcttcgcccgcgtggaggcctccttcgcccgcctgcaccccggggccgagccgctgcgcctctgccgccaggacaacgtgcgctacacggtgagcacccgcgccgggccccggaccccggtcccgctgccgccgcgcgcctaccgccagcgcgtgctccccaccgtggacggctgcaaggacatggcccgccagcgctccgcgctcgggctcggggacccggactttgacgcgggcgccgccttcgggcaccgcgcggccaaccgctgggggctcggcgccccgctgcggcccgtcttcgtctcctgcggtcgccggggcctcgccgagctccgcggccccgagggcctgccggccgagctgcgcgccttctgcgccgcggcgctgctggagcccgacgccgaggccgccccgctggtgctggccccgggcgccctcgcggccgcgggcgccccgccggccgtgcgctgggactttgcgcccttcgagaccagcgtccgcgccgccggcggcgccgtggagacccaccggccctcgggcgcccgggggcccggcgaggacggggaggactctgccgcggtggagagcgtcgacttccgcggcggcgacggccggccccgcggcccgctcgggcccatcaaggtggaggccatctcggacgacgaggaggccgaggacgccggcaacccctacctgctgctccgctgagcggggcgccccctcggcccggctggactctgactctgactctccggcccctccaccggctcctcgaggcccttctcctccgtcttctctcccctcgcccccggcccggtcctcgtcctcgtccccgtcctcctcctcctcctcctcctccgcgtccgcggcctcctcctcctcctcctcctccgcgtccgcggcggcggcctcggtcccctcggcccggcggcgcttgcctccccggcgcctgcctccccggcccggcggccctcctcctcctcctcctctccttctcctccgcgcggatcccccggccggaggcggctgcggcggcggcggaggtggcggcggtggtggcggcggtggtggcggcggtggtggcggcggtggtggcggcggtggtggaagcggcggcggcggccgcggcggagggctcggcggcggaggatcgtcccggtccccttctcctcctcctcctcctcccgcggtcccccggtccccttctcctccttctcccatcgggtgagaaaagagtttattttcagagtgagaaaataaagtttgtgctgtattttctgaaccagctcgagtctctgagattttttggggagatggaggcggccatcttggcggtggtctctggggcggaggtggtcttgtggatgggggtccctggtgggaggaagaagaagaggtggagggtcttggtgggggtgatgggggtcctcctcctggagggtcttggtggtggtgatgggaagaagtggatgggggtcctcctcctggtgggtcttggtggtggtgggtctcagcagatgggggtcctggtgggtcctggtgggtctcagcagatgggggtcctcctcctggagggtcttggtggtggtggtgggtctcagcagatgagggtcccggtgggtcctggtgggtctcagcagatgggggtcctcctcctggtgggtctgggtgggaagaagtagagggtcttggggatgttgggggtccttgatggtggtggtggtgggaggtggacggtgttggtggtcccggcgggtcctggtgggaggtagatggtcccgagggtcccggtggtcccgggtgggagttagacgatggtggtcctgcggtggtcgagggtggaggaggggggggggggggggggaggaaagagagagagagaaaaaaacccgggggtgggcagtacagggaactgccattgcaaccatcgcccccggggggggagagtaggaaagtgtgtgcgttggggggggggggtggtgttgttggtggtggtggtcccgggtggtcgtcgtggaggggcgggaaaggcggtgggagagcgggcggaacaggttcatctggcttccgtcttccgtccccactcccagcctcccccgctgccacccgattggccggcgggacgatgacgacggcgaccgcgtggcgcgaggggagccaatggggcgggtgccgcggagcggaagcccccgcccgcggacccccggtgcgacggaaggggcaggctcggcgcgccgatgcgatccgcggagcccgccccgtccgcgccccggaagccggcgtcacgtccgggggaagcggaaggggccggcgcgatatgcagatgagatccgtggggaccggcgtcacgtccggcggaagttgcgcgcgggaagcggtcatacgcaaatgaggcggaagtcgtcatcggcaaaaacgtcacgtccgggaagcggtcatatgcaaatggggggggggtgggagcgatggggtccgcggagcccgccccgtccgcgccccggaagccggcgtcacgtccgggggaagcggtcatatgtaaatgaggggggaggggccagagcgtcacttccggaggaagttgtcatatgcagattagggggaggggttatgcagattagggggaggggttatgcagattagggggaggggttatgcagaggaggggggggggttatgaagattaggggggggggttatgtaaatgcatgcaaatgagtgggaggggttatgtaaatgaggggggggggccatgcaaattgggggaggggccatgctaatttatgcaaatgggggggaggggccatgctaatttatgcaaatgagggggcggggctatgcaaatgcatgcaaatttatgcaaattagccccccccctttaagccccgcccccttttttcgcggcccccggggggcgggggggtgggggcggtcccgcggacgccgacgagctaaaagcgcagcccggtccgtagcctccgcagtaccggcgtc

SEQ ID NO: 4 - DNA, 15 bp, Artificial
  fragment of US-1 gene of Pseudorabies virus
ctcctcttcctcgtc

SEQ ID NO: 5 - DNA, 126 bp, Artificial
  Fragment of US-1 gene sequence of Pseudorabies virus
cgaggaagaggaagaggaagaggaagacggggacgaggacgaggaagaggaggacgaggaagaggaggacgaggaagaggaggacgaggaagaggaggacgaggaagaggaggacgaggaagagga

SEQ ID NO: 6 - DNA, 366 bp, Artificial
  fragment of TK gene sequence of Pseudorabies virus
gcggcgcctgcgcgcccgcgcgcgcgccggggagcacgtggacgcgcgcctgctcacggccctgcgcaacgtctacgccatgctggtcaacacgtcgcgctacctgagctcggggcgccgctggcgcgacgactgggggcgcgcgccgcgcttcgaccagaccgtgcgcgactgcctcgcgctcaacgagctctgccgcccgcgcgacgaccccgagctccaggacaccctcttcggcgcgtacaaggcgcccgagctctgcgaccggcgcgggcgcccgctcgaggtgcacgcgtgggcgatggacgcgctcgtggccaagctgctgccgctgcgcgtctccaccgtcgacctggggccctcgcc

SEQ ID NO: 7 - DNA, 80 bp, Artificial
  Fragment of TK gene of Pseudorabies virus
cgcctgctcacggccctgcgcaacgtctacgccatgctggtcaacacgtcgcgctacctgagctcggggcgccgctggcg

SEQ ID NO: 8 - DNA, 444 bp, Artificial
  Fragment of TK gene of Pseudorabies virus
gggcccgcggtcgagggcccgcccgagatgacggtcgtctttgaccgccacccggtggccgcgacggtgtgcttcccgctggcgcgcttcatcgtcggggacatcagcgcggcggccttcgtgggcctggcggccacgctgcccggggagccccccggcggcaacctggtggtggcctcgctggacccggacgagcacctgcggcgcctgcgcgcccgcgcgcgcgccggggagcacgtggacgcgcgcctgctcacggccctgcgcaacgtctacgccatgctggtcaacacgtcgcgctacctgagctcggggcgccgctggcgcgacgactgggggcgcgcgccgcgcttcgaccagaccgtgcgcgactgcctcgcgctcaacgagctctgccgcccgcgcgacgaccccgagctccaggacaccctcttcggcgcgtac

SEQ ID NO: 9 - DNA, 252 bp, Artificial
  Fragment of TK gene of Pseudorabies virus
cgcttcatcgtcggggacatcagcgcggcggccttcgtgggcctggcggccacgctgcccggggagccccccggcggcaacctggtggtggcctcgctggacccggacgagcacctgcggcgcctgcgcgcccgcgcgcgcgccggggagcacgtggacgcgcgcctgctcacggccctgcgcaacgtctacgccatgctggtcaacacgtcgcgctacctgagctcggggcgccgctggcgcgacgactgg

(19) United States
(12) Patent Application Publication
(10) Pub. No.: US 2024/0189420 A1
(43) Pub. Date: Jun. 13, 2024

(54) PSEUDORABIES VIRUS VACCINE

(71) Applicant: Zoetis Services LLC, Parsippany, NJ (US)

(72) Inventors: QiaoRan LIU, Beijing (CN); JinAn HUANG, Kalamazoo, MI (US); YiBo KONG, Kalamazoo, MI (US); Dong SUN, Beijing (CN)

(21) Appl. No.: 18/555,626

(22) PCT Filed: Apr. 15, 2022
(86) PCT No.: PCT/US2022/024941

§ 371 Date: Oct. 16, 2023

(30) Foreign Application Priority Data
Apr. 16, 2021 (CN) 202110412008.9

(51) Int. Cl. A61K 39/245; A61K 39/00; A61P 31/22; A61P 37/04; C12N 15/86

(52) U.S. Cl. CPC A61K 39/245; A61P 31/22; A61P 37/04; C12N 15/86; A61K 2039/5252; A61K 2039/5254; A61K 2039/552; C12N 2710/16622; C12N 2710/16634; C12N 2710/16643; C12N 2710/16662; C12N 2710/16671

(57) ABSTRACT

This disclosure provides an attenuated suid herpesvirus 1 (a Pseudorabies virus) wherein the TK, gI and gE genes thereof are modified relative to a parent field strain, such that the resultant virus is safe and effective for use as a live vaccine that protects swine animals from challenge with a virulent Pseudorabies virus.

### FIELD OF THE INVENTION

This invention is generally in the field of vaccines against pseudorabies virus.

### BACKGROUND

Pseudorabies virus (PRV) is a disease which infects many species of animals worldwide. PRV infections are variously called infectious Bulbar paralysis, Aujeszky's disease, and mad itch. Clinical signs of PRV infection include abortion, high mortality in piglets, and coughing, sneezing, fever, constipation, depression, seizures, ataxia, circling, and excess salivation in piglets and mature pigs. Mortality in piglets less than one month of age is close to 100%, but it is less than 10% in pigs between one and six months of age. Pregnant swine can reabsorb their litters or deliver mummified, stillborn, or weakened piglets. In cattle, symptoms include intense itching followed by neurological signs and death. In dogs, symptoms include intense itching, jaw and pharyngeal paralysis, howling, and death. Any infected secondary host generally only lives two to three days. Pruritus, or itching, is considered a phantom sensation as virus has never been found at the site of pruritus.

Infections are known in important domestic animals such as swine, cattle, dogs, cats, sheep, rats and mink. The host range is very broad and includes most mammals and, experimentally at least, many kinds of birds (for a detailed list of hosts, see D. P. Gustafson, “Pseudorabies”, in Diseases of Swine, 5th ed., A. D. Leman et al., eds., (1981)). Adult swine and possibly rats, however, are not killed by the disease and are therefore carriers. However, for other species the disease is fatal.

Populations of swine are particularly susceptible to PRV. Although the adult swine rarely show symptoms or die from the disease, piglets become acutely ill when infected and death usually ensues in 24 to 48 hours often without specific clinical signs (T. C. Jones and R. D. Hunt, Veterinary Pathology, 5th ed., Lea & Febiger (1983)).

PRV is a herpesvirus. The PRV genome is characterized by two unique regions (UL and US), with the US region flanked by the internal and terminal repeat sequences (IRS and TRS, respectively). The sequence and gene arrangement of the entire PRV genome are known and a map of the likely transcript organization, well supported by experimental data, has been established. Recombination between the inverted repeats can produce two possible isomers of the genome, with the US region in opposite orientation. The functions of the 70 different genes have been identified. For general biology of PRV and its mechanism of action, see Pomeranz et al, Microbiol. And Mol. Biol. Reviews 205, Sept., 462-500.

PRV vaccines have been produced by a variety of techniques and vaccination in endemic areas of Europe has been practiced for more than 15 years. Losses have been reduced by vaccination, but vaccination has maintained the virus in the environment. Vaccinated animals that are exposed to virulent virus may survive the infection and then shed more virulent virus. Vaccinated animals may therefore harbor a latent infection that can flare up again. (See, D. P. Gustafson, supra).

Live attenuated and inactivated vaccines for PRV are available commercially in the United States and have been approved by the USDA (See, C. E. Aronson, ed., Veterinary Pharmaceuticals & Biologicals, (1983)).

Live attenuated and inactivated vaccines for PRV are available commercially in the United States and have been approved by the USDA (See, C. E. Aronson, ed., Veterinary Pharmaceuticals & Biologicals, (1983)). Nevertheless, there is still a need for novel PRV vaccines, and particularly live attenuated vaccines that are both safe and effective.

### SUMMARY OF INVENTION

In one aspect, the invention provides an attenuated suid herpesvirus 1 (a Pseudorabies virus) wherein the TK, gI and gE genes thereof are modified relative to a parent field strain, such that the resultant virus is safe and effective for use as a live vaccine that protects swine animals from challenge with a virulent Pseudorabies virus, and wherein said parent strain is selected from the group consisting of: strain FS18 (SEQ ID NO:1); strain JS2012 (SEQ ID NO:2); strain TJ (GenBank accession KJ789182); strain HeN1 (GenBank accession KP098534); strain HLJ8 (GenBank accession KT824771); strain HN1201 (GenBank accession KP722022), and any strain that is encoded from a nucleotide sequence that is at least 85% identical to SEQ ID: NO:1 or SEQ ID NO:2. In certain embodiments, the virus further comprises attenuating modifications of one or more of the US1, US2 and US9 genes, with the proviso that at least one of US2 and US9 genes is not modified.

In certain embodiments, the virus is encoded by SEQ ID NO:3 or a sequence that is at least 85% identical thereto, and which comprises: a) a deletion of UL23 gene nucleotides 480-846 (Isolate M1707); or b) a deletion of UL23 gene nucleotides 526-607 (Isolate M1705); or c) a deletion of UL23 gene nucleotides 280-723 (Isolate M1708); or d) a deletion of UL23 gene nucleotides 364-615 (Isolate M1710); or e) a deletion in UL23 gene that includes any of the deletions of ‘a’, ‘b’, ‘c’, or ‘d’.

In another aspect, the invention provides an attenuated suid herpesvirus I (Pseudorabies virus) that is derived from strain FS18 (SEQ ID NO:1); strain JS2012 (SEQ ID NO:2), strain TJ (GenBank accession KJ789182), strain HeN1 (GenBank accession KP098534), strain HUJ8 (GenBank accession KT824771) or strain HN1201 (GenBank accession KP722022), or any strain that is encoded from a nucleotides sequence that is at least 85% identical to SEQ ID NO:1 or SEQ ID NO:2, wherein said attenuate is encoded from a DNA sequence that comprises the following deletions: for the gE gene, all of the nucleotides of the ORF are deleted; for the gI gene, at least nucleotides 269-1101 of the 1101 nucleotide ORF are deleted; and for the TK gene, from the 963 nucleotide ORF, a deletion is selected from the nucleotide sequence consisting of positions 526-607, 480-846, 280-723 and 364-615.

In certain embodiments, the virus further comprises a complete deletion of the US2 gene, a complete deletion of the US9 gene, and deletions of at least nucleotides 909-1034 and/or at least nucleotides 301-315 of the 1260 nucleotide ORF of the US1 gene. In other embodiments, US1, US2, and US9 genes are not modified. In certain preferred embodiments, the virus is encoded by SEQ ID NO:3 (M1707) or a sequence at least 85% identical thereto.

In a third aspect, the invention provides an isolated DNA polynucleotide molecule encoding the virus according to any embodiments of the first and/or the second aspect of the invention.

In a fourth aspect, the invention provides a plasmid capable of directly transfecting a host cell, which plasmid comprises a DNA polynucleotide molecule according to the third aspect of the invention, and a promoter capable of permitting transcription of said encoding sequence.

In a fifth aspect of the invention, provided is a vaccine comprising a virus according to any of the embodiments of the first or the second aspect of the invention.

In a sixth aspect, the invention provides a method of protecting swine animals from pseudorabies infection, wherein the method comprises administering to said swine animals the vaccine according to any of the embodiments of the firth aspect of the invention. In certain embodiments, each dose of the vaccine comprises between 10^{4.5} and 10⁹ TCID₅₀, preferably about 10⁷ TCID₅₀ of the virus. Preferably, the virus is isolate M1707 encoded by SEQ ID NO: 3. In different embodiments, said swine animals are boars, sows, gilts, or piglets.

### DETAILED DESCRIPTION

The following definitions and introductory matters are applicable in the specification.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

The term “adjuvant” refers to a compound that enhances the effectiveness of the vaccine, and may be added to the formulation that includes the immunizing agent. Adjuvants provide enhanced immune response even after administration of only a single dose of the vaccine. Adjuvants may include, for example, muramyl dipeptides, pyridine, aluminum hydroxide, dimethyldioctadecyl ammonium bromide (DDA), oils, oil-in-water emulsions, saponins, cytokines, and other substances known in the art. Examples of suitable adjuvants are described in U.S. Patent Application Publication No. US2004/0213817 A1. “Adjuvanted” refers to a composition that incorporates or is combined with an adjuvant.

“Antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

An “attenuated” PRV as used herein refers to a PRV which is capable of infecting and/or replicating in a susceptible host, but is non-pathogenic or less-pathogenic to the susceptible host. For example, the attenuated virus may cause no observable/detectable clinical manifestations, or less clinical manifestations, or less severe clinical manifestations, or exhibit a reduction in virus replication efficiency and/or infectivity, as compared with the related field isolated strains. The clinical manifestations of PRV infection can include, without limitations, coughing, sneezing, fever, constipation, depression, seizures, ataxia, circling, and excess salivation in piglets and mature pigs.

An “epitope” is an antigenic determinant that is immunologically active in the sense that once administered to the host, it is able to evoke an immune response of the humoral (B cells) and/or cellular type (T cells). These are particular chemical groups or peptide sequences on a molecule that are antigenic. An antibody specifically binds a particular antigenic epitope on a polypeptide. In the animal most antigens will present several or even many antigenic determinants simultaneously. Such a polypeptide may also be qualified as an immunogenic polypeptide and the epitope may be identified as described further.

For purposes of the present invention, the nucleotide sequence of a second polynucleotide molecule (either RNA or DNA) is “identical” to the nucleotide sequence of a first polynucleotide molecule, where the nucleotide sequence of the second polynucleotide molecule encodes the same polyaminoacid as the nucleotide sequence of the first polynucleotide molecule as based on the degeneracy of the genetic code, or when it encodes a polyaminoacid that is sufficiently similar to the polyaminoacid encoded by the nucleotide sequence of the first polynucleotide molecule. Generally, the nucleotide sequence of a second polynucleotide molecule is identical to the nucleotide sequence of a first polynucleotide molecule if it has at least about 85% nucleotide sequence identity to the nucleotide sequence of the first polynucleotide molecule as based on the BLASTN algorithm (National Center for Biotechnology Information, otherwise known as NCBI, (Bethesda, Md., USA) of the United States National Institute of Health). In a specific example for calculations according to the practice of the present invention, reference is made to BLASTP 2.2.6 [Tatusova TA and TL Madden, “BLAST 2 sequences—a new tool for comparing protein and nucleotide sequences.” (1999) FEMS Microbiol Lett. 174:247-250.]. Briefly, two amino acid sequences are aligned to optimize the alignment scores using a gap opening penalty of 10, a gap extension penalty of 0.1, and the “blosum62” scoring matrix of Henikoff and Henikoff (Proc. Nat. Acad. Sci. USA 325 89:10915-10919. 1992). The percent identity is then calculated as: Total number of identical matches×100/divided by the length of the longer sequence+number of gaps introduced into the longer sequence to align the two sequences.

The term “isolated” is used to indicate that a cell, peptide or nucleic acid is separated from its native environment. Isolated peptides and nucleic acids may be substantially pure, i.e. essentially free of other substances with which they may bound in nature.

The phrase “lacks functional proteins” means that the amount and/or activity of the protein encoded by the modified gene is decreased by at least 95% compared to the protein encoded by the non-modified gene. In certain aspects, the amount and/or the activity of the protein encoded by the modified gene is decreased by at least 96%, or by at least 97%, or by at least 98%, or by at least 99%, or by at least 99.5%, or by at least 99.9%. In certain aspects, the amount and/or the activity of the protein encoded by the modified gene is completely eliminated.

A “pharmaceutically acceptable carrier” means any conventional pharmaceutically acceptable carrier, vehicle, or excipient that is used in the art for production and administration of vaccines. Pharmaceutically acceptable carriers are typically non-toxic, inert, solid or liquid carriers.

The terms “porcine” and “swine” are used interchangeably herein and refer to any animal that is a member of the family Suidae such as, for example, a pig.

A “susceptible” host as used herein refers to a cell or an animal that can be infected by PEDV. When introduced to a susceptible animal, an attenuated PEDV may also induce an immunological response against the PEDV or its antigen, and thereby render the animal immunity against PEDV infection.

The term “vaccine” refers to an antigenic preparation used to produce immunity to a disease, in order to prevent or ameliorate the effects of infection. Vaccines are typically prepared using a combination of an immunologically effective amount of an immunogen together with an adjuvant effective for enhancing the immune response of the vaccinated subject against the immunogen.

Vaccine formulations will contain a “therapeutically effective amount” of the active ingredient, that is, an amount capable of eliciting an induction of an immunoprotective response in a subject to which the composition is administered. In the treatment and prevention of PEDV disease, for example, a “therapeutically effective amount” would preferably be an amount that enhances resistance of the vaccinated subject to new infection and/or reduces the clinical severity of the disease. Such protection will be demonstrated by either a reduction or lack of symptoms normally displayed by a subject infected with PRV, a quicker recovery time and/or a lowered count of virus particles. Vaccines can be administered prior to infection, as a preventative measure against PRV. Alternatively, vaccines can be administered after the subject already has contracted a disease. Vaccines given after exposure to PRV may be able to attenuate the disease, triggering a superior immune response than the natural infection itself.

The instant disclosure provides an attenuated strain of PRV that is safe and effective if used in a vaccine and protects pigs from a challenge with a virulent PRV strain. In certain aspects, the attenuated strain of PRV comprises modifications in Thymidine Kinase (TK), glycoprotein I (gI) and glycoprotein E (gE) genes relative to a parent field strain.

Suitable parent strains include, without limitations FS18 (SEQ ID NO:1); strain JS2012 (SEQ ID NO:2); strain TJ (GenBank accession KJ789182); strain HeN1 (GenBank accession KP098534); strain HLJ8 (GenBank accession KT824771); strain HN1201 (GenBank accession KP722022). Other suitable strains are the strains that are encoded by a nucleotide sequence that is at least 85% identical (i.e., at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.2% identical, at least 99.4% identical, at least 99.6% identical, at least 99.8% identical, at least 99.9% identical) to the full length SEQ ID: NO:1 or SEQ ID NO:2.

In certain aspects, the parent strain is at least 85% identical to SEQ ID NO: 1 or 2, as recited in the previous paragraph, and also at least half (e.g., at least 60%, at least 70%, at least 80%, at least 90%, at least 95%, or at least 99%) of the differing bases result in codons encoding similar amino acids, i.e., in conservative amino acid substitutions. Such certain conservative amino acid substitutions are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tyrptophan and phenylalanine. Amino acids can be classified according to physical properties and contribution to secondary and tertiary protein structure. A conservative substitution is thus recognized in the art as a substitution of one amino acid for another amino acid that has similar properties, and exemplary conservative substitutions may be found in WO 97/09433, page 10, published Mar. 13, 1997 (PCT/GB96/02197, filed Sep. 6, 1996. Alternatively, conservative amino acids can be grouped as described in Lehninger, (Biochemistry, Second Edition; Worth Publishers, Inc. NY:NY (1975), pp. 71-77). Protein sequences can be aligned using both Vector NTI Advance 11.5 and CLUSTAL 2.1 multiple sequence alignment. As used herein the recitation of a particular amino acid or nucleotide sequence shall include all silent mutations with respect to nucleic acid sequence and any and all conservatively modified variants with respect to amino acid sequences.

Sequences and functions of gI, gE, and TK proteins of Pseudorabies virus are known. Thymidine kinase is encoded by UL23 gene and participates in nucleotide synthesis. Glycoproteins I and E are virion proteins encoded by US7 and US8 genes, respectively. Pomeranz et al discloses that gI and gE are complexed with each other. For the purposes of this disclosure, genes UL23, US7 and US8 may be referred to as “TK gene”, “gI gene” and “gE gene” respectively.

In certain embodiments, the attenuated strain of PRV further comprises modifications one or more (i.e., one, two or all three) of the US1, US2 and US9 genes. These genes encode RSp40/ICP22, 11K, and 28K proteins, respectively. In certain embodiments, at least one of US2 and US9 genes is not modified. Thus, for example, the virus may comprise an unmodified US2, a modified US9, and a modified or an unmodified US1. Alternatively, the virus may comprise an unmodified US9, a modified US2, and a modified or an unmodified US1.

In certain other embodiments, in the attenuated strain of PRV of the invention, US1, US2 and US9 genes are not modified.

Pomeranz et al disclose that US1 encodes RSp40/ICP22 protein. It is a protein whose function in PRV is not currently known but Pomeranz discloses that its HSV-1 homolog acts as a regulator of gene expression. US2 encodes a protein that is present in the tegument of the virus. US9 encodes an envelope protein that participates in protein sorting in axons and functions as a type II tail anchored membrane protein.

Modifications in the genes encoding TK, gI, gE proteins as well as optional modifications in US1, US2, and US9 genes result in a virus that lacks functional proteins expressed by these genes.

For example, in certain aspects, the virus of the invention that lacks a functional gE protein can be prepared by multiple means. For example, one may introduce a stop codon into the proximal part of the ORF encoding gE protein. In different embodiments, the stop codon may be introduced after the N-terminal 10 amino acids or fewer, e.g, 9, 8, 7, 6, 5, 4, 3, or 2 N-terminal amino acids. In other embodiments, the transcription stat site may be altered so that the transcription does not start. In yet other embodiments, all of the nucleotides in the ORF encoding gE protein are deleted.

The virus of the invention also lacks a functional gI protein. In certain embodiments, at least nucleotides 269-1101 are deleted out of the 1101-nucleotide-ORF encoding the gI protein. In certain embodiments, the deletion starts upstream of nucleotide 269, e.g., at nucleotide 250, 200, 150, 100, 50, or even further upstream. In certain embodiments, all 1101 nucleotides are deleted. In certain embodiments, a stop codon is introduced at position 269 or upstream thereof, without introducing a frameshift mutation.

The virus of the invention also has a modified US23 gene that encodes TK protein. UL23 gene has a 963-nucleotide-long ORF. In certain aspects, this 963-nucleotide-long ORF lacks at least one (or at least two, or at least three, or all four) subsequence(s) selected from sequences defined by nucleotides 526-607, 480-846, 280-723 and 364-615 of this 963-nucleotide-long ORF. Of course, longer deletions may also be present, e.g., a deletion defined by positions 280-846 that would incorporate all four subsequences, or a deletion defined by positions 300-650 that would contain two subsequences, and so on. Alternatively, the all of the 963 nucleotides of the ORF may be deleted. Alternatively, a stop codon may be introduced (without causing a frame shift) at a position upstream of 526, or upstream of position 364, or upstream of position 480 or upstream of position 280, and so on. A mutation of the transcription start site is also possible in certain aspects.

Optional modifications to any one of the US1, US2, and US9 genes preferably render the resulting virus lacking the protein encoded by the modified gene. Suitable mutations include gene deletions, insertions, substituions, and so on. As described above, frameshift mutations may be introduced thus producing proteins that have minimal similarity to the proteins encoded by non-modified genes. In-frame mutations may include introduction of stop codons into the proximal part of the gene (e.g., within the N-terminal 20, 15, 10, 5, 3 amino acids), or mutation of the transcription start site so that the corresponding ORF is not transcribed.

In certain aspects, the attenuated virus of the invention has a genome that is at least 85% identical to SEQ ID NO: 1 or 2 and has the following modifications:
a) at least 90% (at least 91%, at least 92%, at least 93%, at least 94%, at least 95%, at least 96%, at least 97%, at least 98%, at least 99%, or 100%) of the ORF encoding the gI protein;
b) at least 90% (at least 91%, at least 92%, at least 93%, at least 94%, at least 95%, at least 96%, at least 97%, at least 98%, at least 99%, or 100%) of the ORF encoding the gE protein;
c) the ORF encoding the TK protein lacks at least one (i.e, at least two, at least three, or all four) of the subsequences defined by nucleotides at positions 526-607, 480-846, 280-723 and 364-615 of this 963-nucleotide-long ORF.

In certain other aspects, the modified live virus is encoded by SEQ ID NO: 3 or a sequence that is at least 85% identical thereto, with a proviso that the sequence encoding the virus comprises modifications to the UL23 gene (encoding TK), US7 gene (encoding gI), US8 gene (encoding gE). Some modified life viruses according to this aspect of the invention may comprise non-modified US1, US2, and US9 genes. Some other modified live viruses according to this aspect of the invention further comprise optional modifications to the US1, US2 and US9 genes, as described above, e.g., the modified US2, the unmodified US9 and the modified or unmodified US1 or the modified US9, the unmodified US2 and the modified or unmodified US1. In other aspect, all three of these genes (US1, US2, US9) are modified

The methods of modifying the genes in such a say that the resulting virus would lack functional proteins encoded by these genes are well known. These methods include, without limitations, full or partial deletions, frameshift mutations, nucleotide replacements, or insertions. For example, one can modify promoter regulating the gene, or the transcription start site. Alternatively (or additionally), one may insert a mutation resulting in a premature stop codon into the coding sequence. Other suitable methods are within the expertise of one of ordinary skill in the art.

The modifications described above can be introduced into the genome of the virus by multiple methods, including, without limitations, targeted mutagenesis and homologous recombination.

The first step of this technique includes the construction of a recombinant DNA molecule for recombination with PrV genomic DNA. Such a recombinant DNA molecule may be derived from any suitable plasmid, cosmid or phage, plasmids being most preferred, and comprises a fragment of PrV DNA containing DNA of the part of the PrV genome as defined above. The DNA sequence of the part of the PrV genome as defined above preferably is flanked by PrV nucleic acid sequences which should be of appropriate length, e.g. 50-3000 bp, as to allow in vivo homologous recombination with the viral PrV genome to occur.

The recombinant DNA molecule obtained in this way is suitable for introducing the mutation into the PrV genome.

Next, cells, e.g. swine kidney cells or VERO cells, can be transfected with PrV DNA or infected with a wild type PrV in the presence of the recombinant DNA molecule as described above whereby recombination occurs between the sequences in the recombinant DNA molecule and the corresponding sequences in the PrV genome.

Recombination can also be induced by co-transfecting the cells with a nucleic acid sequence containing the mutation sequence flanked by appropriate flanking PrV sequences without plasmid sequences. Recombinant viral progeny is thereafter produced in cell culture and can be selected for example genotypically or phenotypically. Another possibility is the detection of the absence of the polypeptide for which the nucleic acid sequence in which the mutation was localized was coding. In the same way the presence of the polypeptide coded for by an inserted heterologous nucleic acid sequence can be detected. Recombinant virus can also be selected positively based on resistance to compounds such as neomycin, gentamycin or mycophenolic acid.

The selected recombinant PrV can be cultured on a large scale in cell culture after which recombinant PrV containing material or heterologous polypeptides expressed by said PrV can be collected thereof.

Alternatively or additionally to the recombinant DNA techniques, cell culture passing combined with clonal enrichment and clonal selection may be used to prepare the virus of the invention. For example, clones with deletions in one of the genes, e.g., a gI or gE may be selected for further propagation, and it has been known that TK natural gene deletion mutants may result from virus replication defect.

Suitable cell lines include, without limitations swine testicle cell line ST, swine kidney cell line PK-15 or MRS-2, rabbit kidney cell line RK, African green monkey kidney cell line Vero, monkey embryonic kidney epithelial cell line Marc-145, bovine kidney cell line MDBK, bovine testicle cell line BT, Chicken Embryo Fibroblast (CEF), and baby hamster kidney cell line BHK-21. In a preferred embodiment, the suitable cell line is Vero (ATCC CCL-81).

An immunologically effective amount of the vaccines of the present invention is administered to a pig in need of protection against viral infection. The immunologically effective amount or the immunogenic amount that inoculates the pig can be easily determined or readily titrated by routine testing. An effective amount is one in which a sufficient immunological response to the vaccine is attained to protect the pig exposed to the PRV virus. Preferably, the pig is protected to an extent in which one to all of the adverse physiological symptoms or effects of the viral disease are significantly reduced, ameliorated or totally prevented.

Vaccines of the present invention can be formulated following accepted convention to include acceptable carriers for animals, such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

Vaccines of the present invention may be non-adjuvanted. Alternatively, the vaccines of the present invention may further comprise one or more additional immunomodulatory components such as, e.g., an adjuvant or cytokine, among others. Non-limiting examples of adjuvants that can be used in the vaccine of the present invention include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, ionic polysaccharides, and Avridine lipid-amine adjuvant. Non-limiting examples of oil-in-water emulsions useful in the vaccine of the invention include modified SEAM62 and SEAM 1/2 formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN® 85 detergent, 0.7% (v/v) Tween 80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines.

Additional adjuvant systems permit for the combination of both T-helper and B-cell epitopes, resulting in one or more types of covalent T-B epitope linked structures, with may be additionally lipidated, such as those described in WO2006/084319, WO2004/014957, and WO2004/014956.

In a preferred embodiment of the present invention, ORFI PEDV protein, or other PEDV proteins or fragments thereof, is formulated with 5% AMPHIGEN® as discussed hereinafter.

### Adjuvant Components

The vaccine compositions of the invention may or may not include adjuvants. In particular, as based on an orally infective virus, the modified live vaccines of the invention may be used adjuvant free, with a sterile carrier. Adjuvants that may be used for oral administration include those based on CT-like immune modulators (rmLT, CT-B, i.e. recombinant-mutant heat labile toxin of E. coli, Cholera toxin-B subunit); or via encapsulation with polymers and alginates, or with mucoadhesives such as chitosan, or via liposomes. A preferred adjuvanted or non adjuvanted vaccine dose at the minimal protective dose through vaccine release may provide between approximately 10 and approximately 10⁶ log ₁₀TCID₅₀ of virus per dose, or higher. “TCID₅₀” refers to “tissue culture infective dose” and is defined as that dilution of a virus required to infect 50% of a given batch of inoculated cell cultures. Various methods may be used to calculate TCID₅₀, including the Spearman-Karber method which is utilized throughout this specification. For a description of the Spearman-Karber method, see B. W. Mahy & H. O. Kangro, Virology Methods Manual, p. 25-46 (1996). Adjuvants, if present, may be provided as emulsions, more commonly if non-oral administration is selected, but should not decrease starting titer by more than 0.7 logs (80% reduction).

In one example, adjuvant components are provided from a combination of lecithin in light mineral oil, and also an aluminum hydroxide component. Details concerning the composition and formulation of AMPHIGEN® (as representative lecithin/mineral oil component) are as follows.

A preferred adjuvanted may be provided as a 2 ML dose in a buffered solution further comprising about 5% (v/v) REHYDRAGEL® (aluminum hydroxide gel) and “20% AMPHIGEN®. at about 25% final (v/v). AMPHIGEN® is generally described in U.S. Pat. No. 5,084,269 and provides de-oiled lecithin (preferably soy) dissolved in a light oil, which is then dispersed into an aqueous solution or suspension of the antigen as an oil-in-water emulsion. Amphigen has been improved according to the protocols of U.S. Pat. No. 6,814,971 (see columns 8-9 thereof) to provide a so-called “20% Amphigen” component for use in the final adjuvanted vaccine compositions of the present invention. Thus, a stock mixture of 10% lecithin and 90% carrier oil (DRAKEOL®, Penreco, Karns City, Pa.) is diluted 1:4 with 0.63% phosphate buffered saline solution, thereby reducing the lecithin and DRAKEOL® components to 2% and 18% respectively (i.e. 20% of their original concentrations). Tween 80 and Span 80 surfactants are added to the composition, with representative and preferable final amounts being 5.6% (v/v) TWEEN®80 and 2.4% (v/v) SPAN®80, wherein the SPAN® is originally provided in the stock DRAKEOL® component, and the TWEEN® is originally provided from the buffered saline component, so that mixture of the saline and DRAKEOL® components results in the finally desired surfactant concentrations. Mixture of the DRAKEOL®/lecithin and saline solutions can be accomplished using an In-Line Slim Emulsifier apparatus, model 405, Charles Ross and Son, Hauppauge, N.Y., USA.

The vaccine composition also includes REHYDRAGEL® LV (about 2% aluminum hydroxide content in the stock material), as additional adjuvant component (available from Reheis, N.J., USA, and ChemTrade Logistics, USA). With further dilution using 0.63% PBS, the final vaccine composition contains the following compositional amounts per 2 ML dose; 5% (v/v) REHYDRAGEL® LV; 25% (v/v) of “20% Amphigen”, i.e. it is further 4-fold diluted); and 0.01% (w/v) of merthiolate.

As is understood in the art, the order of addition of components can be varied to provide the equivalent final vaccine composition. For example, an appropriate dilution of virus in buffer can be prepared. An appropriate amount of REHYDRAGEL® LV (about 2% aluminum hydroxide content) stock solution can then be added, with blending, in order to permit the desired 5% (v/v) concentration of REHYDRAGEL® LV in the actual final product. Once prepared, this intermediate stock material is combined with an appropriate amount of “20% Amphigen” stock (as generally described above, and already containing necessary amounts of Tween 80 and Span 80) to again achieve a final product having 25% (v/v) of “20% Amphigen”. An appropriate amount of 10% merthiolate can finally be added.

The vaccinate compositions of the invention permit variation in all of the ingredients, such that the total dose of antigen may be varied preferably by a factor of 100 (up or down) compared to the antigen dose stated above, and most preferably by a factor of 10 or less (up or down). Similarly, surfactant concentrations (whether TWEEN® or SPAN®) may be varied by up to a factor of 10, independently of each other, or they may be deleted entirely, with replacement by appropriate concentrations of similar materials, as is well understood in the art.

REHYDRAGEL® concentrations in the final product may be varied, first by the use of equivalent materials available from many other manufacturers (i.e. ALHYDROGEL®, Brenntag; Denmark), or by use of additional variations in the REHYDRAGEL® line of products such as CG, HPA or HS. Using LV as an example, final useful concentrations thereof including from 0% to 20%, with 2-12% being more preferred, and 4-8% being most preferred, Similarly, the although the final concentration of AMPHIGEN® (expressed as % of “20% Amphigen”) is preferably 25%, this amount may vary from 5-50%, preferably 20-30% and is most preferably about 24-26%.

According to the practice of the invention, the oil used in the adjuvant formulations of the instant invention is preferably a mineral oil. As used herein, the term “mineral oil” refers to a mixture of liquid hydrocarbons obtained from petrolatum via a distillation technique. The term is synonymous with “liquefied paraffin”, “liquid petrolatum” and “white mineral oil.” The term is also intended to include “light mineral oil,” i.e., oil which is similarly obtained by distillation of petrolatum, but which has a slightly lower specific gravity than white mineral oil. See, e.g., Remington's Pharmaceutical Sciences, 18th Edition (Easton, Pa.: Mack Publishing Company, 1990, at pages 788 and 1323). Mineral oil can be obtained from various commercial sources, for example, J. T. Baker (Phillipsburg, Pa.), USB Corporation (Cleveland, Ohio). Preferred mineral oil is light mineral oil commercially available under the name DRAKEOL®.

The immunogenic and vaccine compositions of the invention can further comprise pharmaceutically acceptable carriers, excipients and/or stabilizers (see e.g. Remington: The Science and practice of Pharmacy, 2005, Lippincott Williams), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as Mercury((o-carboxyphenyl)thio)ethyl sodium salt (THIOMERSAL®), octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), TWEEN® or PLURONICS®.

Vaccines of the present invention can optionally be formulated for sustained release of the virus, infectious DNA molecule, plasmid, or viral vector of the present invention. Examples of such sustained release formulations include virus, infectious DNA molecule, plasmid, or viral vector in combination with composites of biocompatible polymers, such as, e.g., poly (lactic acid), poly (lactic-co-glycolic acid), methylcellulose, hyaluronic acid, collagen and the like. The structure, selection and use of degradable polymers in drug delivery vehicles have been reviewed in several publications, including A. Domb et al., 1992, Polymers for Advanced Technologies 3: 279-292, which is incorporated herein by reference. Additional guidance in selecting and using polymers in pharmaceutical formulations can be found in texts known in the art, for example M. Chasin and R. Langer (eds), 1990, “Biodegradable Polymers as Drug Delivery Systems” in: Drugs and the Pharmaceutical Sciences, Vol. 45, M. Dekker, NY, which is also incorporated herein by reference. Alternatively, or additionally, the virus, plasmid, or viral vector can be microencapsulated to improve administration and efficacy. Methods for microencapsulating antigens are well-known in the art, and include techniques described, e.g., in U.S. Pat. Nos. 3,137,631; 3,959,457; 4,205,060; 4,606,940; 4,744,933; 5,132,117; and International Patent Publication WO 95/28227, all of which are incorporated herein by reference.

Liposomes can also be used to provide for the sustained release of virus, plasmid, viral protein, or viral vector. Details concerning how to make and use liposomal formulations can be found in, among other places, U.S. Pat. Nos. 4,016,100; 4,452,747; 4,921,706; 4,927,637; 4,944,948; 5,008,050; and 5,009,956, all of which are incorporated herein by reference.

An effective amount of any of the above-described vaccines can be determined by conventional means, starting with a low dose of virus, viral protein plasmid or viral vector, and then increasing the dosage while monitoring the effects. An effective amount may be obtained after a single administration of a vaccine or after multiple administrations of a vaccine. Known factors can be taken into consideration when determining an optimal dose per animal. These include the species, size, age and general condition of the animal, the presence of other drugs in the animal, and the like. The actual dosage is preferably chosen after consideration of the results from other animal studies.

One method of detecting whether an adequate immune response has been achieved is to determine seroconversion and antibody titer in the animal after vaccination. The timing of vaccination and the number of boosters, if any, will preferably be determined by a doctor or veterinarian based on analysis of all relevant factors, some of which are described above.

In a preferred example of the invention relating to vaccination of swine, an optimum age target for the animals is between about 1 and 21 days, which at pre-weening, may also correspond with other scheduled vaccinations such as against Mycoplasma hyopneumoniae or Porcine Reproductive and Respiratory Syndrome virus. Additionally, a preferred schedule of vaccination for breeding sows would include similar doses, with an annual revaccination schedule.

### Dosing

A preferred clinical indication is for treatment, control and prevention in both breeding sows and gilts pre-farrowing, followed by vaccination of piglets. In a representative example (applicable to both sows and gilts), a single dose of vaccine is used, although of course, two-dose vaccination regimens are also envisioned, if needed.

Actual volume of the dose is a function of how the vaccine is formulated, with actual dosing amounts ranging from 0.05 to 5 ML, taking also into account the size of the animals. Single dose vaccination is also appropriate. The amount of the pseudorabies virus in the vaccine is between 10^{4.5} TCID₅₀ and 10⁸ TCID₅₀ per dose, preferably between 10⁵ and 10⁷ TCID₅₀ per dose, or more preferably between 10^{5.5} and 10^{5.5} TCID₅₀ per dose.

Preferably, only a single administration is sufficient to confer protection. However, if a two-dose regimen is needed, booster doses can be given two to four weeks prior to any subsequent farrowings. Intramuscular vaccination (all doses) is preferred, although one or more of the doses could be given subcutaneously. Oral administration is also preferred. Vaccination may also be effective in naive animals, and non-naive animals as accomplished by planned or natural infections.

In a further preferred example, the sow or gilt is vaccinated intramuscularly or orally at 5-weeks pre-farrowing and then 2-weeks pre-farrowing. The protocols of the invention are also applicable to the treatment of already seropositive sows and gilts, and also piglets and boars. Booster vaccinations can also be given and these may be via a different route of administration. Although it is preferred to re-vaccinate a mother sow prior to any subsequent farrowings, the vaccine compositions of the invention nonetheless can still provide protection to piglets via ongoing passive transfer of antibodies, even if the mother sow was only vaccinated in association with a previous farrowing.

It should be noted that piglets may then be vaccinated as early as Day 1 of life. For example, piglets can be vaccinated at Day 1, with or without a booster dose at 3 weeks of age, particularly if the parent sow, although vaccinated pre-breeding, was not vaccinated pre-farrowing. Piglet vaccination may also be effective if the parent sow was previously not naive either due to natural or planned infection. Vaccination of piglets when the mother has neither been previously exposed to the virus, nor vaccinated pre-farrowing may also effective.

In other aspects, the vaccine may be administered to piglets that are about six days or older, or about fourteen days or older, or about 21 days or older, or about 28 days or older, or about 35 days or older, or about 42 days or older.

Boars (typically kept for breeding purposes) should be vaccinated once every 6 months. Variation of the dose amounts is well within the practice of the art. It should be noted that the vaccines of the present invention are safe for use in pregnant animals (all trimesters) and neonatal swine. The vaccines of the invention are attenuated to a level of safety (i.e. no mortality, only transient mild clinical signs or signs normal to neonatal swine) that is acceptable for even the most sensitive animals again including neonatal pigs. Of course, from a standpoint of protecting swine herds both from PRV epidemics and persistent low level PRV occurrence, programs of sustained sow vaccination are of great importance. It will be appreciated that sows or gilts immunized with PRV MLV will passively transfer immunity to piglets, including PRV-specific IgA, which will protect piglets from PRV associated disease and mortality. Additionally, generally, pigs that are immunized with PRV MLV will have a decrease in amount and/or duration or be protected from shedding PRV in their feces, and further, pigs that are immunized with PRV MLV will be protected from the clinical signs of PRV including, without limitations, mortality, reproductive, neurological, and respiratory manifestations of PRV, and further, PRV MLV will aid in stopping or controlling the PEDV transmission cycle.

It should also be noted that animals vaccinated with the vaccines of the invention are also immediately safe for human consumption, without any significant slaughter withhold, such as 21 days or less.

When provided therapeutically, the vaccine is provided in an effective amount upon the detection of a sign of actual infection. A composition is said to be “pharmacologically acceptable” if its administration can be tolerated by a recipient. Such a composition is said to be administered in a “therapeutically or prophylactically effective amount” if the amount administered is physiologically significant.

At least one vaccine or immunogenic composition of the present invention can be administered by any means that achieve the intended purpose, using a pharmaceutical composition as described herein. For example, route of administration of such a composition can be by parenteral, oral, oronasal, intranasal, intratracheal, topical, subcutaneous, intramuscular, transcutaneous, intradermal, intraperitoneal, intraocular, and intravenous administration. In one embodiment of the present invention, the composition is administered by intramuscularly. Parenteral administration can be by bolus injection or by gradual perfusion over time. Any suitable device may be used to administer the compositions, including syringes, droppers, needleless injection devices, patches, and the like. The route and device selected for use will depend on the composition of the adjuvant, the antigen, and the subject, and such are well known to the skilled artisan. Administration that is oral, or alternatively, subcutaneous, is preferred. Oral administration may be direct, via water, or via feed (solid or liquid feed). When provided in liquid form, the vaccine may be lyophilized with reconstitution, or provided as a paste, for direct addition to feed (mix in or top dress) or otherwise added to water or liquid feed.

### Diagnostic Kits

The present invention also provides diagnostic kits. The kit can be valuable for differentiating between porcine animals naturally infected with a field strain of a PRV virus and porcine animals vaccinated with any of the PRV vaccines described herein. The kits can also be of value because animals potentially infected with field strains of PRV virus can be detected prior to the existence of clinical symptoms and removed from the herd, or kept in isolation away from naive or vaccinated animals.

The kits include reagents for analyzing a sample from a porcine animal for the presence of antibodies to a particular component of a specified PRV virus. Diagnostic kits of the present invention can include as a component a peptide or peptides from the variant PRV strain of the invention which is present in a field strain but not in a vaccine of interest, or vice versa, and selection of such suitable peptide domains is made possible by the extensive amino acid sequencing. Such peptides may be used in any immunoassay system known in the art including, but not limited to: radioimmunoassays, enzyme-linked immunosorbent assay, “sandwich” assays, precipitin reactions, gel diffusion immunodiffusion assays, agglutination assays, fluorescent immunoassays, protein A immunoassays and immunoelectrophoresis assays, to name but a few U.S. Pat. No. 4,629,783 and patents cited therein also describe suitable assays.

For example, the kit may contain immunogenic peptides that are present in non-modified TK, gE, and/or gI proteins as well as products of the optionally modified US1, US2 and/or US9 genes and absent in the expression products of the modified genes. If, upon contact with a sample of an animal suspected of being infected with PRV, the peptide binds the antibody against one of these proteins, this indicates that the animal has been infected. The absence of the binding indicates that the animal has not been infected but could have been vaccinated with the vaccine of the invention.

The kit may also contain peptides that are present in both the attenuated strain and the wild-type strain of PRV. Suitable non-limiting examples of such peptides include envelope proteins encoded by UL53, UL49.5, UL27, UL34 genes. If, upon contact with a sample of an animal suspected of being infected with PRV, the peptide binds the antibody against one of these proteins, this indicates that the animal has been infected or vaccinated. The absence of the binding indicates that the animal has been neither infected nor vaccinated.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Example 1

Piglets negative for both PRV antigen and antibody were assigned into groups with 7 piglets in each group. Piglets were provided commercial diet and free access to water.

Strain M1707 (comprising SEQ ID NO: 3, and wherein nucleotides 480-846 of the UL23 gene ecnoding TK are deleted) was formulated with MEM, gelatin, NZ amine, glutamine, sucrose, Dextran 40, lactose, sorbitol, and penicillin-streptomycin.

The second group of pigs was vaccinated with strain Bartha K61. The third group of pigs was vaccinated with DMEM. The vaccination method was an intramuscular injection in the neck. The inoculation volumes for the treatment groups were all 1 ml per piglet. After inoculation, clinical observations were conducted every day, including the rectal temperature of the pigs was measured.

Data showed that Strain M1707 at F35 lab product level is safe in 3˜4 weeks old piglets (7 pigs were treated), as well as 7 weeks old target age piglets (14 pigs were treated) with a >10^{6.5}TCID₅₀/pig treatment with 3 different batches lab products. All pigs including control pigs' body temperatures are normal, no clinical signs showed within the observation period of 14 days.

### Example 2

Piglets negative for both PRV antigen and antibody were assigned into groups with 7 piglets in each group. Piglets were provided commercial diet and free access to water.

Three lots (Lot A, Lot B, and Lot C) of PRV strain M1707 were prepared. The virus was formulated with MEM, gelatin, NZ amine, glutamine, sucrose, Dextran 40, lactose, sorbitol, and penicillin-streptomycin.

The pigs were treated with the formulation of one of the lots in the amount of 10^{5.0}TCID₅₀ per dose on day 0, injected intramuscularly. The control group was treated with DMEM only. After inoculation, the rectal temperature of the pigs was measured every day. Observation of the clinical symptoms found that all the pigs had a normal body temperature, good appetite, normal mental state, no respiratory and gastrointestinal symptoms, and no neurological symptoms within the observation period of 21 days. The three vaccinated groups and the control group were intranasally challenged with Strain FS21PF1115, 2 mL (10^{5.0}TCID₅₀), on day 21.

The protection was determined by the severity (or absence) of symptoms. In the three control groups, out of 20 pigs, 19 were protected (7 out of 7 in each of lots A and B, and 6 out of 7 in lot C). In the control group, zero out of seven pigs were protected.

### Example 3

Piglets negative for both PRV antigen and antibody were assigned into groups with 5 piglets in each group. Piglets were provided commercial diet and free access to water.

PRV strain 1707 was formulated with MEM, gelatin, NZ amine, glutamine, sucrose, Dextran 40, lactose, sorbitol, and penicillin-streptomycin. Each vaccinated pig received 10^{5.0} TCID₅₀ of the antigen per dose. The control group was treated with DMEM. The formulations were administered by intramuscular injection. Both the vaccinated and the control group were intranasally challenged with strain FS21PF1115, 2 mL (10^{6.0}TCID₅₀), six months after the vaccination.

The duration of immunity was determined by the severity (or absence) of symptoms. Five out of five pigs in the vaccinated group and none in the control group exhibited the protective titer.

1. An attenuated suid herpesvirus 1 (a Pseudorabies virus) wherein the TK, gI and gE genes thereof are modified relative to a parent field strain, such that the resultant virus is safe and effective for use as a live vaccine that protects swine animals from challenge with a virulent Pseudorabies virus, and wherein said parent strain is selected from the group consisting of: strain FS18 (SEQ ID NO:1); strain JS2012 (SEQ ID NO:2); strain TJ (GenBank accession KJ789182); strain HeN1 (GenBank accession KP098534); strain HUJ8 (GenBank accession KT824771); strain HN1201 (GenBank accession KP722022), and any strain that is encoded from a nucleotide sequence that is at least 85% identical to SEQ ID: NO.1 or SEQ ID NO:2.

2. The virus of claim 1, that further comprises attenuating modifications of one or more of the US1, US2 and US9 genes, with the proviso that at least one of US2 and US9 genes is not modified.

3. The virus of claim 1, wherein US1, US2 and US9 genes are non-modified.

4. The virus of any one of claims 1-3, wherein said attenuating gene modifications include full or partial deletions, frameshift mutations, nucleotide replacements, or insertions.

5. The virus of any one of claims 1-4 derived from the FS18 strain (as encoded by SEQ ID NO:1) or the JS2012 strain (as encoded by SEQ ID NO:2).

6. The virus of claim 1, encoded by SEQ ID NO:3 or a sequence that is at least 85% identical thereto, and which comprises:
a) a deletion of UL23 gene nucleotides 480-846 (Isolate M1707); or
b) a deletion of UL23 gene nucleotides 526-607 (Isolate M1705); or
c) a deletion of UL23 gene nucleotides 280-723 (Isolate M1708); or
d) a deletion of UL23 gene nucleotides 364-615 (Isolate M1710); or
e) a deletion in UL23 gene that includes any of the deletions of ‘a’, ‘b’, ‘c’, or ‘d’.

7. The virus of claim 2, wherein the gE, US9, and US2 genes are fully deleted, the gI and TK genes are at least partially deleted, and the one or more copies of US1 gene are at least partially deleted.

8. An attenuated suid herpesvirus I (Pseudorabies virus) that is derived from strain FS18 (SEQ ID NO:1); strain JS2012 (SEQ ID NO:2), strain TJ (GenBank accession KJ789182), strain HeN1 (GenBank accession KP098534), strain HUJ8 (GenBank accession KT824771) or strain HN1201 (GenBank accession KP722022), or any strain that is encoded from a nucleotides sequence that is at least 85% identical to SEQ ID: NO.1 or SEQ ID NO:2, wherein said attenuate is encoded from a DNA sequence that comprises the following deletions:
for the gE gene, all of the nucleotides of the ORF are deleted;
for the gI gene, at least nucleotides 269-1101 of the 1101 nucleotide ORF are deleted; and
for the TK gene, from the 963 nucleotide ORF, a deletion is selected from the nucleotide sequence consisting of positions 526-607, 480-846, 280-723 and 364-615.

9. The attenuated virus of claim 8, further comprising a complete deletion of the US2 gene, a complete deletion of the US9 gene, and deletions of at least nucleotides 909-1034 and/or at least nucleotides 301-315 of the 1260 nucleotide ORF of the US1 gene.

10. The attenuated virus of claim 9 that is encoded by SEQ ID NO:3 (M1707) or a sequence at least 85% identical thereto.

11. The attenuated virus of claim 8, wherein US1, US2, and US9 genes are not modified.

12. A vaccine composition comprising the live virus of any one of claims 1-11, and a pharmaceutically acceptable carrier.

13. A vaccine composition comprising the virus of any one of claims 1-7, wherein said virus is provided in killed form.

14. A method of vaccinating a swine animal to provide protection against challenge by a virulent Pseudorabies virus, comprising administering one or more doses of the vaccine composition of claim 12 or claim 13.

15. The method of claim 14, wherein a single dose of the virus M1707 is used, wherein said dosage provides between 10^{4.5} and 10⁹ TCID₅₀.

16. The method of claim 14, wherein the vaccine is safe when administered to piglets with a single dose treatment of 10⁷ TCID₅₀.

17. The method according to claim 14 wherein said swine animal is a boar, a sow, a gilt, or a piglet.

18. The virus of claim 2, wherein the deletion in the nucleotide sequence of the US-1 gene is the sequence, ctcctcttcc tcgtc (SEQ ID NO:4), or any larger sequence of the US-1 gene wherein said sequence is contained.

19. The virus of claim 2, wherein the deletion in the nucleotide sequence of the US-1 gene is the sequence, cgag gaagaggaag aggaagagga agacggggac gaggacgaggaagaggagga cgaggaagag gaggacgagg aagaggagga cgaggaagag gaggacgagg aagaggagga cgaggaagag ga (SEQ ID NO:5), or any larger sequence of the US-1 gene wherein said sequence is contained.

20. The virus of claim 2, wherein the deletion in the nucleotide sequence of the TK gene is the sequence, gcggcgcct gcgcgcccgc gegcgcgccg gggagcacgt ggacgcgcgc ctgctcacgg ccctgcgcaa cgtctacgcc atgctggtca acacgtegcg ctacctgagc toggggcgcc gctggcgcga cgactggggg cgcgcgccgc gcttcgacca gaccgtgcgc gactgccteg cgctcaacga gctctgccgc ccgcgcgacg accccgagct ccaggacacc ctcttcggcg cgtacaaggc gcccgagctc tgcgaccggc gcgggcgccc gctcgaggtg cacgcgtggg cgatggacgc gctcgtggcc aagctgctgc cgctgcgcgt ctccaccgtc gacctggggc cctcgcc, (SEQ ID NO:6), or any larger sequence of the TK gene wherein said sequence is contained.

21. The virus of claim 2, wherein the deletion in the nucleotide sequence of the TK gene the sequence, JS2012 nucleotides 526-607, (cgcctgctcacggccctgcgcaacgtctacgccatgctggtcaacacgtcgcgctacctgagctcggggcgccgctggcg, SEQ ID NO:7), or any larger sequence of the TK gene wherein said sequence is contained.

22. The virus of claim 2, wherein the deletion in the nucleotide sequence of the TK gene is the sequence, JS2012 nucleotides 280-723, (gggcccgcggtcgagggcccgcccgagatgacggtcgtctttgaccgccacccggtggccgcgacggtgtgcttcccgctggcgcgctt catcgtcggggacatcagcgcggcggccttcgtgggcctggcggccacgctgcccggggagccccccggcggcaacctggtggtggcct cgctggacccggacgagcacctgcggcgcctgcgcgcccgcgcgcgcgccggggagcacgtggacgcgcgcctgctcacggccctgcg caacgtctacgccatgctggtcaacacgtcgcgctacctgagctcggggcgccgctggcgcgacgactgggggcgcgcgccgcgcttcg accagaccgtgcgcgactgcctcgcgctcaacgagctctgccgcccgcgcgacgaccccgagctccaggacaccctcttcggcgcgtac, SEQ ID NO:8), or any larger sequence of the TK gene wherein said sequence is contained.

23. The virus of claim 2, wherein the deletion in the nucleotide sequence of the TK gene is the sequence, JS2012 nucleotides 364-615, (cgcttcatcgtcggggacatcagcgcggcggccttcgtgggcctggcggccacgctgcccggggagccccccggcggcaacctggtggt ggcctcgctggacccggacgagcacctgcggcgcctgcgcgcccgcgcgcgcgccggggagcacgtggacgcgcgcctgctcacggcc ctgcgcaacgtctacgccatgctggtcaacacgtcgcgctacctgagctcggggcgccgctggcgcgacgactgg, SEQ ID NO:9), or any larger sequence of the TK gene wherein said sequence is contained.

24. The virus of claim 2, wherein the deletion in the nucleotide sequence of the US-1 gene is the sequence, from FS18, analogous to SEQ ID NO: 4, or any larger sequence of the US-1 gene wherein said sequence is contained.

25. The virus of claim 2, wherein the deletion in the nucleotide sequence of the US-1 gene is the sequence, from FS18, analogous to SEQ ID NO: 5, or any larger sequence of the US-1 gene wherein said sequence is contained.

26. The virus of claim 2, wherein the deletion in the nucleotide sequence of the TK gene is the sequence, FS18/M1707 nucleotides 480-846 (SEQ ID NO:6), or any larger sequence of the TK gene wherein said sequence is contained.

27. The virus of claim 2, wherein the deletion in the nucleotide sequence of the TK gene is the sequence, FS18/M1705 nucleotides 526-607, (SEQ ID NO:7), or any larger sequence of the TK gene wherein said sequence is contained.

28. The virus of claim 2, wherein the deletion in the nucleotide sequence of the TK gene is the sequence, FS18/M1708 nucleotides 280-723, (SEQ ID NO:8), or any larger sequence of the TK gene wherein said sequence is contained.

29. The virus of claim 2, wherein the deletion in the nucleotide sequence of the TK gene is the sequence, FS18/M1710 nucleotides 364-615, (SEQ ID NO:9), or any larger sequence of the TK gene wherein said sequence is contained.

30. An isolated DNA polynucleotide molecule encoding the virus of claim 1 or claim 2.

31. A plasmid capable of directly transfecting a host cell, which plasmid comprises a DNA polynucleotide molecule according to claim 30, and a promoter capable of permitting transcription of said encoding sequence.
